SEQUENCE LISTING (645 sequences)

SEQ ID NO: 1 - DNA, 19 bp, synthetic construct
  source 1..19: mol_type = other DNA
ttaacaaaaacacaacatc

SEQ ID NO: 2 - DNA, 18 bp, synthetic construct
  source 1..18: mol_type = other DNA
ttaacaaaaacacaacat

SEQ ID NO: 3 - DNA, 18 bp, synthetic construct
  source 1..18: mol_type = other DNA
taacaaaaacacaacatc

SEQ ID NO: 4 - DNA, 20 bp, synthetic construct
  source 1..20: mol_type = other DNA
acaaaaacacaacatcaaat

SEQ ID NO: 5 - DNA, 19 bp, synthetic construct
  source 1..19: mol_type = other DNA
caaaaacacaacatcaaat

SEQ ID NO: 6 - DNA, 20 bp, synthetic construct
  source 1..20: mol_type = other DNA
caaaaacacaacatcaaatt

SEQ ID NO: 7 - DNA, 20 bp, synthetic construct
  source 1..20: mol_type = other DNA
aaaaacacaacatcaaattc

SEQ ID NO: 8 - DNA, 19 bp, synthetic construct
  source 1..19: mol_type = other DNA
aaaacacaacatcaaattc

SEQ ID NO: 9 - DNA, 20 bp, synthetic construct
  source 1..20: mol_type = other DNA
aaaacacaacatcaaattcc

SEQ ID NO: 10 - DNA, 19 bp, synthetic construct
  source 1..19: mol_type = other DNA
aaacacaacatcaaattcc

SEQ ID NO: 11 - DNA, 18 bp, synthetic construct
  source 1..18: mol_type = other DNA
aaacacaacatcaaattc

SEQ ID NO: 12 - DNA, 20 bp, synthetic construct
  source 1..20: mol_type = other DNA
aaacacaacatcaaattcct

SEQ ID NO: 13 - DNA, 19 bp, synthetic construct
  source 1..19: mol_type = other DNA
aacacaacatcaaattcct

SEQ ID NO: 14 - DNA, 17 bp, synthetic construct
  source 1..17: mol_type = other DNA
aacacaacatcaaattc

SEQ ID NO: 15 - DNA, 18 bp, synthetic construct
  source 1..18: mol_type = other DNA
aacacaacatcaaattcc

SEQ ID NO: 16 - DNA, 20 bp, synthetic construct
  source 1..20: mol_type = other DNA
aacacaacatcaaattcctt

SEQ ID NO: 17 - DNA, 19 bp, synthetic construct
  source 1..19: mol_type = other DNA
acacaacatcaaattcctt

SEQ ID NO: 18 - DNA, 20 bp, synthetic construct
  source 1..20: mol_type = other DNA
acacaacatcaaattccttt

SEQ ID NO: 19 - DNA, 18 bp, synthetic construct
  source 1..18: mol_type = other DNA
cacaacatcaaattcctt

SEQ ID NO: 20 - DNA, 19 bp, synthetic construct
  source 1..19: mol_type = other DNA
cacaacatcaaattccttt

SEQ ID NO: 21 - DNA, 18 bp, synthetic construct
  source 1..18: mol_type = other DNA
acaacatcaaattccttt

SEQ ID NO: 22 - DNA, 17 bp, synthetic construct
  source 1..17: mol_type = other DNA
acaacatcaaattcctt

SEQ ID NO: 23 - DNA, 20 bp, synthetic construct
  source 1..20: mol_type = other DNA
acaacatcaaattcctttac

SEQ ID NO: 24 - DNA, 20 bp, synthetic construct
  source 1..20: mol_type = other DNA
caacatcaaattcctttact

SEQ ID NO: 25 - DNA, 19 bp, synthetic construct
  source 1..19: mol_type = other DNA
caacatcaaattcctttac

SEQ ID NO: 26 - DNA, 19 bp, synthetic construct
  source 1..19: mol_type = other DNA
aacatcaaattcctttact

SEQ ID NO: 27 - DNA, 18 bp, synthetic construct
  source 1..18: mol_type = other DNA
aacatcaaattcctttac

SEQ ID NO: 28 - DNA, 20 bp, synthetic construct
  source 1..20: mol_type = other DNA
aacatcaaattcctttactt

SEQ ID NO: 29 - DNA, 20 bp, synthetic construct
  source 1..20: mol_type = other DNA
acatcaaattcctttacttc

SEQ ID NO: 30 - DNA, 17 bp, synthetic construct
  source 1..17: mol_type = other DNA
acatcaaattcctttac

SEQ ID NO: 31 - DNA, 18 bp, synthetic construct
  source 1..18: mol_type = other DNA
acatcaaattcctttact

SEQ ID NO: 32 - DNA, 19 bp, synthetic construct
  source 1..19: mol_type = other DNA
acatcaaattcctttactt

SEQ ID NO: 33 - DNA, 18 bp, synthetic construct
  source 1..18: mol_type = other DNA
catcaaattcctttactt

SEQ ID NO: 34 - DNA, 20 bp, synthetic construct
  source 1..20: mol_type = other DNA
catcaaattcctttacttct

SEQ ID NO: 35 - DNA, 19 bp, synthetic construct
  source 1..19: mol_type = other DNA
catcaaattcctttacttc

SEQ ID NO: 36 - DNA, 17 bp, synthetic construct
  source 1..17: mol_type = other DNA
catcaaattcctttact

SEQ ID NO: 37 - DNA, 18 bp, synthetic construct
  source 1..18: mol_type = other DNA
atcaaattcctttacttc

SEQ ID NO: 38 - DNA, 19 bp, synthetic construct
  source 1..19: mol_type = other DNA
atcaaattcctttacttct

SEQ ID NO: 39 - DNA, 17 bp, synthetic construct
  source 1..17: mol_type = other DNA
atcaaattcctttactt

SEQ ID NO: 40 - DNA, 18 bp, synthetic construct
  source 1..18: mol_type = other DNA
caaattcctttacttctg

SEQ ID NO: 41 - DNA, 20 bp, synthetic construct
  source 1..20: mol_type = other DNA
gcagaaatagaacattcttt

SEQ ID NO: 42 - DNA, 17 bp, synthetic construct
  source 1..17: mol_type = other DNA
gcagaaatagaacattc

SEQ ID NO: 43 - DNA, 18 bp, synthetic construct
  source 1..18: mol_type = other DNA
gcagaaatagaacattct

SEQ ID NO: 44 - DNA, 19 bp, synthetic construct
  source 1..19: mol_type = other DNA
cagaaatagaacattcttt

SEQ ID NO: 45 - DNA, 20 bp, synthetic construct
  source 1..20: mol_type = other DNA
cagaaatagaacattcttta

SEQ ID NO: 46 - DNA, 18 bp, synthetic construct
  source 1..18: mol_type = other DNA
agaaatagaacattcttt

SEQ ID NO: 47 - DNA, 19 bp, synthetic construct
  source 1..19: mol_type = other DNA
agaaatagaacattcttta

SEQ ID NO: 48 - DNA, 20 bp, synthetic construct
  source 1..20: mol_type = other DNA
agaaatagaacattctttat

SEQ ID NO: 49 - DNA, 19 bp, synthetic construct
  source 1..19: mol_type = other DNA
gaaatagaacattctttat

SEQ ID NO: 50 - DNA, 20 bp, synthetic construct
  source 1..20: mol_type = other DNA
gaaatagaacattctttatc

SEQ ID NO: 51 - DNA, 18 bp, synthetic construct
  source 1..18: mol_type = other DNA
gaaatagaacattcttta

SEQ ID NO: 52 - DNA, 17 bp, synthetic construct
  source 1..17: mol_type = other DNA
gaaatagaacattcttt

SEQ ID NO: 53 - DNA, 19 bp, synthetic construct
  source 1..19: mol_type = other DNA
aaatagaacattctttatc

SEQ ID NO: 54 - DNA, 18 bp, synthetic construct
  source 1..18: mol_type = other DNA
gtataattagttaaatac

SEQ ID NO: 55 - DNA, 19 bp, synthetic construct
  source 1..19: mol_type = other DNA
gtataattagttaaatact

SEQ ID NO: 56 - DNA, 15 bp, synthetic construct
  source 1..15: mol_type = other DNA
accaccgcaggaaat

SEQ ID NO: 57 - DNA, 19 bp, synthetic construct
  source 1..19: mol_type = other DNA
aaatcgcgaatgaacttcc

SEQ ID NO: 58 - DNA, 16 bp, synthetic construct
  source 1..16: mol_type = other DNA
aaatcgcgaatgaact

SEQ ID NO: 59 - DNA, 17 bp, synthetic construct
  source 1..17: mol_type = other DNA
aaatcgcgaatgaactt

SEQ ID NO: 60 - DNA, 18 bp, synthetic construct
  source 1..18: mol_type = other DNA
aaatcgcgaatgaacttc

SEQ ID NO: 61 - DNA, 18 bp, synthetic construct
  source 1..18: mol_type = other DNA
aatcgcgaatgaacttcc

SEQ ID NO: 62 - DNA, 15 bp, synthetic construct
  source 1..15: mol_type = other DNA
aatcgcgaatgaact

SEQ ID NO: 63 - DNA, 16 bp, synthetic construct
  source 1..16: mol_type = other DNA
aatcgcgaatgaactt

SEQ ID NO: 64 - DNA, 17 bp, synthetic construct
  source 1..17: mol_type = other DNA
aatcgcgaatgaacttc

SEQ ID NO: 65 - DNA, 16 bp, synthetic construct
  source 1..16: mol_type = other DNA
atcgcgaatgaacttc

SEQ ID NO: 66 - DNA, 14 bp, synthetic construct
  source 1..14: mol_type = other DNA
atcgcgaatgaact

SEQ ID NO: 67 - DNA, 15 bp, synthetic construct
  source 1..15: mol_type = other DNA
atcgcgaatgaactt

SEQ ID NO: 68 - DNA, 17 bp, synthetic construct
  source 1..17: mol_type = other DNA
atcgcgaatgaacttcc

SEQ ID NO: 69 - DNA, 15 bp, synthetic construct
  source 1..15: mol_type = other DNA
tcgcgaatgaacttc

SEQ ID NO: 70 - DNA, 16 bp, synthetic construct
  source 1..16: mol_type = other DNA
tcgcgaatgaacttcc

SEQ ID NO: 71 - DNA, 14 bp, synthetic construct
  source 1..14: mol_type = other DNA
tcgcgaatgaactt

SEQ ID NO: 72 - DNA, 14 bp, synthetic construct
  source 1..14: mol_type = other DNA
cgcgaatgaacttc

SEQ ID NO: 73 - DNA, 15 bp, synthetic construct
  source 1..15: mol_type = other DNA
cgcgaatgaacttcc

SEQ ID NO: 74 - DNA, 18 bp, synthetic construct
  source 1..18: mol_type = other DNA
aatgaacttccagagctt

SEQ ID NO: 75 - DNA, 16 bp, synthetic construct
  source 1..16: mol_type = other DNA
atgaacttccagagct

SEQ ID NO: 76 - DNA, 16 bp, synthetic construct
  source 1..16: mol_type = other DNA
tgaacttccagagctt

SEQ ID NO: 77 - DNA, 15 bp, synthetic construct
  source 1..15: mol_type = other DNA
tgcccgatgctcttc

SEQ ID NO: 78 - DNA, 15 bp, synthetic construct
  source 1..15: mol_type = other DNA
tagagcacgtcctcc

SEQ ID NO: 79 - DNA, 17 bp, synthetic construct
  source 1..17: mol_type = other DNA
ctctcattcatggccac

SEQ ID NO: 80 - DNA, 17 bp, synthetic construct
  source 1..17: mol_type = other DNA
taccatgaatcagtatc

SEQ ID NO: 81 - DNA, 16 bp, synthetic construct
  source 1..16: mol_type = other DNA
tgccattttataggac

SEQ ID NO: 82 - DNA, 20 bp, synthetic construct
  source 1..20: mol_type = other DNA
ccattttataggacaaaaat

SEQ ID NO: 83 - DNA, 18 bp, synthetic construct
  source 1..18: mol_type = other DNA
agaaatttgcagtttcct

SEQ ID NO: 84 - DNA, 19 bp, synthetic construct
  source 1..19: mol_type = other DNA
agaaatttgcagtttcctc

SEQ ID NO: 85 - DNA, 17 bp, synthetic construct
  source 1..17: mol_type = other DNA
gaaatttgcagtttcct

SEQ ID NO: 86 - DNA, 16 bp, synthetic construct
  source 1..16: mol_type = other DNA
acagacagtgttacct

SEQ ID NO: 87 - DNA, 18 bp, synthetic construct
  source 1..18: mol_type = other DNA
acagtgttacctgattct

SEQ ID NO: 88 - DNA, 19 bp, synthetic construct
  source 1..19: mol_type = other DNA
acagtgttacctgattctc

SEQ ID NO: 89 - DNA, 18 bp, synthetic construct
  source 1..18: mol_type = other DNA
cagtgttacctgattctc

SEQ ID NO: 90 - DNA, 17 bp, synthetic construct
  source 1..17: mol_type = other DNA
agtgttacctgattctc

SEQ ID NO: 91 - DNA, 16 bp, synthetic construct
  source 1..16: mol_type = other DNA
gtgttacctgattctc

SEQ ID NO: 92 - DNA, 17 bp, synthetic construct
  source 1..17: mol_type = other DNA
tgttacctgattctcat

SEQ ID NO: 93 - DNA, 17 bp, synthetic construct
  source 1..17: mol_type = other DNA
acctgattctcatgctc

SEQ ID NO: 94 - DNA, 18 bp, synthetic construct
  source 1..18: mol_type = other DNA
aaaggctctgtttttatc

SEQ ID NO: 95 - DNA, 19 bp, synthetic construct
  source 1..19: mol_type = other DNA
aaaggctctgtttttatcc

SEQ ID NO: 96 - DNA, 18 bp, synthetic construct
  source 1..18: mol_type = other DNA
aaggctctgtttttatcc

SEQ ID NO: 97 - DNA, 17 bp, synthetic construct
  source 1..17: mol_type = other DNA
aaggctctgtttttatc

SEQ ID NO: 98 - DNA, 18 bp, synthetic construct
  source 1..18: mol_type = other DNA
tttttatccccacttcac

SEQ ID NO: 99 - DNA, 16 bp, synthetic construct
  source 1..16: mol_type = other DNA
aatttctgtctggcac

SEQ ID NO: 100 - DNA, 16 bp, synthetic construct
  source 1..16: mol_type = other DNA
acctacacccatcatc

SEQ ID NO: 101 - DNA, 20 bp, synthetic construct
  source 1..20: mol_type = other DNA
gaaggttaaaatttaaaaat

SEQ ID NO: 102 - DNA, 20 bp, synthetic construct
  source 1..20: mol_type = other DNA
tttaaaaattaaaatgaacc

SEQ ID NO: 103 - DNA, 19 bp, synthetic construct
  source 1..19: mol_type = other DNA
ttaaaaattaaaatgaacc

SEQ ID NO: 104 - DNA, 18 bp, synthetic construct
  source 1..18: mol_type = other DNA
taaaaattaaaatgaacc

SEQ ID NO: 105 - DNA, 16 bp, synthetic construct
  source 1..16: mol_type = other DNA
gccccaaactttccac

SEQ ID NO: 106 - DNA, 17 bp, synthetic construct
  source 1..17: mol_type = other DNA
caaactttccactcctt

SEQ ID NO: 107 - DNA, 19 bp, synthetic construct
  source 1..19: mol_type = other DNA
caaactttccactcctttt

SEQ ID NO: 108 - DNA, 18 bp, synthetic construct
  source 1..18: mol_type = other DNA
caaactttccactccttt

SEQ ID NO: 109 - DNA, 20 bp, synthetic construct
  source 1..20: mol_type = other DNA
aaactttccactccttttaa

SEQ ID NO: 110 - DNA, 18 bp, synthetic construct
  source 1..18: mol_type = other DNA
aaactttccactcctttt

SEQ ID NO: 111 - DNA, 17 bp, synthetic construct
  source 1..17: mol_type = other DNA
aaactttccactccttt

SEQ ID NO: 112 - DNA, 18 bp, synthetic construct
  source 1..18: mol_type = other DNA
aactttccactcctttta

SEQ ID NO: 113 - DNA, 19 bp, synthetic construct
  source 1..19: mol_type = other DNA
aactttccactccttttaa

SEQ ID NO: 114 - DNA, 20 bp, synthetic construct
  source 1..20: mol_type = other DNA
actttccactccttttaatt

SEQ ID NO: 115 - DNA, 18 bp, synthetic construct
  source 1..18: mol_type = other DNA
actttccactccttttaa

SEQ ID NO: 116 - DNA, 19 bp, synthetic construct
  source 1..19: mol_type = other DNA
ctttccactccttttaatt

SEQ ID NO: 117 - DNA, 18 bp, synthetic construct
  source 1..18: mol_type = other DNA
tttccactccttttaatt

SEQ ID NO: 118 - DNA, 20 bp, synthetic construct
  source 1..20: mol_type = other DNA
tttccactccttttaatttt

SEQ ID NO: 119 - DNA, 19 bp, synthetic construct
  source 1..19: mol_type = other DNA
tttccactccttttaattt

SEQ ID NO: 120 - DNA, 18 bp, synthetic construct
  source 1..18: mol_type = other DNA
ttccactccttttaattt

SEQ ID NO: 121 - DNA, 17 bp, synthetic construct
  source 1..17: mol_type = other DNA
ttccactccttttaatt

SEQ ID NO: 122 - DNA, 20 bp, synthetic construct
  source 1..20: mol_type = other DNA
ttccactccttttaattttt

SEQ ID NO: 123 - DNA, 19 bp, synthetic construct
  source 1..19: mol_type = other DNA
ttccactccttttaatttt

SEQ ID NO: 124 - DNA, 19 bp, synthetic construct
  source 1..19: mol_type = other DNA
tccactccttttaattttt

SEQ ID NO: 125 - DNA, 18 bp, synthetic construct
  source 1..18: mol_type = other DNA
tccactccttttaatttt

SEQ ID NO: 126 - DNA, 17 bp, synthetic construct
  source 1..17: mol_type = other DNA
tccactccttttaattt

SEQ ID NO: 127 - DNA, 18 bp, synthetic construct
  source 1..18: mol_type = other DNA
ccactccttttaattttt

SEQ ID NO: 128 - DNA, 17 bp, synthetic construct
  source 1..17: mol_type = other DNA
cactccttttaattttt

SEQ ID NO: 129 - DNA, 19 bp, synthetic construct
  source 1..19: mol_type = other DNA
actccttttaatttttaag

SEQ ID NO: 130 - DNA, 18 bp, synthetic construct
  source 1..18: mol_type = other DNA
ctccttttaatttttaag

SEQ ID NO: 131 - DNA, 18 bp, synthetic construct
  source 1..18: mol_type = other DNA
tccttttaatttttaagc

SEQ ID NO: 132 - DNA, 20 bp, synthetic construct
  source 1..20: mol_type = other DNA
ttttcttctgtttctttttc

SEQ ID NO: 133 - DNA, 20 bp, synthetic construct
  source 1..20: mol_type = other DNA
ctgtttctttttcttgtatt

SEQ ID NO: 134 - DNA, 20 bp, synthetic construct
  source 1..20: mol_type = other DNA
tgtttctttttcttgtattt

SEQ ID NO: 135 - DNA, 19 bp, synthetic construct
  source 1..19: mol_type = other DNA
tgtttctttttcttgtatt

SEQ ID NO: 136 - DNA, 20 bp, synthetic construct
  source 1..20: mol_type = other DNA
gtttctttttcttgtatttt

SEQ ID NO: 137 - DNA, 18 bp, synthetic construct
  source 1..18: mol_type = other DNA
gtttctttttcttgtatt

SEQ ID NO: 138 - DNA, 19 bp, synthetic construct
  source 1..19: mol_type = other DNA
gtttctttttcttgtattt

SEQ ID NO: 139 - DNA, 19 bp, synthetic construct
  source 1..19: mol_type = other DNA
cttcaaattctcccagatc

SEQ ID NO: 140 - DNA, 20 bp, synthetic construct
  source 1..20: mol_type = other DNA
cataatgaaattatttcctt

SEQ ID NO: 141 - DNA, 20 bp, synthetic construct
  source 1..20: mol_type = other DNA
ataatgaaattatttccttt

SEQ ID NO: 142 - DNA, 19 bp, synthetic construct
  source 1..19: mol_type = other DNA
ataatgaaattatttcctt

SEQ ID NO: 143 - DNA, 19 bp, synthetic construct
  source 1..19: mol_type = other DNA
taatgaaattatttccttt

SEQ ID NO: 144 - DNA, 18 bp, synthetic construct
  source 1..18: mol_type = other DNA
taatgaaattatttcctt

SEQ ID NO: 145 - DNA, 20 bp, synthetic construct
  source 1..20: mol_type = other DNA
taatgaaattatttcctttt

SEQ ID NO: 146 - DNA, 20 bp, synthetic construct
  source 1..20: mol_type = other DNA
aatgaaattatttccttttt

SEQ ID NO: 147 - DNA, 19 bp, synthetic construct
  source 1..19: mol_type = other DNA
aatgaaattatttcctttt

SEQ ID NO: 148 - DNA, 19 bp, synthetic construct
  source 1..19: mol_type = other DNA
atgaaattatttccttttt

SEQ ID NO: 149 - DNA, 18 bp, synthetic construct
  source 1..18: mol_type = other DNA
atgaaattatttcctttt

SEQ ID NO: 150 - DNA, 20 bp, synthetic construct
  source 1..20: mol_type = other DNA
atgaaattatttcctttttt

SEQ ID NO: 151 - DNA, 19 bp, synthetic construct
  source 1..19: mol_type = other DNA
tgaaattatttcctttttt

SEQ ID NO: 152 - DNA, 20 bp, synthetic construct
  source 1..20: mol_type = other DNA
tatctctacaaaacaaaatt

SEQ ID NO: 153 - DNA, 20 bp, synthetic construct
  source 1..20: mol_type = other DNA
ttaaaaaaattcaccaggcg

SEQ ID NO: 154 - DNA, 17 bp, synthetic construct
  source 1..17: mol_type = other DNA
aaaagaagaccctctcc

SEQ ID NO: 155 - DNA, 17 bp, synthetic construct
  source 1..17: mol_type = other DNA
aaaagaagaccctctcc

SEQ ID NO: 156 - DNA, 18 bp, synthetic construct
  source 1..18: mol_type = other DNA
aaaagaagaccctctccc

SEQ ID NO: 157 - DNA, 17 bp, synthetic construct
  source 1..17: mol_type = other DNA
aaagaagaccctctccc

SEQ ID NO: 158 - DNA, 17 bp, synthetic construct
  source 1..17: mol_type = other DNA
ctgaatccttcaccttt

SEQ ID NO: 159 - DNA, 17 bp, synthetic construct
  source 1..17: mol_type = other DNA
tgaatccttcacctttc

SEQ ID NO: 160 - DNA, 16 bp, synthetic construct
  source 1..16: mol_type = other DNA
gaatccttcacctttc

SEQ ID NO: 161 - DNA, 19 bp, synthetic construct
  source 1..19: mol_type = other DNA
ttcacctttcagaaggcac

SEQ ID NO: 162 - DNA, 18 bp, synthetic construct
  source 1..18: mol_type = other DNA
tcacctttcagaaggcac

SEQ ID NO: 163 - DNA, 17 bp, synthetic construct
  source 1..17: mol_type = other DNA
tgaatacaattttttac

SEQ ID NO: 164 - DNA, 19 bp, synthetic construct
  source 1..19: mol_type = other DNA
aaaaattattccaaggctg

SEQ ID NO: 165 - DNA, 18 bp, synthetic construct
  source 1..18: mol_type = other DNA
ttgttatttccttatttt

SEQ ID NO: 166 - DNA, 20 bp, synthetic construct
  source 1..20: mol_type = other DNA
ttattatttttgagatgccc

SEQ ID NO: 167 - DNA, 19 bp, synthetic construct
  source 1..19: mol_type = other DNA
tattatttttgagatgccc

SEQ ID NO: 168 - DNA, 18 bp, synthetic construct
  source 1..18: mol_type = other DNA
tattatttttgagatgcc

SEQ ID NO: 169 - DNA, 18 bp, synthetic construct
  source 1..18: mol_type = other DNA
attatttttgagatgccc

SEQ ID NO: 170 - DNA, 17 bp, synthetic construct
  source 1..17: mol_type = other DNA
ttatttttgagatgccc

SEQ ID NO: 171 - DNA, 16 bp, synthetic construct
  source 1..16: mol_type = other DNA
tatttttgagatgccc

SEQ ID NO: 172 - DNA, 17 bp, synthetic construct
  source 1..17: mol_type = other DNA
ccagcctgttgttattt

SEQ ID NO: 173 - DNA, 15 bp, synthetic construct
  source 1..15: mol_type = other DNA
cgtggcaaccctatc

SEQ ID NO: 174 - DNA, 17 bp, synthetic construct
  source 1..17: mol_type = other DNA
caaactttctactcttg

SEQ ID NO: 175 - DNA, 19 bp, synthetic construct
  source 1..19: mol_type = other DNA
tactcttgtcccgaacttt

SEQ ID NO: 176 - DNA, 18 bp, synthetic construct
  source 1..18: mol_type = other DNA
actcttgtcccgaacttt

SEQ ID NO: 177 - DNA, 17 bp, synthetic construct
  source 1..17: mol_type = other DNA
ctcttgtcccgaacttt

SEQ ID NO: 178 - DNA, 18 bp, synthetic construct
  source 1..18: mol_type = other DNA
ctcttgtcccgaactttc

SEQ ID NO: 179 - DNA, 16 bp, synthetic construct
  source 1..16: mol_type = other DNA
ctcttgtcccgaactt

SEQ ID NO: 180 - DNA, 17 bp, synthetic construct
  source 1..17: mol_type = other DNA
tcttgtcccgaactttc

SEQ ID NO: 181 - DNA, 16 bp, synthetic construct
  source 1..16: mol_type = other DNA
cttgtcccgaactttc

SEQ ID NO: 182 - DNA, 17 bp, synthetic construct
  source 1..17: mol_type = other DNA
cttgtcccgaactttcc

SEQ ID NO: 183 - DNA, 16 bp, synthetic construct
  source 1..16: mol_type = other DNA
ttgtcccgaactttcc

SEQ ID NO: 184 - DNA, 18 bp, synthetic construct
  source 1..18: mol_type = other DNA
ttgtcccgaactttccac

SEQ ID NO: 185 - DNA, 17 bp, synthetic construct
  source 1..17: mol_type = other DNA
ttgtcccgaactttcca

SEQ ID NO: 186 - DNA, 16 bp, synthetic construct
  source 1..16: mol_type = other DNA
tgtcccgaactttcca

SEQ ID NO: 187 - DNA, 17 bp, synthetic construct
  source 1..17: mol_type = other DNA
tgtcccgaactttccac

SEQ ID NO: 188 - DNA, 18 bp, synthetic construct
  source 1..18: mol_type = other DNA
tcccgaactttccacttc

SEQ ID NO: 189 - DNA, 17 bp, synthetic construct
  source 1..17: mol_type = other DNA
cccgaactttccacttc

SEQ ID NO: 190 - DNA, 16 bp, synthetic construct
  source 1..16: mol_type = other DNA
ccgaactttccacttc

SEQ ID NO: 191 - DNA, 20 bp, synthetic construct
  source 1..20: mol_type = other DNA
aagtcacatgtgctaataac

SEQ ID NO: 192 - DNA, 20 bp, synthetic construct
  source 1..20: mol_type = other DNA
agtcacatgtgctaataact

SEQ ID NO: 193 - DNA, 19 bp, synthetic construct
  source 1..19: mol_type = other DNA
agtcacatgtgctaataac

SEQ ID NO: 194 - DNA, 19 bp, synthetic construct
  source 1..19: mol_type = other DNA
gtcacatgtgctaataact

SEQ ID NO: 195 - DNA, 18 bp, synthetic construct
  source 1..18: mol_type = other DNA
gtcacatgtgctaataac

SEQ ID NO: 196 - DNA, 17 bp, synthetic construct
  source 1..17: mol_type = other DNA
tcacatgtgctaataac

SEQ ID NO: 197 - DNA, 18 bp, synthetic construct
  source 1..18: mol_type = other DNA
tcacatgtgctaataact

SEQ ID NO: 198 - DNA, 20 bp, synthetic construct
  source 1..20: mol_type = other DNA
tcacatgtgctaataactac

SEQ ID NO: 199 - DNA, 19 bp, synthetic construct
  source 1..19: mol_type = other DNA
cacatgtgctaataactac

SEQ ID NO: 200 - DNA, 17 bp, synthetic construct
  source 1..17: mol_type = other DNA
cacatgtgctaataact

SEQ ID NO: 201 - DNA, 18 bp, synthetic construct
  source 1..18: mol_type = other DNA
cacatgtgctaataacta

SEQ ID NO: 202 - DNA, 16 bp, synthetic construct
  source 1..16: mol_type = other DNA
acatgtgctaataact

SEQ ID NO: 203 - DNA, 18 bp, synthetic construct
  source 1..18: mol_type = other DNA
acatgtgctaataactac

SEQ ID NO: 204 - DNA, 17 bp, synthetic construct
  source 1..17: mol_type = other DNA
catgtgctaataactac

SEQ ID NO: 205 - DNA, 18 bp, synthetic construct
  source 1..18: mol_type = other DNA
catgtgctaataactaca

SEQ ID NO: 206 - DNA, 19 bp, synthetic construct
  source 1..19: mol_type = other DNA
catgtgctaataactacat

SEQ ID NO: 207 - DNA, 18 bp, synthetic construct
  source 1..18: mol_type = other DNA
atgtgctaataactacat

SEQ ID NO: 208 - DNA, 19 bp, synthetic construct
  source 1..19: mol_type = other DNA
tgctaataactacatgcat

SEQ ID NO: 209 - DNA, 19 bp, synthetic construct
  source 1..19: mol_type = other DNA
gctaataactacatgcatg

SEQ ID NO: 210 - DNA, 18 bp, synthetic construct
  source 1..18: mol_type = other DNA
gctaataactacatgcat

SEQ ID NO: 211 - DNA, 17 bp, synthetic construct
  source 1..17: mol_type = other DNA
attatcaccactgtccc

SEQ ID NO: 212 - DNA, 17 bp, synthetic construct
  source 1..17: mol_type = other DNA
tcacacacactatgccc

SEQ ID NO: 213 - DNA, 18 bp, synthetic construct
  source 1..18: mol_type = other DNA
tcacacacactatgccct

SEQ ID NO: 214 - DNA, 19 bp, synthetic construct
  source 1..19: mol_type = other DNA
aacaaatgtgttagttaat

SEQ ID NO: 215 - DNA, 20 bp, synthetic construct
  source 1..20: mol_type = other DNA
aacaaatgtgttagttaatt

SEQ ID NO: 216 - DNA, 19 bp, synthetic construct
  source 1..19: mol_type = other DNA
acaaatgtgttagttaatt

SEQ ID NO: 217 - DNA, 20 bp, synthetic construct
  source 1..20: mol_type = other DNA
acaaatgtgttagttaattt

SEQ ID NO: 218 - DNA, 19 bp, synthetic construct
  source 1..19: mol_type = other DNA
caaatgtgttagttaattt

SEQ ID NO: 219 - DNA, 18 bp, synthetic construct
  source 1..18: mol_type = other DNA
caaatgtgttagttaatt

SEQ ID NO: 220 - DNA, 19 bp, synthetic construct
  source 1..19: mol_type = other DNA
tgttagttaatttgttact

SEQ ID NO: 221 - DNA, 20 bp, synthetic construct
  source 1..20: mol_type = other DNA
tgttagttaatttgttactt

SEQ ID NO: 222 - DNA, 18 bp, synthetic construct
  source 1..18: mol_type = other DNA
tgttagttaatttgttac

SEQ ID NO: 223 - DNA, 17 bp, synthetic construct
  source 1..17: mol_type = other DNA
gttagttaatttgttac

SEQ ID NO: 224 - DNA, 18 bp, synthetic construct
  source 1..18: mol_type = other DNA
gttagttaatttgttact

SEQ ID NO: 225 - DNA, 19 bp, synthetic construct
  source 1..19: mol_type = other DNA
gttagttaatttgttactt

SEQ ID NO: 226 - DNA, 18 bp, synthetic construct
  source 1..18: mol_type = other DNA
ttagttaatttgttactt

SEQ ID NO: 227 - DNA, 17 bp, synthetic construct
  source 1..17: mol_type = other DNA
tagttaatttgttactt

SEQ ID NO: 228 - DNA, 20 bp, synthetic construct
  source 1..20: mol_type = other DNA
tagttaatttgttacttaac

SEQ ID NO: 229 - DNA, 20 bp, synthetic construct
  source 1..20: mol_type = other DNA
gttaatttgttacttaacac

SEQ ID NO: 230 - DNA, 19 bp, synthetic construct
  source 1..19: mol_type = other DNA
ttaatttgttacttaacac

SEQ ID NO: 231 - DNA, 20 bp, synthetic construct
  source 1..20: mol_type = other DNA
cttaacacatttaagtaaca

SEQ ID NO: 232 - DNA, 19 bp, synthetic construct
  source 1..19: mol_type = other DNA
cttaacacatttaagtaac

SEQ ID NO: 233 - DNA, 19 bp, synthetic construct
  source 1..19: mol_type = other DNA
ttaacacatttaagtaaca

SEQ ID NO: 234 - DNA, 20 bp, synthetic construct
  source 1..20: mol_type = other DNA
agtaacaaatcagaaagctt

SEQ ID NO: 235 - DNA, 19 bp, synthetic construct
  source 1..19: mol_type = other DNA
gtaacaaatcagaaagctt

SEQ ID NO: 236 - DNA, 18 bp, synthetic construct
  source 1..18: mol_type = other DNA
taacaaatcagaaagctt

SEQ ID NO: 237 - DNA, 17 bp, synthetic construct
  source 1..17: mol_type = other DNA
aacaaatcagaaagctt

SEQ ID NO: 238 - DNA, 16 bp, synthetic construct
  source 1..16: mol_type = other DNA
caaagcggaataaacc

SEQ ID NO: 239 - DNA, 15 bp, synthetic construct
  source 1..15: mol_type = other DNA
aaagcggaataaacc

SEQ ID NO: 240 - DNA, 16 bp, synthetic construct
  source 1..16: mol_type = other DNA
aaagcggaataaaccc

SEQ ID NO: 241 - DNA, 15 bp, synthetic construct
  source 1..15: mol_type = other DNA
aagcggaataaaccc

SEQ ID NO: 242 - DNA, 17 bp, synthetic construct
  source 1..17: mol_type = other DNA
tgtttcaatccagcact

SEQ ID NO: 243 - DNA, 17 bp, synthetic construct
  source 1..17: mol_type = other DNA
caagcaaccagcacata

SEQ ID NO: 244 - DNA, 17 bp, synthetic construct
  source 1..17: mol_type = other DNA
gcccacaaagcacaaac

SEQ ID NO: 245 - DNA, 18 bp, synthetic construct
  source 1..18: mol_type = other DNA
aaagcacaaacctgcctc

SEQ ID NO: 246 - DNA, 17 bp, synthetic construct
  source 1..17: mol_type = other DNA
aaagcacaaacctgcct

SEQ ID NO: 247 - DNA, 16 bp, synthetic construct
  source 1..16: mol_type = other DNA
aagcacaaacctgcct

SEQ ID NO: 248 - DNA, 16 bp, synthetic construct
  source 1..16: mol_type = other DNA
cgacccagcagaaaac

SEQ ID NO: 249 - DNA, 20 bp, synthetic construct
  source 1..20: mol_type = other DNA
cagatcatagtccaaaactc

SEQ ID NO: 250 - DNA, 18 bp, synthetic construct
  source 1..18: mol_type = other DNA
agatcatagtccaaaact

SEQ ID NO: 251 - DNA, 17 bp, synthetic construct
  source 1..17: mol_type = other DNA
gatcatagtccaaaact

SEQ ID NO: 252 - DNA, 16 bp, synthetic construct
  source 1..16: mol_type = other DNA
atcatagtccaaaact

SEQ ID NO: 253 - DNA, 19 bp, synthetic construct
  source 1..19: mol_type = other DNA
ctcagtttctctgacttaa

SEQ ID NO: 254 - DNA, 18 bp, synthetic construct
  source 1..18: mol_type = other DNA
ctcagtttctctgactta

SEQ ID NO: 255 - DNA, 20 bp, synthetic construct
  source 1..20: mol_type = other DNA
ctcagtttctctgacttaaa

SEQ ID NO: 256 - DNA, 19 bp, synthetic construct
  source 1..19: mol_type = other DNA
tcagtttctctgacttaaa

SEQ ID NO: 257 - DNA, 18 bp, synthetic construct
  source 1..18: mol_type = other DNA
tcagtttctctgacttaa

SEQ ID NO: 258 - DNA, 17 bp, synthetic construct
  source 1..17: mol_type = other DNA
tttcattctcccatttg

SEQ ID NO: 259 - DNA, 17 bp, synthetic construct
  source 1..17: mol_type = other DNA
tctcccatttgctcttt

SEQ ID NO: 260 - DNA, 17 bp, synthetic construct
  source 1..17: mol_type = other DNA
ctcccatttgctctttt

SEQ ID NO: 261 - DNA, 18 bp, synthetic construct
  source 1..18: mol_type = other DNA
ccatttgctcttttgttt

SEQ ID NO: 262 - DNA, 20 bp, synthetic construct
  source 1..20: mol_type = other DNA
atttgctcttttgtttgttt

SEQ ID NO: 263 - DNA, 20 bp, synthetic construct
  source 1..20: mol_type = other DNA
cactcaagtaatttttgcat

SEQ ID NO: 264 - DNA, 20 bp, synthetic construct
  source 1..20: mol_type = other DNA
actcaagtaatttttgcatt

SEQ ID NO: 265 - DNA, 19 bp, synthetic construct
  source 1..19: mol_type = other DNA
ctcaagtaatttttgcatt

SEQ ID NO: 266 - DNA, 20 bp, synthetic construct
  source 1..20: mol_type = other DNA
ctcaagtaatttttgcattt

SEQ ID NO: 267 - DNA, 18 bp, synthetic construct
  source 1..18: mol_type = other DNA
tcaagtaatttttgcatt

SEQ ID NO: 268 - DNA, 20 bp, synthetic construct
  source 1..20: mol_type = other DNA
tcaagtaatttttgcatttt

SEQ ID NO: 269 - DNA, 19 bp, synthetic construct
  source 1..19: mol_type = other DNA
tcaagtaatttttgcattt

SEQ ID NO: 270 - DNA, 20 bp, synthetic construct
  source 1..20: mol_type = other DNA
caagtaatttttgcatttta

SEQ ID NO: 271 - DNA, 19 bp, synthetic construct
  source 1..19: mol_type = other DNA
aagtaatttttgcatttta

SEQ ID NO: 272 - DNA, 20 bp, synthetic construct
  source 1..20: mol_type = other DNA
tttgctatttctcaaagctt

SEQ ID NO: 273 - DNA, 19 bp, synthetic construct
  source 1..19: mol_type = other DNA
ttgctatttctcaaagctt

SEQ ID NO: 274 - DNA, 18 bp, synthetic construct
  source 1..18: mol_type = other DNA
tgctatttctcaaagctt

SEQ ID NO: 275 - DNA, 17 bp, synthetic construct
  source 1..17: mol_type = other DNA
gctatttctcaaagctt

SEQ ID NO: 276 - DNA, 17 bp, synthetic construct
  source 1..17: mol_type = other DNA
atccctcctagttttac

SEQ ID NO: 277 - DNA, 16 bp, synthetic construct
  source 1..16: mol_type = other DNA
tccctcctagttttac

SEQ ID NO: 278 - DNA, 15 bp, synthetic construct
  source 1..15: mol_type = other DNA
ccctcctagttttac

SEQ ID NO: 279 - DNA, 17 bp, synthetic construct
  source 1..17: mol_type = other DNA
gttttacttttgagccc

SEQ ID NO: 280 - DNA, 17 bp, synthetic construct
  source 1..17: mol_type = other DNA
agagcatctaactgacc

SEQ ID NO: 281 - DNA, 16 bp, synthetic construct
  source 1..16: mol_type = other DNA
gagcatctaactgacc

SEQ ID NO: 282 - DNA, 16 bp, synthetic construct
  source 1..16: mol_type = other DNA
gcatctaactgaccct

SEQ ID NO: 283 - DNA, 18 bp, synthetic construct
  source 1..18: mol_type = other DNA
gcatctaactgaccctac

SEQ ID NO: 284 - DNA, 18 bp, synthetic construct
  source 1..18: mol_type = other DNA
atctaactgaccctactc

SEQ ID NO: 285 - DNA, 17 bp, synthetic construct
  source 1..17: mol_type = other DNA
tctaactgaccctactc

SEQ ID NO: 286 - DNA, 16 bp, synthetic construct
  source 1..16: mol_type = other DNA
tctaactgaccctact

SEQ ID NO: 287 - DNA, 16 bp, synthetic construct
  source 1..16: mol_type = other DNA
ctaactgaccctactc

SEQ ID NO: 288 - DNA, 17 bp, synthetic construct
  source 1..17: mol_type = other DNA
tgaccctactcagcaac

SEQ ID NO: 289 - DNA, 20 bp, synthetic construct
  source 1..20: mol_type = other DNA
tttttaaaaagacaggatct

SEQ ID NO: 290 - DNA, 20 bp, synthetic construct
  source 1..20: mol_type = other DNA
ttttaaaaagacaggatctt

SEQ ID NO: 291 - DNA, 19 bp, synthetic construct
  source 1..19: mol_type = other DNA
tttaaaaagacaggatctt

SEQ ID NO: 292 - DNA, 18 bp, synthetic construct
  source 1..18: mol_type = other DNA
ttaaaaagacaggatctt

SEQ ID NO: 293 - DNA, 18 bp, synthetic construct
  source 1..18: mol_type = other DNA
caagtgcataccacccat

SEQ ID NO: 294 - DNA, 15 bp, synthetic construct
  source 1..15: mol_type = other DNA
ataatgcacgtaccc

SEQ ID NO: 295 - DNA, 16 bp, synthetic construct
  source 1..16: mol_type = other DNA
ataatgcacgtaccca

SEQ ID NO: 296 - DNA, 18 bp, synthetic construct
  source 1..18: mol_type = other DNA
tgatgaatgacttcccat

SEQ ID NO: 297 - DNA, 16 bp, synthetic construct
  source 1..16: mol_type = other DNA
tgatgaatgacttccc

SEQ ID NO: 298 - DNA, 17 bp, synthetic construct
  source 1..17: mol_type = other DNA
atgaatgacttcccatg

SEQ ID NO: 299 - DNA, 16 bp, synthetic construct
  source 1..16: mol_type = other DNA
tccaggcagacaacac

SEQ ID NO: 300 - DNA, 17 bp, synthetic construct
  source 1..17: mol_type = other DNA
caggcagacaacacctc

SEQ ID NO: 301 - DNA, 16 bp, synthetic construct
  source 1..16: mol_type = other DNA
aggcagacaacacctc

SEQ ID NO: 302 - DNA, 16 bp, synthetic construct
  source 1..16: mol_type = other DNA
gcagacaacacctcac

SEQ ID NO: 303 - DNA, 17 bp, synthetic construct
  source 1..17: mol_type = other DNA
gcagacaacacctcact

SEQ ID NO: 304 - DNA, 17 bp, synthetic construct
  source 1..17: mol_type = other DNA
aacacctcactgagaca

SEQ ID NO: 305 - DNA, 18 bp, synthetic construct
  source 1..18: mol_type = other DNA
aacacctcactgagacac

SEQ ID NO: 306 - DNA, 17 bp, synthetic construct
  source 1..17: mol_type = other DNA
acacctcactgagacac

SEQ ID NO: 307 - DNA, 19 bp, synthetic construct
  source 1..19: mol_type = other DNA
tgaaatctgaaatgtccct

SEQ ID NO: 308 - DNA, 18 bp, synthetic construct
  source 1..18: mol_type = other DNA
gaaatctgaaatgtccct

SEQ ID NO: 309 - DNA, 17 bp, synthetic construct
  source 1..17: mol_type = other DNA
aaatctgaaatgtccct

SEQ ID NO: 310 - DNA, 19 bp, synthetic construct
  source 1..19: mol_type = other DNA
aaatctgaaatgtccctaa

SEQ ID NO: 311 - DNA, 20 bp, synthetic construct
  source 1..20: mol_type = other DNA
aaatctgaaatgtccctaaa

SEQ ID NO: 312 - DNA, 18 bp, synthetic construct
  source 1..18: mol_type = other DNA
aatctgaaatgtccctaa

SEQ ID NO: 313 - DNA, 20 bp, synthetic construct
  source 1..20: mol_type = other DNA
aatctgaaatgtccctaaat

SEQ ID NO: 314 - DNA, 19 bp, synthetic construct
  source 1..19: mol_type = other DNA
atctgaaatgtccctaaat

SEQ ID NO: 315 - DNA, 20 bp, synthetic construct
  source 1..20: mol_type = other DNA
atctgaaatgtccctaaatc

SEQ ID NO: 316 - DNA, 19 bp, synthetic construct
  source 1..19: mol_type = other DNA
tctgaaatgtccctaaatc

SEQ ID NO: 317 - DNA, 18 bp, synthetic construct
  source 1..18: mol_type = other DNA
tctgaaatgtccctaaat

SEQ ID NO: 318 - DNA, 18 bp, synthetic construct
  source 1..18: mol_type = other DNA
tgaaatgtccctaaatcc

SEQ ID NO: 319 - DNA, 18 bp, synthetic construct
  source 1..18: mol_type = other DNA
gaaatgtccctaaatcca

SEQ ID NO: 320 - DNA, 18 bp, synthetic construct
  source 1..18: mol_type = other DNA
tgaatgcctacatgacac

SEQ ID NO: 321 - DNA, 17 bp, synthetic construct
  source 1..17: mol_type = other DNA
gaatgcctacatgacac

SEQ ID NO: 322 - DNA, 17 bp, synthetic construct
  source 1..17: mol_type = other DNA
aatgcctacatgacacc

SEQ ID NO: 323 - DNA, 19 bp, synthetic construct
  source 1..19: mol_type = other DNA
aatgcctacatgacaccac

SEQ ID NO: 324 - DNA, 18 bp, synthetic construct
  source 1..18: mol_type = other DNA
aatgcctacatgacacca

SEQ ID NO: 325 - DNA, 18 bp, synthetic construct
  source 1..18: mol_type = other DNA
cctacatgacaccacaaa

SEQ ID NO: 326 - DNA, 18 bp, synthetic construct
  source 1..18: mol_type = other DNA
gaaaaataaaactacctt

SEQ ID NO: 327 - DNA, 19 bp, synthetic construct
  source 1..19: mol_type = other DNA
gaaaaataaaactaccttg

SEQ ID NO: 328 - DNA, 18 bp, synthetic construct
  source 1..18: mol_type = other DNA
tgtataagaaacatacat

SEQ ID NO: 329 - DNA, 18 bp, synthetic construct
  source 1..18: mol_type = other DNA
agcttaaacaataaaatc

SEQ ID NO: 330 - DNA, 20 bp, synthetic construct
  source 1..20: mol_type = other DNA
agcttaaacaataaaatcaa

SEQ ID NO: 331 - DNA, 17 bp, synthetic construct
  source 1..17: mol_type = other DNA
gaaaaatctgaaatccg

SEQ ID NO: 332 - DNA, 17 bp, synthetic construct
  source 1..17: mol_type = other DNA
tgcagatcctctgcatt

SEQ ID NO: 333 - DNA, 16 bp, synthetic construct
  source 1..16: mol_type = other DNA
gcagatcctctgcatt

SEQ ID NO: 334 - DNA, 17 bp, synthetic construct
  source 1..17: mol_type = other DNA
gcagatcctctgcattc

SEQ ID NO: 335 - DNA, 16 bp, synthetic construct
  source 1..16: mol_type = other DNA
cagatcctctgcattc

SEQ ID NO: 336 - DNA, 16 bp, synthetic construct
  source 1..16: mol_type = other DNA
agatcctctgcattcc

SEQ ID NO: 337 - DNA, 18 bp, synthetic construct
  source 1..18: mol_type = other DNA
ttcctgcccttctagatt

SEQ ID NO: 338 - DNA, 17 bp, synthetic construct
  source 1..17: mol_type = other DNA
ctgcccttctagattac

SEQ ID NO: 339 - DNA, 17 bp, synthetic construct
  source 1..17: mol_type = other DNA
cccacatcatgcacaaa

SEQ ID NO: 340 - DNA, 18 bp, synthetic construct
  source 1..18: mol_type = other DNA
cttagagcatcaccacca

SEQ ID NO: 341 - DNA, 17 bp, synthetic construct
  source 1..17: mol_type = other DNA
cttagagcatcaccacc

SEQ ID NO: 342 - DNA, 16 bp, synthetic construct
  source 1..16: mol_type = other DNA
ttagagcatcaccacc

SEQ ID NO: 343 - DNA, 16 bp, synthetic construct
  source 1..16: mol_type = other DNA
ggtatcctactttccc

SEQ ID NO: 344 - DNA, 19 bp, synthetic construct
  source 1..19: mol_type = other DNA
tatcctactttccctgtaa

SEQ ID NO: 345 - DNA, 18 bp, synthetic construct
  source 1..18: mol_type = other DNA
atcctactttccctgtaa

SEQ ID NO: 346 - DNA, 17 bp, synthetic construct
  source 1..17: mol_type = other DNA
cctactttccctgtaac

SEQ ID NO: 347 - DNA, 18 bp, synthetic construct
  source 1..18: mol_type = other DNA
aaaagagacacactcacc

SEQ ID NO: 348 - DNA, 19 bp, synthetic construct
  source 1..19: mol_type = other DNA
aaaagagacacactcacct

SEQ ID NO: 349 - DNA, 18 bp, synthetic construct
  source 1..18: mol_type = other DNA
aaagagacacactcacct

SEQ ID NO: 350 - DNA, 19 bp, synthetic construct
  source 1..19: mol_type = other DNA
aaagagacacactcacctc

SEQ ID NO: 351 - DNA, 18 bp, synthetic construct
  source 1..18: mol_type = other DNA
aagagacacactcacctc

SEQ ID NO: 352 - DNA, 15 bp, synthetic construct
  source 1..15: mol_type = other DNA
tgagccacgtccaac

SEQ ID NO: 353 - DNA, 17 bp, synthetic construct
  source 1..17: mol_type = other DNA
aatgctccaatgacacc

SEQ ID NO: 354 - DNA, 18 bp, synthetic construct
  source 1..18: mol_type = other DNA
aatgctccaatgacaccc

SEQ ID NO: 355 - DNA, 18 bp, synthetic construct
  source 1..18: mol_type = other DNA
atattcatgagtttgctt

SEQ ID NO: 356 - DNA, 17 bp, synthetic construct
  source 1..17: mol_type = other DNA
caagcagctctttcatc

SEQ ID NO: 357 - DNA, 17 bp, synthetic construct
  source 1..17: mol_type = other DNA
aagcagctctttcatcc

SEQ ID NO: 358 - DNA, 19 bp, synthetic construct
  source 1..19: mol_type = other DNA
aagcagctctttcatccaa

SEQ ID NO: 359 - DNA, 17 bp, synthetic construct
  source 1..17: mol_type = other DNA
agcagctctttcatcca

SEQ ID NO: 360 - DNA, 18 bp, synthetic construct
  source 1..18: mol_type = other DNA
agcagctctttcatccaa

SEQ ID NO: 361 - DNA, 17 bp, synthetic construct
  source 1..17: mol_type = other DNA
gcagctctttcatccaa

SEQ ID NO: 362 - DNA, 19 bp, synthetic construct
  source 1..19: mol_type = other DNA
tgcatttcatttaagtctc

SEQ ID NO: 363 - DNA, 19 bp, synthetic construct
  source 1..19: mol_type = other DNA
catttcatttaagtctcac

SEQ ID NO: 364 - DNA, 20 bp, synthetic construct
  source 1..20: mol_type = other DNA
atttcatttaagtctcacag

SEQ ID NO: 365 - DNA, 18 bp, synthetic construct
  source 1..18: mol_type = other DNA
aacaactaactcagcctg

SEQ ID NO: 366 - DNA, 18 bp, synthetic construct
  source 1..18: mol_type = other DNA
tctggcaagatttcactt

SEQ ID NO: 367 - DNA, 17 bp, synthetic construct
  source 1..17: mol_type = other DNA
cttcctgagctctaaat

SEQ ID NO: 368 - DNA, 16 bp, synthetic construct
  source 1..16: mol_type = other DNA
acactcgaccagctct

SEQ ID NO: 369 - DNA, 16 bp, synthetic construct
  source 1..16: mol_type = other DNA
tcaggcacttgaatct

SEQ ID NO: 370 - DNA, 18 bp, synthetic construct
  source 1..18: mol_type = other DNA
ccaccatcaattaataat

SEQ ID NO: 371 - DNA, 20 bp, synthetic construct
  source 1..20: mol_type = other DNA
accatcaattaataatgatc

SEQ ID NO: 372 - DNA, 20 bp, synthetic construct
  source 1..20: mol_type = other DNA
caattaataatgatcattat

SEQ ID NO: 373 - DNA, 18 bp, synthetic construct
  source 1..18: mol_type = other DNA
tgacagtcattcatccca

SEQ ID NO: 374 - DNA, 17 bp, synthetic construct
  source 1..17: mol_type = other DNA
tgacagtcattcatccc

SEQ ID NO: 375 - DNA, 17 bp, synthetic construct
  source 1..17: mol_type = other DNA
gacagtcattcatccca

SEQ ID NO: 376 - DNA, 18 bp, synthetic construct
  source 1..18: mol_type = other DNA
gacagtcattcatcccac

SEQ ID NO: 377 - DNA, 17 bp, synthetic construct
  source 1..17: mol_type = other DNA
acagtcattcatcccac

SEQ ID NO: 378 - DNA, 16 bp, synthetic construct
  source 1..16: mol_type = other DNA
atcccacactaagcac

SEQ ID NO: 379 - DNA, 18 bp, synthetic construct
  source 1..18: mol_type = other DNA
tttccctcaaagcctcac

SEQ ID NO: 380 - DNA, 18 bp, synthetic construct
  source 1..18: mol_type = other DNA
tcaaagcctcaccagtct

SEQ ID NO: 381 - DNA, 16 bp, synthetic construct
  source 1..16: mol_type = other DNA
agtcgccttctctcta

SEQ ID NO: 382 - DNA, 19 bp, synthetic construct
  source 1..19: mol_type = other DNA
caaaaactttttcaatgat

SEQ ID NO: 383 - DNA, 18 bp, synthetic construct
  source 1..18: mol_type = other DNA
catcaccatcacaggatt

SEQ ID NO: 384 - DNA, 19 bp, synthetic construct
  source 1..19: mol_type = other DNA
catcaccatcacaggattt

SEQ ID NO: 385 - DNA, 20 bp, synthetic construct
  source 1..20: mol_type = other DNA
atcaccatcacaggatttct

SEQ ID NO: 386 - DNA, 18 bp, synthetic construct
  source 1..18: mol_type = other DNA
atcaccatcacaggattt

SEQ ID NO: 387 - DNA, 17 bp, synthetic construct
  source 1..17: mol_type = other DNA
atcaccatcacaggatt

SEQ ID NO: 388 - DNA, 19 bp, synthetic construct
  source 1..19: mol_type = other DNA
atcaccatcacaggatttc

SEQ ID NO: 389 - DNA, 19 bp, synthetic construct
  source 1..19: mol_type = other DNA
tcaccatcacaggatttct

SEQ ID NO: 390 - DNA, 18 bp, synthetic construct
  source 1..18: mol_type = other DNA
tcaccatcacaggatttc

SEQ ID NO: 391 - DNA, 17 bp, synthetic construct
  source 1..17: mol_type = other DNA
caccatcacaggatttc

SEQ ID NO: 392 - DNA, 20 bp, synthetic construct
  source 1..20: mol_type = other DNA
gaaactgtttttgttttttt

SEQ ID NO: 393 - DNA, 20 bp, synthetic construct
  source 1..20: mol_type = other DNA
actgtttttgtttttttgac

SEQ ID NO: 394 - DNA, 17 bp, synthetic construct
  source 1..17: mol_type = other DNA
gacatattttgttaact

SEQ ID NO: 395 - DNA, 19 bp, synthetic construct
  source 1..19: mol_type = other DNA
tgttaacttgcttttttcc

SEQ ID NO: 396 - DNA, 19 bp, synthetic construct
  source 1..19: mol_type = other DNA
gttaacttgcttttttccc

SEQ ID NO: 397 - DNA, 18 bp, synthetic construct
  source 1..18: mol_type = other DNA
gttaacttgcttttttcc

SEQ ID NO: 398 - DNA, 18 bp, synthetic construct
  source 1..18: mol_type = other DNA
ttaacttgcttttttccc

SEQ ID NO: 399 - DNA, 19 bp, synthetic construct
  source 1..19: mol_type = other DNA
ttaacttgcttttttccca

SEQ ID NO: 400 - DNA, 18 bp, synthetic construct
  source 1..18: mol_type = other DNA
ttttaaattgatccatcc

SEQ ID NO: 401 - DNA, 19 bp, synthetic construct
  source 1..19: mol_type = other DNA
ttaaattgatccatcccac

SEQ ID NO: 402 - DNA, 16 bp, synthetic construct
  source 1..16: mol_type = other DNA
agaaaacacctgatcc

SEQ ID NO: 403 - DNA, 16 bp, synthetic construct
  source 1..16: mol_type = other DNA
tgtggtctccaatttc

SEQ ID NO: 404 - DNA, 19 bp, synthetic construct
  source 1..19: mol_type = other DNA
caatttccaaagacagtcc

SEQ ID NO: 405 - DNA, 18 bp, synthetic construct
  source 1..18: mol_type = other DNA
atttccaaagacagtccc

SEQ ID NO: 406 - DNA, 18 bp, synthetic construct
  source 1..18: mol_type = other DNA
gtctcctttcccatttac

SEQ ID NO: 407 - DNA, 16 bp, synthetic construct
  source 1..16: mol_type = other DNA
gcagctagcattaact

SEQ ID NO: 408 - DNA, 15 bp, synthetic construct
  source 1..15: mol_type = other DNA
gtatgaacgtaccac

SEQ ID NO: 409 - DNA, 18 bp, synthetic construct
  source 1..18: mol_type = other DNA
caagaaacaaaagccatt

SEQ ID NO: 410 - DNA, 18 bp, synthetic construct
  source 1..18: mol_type = other DNA
aaacaaaagccattcacc

SEQ ID NO: 411 - DNA, 20 bp, synthetic construct
  source 1..20: mol_type = other DNA
aacaaaagccattcaccaac

SEQ ID NO: 412 - DNA, 16 bp, synthetic construct
  source 1..16: mol_type = other DNA
ttgctcatatgcacat

SEQ ID NO: 413 - DNA, 16 bp, synthetic construct
  source 1..16: mol_type = other DNA
tgacagcagctattac

SEQ ID NO: 414 - DNA, 20 bp, synthetic construct
  source 1..20: mol_type = other DNA
tcaaagataatctaacaatc

SEQ ID NO: 415 - DNA, 20 bp, synthetic construct
  source 1..20: mol_type = other DNA
gataatctaacaatcaatct

SEQ ID NO: 416 - DNA, 16 bp, synthetic construct
  source 1..16: mol_type = other DNA
acaatcaatctgagat

SEQ ID NO: 417 - DNA, 18 bp, synthetic construct
  source 1..18: mol_type = other DNA
atttcctccagtgctcac

SEQ ID NO: 418 - DNA, 17 bp, synthetic construct
  source 1..17: mol_type = other DNA
ctcacagagcctttacc

SEQ ID NO: 419 - DNA, 17 bp, synthetic construct
  source 1..17: mol_type = other DNA
aaagagttgcctaatat

SEQ ID NO: 420 - DNA, 17 bp, synthetic construct
  source 1..17: mol_type = other DNA
aagagttgcctaatatt

SEQ ID NO: 421 - DNA, 15 bp, synthetic construct
  source 1..15: mol_type = other DNA
gcctaatattaagcc

SEQ ID NO: 422 - DNA, 20 bp, synthetic construct
  source 1..20: mol_type = other DNA
tatggaataaacttattcat

SEQ ID NO: 423 - DNA, 15 bp, synthetic construct
  source 1..15: mol_type = other DNA
tacattcgtacatgt

SEQ ID NO: 424 - DNA, 18 bp, synthetic construct
  source 1..18: mol_type = other DNA
gacacccagttataaatt

SEQ ID NO: 425 - DNA, 17 bp, synthetic construct
  source 1..17: mol_type = other DNA
gacacccagttataaat

SEQ ID NO: 426 - DNA, 17 bp, synthetic construct
  source 1..17: mol_type = other DNA
acacccagttataaatt

SEQ ID NO: 427 - DNA, 20 bp, synthetic construct
  source 1..20: mol_type = other DNA
tcactttctggaacatattt

SEQ ID NO: 428 - DNA, 19 bp, synthetic construct
  source 1..19: mol_type = other DNA
tcactttctggaacatatt

SEQ ID NO: 429 - DNA, 18 bp, synthetic construct
  source 1..18: mol_type = other DNA
tcactttctggaacatat

SEQ ID NO: 430 - DNA, 20 bp, synthetic construct
  source 1..20: mol_type = other DNA
cactttctggaacatatttt

SEQ ID NO: 431 - DNA, 18 bp, synthetic construct
  source 1..18: mol_type = other DNA
cactttctggaacatatt

SEQ ID NO: 432 - DNA, 19 bp, synthetic construct
  source 1..19: mol_type = other DNA
cactttctggaacatattt

SEQ ID NO: 433 - DNA, 20 bp, synthetic construct
  source 1..20: mol_type = other DNA
agataaataaagatatttcc

SEQ ID NO: 434 - DNA, 20 bp, synthetic construct
  source 1..20: mol_type = other DNA
gataaataaagatatttcca

SEQ ID NO: 435 - DNA, 19 bp, synthetic construct
  source 1..19: mol_type = other DNA
gataaataaagatatttcc

SEQ ID NO: 436 - DNA, 20 bp, synthetic construct
  source 1..20: mol_type = other DNA
ataaataaagatatttccac

SEQ ID NO: 437 - DNA, 19 bp, synthetic construct
  source 1..19: mol_type = other DNA
ataaataaagatatttcca

SEQ ID NO: 438 - DNA, 19 bp, synthetic construct
  source 1..19: mol_type = other DNA
taaataaagatatttccac

SEQ ID NO: 439 - DNA, 20 bp, synthetic construct
  source 1..20: mol_type = other DNA
taaataaagatatttccact

SEQ ID NO: 440 - DNA, 20 bp, synthetic construct
  source 1..20: mol_type = other DNA
aaataaagatatttccactt

SEQ ID NO: 441 - DNA, 19 bp, synthetic construct
  source 1..19: mol_type = other DNA
aaataaagatatttccact

SEQ ID NO: 442 - DNA, 20 bp, synthetic construct
  source 1..20: mol_type = other DNA
aataaagatatttccacttt

SEQ ID NO: 443 - DNA, 19 bp, synthetic construct
  source 1..19: mol_type = other DNA
ataaagatatttccacttt

SEQ ID NO: 444 - DNA, 20 bp, synthetic construct
  source 1..20: mol_type = other DNA
gatatttccactttgaaaat

SEQ ID NO: 445 - DNA, 20 bp, synthetic construct
  source 1..20: mol_type = other DNA
atatttccactttgaaaatc

SEQ ID NO: 446 - DNA, 19 bp, synthetic construct
  source 1..19: mol_type = other DNA
tatttccactttgaaaatc

SEQ ID NO: 447 - DNA, 20 bp, synthetic construct
  source 1..20: mol_type = other DNA
tatttccactttgaaaatct

SEQ ID NO: 448 - DNA, 19 bp, synthetic construct
  source 1..19: mol_type = other DNA
atttccactttgaaaatct

SEQ ID NO: 449 - DNA, 18 bp, synthetic construct
  source 1..18: mol_type = other DNA
cctagaagcttcctcaat

SEQ ID NO: 450 - DNA, 17 bp, synthetic construct
  source 1..17: mol_type = other DNA
ctagaagcttcctcaat

SEQ ID NO: 451 - DNA, 18 bp, synthetic construct
  source 1..18: mol_type = other DNA
ctagaagcttcctcaatc

SEQ ID NO: 452 - DNA, 18 bp, synthetic construct
  source 1..18: mol_type = other DNA
tagaagcttcctcaatct

SEQ ID NO: 453 - DNA, 17 bp, synthetic construct
  source 1..17: mol_type = other DNA
tagaagcttcctcaatc

SEQ ID NO: 454 - DNA, 18 bp, synthetic construct
  source 1..18: mol_type = other DNA
gaggaccacttccaaaat

SEQ ID NO: 455 - DNA, 17 bp, synthetic construct
  source 1..17: mol_type = other DNA
accacttccaaaatgtc

SEQ ID NO: 456 - DNA, 18 bp, synthetic construct
  source 1..18: mol_type = other DNA
ctctctctgctgtttcaa

SEQ ID NO: 457 - DNA, 18 bp, synthetic construct
  source 1..18: mol_type = other DNA
tctctctgctgtttcaaa

SEQ ID NO: 458 - DNA, 16 bp, synthetic construct
  source 1..16: mol_type = other DNA
accttgacagacactc

SEQ ID NO: 459 - DNA, 18 bp, synthetic construct
  source 1..18: mol_type = other DNA
caagtcacagcttcacca

SEQ ID NO: 460 - DNA, 17 bp, synthetic construct
  source 1..17: mol_type = other DNA
caagtcacagcttcacc

SEQ ID NO: 461 - DNA, 19 bp, synthetic construct
  source 1..19: mol_type = other DNA
caagtcacagcttcaccaa

SEQ ID NO: 462 - DNA, 18 bp, synthetic construct
  source 1..18: mol_type = other DNA
aagtcacagcttcaccaa

SEQ ID NO: 463 - DNA, 17 bp, synthetic construct
  source 1..17: mol_type = other DNA
agtcacagcttcaccaa

SEQ ID NO: 464 - DNA, 18 bp, synthetic construct
  source 1..18: mol_type = other DNA
tcactttgtcattagctc

SEQ ID NO: 465 - DNA, 17 bp, synthetic construct
  source 1..17: mol_type = other DNA
cactttgtcattagctc

SEQ ID NO: 466 - DNA, 18 bp, synthetic construct
  source 1..18: mol_type = other DNA
ctttgtcattagctccaa

SEQ ID NO: 467 - DNA, 17 bp, synthetic construct
  source 1..17: mol_type = other DNA
tttgtcattagctccaa

SEQ ID NO: 468 - DNA, 18 bp, synthetic construct
  source 1..18: mol_type = other DNA
tttgtcattagctccaaa

SEQ ID NO: 469 - DNA, 20 bp, synthetic construct
  source 1..20: mol_type = other DNA
tttgtcattagctccaaaaa

SEQ ID NO: 470 - DNA, 20 bp, synthetic construct
  source 1..20: mol_type = other DNA
ttgtcattagctccaaaaat

SEQ ID NO: 471 - DNA, 19 bp, synthetic construct
  source 1..19: mol_type = other DNA
tgtcattagctccaaaaat

SEQ ID NO: 472 - DNA, 18 bp, synthetic construct
  source 1..18: mol_type = other DNA
gtcattagctccaaaaat

SEQ ID NO: 473 - DNA, 17 bp, synthetic construct
  source 1..17: mol_type = other DNA
tcattagctccaaaaat

SEQ ID NO: 474 - DNA, 16 bp, synthetic construct
  source 1..16: mol_type = other DNA
tgataatatcagtctc

SEQ ID NO: 475 - DNA, 20 bp, synthetic construct
  source 1..20: mol_type = other DNA
tcagtctctgtaccttacaa

SEQ ID NO: 476 - DNA, 19 bp, synthetic construct
  source 1..19: mol_type = other DNA
cagtctctgtaccttacaa

SEQ ID NO: 477 - DNA, 17 bp, synthetic construct
  source 1..17: mol_type = other DNA
cagtctctgtaccttac

SEQ ID NO: 478 - DNA, 17 bp, synthetic construct
  source 1..17: mol_type = other DNA
agtctctgtaccttaca

SEQ ID NO: 479 - DNA, 18 bp, synthetic construct
  source 1..18: mol_type = other DNA
agtctctgtaccttacaa

SEQ ID NO: 480 - DNA, 18 bp, synthetic construct
  source 1..18: mol_type = other DNA
aacatgcctgaaatattt

SEQ ID NO: 481 - DNA, 17 bp, synthetic construct
  source 1..17: mol_type = other DNA
gcttatttgcctaaaac

SEQ ID NO: 482 - DNA, 19 bp, synthetic construct
  source 1..19: mol_type = other DNA
cctaaaactatgtaacaat

SEQ ID NO: 483 - DNA, 19 bp, synthetic construct
  source 1..19: mol_type = other DNA
ccttggtttcctaaaaaat

SEQ ID NO: 484 - DNA, 18 bp, synthetic construct
  source 1..18: mol_type = other DNA
cttggtttcctaaaaaat

SEQ ID NO: 485 - DNA, 17 bp, synthetic construct
  source 1..17: mol_type = other DNA
agagcccttcacacact

SEQ ID NO: 486 - DNA, 19 bp, synthetic construct
  source 1..19: mol_type = other DNA
cttcacacactcagcgtta

SEQ ID NO: 487 - DNA, 18 bp, synthetic construct
  source 1..18: mol_type = other DNA
ttcacacactcagcgtta

SEQ ID NO: 488 - DNA, 17 bp, synthetic construct
  source 1..17: mol_type = other DNA
tcacacactcagcgtta

SEQ ID NO: 489 - DNA, 17 bp, synthetic construct
  source 1..17: mol_type = other DNA
gctcatccatattcctc

SEQ ID NO: 490 - DNA, 18 bp, synthetic construct
  source 1..18: mol_type = other DNA
ctcatccatattcctcac

SEQ ID NO: 491 - DNA, 19 bp, synthetic construct
  source 1..19: mol_type = other DNA
attccatttgtgcatcatt

SEQ ID NO: 492 - DNA, 18 bp, synthetic construct
  source 1..18: mol_type = other DNA
attccatttgtgcatcat

SEQ ID NO: 493 - DNA, 17 bp, synthetic construct
  source 1..17: mol_type = other DNA
attccatttgtgcatca

SEQ ID NO: 494 - DNA, 17 bp, synthetic construct
  source 1..17: mol_type = other DNA
ttccatttgtgcatcat

SEQ ID NO: 495 - DNA, 17 bp, synthetic construct
  source 1..17: mol_type = other DNA
tccatttgtgcatcatt

SEQ ID NO: 496 - DNA, 16 bp, synthetic construct
  source 1..16: mol_type = other DNA
tccatttgtgcatcat

SEQ ID NO: 497 - DNA, 18 bp, synthetic construct
  source 1..18: mol_type = other DNA
tccatttgtgcatcattc

SEQ ID NO: 498 - DNA, 17 bp, synthetic construct
  source 1..17: mol_type = other DNA
ccatttgtgcatcattc

SEQ ID NO: 499 - DNA, 17 bp, synthetic construct
  source 1..17: mol_type = other DNA
catttgtgcatcattct

SEQ ID NO: 500 - DNA, 18 bp, synthetic construct
  source 1..18: mol_type = other DNA
catttgtgcatcattctc

SEQ ID NO: 501 - DNA, 17 bp, synthetic construct
  source 1..17: mol_type = other DNA
atttgtgcatcattctc

SEQ ID NO: 502 - DNA, 18 bp, synthetic construct
  source 1..18: mol_type = other DNA
tttgtgcatcattctctc

SEQ ID NO: 503 - DNA, 17 bp, synthetic construct
  source 1..17: mol_type = other DNA
ttgtgcatcattctctc

SEQ ID NO: 504 - DNA, 19 bp, synthetic construct
  source 1..19: mol_type = other DNA
catcattctctcaggttaa

SEQ ID NO: 505 - DNA, 18 bp, synthetic construct
  source 1..18: mol_type = other DNA
catcattctctcaggtta

SEQ ID NO: 506 - DNA, 19 bp, synthetic construct
  source 1..19: mol_type = other DNA
atcattctctcaggttaat

SEQ ID NO: 507 - DNA, 18 bp, synthetic construct
  source 1..18: mol_type = other DNA
tcattctctcaggttaat

SEQ ID NO: 508 - DNA, 17 bp, synthetic construct
  source 1..17: mol_type = other DNA
attctctcaggttaatc

SEQ ID NO: 509 - DNA, 19 bp, synthetic construct
  source 1..19: mol_type = other DNA
attctctcaggttaatcat

SEQ ID NO: 510 - DNA, 18 bp, synthetic construct
  source 1..18: mol_type = other DNA
attctctcaggttaatca

SEQ ID NO: 511 - DNA, 19 bp, synthetic construct
  source 1..19: mol_type = other DNA
ttctctcaggttaatcatt

SEQ ID NO: 512 - DNA, 16 bp, synthetic construct
  source 1..16: mol_type = other DNA
ttctctcaggttaatc

SEQ ID NO: 513 - DNA, 17 bp, synthetic construct
  source 1..17: mol_type = other DNA
ttctctcaggttaatca

SEQ ID NO: 514 - DNA, 18 bp, synthetic construct
  source 1..18: mol_type = other DNA
tctctcaggttaatcatt

SEQ ID NO: 515 - DNA, 17 bp, synthetic construct
  source 1..17: mol_type = other DNA
ctctcaggttaatcatt

SEQ ID NO: 516 - DNA, 17 bp, synthetic construct
  source 1..17: mol_type = other DNA
taatcattgcctctttc

SEQ ID NO: 517 - DNA, 18 bp, synthetic construct
  source 1..18: mol_type = other DNA
aatcattgcctctttcac

SEQ ID NO: 518 - DNA, 17 bp, synthetic construct
  source 1..17: mol_type = other DNA
acccagtccctcagaac

SEQ ID NO: 519 - DNA, 17 bp, synthetic construct
  source 1..17: mol_type = other DNA
ggatccatcccttacta

SEQ ID NO: 520 - DNA, 15 bp, synthetic construct
  source 1..15: mol_type = other DNA
cttactatggtctcc

SEQ ID NO: 521 - DNA, 18 bp, synthetic construct
  source 1..18: mol_type = other DNA
attaatcattaacgcact

SEQ ID NO: 522 - RNA, 36 nt, synthetic construct
  source 1..36: mol_type = other RNA
cagaagtaaaggaatttgatgttgtgtttttgttaa

SEQ ID NO: 523 - RNA, 23 nt, synthetic construct
  source 1..23: mol_type = other RNA
gataaagaatgttctatttctgc

SEQ ID NO: 524 - RNA, 19 nt, synthetic construct
  source 1..19: mol_type = other RNA
agtatttaactaattatac

SEQ ID NO: 525 - RNA, 37 nt, synthetic construct
  source 1..37: mol_type = other RNA
aagctctggaagttcattcgcgatttcctgcggtggt

SEQ ID NO: 526 - RNA, 15 nt, synthetic construct
  source 1..15: mol_type = other RNA
gaagagcatcgggca

SEQ ID NO: 527 - RNA, 15 nt, synthetic construct
  source 1..15: mol_type = other RNA
ggaggacgtgctcta

SEQ ID NO: 528 - RNA, 17 nt, synthetic construct
  source 1..17: mol_type = other RNA
gtggccatgaatgagag

SEQ ID NO: 529 - RNA, 17 nt, synthetic construct
  source 1..17: mol_type = other RNA
gatactgattcatggta

SEQ ID NO: 530 - RNA, 22 nt, synthetic construct
  source 1..22: mol_type = other RNA
atttttgtcctataaaatggca

SEQ ID NO: 531 - RNA, 19 nt, synthetic construct
  source 1..19: mol_type = other RNA
gaggaaactgcaaatttct

SEQ ID NO: 532 - RNA, 29 nt, synthetic construct
  source 1..29: mol_type = other RNA
gagcatgagaatcaggtaacactgtctgt

SEQ ID NO: 533 - RNA, 28 nt, synthetic construct
  source 1..28: mol_type = other RNA
gtgaagtggggataaaaacagagccttt

SEQ ID NO: 534 - RNA, 16 nt, synthetic construct
  source 1..16: mol_type = other RNA
gtgccagacagaaatt

SEQ ID NO: 535 - RNA, 16 nt, synthetic construct
  source 1..16: mol_type = other RNA
gatgatgggtgtaggt

SEQ ID NO: 536 - RNA, 31 nt, synthetic construct
  source 1..31: mol_type = other RNA
ggttcattttaatttttaaattttaaccttc

SEQ ID NO: 537 - RNA, 34 nt, synthetic construct
  source 1..34: mol_type = other RNA
gcttaaaaattaaaaggagtggaaagtttggggc

SEQ ID NO: 538 - RNA, 29 nt, synthetic construct
  source 1..29: mol_type = other RNA
aaaatacaagaaaaagaaacagaagaaaa

SEQ ID NO: 539 - RNA, 19 nt, synthetic construct
  source 1..19: mol_type = other RNA
gatctgggagaatttgaag

SEQ ID NO: 540 - RNA, 24 nt, synthetic construct
  source 1..24: mol_type = other RNA
aaaaaaggaaataatttcattatg

SEQ ID NO: 541 - RNA, 39 nt, synthetic construct
  source 1..39: mol_type = other RNA
cgcctggtgaatttttttaaattttgttttgtagagata

SEQ ID NO: 542 - RNA, 19 nt, synthetic construct
  source 1..19: mol_type = other RNA
gggagagggtcttcttttc

SEQ ID NO: 543 - RNA, 27 nt, synthetic construct
  source 1..27: mol_type = other RNA
gtgccttctgaaaggtgaaggattcag

SEQ ID NO: 544 - RNA, 17 nt, synthetic construct
  source 1..17: mol_type = other RNA
gtaaaaaattgtattca

SEQ ID NO: 545 - RNA, 19 nt, synthetic construct
  source 1..19: mol_type = other RNA
cagccttggaataattttt

SEQ ID NO: 546 - RNA, 18 nt, synthetic construct
  source 1..18: mol_type = other RNA
aaaataaggaaataacaa

SEQ ID NO: 547 - RNA, 20 nt, synthetic construct
  source 1..20: mol_type = other RNA
gggcatctcaaaaataataa

SEQ ID NO: 548 - RNA, 17 nt, synthetic construct
  source 1..17: mol_type = other RNA
aaataacaacaggctgg

SEQ ID NO: 549 - RNA, 15 nt, synthetic construct
  source 1..15: mol_type = other RNA
gatagggttgccacg

SEQ ID NO: 550 - RNA, 35 nt, synthetic construct
  source 1..35: mol_type = other RNA
gaagtggaaagttcgggacaagagtagaaagtttg

SEQ ID NO: 551 - RNA, 30 nt, synthetic construct
  source 1..30: mol_type = other RNA
catgcatgtagttattagcacatgtgactt

SEQ ID NO: 552 - RNA, 17 nt, synthetic construct
  source 1..17: mol_type = other RNA
gggacagtggtgataat

SEQ ID NO: 553 - RNA, 18 nt, synthetic construct
  source 1..18: mol_type = other RNA
agggcatagtgtgtgtga

SEQ ID NO: 554 - RNA, 58 nt, synthetic construct
  source 1..58: mol_type = other RNA
aagctttctgatttgttacttaaatgtgttaagtaacaaattaactaacacatttgtt

SEQ ID NO: 555 - RNA, 17 nt, synthetic construct
  source 1..17: mol_type = other RNA
gggtttattccgctttg

SEQ ID NO: 556 - RNA, 17 nt, synthetic construct
  source 1..17: mol_type = other RNA
agtgctggattgaaaca

SEQ ID NO: 557 - RNA, 17 nt, synthetic construct
  source 1..17: mol_type = other RNA
tatgtgctggttgcttg

SEQ ID NO: 558 - RNA, 24 nt, synthetic construct
  source 1..24: mol_type = other RNA
gaggcaggtttgtgctttgtgggc

SEQ ID NO: 559 - RNA, 16 nt, synthetic construct
  source 1..16: mol_type = other RNA
gttttctgctgggtcg

SEQ ID NO: 560 - RNA, 20 nt, synthetic construct
  source 1..20: mol_type = other RNA
gagttttggactatgatctg

SEQ ID NO: 561 - RNA, 20 nt, synthetic construct
  source 1..20: mol_type = other RNA
tttaagtcagagaaactgag

SEQ ID NO: 562 - RNA, 32 nt, synthetic construct
  source 1..32: mol_type = other RNA
aaacaaacaaaagagcaaatgggagaatgaaa

SEQ ID NO: 563 - RNA, 24 nt, synthetic construct
  source 1..24: mol_type = other RNA
taaaatgcaaaaattacttgagtg

SEQ ID NO: 564 - RNA, 20 nt, synthetic construct
  source 1..20: mol_type = other RNA
aagctttgagaaatagcaaa

SEQ ID NO: 565 - RNA, 27 nt, synthetic construct
  source 1..27: mol_type = other RNA
gggctcaaaagtaaaactaggagggat

SEQ ID NO: 566 - RNA, 29 nt, synthetic construct
  source 1..29: mol_type = other RNA
gttgctgagtagggtcagttagatgctct

SEQ ID NO: 567 - RNA, 21 nt, synthetic construct
  source 1..21: mol_type = other RNA
aagatcctgtctttttaaaaa

SEQ ID NO: 568 - RNA, 18 nt, synthetic construct
  source 1..18: mol_type = other RNA
atgggtggtatgcacttg

SEQ ID NO: 569 - RNA, 16 nt, synthetic construct
  source 1..16: mol_type = other RNA
tgggtacgtgcattat

SEQ ID NO: 570 - RNA, 19 nt, synthetic construct
  source 1..19: mol_type = other RNA
catgggaagtcattcatca

SEQ ID NO: 571 - RNA, 29 nt, synthetic construct
  source 1..29: mol_type = other RNA
gtgtctcagtgaggtgttgtctgcctgga

SEQ ID NO: 572 - RNA, 26 nt, synthetic construct
  source 1..26: mol_type = other RNA
tggatttagggacatttcagatttca

SEQ ID NO: 573 - RNA, 24 nt, synthetic construct
  source 1..24: mol_type = other RNA
tttgtggtgtcatgtaggcattca

SEQ ID NO: 574 - RNA, 19 nt, synthetic construct
  source 1..19: mol_type = other RNA
caaggtagttttatttttc

SEQ ID NO: 575 - RNA, 18 nt, synthetic construct
  source 1..18: mol_type = other RNA
atgtatgtttcttataca

SEQ ID NO: 576 - RNA, 20 nt, synthetic construct
  source 1..20: mol_type = other RNA
ttgattttattgtttaagct

SEQ ID NO: 577 - RNA, 17 nt, synthetic construct
  source 1..17: mol_type = other RNA
cggatttcagatttttc

SEQ ID NO: 578 - RNA, 19 nt, synthetic construct
  source 1..19: mol_type = other RNA
ggaatgcagaggatctgca

SEQ ID NO: 579 - RNA, 20 nt, synthetic construct
  source 1..20: mol_type = other RNA
gtaatctagaagggcaggaa

SEQ ID NO: 580 - RNA, 17 nt, synthetic construct
  source 1..17: mol_type = other RNA
tttgtgcatgatgtggg

SEQ ID NO: 581 - RNA, 18 nt, synthetic construct
  source 1..18: mol_type = other RNA
tggtggtgatgctctaag

SEQ ID NO: 582 - RNA, 22 nt, synthetic construct
  source 1..22: mol_type = other RNA
gttacagggaaagtaggatacc

SEQ ID NO: 583 - RNA, 20 nt, synthetic construct
  source 1..20: mol_type = other RNA
gaggtgagtgtgtctctttt

SEQ ID NO: 584 - RNA, 15 nt, synthetic construct
  source 1..15: mol_type = other RNA
gttggacgtggctca

SEQ ID NO: 585 - RNA, 18 nt, synthetic construct
  source 1..18: mol_type = other RNA
gggtgtcattggagcatt

SEQ ID NO: 586 - RNA, 18 nt, synthetic construct
  source 1..18: mol_type = other RNA
aagcaaactcatgaatat

SEQ ID NO: 587 - RNA, 20 nt, synthetic construct
  source 1..20: mol_type = other RNA
ttggatgaaagagctgcttg

SEQ ID NO: 588 - RNA, 23 nt, synthetic construct
  source 1..23: mol_type = other RNA
ctgtgagacttaaatgaaatgca

SEQ ID NO: 589 - RNA, 18 nt, synthetic construct
  source 1..18: mol_type = other RNA
caggctgagttagttgtt

SEQ ID NO: 590 - RNA, 32 nt, synthetic construct
  source 1..32: mol_type = other RNA
atttagagctcaggaagtgaaatcttgccaga

SEQ ID NO: 591 - RNA, 16 nt, synthetic construct
  source 1..16: mol_type = other RNA
agagctggtcgagtgt

SEQ ID NO: 592 - RNA, 16 nt, synthetic construct
  source 1..16: mol_type = other RNA
agattcaagtgcctga

SEQ ID NO: 593 - RNA, 27 nt, synthetic construct
  source 1..27: mol_type = other RNA
ataatgatcattattaattgatggtgg

SEQ ID NO: 594 - RNA, 28 nt, synthetic construct
  source 1..28: mol_type = other RNA
gtgcttagtgtgggatgaatgactgtca

SEQ ID NO: 595 - RNA, 24 nt, synthetic construct
  source 1..24: mol_type = other RNA
agactggtgaggctttgagggaaa

SEQ ID NO: 596 - RNA, 16 nt, synthetic construct
  source 1..16: mol_type = other RNA
tagagagaaggcgact

SEQ ID NO: 597 - RNA, 19 nt, synthetic construct
  source 1..19: mol_type = other RNA
atcattgaaaaagtttttg

SEQ ID NO: 598 - RNA, 21 nt, synthetic construct
  source 1..21: mol_type = other RNA
agaaatcctgtgatggtgatg

SEQ ID NO: 599 - RNA, 23 nt, synthetic construct
  source 1..23: mol_type = other RNA
gtcaaaaaaacaaaaacagtttc

SEQ ID NO: 600 - RNA, 30 nt, synthetic construct
  source 1..30: mol_type = other RNA
tgggaaaaaagcaagttaacaaaatatgtc

SEQ ID NO: 601 - RNA, 21 nt, synthetic construct
  source 1..21: mol_type = other RNA
gtgggatggatcaatttaaaa

SEQ ID NO: 602 - RNA, 16 nt, synthetic construct
  source 1..16: mol_type = other RNA
ggatcaggtgttttct

SEQ ID NO: 603 - RNA, 29 nt, synthetic construct
  source 1..29: mol_type = other RNA
gggactgtctttggaaattggagaccaca

SEQ ID NO: 604 - RNA, 18 nt, synthetic construct
  source 1..18: mol_type = other RNA
gtaaatgggaaaggagac

SEQ ID NO: 605 - RNA, 16 nt, synthetic construct
  source 1..16: mol_type = other RNA
agttaatgctagctgc

SEQ ID NO: 606 - RNA, 15 nt, synthetic construct
  source 1..15: mol_type = other RNA
gtggtacgttcatac

SEQ ID NO: 607 - RNA, 25 nt, synthetic construct
  source 1..25: mol_type = other RNA
gttggtgaatggcttttgtttcttg

SEQ ID NO: 608 - RNA, 16 nt, synthetic construct
  source 1..16: mol_type = other RNA
atgtgcatatgagcaa

SEQ ID NO: 609 - RNA, 16 nt, synthetic construct
  source 1..16: mol_type = other RNA
gtaatagctgctgtca

SEQ ID NO: 610 - RNA, 30 nt, synthetic construct
  source 1..30: mol_type = other RNA
atctcagattgattgttagattatctttga

SEQ ID NO: 611 - RNA, 30 nt, synthetic construct
  source 1..30: mol_type = other RNA
ggtaaaggctctgtgagcactggaggaaat

SEQ ID NO: 612 - RNA, 23 nt, synthetic construct
  source 1..23: mol_type = other RNA
ggcttaatattaggcaactcttt

SEQ ID NO: 613 - RNA, 20 nt, synthetic construct
  source 1..20: mol_type = other RNA
atgaataagtttattccata

SEQ ID NO: 614 - RNA, 15 nt, synthetic construct
  source 1..15: mol_type = other RNA
acatgtacgaatgta

SEQ ID NO: 615 - RNA, 18 nt, synthetic construct
  source 1..18: mol_type = other RNA
aatttataactgggtgtc

SEQ ID NO: 616 - RNA, 21 nt, synthetic construct
  source 1..21: mol_type = other RNA
aaaatatgttccagaaagtga

SEQ ID NO: 617 - RNA, 33 nt, synthetic construct
  source 1..33: mol_type = other RNA
agattttcaaagtggaaatatctttatttatct

SEQ ID NO: 618 - RNA, 20 nt, synthetic construct
  source 1..20: mol_type = other RNA
agattgaggaagcttctagg

SEQ ID NO: 619 - RNA, 21 nt, synthetic construct
  source 1..21: mol_type = other RNA
gacattttggaagtggtcctc

SEQ ID NO: 620 - RNA, 19 nt, synthetic construct
  source 1..19: mol_type = other RNA
tttgaaacagcagagagag

SEQ ID NO: 621 - RNA, 16 nt, synthetic construct
  source 1..16: mol_type = other RNA
gagtgtctgtcaaggt

SEQ ID NO: 622 - RNA, 19 nt, synthetic construct
  source 1..19: mol_type = other RNA
ttggtgaagctgtgacttg

SEQ ID NO: 623 - RNA, 25 nt, synthetic construct
  source 1..25: mol_type = other RNA
atttttggagctaatgacaaagtga

SEQ ID NO: 624 - RNA, 44 nt, synthetic construct
  source 1..44: mol_type = other RNA
aaatatttcaggcatgttgtaaggtacagagactgatattatca

SEQ ID NO: 625 - RNA, 28 nt, synthetic construct
  source 1..28: mol_type = other RNA
attgttacatagttttaggcaaataagc

SEQ ID NO: 626 - RNA, 19 nt, synthetic construct
  source 1..19: mol_type = other RNA
attttttaggaaaccaagg

SEQ ID NO: 627 - RNA, 25 nt, synthetic construct
  source 1..25: mol_type = other RNA
taacgctgagtgtgtgaagggctct

SEQ ID NO: 628 - RNA, 19 nt, synthetic construct
  source 1..19: mol_type = other RNA
gtgaggaatatggatgagc

SEQ ID NO: 629 - RNA, 47 nt, synthetic construct
  source 1..47: mol_type = other RNA
gtgaaagaggcaatgattaacctgagagaatgatgcacaaatggaat

SEQ ID NO: 630 - RNA, 17 nt, synthetic construct
  source 1..17: mol_type = other RNA
gttctgagggactgggt

SEQ ID NO: 631 - RNA, 25 nt, synthetic construct
  source 1..25: mol_type = other RNA
ggagaccatagtaagggatggatcc

SEQ ID NO: 632 - RNA, 18 nt, synthetic construct
  source 1..18: mol_type = other RNA
agtgcgttaatgattaat

SEQ ID NO: 633 - RNA, 20031 nt, synthetic construct
  source 1..20031: mol_type = other RNA
aggctggcggcggcacctggccgggagcgggcgcggcgcgggagcgggcgcggcgcggcagcggcggcgggggcgggggcatggaccaggcctggtggagcggggggcccggccgggtgcgccccgccgcccagtgagcgcccgggcggcgcggcccgggccgagccagccacgctgaggcggaggaggacggggacaaggtacgtcccgggctccagcacccactgccagcttgagccctgcctcagtctactccctggggataccgcccacattacgctaacccccaaactgggaccacgcgccggaggaggggagtaggagtgcagtaccgctgttctaagcgctgggtacccctgcccactgccagagggtggcctggtaggggccagggagggcacaggttggtgccccccatgcccttggttttttcccctgtcccccaaccccccttccagggcttcccagtccctgaaggtgggatgggtggggcagaaggcaggctgctggggctgagctggagctgggagtgcgttaatgattaatcttggacacagtggccagagccctaggggaggaggggcagagatctcccctgtctccatcagcccttcctagaaaggggatcagagcaggaaccggagaccatagtaagggatggatcccctctgctgggggaaggccctggcctgtaccccagaggaagcaaagccaccaaggagaggtttgggagagtgggacaaaccctggcatccaggaggggtcctataggaaatcctacagttcctgtttcctagcccagtgcccaggaagggggtccagcttgatctgccccttcggtgccaggccagcattttggtaccagcacagagcacactacagaccgagccctgcccagtgggaactgccccgcccagctgcccagagccagagaggtgatgggcatgtctggctgctgtaacccacaccagggggaaggaaagggagagaaggcagcatgcctgacacctgatccccgggctgccgcggtgacaggtgcatgagaatcctccagaggggagagaggtgtagaggactgtccattccagttgggaaaacccaatctcttcctttactgacggggtgacctgaggcccagagagggtgaggctcatccaggcccacacagggggcacaggagggcagtcctggaccagagcagtcactgtgtacgcactgttccttgtcctgtgaaggaggcattgctacctatccccattatccagagggggatactgaggctcagggaaattcaatgacttgtccaaggtcacatggctcagaggtagctgagctgactccaaatcataggagtcttgccctggcccccatttcaggaccccagcccaacccatgacctctctctgctcctttggggtcctgccttcttggatctgcctgggctgccccaaaccctcagctctggacaccccccctcgcctccctggctcccagctccgcctgacacttcagcgggtgagcaggctgctaccccgggagccgagacaacgaagccctgttcccagctgccacctcagcctgctttattccctgcttgctcaggcagagcccttcctggaggaagctccttgcggacagagaatgcctggtccccatgcatccccttcccttaatgggtccgatgccctcttgtcgcccagcaagggaggagtgtgtcccccacagcccctcccactgtgttctgagggactgggtgtgccatccagtgaaagaggcaatgattaacctgagagaatgatgcacaaatggaattctgggcccggctgcaggctcctgaagggctccttgcttcactttgactgggtgaggaatatggatgagcccttttaggggctccacacgactccaaacagggcagagcaggatggaacacaggtgcccatgggggtcatcaggctggggactgctggaatctccaggtgaggggtgatggtgggaggaagtgggggcccagaagatgcctgccttccctcctgctagttctggctccttggatggggatggtatccttctagaagtctggacagagcccgcttcccctaccaggtgggatctaggaggatcacaatacaatcagaggagctaacgctgagtgtgtgaagggctctgcgtgcctcacagcaggcctgtgaaagcatcatagtacctgccattttttaggaaaccaaggctcagagaggggaagtcacttgcccaaggtcacccagcttggggggtggtggagccagcatttgttaccaggcgagtttccaggaatggagaggtgggtgtatgtatctccggcattgtgcattgcaagtgtcaataccttttcacaaacctttatattgttacatagttttaggcaaataagcgcataagaatccttaaaaaatatttcaggcatgttgtaaggtacagagactgatattatcaactcctacatactcagctctggggttctcagcatttttggagctaatgacaaagtgactgacttcttggtgaagctgtgacttgcaacgccccagttagcgcttctctaaggctactcaaatccccccctcaggaagcggggtgatttacccagacaaggcagtgtagcaggaagagctgtggccttgctcctcattttggctttgccgtgtaggcaaaaaaactgagcctcagtttccccacacatcacatggggctagcagaggagactgggagtgtctgtcaaggtcacaggcatggatgccctttgtttttgtttttgtttttgagacggagtcttgctctgtcacccaggctggagtgcagtggtgcaatctcggctcactgcaagctccgcctcccaggttcacaccattctcctgcctcagcctcccgagtagctgggactataggcgcccaccaccaagcctggctaatttttttataattttagtagagacgggatttcactgtgttagccaggatggtctcaatctcctgacctcgtgatctgcccgcctcggcctcccaaagtgctgggattacaggcgtgagccactgcgcccggccgtggatgccctttgaaacagcagagagagatgaccccgaaagttcaaaaagtgctttctgaatcatcaaagaacattaccctctctcaactgctgggccagctaccacctgcatccacccctgcaggctaggtcgcagcaggggccagatgtctgggctgacattttggaagtggtcctctgagccagcgagttgggatccagctgaaatgctccaataggtggtcttccccctctctgctccagcaccccattctagaggggctcagattgaggaagcttctaggtgcttgcaagtcacttccccagtacccctggatggagggttaacctatcccatccgccccttctgctgtgccacccacacttcccagctccttctcagtcccaggcccctgttgccccttcctcacttggtgcactgaggttcagagagggcagagtgtcttgcctcggccacacagtgaggagaggtggggctcatcttcctccagccaggctgcagattttcaaagtggaaatatctttatttatctttccaaagtaatataagctgttttgtaaagagtgtgtgtgtgtgtgtgtgtgtgtgtgtgtgtgtgtgtatcatcacagaatcaaaatatgttccagaaagtgaatgggcttactccccagagagctacaatttataactgggtgtctttccagacccttatacacatgtacgaatgtacaacttttctctacatgaataagtttattccataaacagtgcattccaggcttaatattaggcaactctttttttttttttgagacggagtttcgttctttttgcctaggctggagtgcaatggcacgatattggctcattgcaacctccgcctcccaggttcaagtgattctcctgcctcagccttaggattaaaggcatgcgccaccatgcccagctaatttttgtatttttagtagagatggggtttctccatgttgtcaggctggtctcaaactcccgacctcaggtgatccacccgccttggcctcccaaagtgctgggattacaggcgtgagccaccacatccggtcaggcaactcttttttaatctctccgagcctcagtttcctcatcttctaaatgggggtaaaggctctgtgagcactggaggaaatgatactgcagtggccacttttatatgcccatctcagattgattgttagattatctttgagccacattcctgcaggtggattgctgggtctgagaacaggcacactcagaactgtaatagctgctgtcaaatctgcactacgaacatcatagggagagttgttttgctccactcttgaaagctccttccatatcccgatgggattccactccagctacagaaagccctgggatacctcaccccaccaccttctctgccagagccctgttcctgtatgtgcatatgagcaaacagcacggatgtgcctcagttggtgaatggcttttgtttcttggagtttctttttttaagatggggtctcacactgttgcccaggcttggagtgcagtggtacgttcatacctctctgtaaccttgaacttctgggttcaaggggtcctcttgcctcagcctcctgagtagctggggctacaggcgtgcaccaccacacagggctaatgtttaaattttttatacagatggggtctcactgtgttgcccaggcaagtctcgaactcctgggctcaagcaatctgcctgtcttggcctccccaaaatgctgggtggcgcacgccaccgtgcccggctggggaatggtttatgggggaatagcggtgctgcaaactctgatacagccctagctgccctgttttgtctccttagggagctcttcttcaccctgctccgtaagcacaggctccctgtccttcctcacccttccctggtgactctagttattcctatggcctcaattcccactttggtgcccagctccccgcctacctctttccctcccccaagacccccctctttgctcctactgctcttcaggcattctatgatcatcccaaacatccctcagactcagagtattctaaacagaaccagacaccacctgcttcacccagcattcaacctggccctttctcagcctcccagactcagggactagctctacctccagccagccagtcacccaacagcaactcttccctcctctcctccatacctgctgccaataggatgctacctttgcatggtctacagacagctctggtacttccccagccttcttgttccaggtcacacctgcacagctgtaacaactactatggaggtcggcctgctcctccctctccttccccatccctacagttcatttcttacccagtagcccaatgggaatcccatctgctccctcctctgctgaagaccctcctgtgggtctgtccctacagcccacaggatctaggtgagactcagtagcatgcttcctgaaagccctgcatgtccctctttttttttttttttttttttgagatggagtctcgctctgttgcccaggctggagtgcagtggcgcgatctctgctcactgcaaactctgcctcccaggttcaagcgattatcctgcctcagcctcccgagtagctggtattacatgcgcctgccatcacgcccggctaatttctgtatttttagtacagacggggtttcaccatgttggccaggctggtctcggacccctaacctcaggtgatctgcccgccttagcctcccaaagtgctggtattacaggcgtgagccactgcgccaggcctctgcatgtccctcttaaccctgccctagtttctggctgtctctgtagtctctcccctgcccacccagcctattacacgcaaacacaccaaacactccagccaggtggaaagtttgtttctcaattgccctgggcctttgcatgtgcttttctctctgtgtggctcattctagggtaacaggcatattagttaatgctagctgctgtaacagacaaacctgaagttctaaaaggcttaatacagttctcgttttcttgaaatccagtcagtgtctggtgggtggagatgcaaagacctaggctcctcccagctccgcccatgcccagatccttggagaccaccccgttcatccagtaaatgggaaaggagaccacagaaaaggtccacccacttaacctcatcagctggcattctgttcacatatcattggccaggacccagttatgtgaggacactgaactgcaagggcgcatgggaaatatagtctagctgtgctgccgtggaaaaaaggggactgtctttggaaattggagaccacacaacagcttctgccaccagaggcccctaccccaggaagccttcctgatactgctgtctccgccccaaggctggatcaggtgttttcttcgtgcttctgcaagcctcagacaatccgttgtccatcacacgcctcacagctgtgtgttcctgtgtctccccagttaggactgtgagccctgagttctctgcaccgggttatcatgaactctagtcctggagtgcaggtgcccagtaaatacctgtgggatggatcaatttaaaactatcacgggctgggcgcggtggctcacgtctgtaatcccagcactttgggaggctgaggcaggtggatcacttgaggtcagaagtttgagaccagcctgcccaaggtggtgaaaccctgtctctactaaaaatacaaaattagctgggtgtggtggcgggtgcccgtaatcccagctacttggaagggtgaggcaggagaattgcttgaacccagggggcagaggttgcagtgggccgagattgccccagtgcactccagcatgggtgacaagagtgcaactctgtctcaaacaacaacaacaacaaaaaacaaccaaacaaaaaactatcgtggctgcaagatgtccacaatttatcgataggtgggaaaaaagcaagttaacaaaatatgtccccaaagaagcacgtgcatctgggctccatggctcacacctgtaatcccagcacttggggaggctgaggcaggagggccacttgagcccaggagttcaagaccagcctggacaacatagcaagaccccatctctacaaaaaatcaaaatgccaaaattagccaggcgtggtggtgcgtacctgtagtcccagctacctgggaggctgaggtggaagggtctcttgagcccaggagtttgaggctgcagtgagtcatgatcacgccactgtactccagcctgggtgacagagcaagaccttgtgtcaaaaaaacaaaaacagtttccattccatcatctatataaatatatggccagaaatcctgtgatggtgatgacctctggggttggggttcttttaatcatcttctttatactcttctgtattgtttgaatttttttttcagtatactgcttttatcattgaaaaagtttttgctgggcaccatggtgcatgtctgtaatcccagctacttgggtggctgaggcaggagaattgcttgaacccaaggggcagaggttgcagtgagccaagatcttgccactacactccagcctgggcaacagagtgagaccctgtctcaaataaaataaaataaaataaaaatttcctgggggtgggggtagagaaccagccaaagatgtcatcggctgggcgtggtggctcacgcctgtaatcccagcactttgggaggctaaggtgggcagatcatgaggtcaggagatcgagaccatcctggctaacacggtgaaaccccgtctctactaaaaatacaaaaaattagccgagcatggtggagggtgcctgtagtcccagctactcaggaggctgaggcaggagaataacatgaacctgggaggtggagtttgcagtgagctgagatggcgccactgcactccagcctaggcgacagagcgagactctgtctcaagaaaaaaaaaaagatgttatcatcaatactctggaaagagacccctctggcagggctggaagcacaggtctaccatcagtgctccctactctggaaatctttgcttcccttggctagttgctcacctcctcagccacgatctcacttcttgttcaaaagcctgcacttcactctcacctagatgaagttctcactcctctataaaatggaggtgataacaagagctccccagagaggactatgaggctgcatgatgcatgacaggtcccaagcacagaagtggaccgtgttgggttcagagtccacgctaataaactacagcccctgcggtttgatgccagctccgccctgactcaccgtcagaaagctgtcttcattctagctcagctcaaaaggcagaaggtcaatgggatgttaggctggataaaacaactccataccatgtcccaaacctggttccagtacctagagagaaggcgactctggtctgccttgtggcagtttaagctctggtgtttcgtgggtgcttgggggctgctcccagactggtgaggctttgagggaaagggggactcagagcaagtgaggatgcctgctgttccctgggagaggctggcagctgcttagcctatgaaagtgcttagtgtgggatgaatgactgtcacaataatgatcattattaattgatggtggcacctgggtccctaaatggtgacacctgcctggtccatgatggggttctaggcagcagtgctgaagggaggatggaggaggcatttctgggcacaaggagaccctctgcccatgggcaaagctgagacaactgacctgaacctaagattcaagtgcctgaactgagtcctagggttcctggggcttagggccagggcacctagaactgcctcttctgccaaggccttggggaagatctgcatctccaatcagcccaaatcctgcagagctggtcgagtgtctgggtatcaccattcctcacccagggatagaggcaggacaccctctctgatgaaagatttagagctcaggaagtgaaatcttgccagactcatcttctgagccctggtcagatgctctgcctcctggaagccttccaggctgagttagttgttcattcttctgggttcccacagattttagataaaccttggtccctggttgtgtctcagcatctttaagccctggagtttggcaagtctgcgtatgtactccagctctgtctgccctctactagctgcgtgaccttgggcaggtgacgttacctctctgagctttggtttctttaccaggagggtagactaacaggggtgcttgatgcatcagctgtgagacttaaatgaaatgcaataggtggggcaattagccctctggcaccccagcccacctgcacctctctgtccctcactcagccttagagcctgttctgggctctctgcaggcgtctgcggttgctgtgtccttggcccaaggatacctggaatctttctgtgcccttggatgaaagagctgcttgcttgttggggtccaaagagaggtgaggactggccagagctgtccagtggaggcagaaggcctgggcaacacctgcagggtactggggtgtgccacctgctatccccaggaagcaaactcatgaatattcacaccactggagtgggagggagccagccagcttgctgctgggtgtcattggagcattcctgtctcccagtgatcgcagaggcagcctgggagttggacgtggctcaggcagtgggtagaaaggggcagccagccacagcccgaggtgagtgtgtctcttttgctccaccacaggggttacagggaaagtaggataccctgagacctgactggtggtgatgctctaagccagaaggtttaccatcctgggctgagtttccctttgtgcatgatgtgggggaataagtcctgtttctccttcattccctggagggcatcagttcagctaaacaggaggatggctgggcctggcctgctgccagctgcctgcactgatgcaggccaatgtgatgggcgtaatctagaagggcaggaatagatggagtgggctggtgacggggggaccgtccatcctgagtgcctgcagttgtcactactgtgggtggaggaggtgggagacccaggttctagtcctgtctctgctgctgctgggctgtgttaccccaaaggtatccttgccctctctgagtctgagcctccctgcctctttgtcttctgaccctgaggctaggagtctctggctacattgagatcatccctggacttggctgcctgcaagcctccatcgtgggccagtgggcacacactttgatggggttgtggctgggaccctgggaacgtaatgatgataatgatgataacaatgatggcaggccctgctgagtgactgtctgctctgcaccagcctcgggctgggtgctttccctgtggaatgcagaggatctgcacagcaccctgtgatggagggtcagctggtacaggttgagtatcccttatccgaaatgcttgggaccagaagtgtttcggatttcagatttttcagattttgaaatatatgcatatatgtaatgagatatcttggagatggaacccaagtctaaacatttaagtttagaaagaatttatgtttctttctttcttattgattttattgtttaagcttttttttttttttgagacggggtctcgctctgtcgcccaggctggagtgcagtggagtgatctcggctcgctgcaagctccgcctcctgggttcacgccgttctcctgcctcagcctcctgagtagctgggactacaggtgcctgccaccaagcctggctaatttttttttgtatttttagtagagatggggtttcactgtgttagccaggatggtctcgatcttctgacctcatgatccactcacctcggcctcccaaagagctgggattacaggcatgagccactgtgcccagcctaattttaatttttttgagatagggtcttgctctgtagcccaggctggagtttagtggcacgatcatggctcactgcagcctcgacctcccaggttcaggtgatacttccgcctcagcctccccagtagctgggactacaggcatgcaccaccatgcctggctaatttttgtattttttgtagagatgaggtcttgccatgttggtcaggctggcctcaaactcctgagctcaagtgatcctcctgtgtctgtctttcaaagtgctggaattacaggcatgagccaccacaccccatcttcatgtatgtttcttatacacatagcctcaaggtagttttatttttcccttcaggatgctaaataaacctgcgctttgactgagacctgtcacgtgaggtcaggtatggaattttccatttgtggtgtcatgtaggcattcaaaaagttttggatttagggacatttcagatttcaatttttatttttattattttttaatccatttatttatttttgagaaagagtctctctctgtcacccaggctggagtgcaatggcgcaatctcagctcactgcaacctctgtctcccaggttcaagcaattctcctgcctcagcctcccaagtagctgggattacaggtgcgcaccaccacacccggctaattttttgtatctttagtagagacggtgtttcagcatgttggccaggctggtcttgaactcctgacctcgtgatctgcctgtctcggcctcccaaagtgctgggattacaccgggcctggcccatgggagcttttacatgcatgtgcctcacatgtattactttcgtctgtaggtctcctacacaggatctctcctgtgtctcagtgaggtgttgtctgcctggaccatgtcctcatccacattctctgcacgggctctatagccctggacaccttctgccctgagggtcatgggaagtcattcatcacacattcaccaggtgccaggctcggggagctcagggatgagtcactggtaggcttgcctgcagcggggcatggaagggacctcacctctggcacgccggtgaaccaaggctgggtcagagtcaatggtatgataataattaatatgagttgatctcctgggtacgtgcattatctcagtctcacagccttagggcatggcatggtggcttacacccagcactttgggaggtggaggtgggaggatcacttgaggccaggaactcaagatcagcctgcaacatagtgagagcccccatctctacaaaaaattaaaaacttagctgggcatgggtggtatgcacttgagcggccgaggtgggaaattcacttgaggccaggtgttcgagaccagcctgggcagtagagtgagacccccatctctacaaaattttttttaaaaagtagccaggcatggtggtgcatgcctgttagtcctagctactcaggaggccgaggcaagaagatccactgagcccaggatttcaaggctgcaatgagctatgatcgtgccacttcacttcagcctgggtgatggagcaagatcctgtctttttaaaaaaattatctcatagcctagtgactgttctccctcaagccaggctcctggctgccagctagctcctctcccgacctcagtctgcccaggggctctgcctgtgattacagagcgctctgtgtgccttgatcttgctgttgtgtgtccattgcacacacgaggagactgaggcttagggagggtgaggttcatagcttcatgcctaagtttacagtagctaggcaggaaaggcacaaccagccttggacccctcaggtggctggcacgggagggacggacaccagggccccctctgctttccctccccttatcctggccgcaggctcagcccacagagacgggaccctgctgtgctcccaggatagagcttcctgtccaaccccacatcccccacttcctgcaatgcaactttccctccttccttcccctgggcttgggacagggcttaggacacaccaggcaggggctgccatggacaaagcaggaagcagcacattggagttccatagacctgggctctaattccagccctgcccccgctttgctgaatgaccctggacaagttatgtcacctcctggaacctcacctttcccatctaccaaatgggggtgacagcagggtggggggtggagtggtatccactgagtggttgctgagtagggtcagttagatgctctggagaggcaggcccctgctttgggcctggctcctaacaggggctcaaaagtaaaactaggagggatagcccaaggttcagtggctggcagcacccagcacgtgtggcaggggcctggcaagctttgagaaatagcaaatgggggccaggctcagtggctcacgcctgtaatcccaacactttggaaggccaaggcgggtggatcgcttgaagtcaggagtttgagaccagcctggccaacatggtgaaaccccggctctactaaaatgcaaaaattacttgagtgtggtggtgggcacctgtagtcccagctactcgagaggctgaggcaggagaatcacttgaacccaggaggtggaggctgtagtgagccgagatcacaccactgcactccagcctgggcaacatagggagacaccgtctcaaaaacaaacaaaagagcaaatgggagaatgaaaccagtgagtgaatgagtgaatgagtgtaccagtaatggggttgggggggtgcgggtggaggaactgtcctgggcggggccaggtggagcctgattggcggggcagggaggctggtctgccccctgctgctgggcctgaacctcagcgcctccactattgcagcctccctgattccagaaggtaccttggtgagtggggctgctggggccggggtgggtccttgggatttccagagggcaagccacaggaatcctaatccctcctcatctttaagtcagagaaactgaggcccagagttttggactatgatctggtccacgactctgccctgttttctgctgggtcgctgagaagtgggaggctgacaggggtgcttgaggctgaggcaggtttgtgctttgtgggcggcccagcccctactccttttggggttgaaggcaggccccccgggcctcaggaggggtacagataacactctaagtgatgactatgtgctggttgcttgcttagaaaagttctccagagaatggctaggggcgcctccagctttctcattttacagatgaaaaactgagactccatgagggttcttggcttgtctgaaatcacagtgctggattgaaacacaggcttgagggtcccagagcctgaggttggtgctggggcccctccatggctgccgggtttattccgctttgcaagctttctgatttgttacttaaatgtgttaagtaacaaattaactaacacatttgttaacgtgttgacaattattgcctttttttgagatggggcctcactctggtcacccaggctgaagtgcagtggtgcgaccttggctcacggtaacctctgccaacctctgccccccaggctcaagcaatcctcccacctcagcataacttttccagggcatagtgtgtgtgaccctcccccagggggactgactccagggacagtggtgataatgcatgcatgtagttattagcacatgtgacttttcctggcctcaggggcccagggcgcagtgcagtgtgagccaggacagaagtggaaagttcgggacaagagtagaaagtttggggacctggttttgcagctaaggggaggtgggaaggccctccaagccgcagctctctcctggactccagttcctcagcctgaactgcccccgttacagtccatgtctcagtcccctgctgatagggttgccacgcttagatcatcccccaaaacaaataacaacaggctgggcacagtggctcatgcctataatcccagcactttgggaggctgaggtgggaggattgcttgagtccaggagttcaataccagcctgggcatctcaaaaataataatataaaataaatattaaaaatgtataaaaatgggccaggcgcggtggctcatgcctgtaactctagcactttgggaggccaaggtgggcagatcacaaggtcaggagatcaagatgatcctggccaacatggtgaaactccatctctactaaaaatacaaaaattagctgggtgtggtggcgtgtgcctgtaatcccagctacttgggaggctgaggcaggagaatcgcttgaaccggggagtcaaaggttgcagggagccgagattgcgccacagcactccagcctggcgacagagcgagactccgtctcaaaaaaataaaataaacatataaaaatgaaaaaataaggaaataacaacaaaacctccaggacatctagttaagtttgaagtttgaataattttttttttttgagacagagtctcactctgtcacccaggcaggaatacagtggcaccatcttggctcactgcaacctctacctcctgggttcaagcgattcttctacctcagcctcctgagtaactgggactataggggcatgccaccacacccagctagtttttgtattttcagtagagacagaatttcaccatattggtcaggcttgtctcaaactcctgacctcaggtgatccaccctcctcggcctcccaaagtgctaggattacaggcgtgagccaccgtgcccagccttggaataatttttagtataactatgtcccatgcaatatttggaatatacttatactgtaaaaaattgtattcattgtttatctgaaatttgaatttaactgagcaaccctacaggctggctttggaaagggggtgggtatgtgagcagaggcctggccagctgtgtgacttgagccttcttcttcccagctgtgagcctcagtttctccatcagccaccctacagactctaggggcacaagggccagtgaggagagaagagccctgggctcttactgggcacctcacctgtaccactgtggggtgggtggcacaggggacaggtagggctggctgtgccttctgaaaggtgaaggattcaggccccagagagcccttggccttccctatagacagccctggcccaggcctgggggagggagggcccagggcacggggagagggtcttcttttccttcttatttatttgtttatttatttgagacagggtctcactgtgtcatccagagctggagtgcagcggcacagtcatggctcactggaactcaggctcaagcaatcctcccgcctcagccttccaagtaactaggactacaggcatgtgccaccacgcctggtgaatttttttaaattttgttttgtagagataggttctcaccatatgcccaggctggtcttgaactcctggctcaagcactccgcctgcctcatcctcccaaagtgctgggatgacaggtgtgagccactgtgcccaggctttttccttttgccttagaaaagtgtttcatggtcgggcgcgatggctcacgtctgtcatcccagcaatttgggaggccaaggcgggcggattgcttgagcccaggagttcgagaccagcctaggcaacgtggcaaaactgtctctatttttaaaaaaattaaaatataaaaaaggaaataatttcattatgatgccctaaaaggtataaaaaataacttcaggatctgggagaatttgaagtaaaattgaagaatgttttaaatatatatttttataaaaaatacaagaaaaagaaacagaagaaaaaataagataactaaaaaaaaaaaggtaataagtattagttgtctcattttttcttctgtttctttttcttttatttcttatcagccagcttaaaaattaaaaggagtggaaagtttggggccctggttctctacccggttcattttaatttttaaattttaaccttctccctgccccattccccaacctctctttgagttgcttcacccaccgtctctgcaaccctggggctgtctctttccaggtggttgtcagtctatctctccagtgcctctctgtccctgtctggaccctgctggggggccacagagcaggcaaaggcagggctcaactgccaggcctgggcagccttgggggctccctggggccagcttcctgacacttgccctactctgtgctgcccctgcaggtgtctgagatgctgccaccgcagaagaagccctgggagtccatggctaaggggctggtgctgggcgcgctcttcactagtttcctgctgctggtgtactcctatgccgtgcccccgctgcatgccggcctggcctccacgtgagtggcccagctgggcagatgatgggtgtaggtgggggagggatggcaccccacccatggggccctgtaattaccacagggcctgccccagtgatcttgggtagccaccctgcaccctgcaccatgctgtgccagacagaaattccactgatggcagagctcgccgtgtaactctgagccttggtgtgcccatctgtgaagtggggataaaaacagagcctttcggctgggcacggcaactcacgcctgtaatcctagcactttgggaggcccaggcgggcagatcgtctgaggtcaggagttcgagaccagcctggccaacatggtgaaaccccatctctactaaaaatacaaaaattagctgggcatggtggtgtgtgtctgtaatctcagctacttgggagtctgaggcaggagaattgcttgaacccggaaggcagaagttgcagtgagccgagatggcaccactgtactccaggctgggccaacggagtgagactgtctcaaaacaaaaagaaaaacaaacaaacaaaaaaaacccagagccttcctcatagagcatgagaatcaggtaacactgtctgtacagggctcagatttgcatcatctgagatgccaacggggccctggtgggggcgacctgtccatgggcatggattcaaacgggtcggtctgactccaggcccagctcctgagaaccaggctctcccgtttcctcatggggctgctctgcacagacccagcacagcctgtagctggtgcaataccagtggttggtgatattaacttgacacagaggaaactgcaaatttctgcaaaaatggaccatttttgtcctataaaatggcagtttcatgtggcccaagctgatactgattcatggtacatgcaacacagtggccatgaatgagaggagagcctgtgtgcctcatctcacaacaccgtgaatagggcctgttatcgtccccatttcacagaagaggaacatgaggctagagaggccaaaccacccgcgcaggcacccagcctggactcaggccccacaggtctcactatcccagctcctttgccaccactgttcccaataatctccctctctaactaccagagtcctgagggccccagcacccccctgaggctctccccacctctctgtccccagcaggaccccggaggccgcagcgtcctgctctccacctgcactcgagccagaggcagtgatccgggccaacggctcggcgggggagtgccagccgcggcgcaacatcgtgttcttgaagacgcacaagacggccagcagcaccctgctcaacatcctgttccgcttcggccagaagcaccggctcaagttcgccttccctaacggccgcaatgacttcgactacccgaccttcttcgcccgcagcctggtgcaggactatcggcccggggcctgcttcaacatcatctgcaaccacatgcgcttccactacgacgaggtgcgcggcctggtgccgaccaacgccatcttcatcacggtgctccgcgaccccgcccgcttgttcgagtcctccttccactacttcgggccggtggtgcccctcacgtggaagctctcggccggcgacaagctgaccgagttcctgcaagacccggatcgctactacgaccccaacggcttcaatgcccactacctccgaaacctgctcttcttcgacctgggctatgacaacagcctggaccccagcagcccgcaggtgcaggagcacatcctggaggtggagcgtcgcttccacctggtgctccttcaagagtacttcgacgagtcgctggtgctgctgaaggacctgctgtgctgggagctggaggacgtgctctacttcaagctcaacgcccgccgcgactcgcccgtgccgcggctctcgggggagctgtatgggcgcgccaccgcctggaacatgctggactcccacctctaccgccacttcaacgccagcttctggcgcaaggtggaggccttcgggcgggagcgcatggcccgcgaggtggccgccctgcgccatgccaacgagcgcatgcggaccatctgcatcgacgggggccacgccgtggacgccgccgccatccaggacgaggccatgcagccctggcagccgctgggcaccaagtccatcctgggctacaacctcaagaagagcatcgggcagcggcacgcgcagctctgccggcgcatgctcacgcccgagatccagtacctgatggacctcggcgccaacctgtgggtcaccaagctctggaagttcattcgcgatttcctgcggtggtgacgtcccaccgcccagcggcttgcctgcctgctcgctccctgcagaggggctgagcaggacgccgctggtgctggccgcccccagccccctcctggtgccacctcagaccccggggtgagggggggctccctggggggaggcagccagccaagactgggcccatgaacacagagagggcctaaccgagatcagtatttaactaattataccagtttttattaaacccctttccctccccgataaagaatgttctatttctgcctccccttaaaggggagacctcagaagtaaaggaatttgatgttgtgtttttgttaa

SEQ ID NO: 634 - RNA, 19944 nt, synthetic construct
  source 1..19944: mol_type = other RNA
aggcctggtggagcggggggcccggccgggtgcgccccgccgcccagtgagcgcccgggcggcgcggcccgggccgagccagccacgctgaggcggaggaggacggggacaaggtacgtcccgggctccagcacccactgccagcttgagccctgcctcagtctactccctggggataccgcccacattacgctaacccccaaactgggaccacgcgccggaggaggggagtaggagtgcagtaccgctgttctaagcgctgggtacccctgcccactgccagagggtggcctggtaggggccagggagggcacaggttggtgccccccatgcccttggttttttcccctgtcccccaaccccccttccagggcttcccagtccctgaaggtgggatgggtggggcagaaggcaggctgctggggctgagctggagctgggagtgcgttaatgattaatcttggacacagtggccagagccctaggggaggaggggcagagatctcccctgtctccatcagcccttcctagaaaggggatcagagcaggaaccggagaccatagtaagggatggatcccctctgctgggggaaggccctggcctgtaccccagaggaagcaaagccaccaaggagaggtttgggagagtgggacaaaccctggcatccaggaggggtcctataggaaatcctacagttcctgtttcctagcccagtgcccaggaagggggtccagcttgatctgccccttcggtgccaggccagcattttggtaccagcacagagcacactacagaccgagccctgcccagtgggaactgccccgcccagctgcccagagccagagaggtgatgggcatgtctggctgctgtaacccacaccagggggaaggaaagggagagaaggcagcatgcctgacacctgatccccgggctgccgcggtgacaggtgcatgagaatcctccagaggggagagaggtgtagaggactgtccattccagttgggaaaacccaatctcttcctttactgacggggtgacctgaggcccagagagggtgaggctcatccaggcccacacagggggcacaggagggcagtcctggaccagagcagtcactgtgtacgcactgttccttgtcctgtgaaggaggcattgctacctatccccattatccagagggggatactgaggctcagggaaattcaatgacttgtccaaggtcacatggctcagaggtagctgagctgactccaaatcataggagtcttgccctggcccccatttcaggaccccagcccaacccatgacctctctctgctcctttggggtcctgccttcttggatctgcctgggctgccccaaaccctcagctctggacaccccccctcgcctccctggctcccagctccgcctgacacttcagcgggtgagcaggctgctaccccgggagccgagacaacgaagccctgttcccagctgccacctcagcctgctttattccctgcttgctcaggcagagcccttcctggaggaagctccttgcggacagagaatgcctggtccccatgcatccccttcccttaatgggtccgatgccctcttgtcgcccagcaagggaggagtgtgtcccccacagcccctcccactgtgttctgagggactgggtgtgccatccagtgaaagaggcaatgattaacctgagagaatgatgcacaaatggaattctgggcccggctgcaggctcctgaagggctccttgcttcactttgactgggtgaggaatatggatgagcccttttaggggctccacacgactccaaacagggcagagcaggatggaacacaggtgcccatgggggtcatcaggctggggactgctggaatctccaggtgaggggtgatggtgggaggaagtgggggcccagaagatgcctgccttccctcctgctagttctggctccttggatggggatggtatccttctagaagtctggacagagcccgcttcccctaccaggtgggatctaggaggatcacaatacaatcagaggagctaacgctgagtgtgtgaagggctctgcgtgcctcacagcaggcctgtgaaagcatcatagtacctgccattttttaggaaaccaaggctcagagaggggaagtcacttgcccaaggtcacccagcttggggggtggtggagccagcatttgttaccaggcgagtttccaggaatggagaggtgggtgtatgtatctccggcattgtgcattgcaagtgtcaataccttttcacaaacctttatattgttacatagttttaggcaaataagcgcataagaatccttaaaaaatatttcaggcatgttgtaaggtacagagactgatattatcaactcctacatactcagctctggggttctcagcatttttggagctaatgacaaagtgactgacttcttggtgaagctgtgacttgcaacgccccagttagcgcttctctaaggctactcaaatccccccctcaggaagcggggtgatttacccagacaaggcagtgtagcaggaagagctgtggccttgctcctcattttggctttgccgtgtaggcaaaaaaactgagcctcagtttccccacacatcacatggggctagcagaggagactgggagtgtctgtcaaggtcacaggcatggatgccctttgtttttgtttttgtttttgagacggagtcttgctctgtcacccaggctggagtgcagtggtgcaatctcggctcactgcaagctccgcctcccaggttcacaccattctcctgcctcagcctcccgagtagctgggactataggcgcccaccaccaagcctggctaatttttttataattttagtagagacgggatttcactgtgttagccaggatggtctcaatctcctgacctcgtgatctgcccgcctcggcctcccaaagtgctgggattacaggcgtgagccactgcgcccggccgtggatgccctttgaaacagcagagagagatgaccccgaaagttcaaaaagtgctttctgaatcatcaaagaacattaccctctctcaactgctgggccagctaccacctgcatccacccctgcaggctaggtcgcagcaggggccagatgtctgggctgacattttggaagtggtcctctgagccagcgagttgggatccagctgaaatgctccaataggtggtcttccccctctctgctccagcaccccattctagaggggctcagattgaggaagcttctaggtgcttgcaagtcacttccccagtacccctggatggagggttaacctatcccatccgccccttctgctgtgccacccacacttcccagctccttctcagtcccaggcccctgttgccccttcctcacttggtgcactgaggttcagagagggcagagtgtcttgcctcggccacacagtgaggagaggtggggctcatcttcctccagccaggctgcagattttcaaagtggaaatatctttatttatctttccaaagtaatataagctgttttgtaaagagtgtgtgtgtgtgtgtgtgtgtgtgtgtgtgtgtgtgtatcatcacagaatcaaaatatgttccagaaagtgaatgggcttactccccagagagctacaatttataactgggtgtctttccagacccttatacacatgtacgaatgtacaacttttctctacatgaataagtttattccataaacagtgcattccaggcttaatattaggcaactctttttttttttttgagacggagtttcgttctttttgcctaggctggagtgcaatggcacgatattggctcattgcaacctccgcctcccaggttcaagtgattctcctgcctcagccttaggattaaaggcatgcgccaccatgcccagctaatttttgtatttttagtagagatggggtttctccatgttgtcaggctggtctcaaactcccgacctcaggtgatccacccgccttggcctcccaaagtgctgggattacaggcgtgagccaccacatccggtcaggcaactcttttttaatctctccgagcctcagtttcctcatcttctaaatgggggtaaaggctctgtgagcactggaggaaatgatactgcagtggccacttttatatgcccatctcagattgattgttagattatctttgagccacattcctgcaggtggattgctgggtctgagaacaggcacactcagaactgtaatagctgctgtcaaatctgcactacgaacatcatagggagagttgttttgctccactcttgaaagctccttccatatcccgatgggattccactccagctacagaaagccctgggatacctcaccccaccaccttctctgccagagccctgttcctgtatgtgcatatgagcaaacagcacggatgtgcctcagttggtgaatggcttttgtttcttggagtttctttttttaagatggggtctcacactgttgcccaggcttggagtgcagtggtacgttcatacctctctgtaaccttgaacttctgggttcaaggggtcctcttgcctcagcctcctgagtagctggggctacaggcgtgcaccaccacacagggctaatgtttaaattttttatacagatggggtctcactgtgttgcccaggcaagtctcgaactcctgggctcaagcaatctgcctgtcttggcctccccaaaatgctgggtggcgcacgccaccgtgcccggctggggaatggtttatgggggaatagcggtgctgcaaactctgatacagccctagctgccctgttttgtctccttagggagctcttcttcaccctgctccgtaagcacaggctccctgtccttcctcacccttccctggtgactctagttattcctatggcctcaattcccactttggtgcccagctccccgcctacctctttccctcccccaagacccccctctttgctcctactgctcttcaggcattctatgatcatcccaaacatccctcagactcagagtattctaaacagaaccagacaccacctgcttcacccagcattcaacctggccctttctcagcctcccagactcagggactagctctacctccagccagccagtcacccaacagcaactcttccctcctctcctccatacctgctgccaataggatgctacctttgcatggtctacagacagctctggtacttccccagccttcttgttccaggtcacacctgcacagctgtaacaactactatggaggtcggcctgctcctccctctccttccccatccctacagttcatttcttacccagtagcccaatgggaatcccatctgctccctcctctgctgaagaccctcctgtgggtctgtccctacagcccacaggatctaggtgagactcagtagcatgcttcctgaaagccctgcatgtccctctttttttttttttttttttttgagatggagtctcgctctgttgcccaggctggagtgcagtggcgcgatctctgctcactgcaaactctgcctcccaggttcaagcgattatcctgcctcagcctcccgagtagctggtattacatgcgcctgccatcacgcccggctaatttctgtatttttagtacagacggggtttcaccatgttggccaggctggtctcggacccctaacctcaggtgatctgcccgccttagcctcccaaagtgctggtattacaggcgtgagccactgcgccaggcctctgcatgtccctcttaaccctgccctagtttctggctgtctctgtagtctctcccctgcccacccagcctattacacgcaaacacaccaaacactccagccaggtggaaagtttgtttctcaattgccctgggcctttgcatgtgcttttctctctgtgtggctcattctagggtaacaggcatattagttaatgctagctgctgtaacagacaaacctgaagttctaaaaggcttaatacagttctcgttttcttgaaatccagtcagtgtctggtgggtggagatgcaaagacctaggctcctcccagctccgcccatgcccagatccttggagaccaccccgttcatccagtaaatgggaaaggagaccacagaaaaggtccacccacttaacctcatcagctggcattctgttcacatatcattggccaggacccagttatgtgaggacactgaactgcaagggcgcatgggaaatatagtctagctgtgctgccgtggaaaaaaggggactgtctttggaaattggagaccacacaacagcttctgccaccagaggcccctaccccaggaagccttcctgatactgctgtctccgccccaaggctggatcaggtgttttcttcgtgcttctgcaagcctcagacaatccgttgtccatcacacgcctcacagctgtgtgttcctgtgtctccccagttaggactgtgagccctgagttctctgcaccgggttatcatgaactctagtcctggagtgcaggtgcccagtaaatacctgtgggatggatcaatttaaaactatcacgggctgggcgcggtggctcacgtctgtaatcccagcactttgggaggctgaggcaggtggatcacttgaggtcagaagtttgagaccagcctgcccaaggtggtgaaaccctgtctctactaaaaatacaaaattagctgggtgtggtggcgggtgcccgtaatcccagctacttggaagggtgaggcaggagaattgcttgaacccagggggcagaggttgcagtgggccgagattgccccagtgcactccagcatgggtgacaagagtgcaactctgtctcaaacaacaacaacaacaaaaaacaaccaaacaaaaaactatcgtggctgcaagatgtccacaatttatcgataggtgggaaaaaagcaagttaacaaaatatgtccccaaagaagcacgtgcatctgggctccatggctcacacctgtaatcccagcacttggggaggctgaggcaggagggccacttgagcccaggagttcaagaccagcctggacaacatagcaagaccccatctctacaaaaaatcaaaatgccaaaattagccaggcgtggtggtgcgtacctgtagtcccagctacctgggaggctgaggtggaagggtctcttgagcccaggagtttgaggctgcagtgagtcatgatcacgccactgtactccagcctgggtgacagagcaagaccttgtgtcaaaaaaacaaaaacagtttccattccatcatctatataaatatatggccagaaatcctgtgatggtgatgacctctggggttggggttcttttaatcatcttctttatactcttctgtattgtttgaatttttttttcagtatactgcttttatcattgaaaaagtttttgctgggcaccatggtgcatgtctgtaatcccagctacttgggtggctgaggcaggagaattgcttgaacccaaggggcagaggttgcagtgagccaagatcttgccactacactccagcctgggcaacagagtgagaccctgtctcaaataaaataaaataaaataaaaatttcctgggggtgggggtagagaaccagccaaagatgtcatcggctgggcgtggtggctcacgcctgtaatcccagcactttgggaggctaaggtgggcagatcatgaggtcaggagatcgagaccatcctggctaacacggtgaaaccccgtctctactaaaaatacaaaaaattagccgagcatggtggagggtgcctgtagtcccagctactcaggaggctgaggcaggagaataacatgaacctgggaggtggagtttgcagtgagctgagatggcgccactgcactccagcctaggcgacagagcgagactctgtctcaagaaaaaaaaaaagatgttatcatcaatactctggaaagagacccctctggcagggctggaagcacaggtctaccatcagtgctccctactctggaaatctttgcttcccttggctagttgctcacctcctcagccacgatctcacttcttgttcaaaagcctgcacttcactctcacctagatgaagttctcactcctctataaaatggaggtgataacaagagctccccagagaggactatgaggctgcatgatgcatgacaggtcccaagcacagaagtggaccgtgttgggttcagagtccacgctaataaactacagcccctgcggtttgatgccagctccgccctgactcaccgtcagaaagctgtcttcattctagctcagctcaaaaggcagaaggtcaatgggatgttaggctggataaaacaactccataccatgtcccaaacctggttccagtacctagagagaaggcgactctggtctgccttgtggcagtttaagctctggtgtttcgtgggtgcttgggggctgctcccagactggtgaggctttgagggaaagggggactcagagcaagtgaggatgcctgctgttccctgggagaggctggcagctgcttagcctatgaaagtgcttagtgtgggatgaatgactgtcacaataatgatcattattaattgatggtggcacctgggtccctaaatggtgacacctgcctggtccatgatggggttctaggcagcagtgctgaagggaggatggaggaggcatttctgggcacaaggagaccctctgcccatgggcaaagctgagacaactgacctgaacctaagattcaagtgcctgaactgagtcctagggttcctggggcttagggccagggcacctagaactgcctcttctgccaaggccttggggaagatctgcatctccaatcagcccaaatcctgcagagctggtcgagtgtctgggtatcaccattcctcacccagggatagaggcaggacaccctctctgatgaaagatttagagctcaggaagtgaaatcttgccagactcatcttctgagccctggtcagatgctctgcctcctggaagccttccaggctgagttagttgttcattcttctgggttcccacagattttagataaaccttggtccctggttgtgtctcagcatctttaagccctggagtttggcaagtctgcgtatgtactccagctctgtctgccctctactagctgcgtgaccttgggcaggtgacgttacctctctgagctttggtttctttaccaggagggtagactaacaggggtgcttgatgcatcagctgtgagacttaaatgaaatgcaataggtggggcaattagccctctggcaccccagcccacctgcacctctctgtccctcactcagccttagagcctgttctgggctctctgcaggcgtctgcggttgctgtgtccttggcccaaggatacctggaatctttctgtgcccttggatgaaagagctgcttgcttgttggggtccaaagagaggtgaggactggccagagctgtccagtggaggcagaaggcctgggcaacacctgcagggtactggggtgtgccacctgctatccccaggaagcaaactcatgaatattcacaccactggagtgggagggagccagccagcttgctgctgggtgtcattggagcattcctgtctcccagtgatcgcagaggcagcctgggagttggacgtggctcaggcagtgggtagaaaggggcagccagccacagcccgaggtgagtgtgtctcttttgctccaccacaggggttacagggaaagtaggataccctgagacctgactggtggtgatgctctaagccagaaggtttaccatcctgggctgagtttccctttgtgcatgatgtgggggaataagtcctgtttctccttcattccctggagggcatcagttcagctaaacaggaggatggctgggcctggcctgctgccagctgcctgcactgatgcaggccaatgtgatgggcgtaatctagaagggcaggaatagatggagtgggctggtgacggggggaccgtccatcctgagtgcctgcagttgtcactactgtgggtggaggaggtgggagacccaggttctagtcctgtctctgctgctgctgggctgtgttaccccaaaggtatccttgccctctctgagtctgagcctccctgcctctttgtcttctgaccctgaggctaggagtctctggctacattgagatcatccctggacttggctgcctgcaagcctccatcgtgggccagtgggcacacactttgatggggttgtggctgggaccctgggaacgtaatgatgataatgatgataacaatgatggcaggccctgctgagtgactgtctgctctgcaccagcctcgggctgggtgctttccctgtggaatgcagaggatctgcacagcaccctgtgatggagggtcagctggtacaggttgagtatcccttatccgaaatgcttgggaccagaagtgtttcggatttcagatttttcagattttgaaatatatgcatatatgtaatgagatatcttggagatggaacccaagtctaaacatttaagtttagaaagaatttatgtttctttctttcttattgattttattgtttaagcttttttttttttttgagacggggtctcgctctgtcgcccaggctggagtgcagtggagtgatctcggctcgctgcaagctccgcctcctgggttcacgccgttctcctgcctcagcctcctgagtagctgggactacaggtgcctgccaccaagcctggctaatttttttttgtatttttagtagagatggggtttcactgtgttagccaggatggtctcgatcttctgacctcatgatccactcacctcggcctcccaaagagctgggattacaggcatgagccactgtgcccagcctaattttaatttttttgagatagggtcttgctctgtagcccaggctggagtttagtggcacgatcatggctcactgcagcctcgacctcccaggttcaggtgatacttccgcctcagcctccccagtagctgggactacaggcatgcaccaccatgcctggctaatttttgtattttttgtagagatgaggtcttgccatgttggtcaggctggcctcaaactcctgagctcaagtgatcctcctgtgtctgtctttcaaagtgctggaattacaggcatgagccaccacaccccatcttcatgtatgtttcttatacacatagcctcaaggtagttttatttttcccttcaggatgctaaataaacctgcgctttgactgagacctgtcacgtgaggtcaggtatggaattttccatttgtggtgtcatgtaggcattcaaaaagttttggatttagggacatttcagatttcaatttttatttttattattttttaatccatttatttatttttgagaaagagtctctctctgtcacccaggctggagtgcaatggcgcaatctcagctcactgcaacctctgtctcccaggttcaagcaattctcctgcctcagcctcccaagtagctgggattacaggtgcgcaccaccacacccggctaattttttgtatctttagtagagacggtgtttcagcatgttggccaggctggtcttgaactcctgacctcgtgatctgcctgtctcggcctcccaaagtgctgggattacaccgggcctggcccatgggagcttttacatgcatgtgcctcacatgtattactttcgtctgtaggtctcctacacaggatctctcctgtgtctcagtgaggtgttgtctgcctggaccatgtcctcatccacattctctgcacgggctctatagccctggacaccttctgccctgagggtcatgggaagtcattcatcacacattcaccaggtgccaggctcggggagctcagggatgagtcactggtaggcttgcctgcagcggggcatggaagggacctcacctctggcacgccggtgaaccaaggctgggtcagagtcaatggtatgataataattaatatgagttgatctcctgggtacgtgcattatctcagtctcacagccttagggcatggcatggtggcttacacccagcactttgggaggtggaggtgggaggatcacttgaggccaggaactcaagatcagcctgcaacatagtgagagcccccatctctacaaaaaattaaaaacttagctgggcatgggtggtatgcacttgagcggccgaggtgggaaattcacttgaggccaggtgttcgagaccagcctgggcagtagagtgagacccccatctctacaaaattttttttaaaaagtagccaggcatggtggtgcatgcctgttagtcctagctactcaggaggccgaggcaagaagatccactgagcccaggatttcaaggctgcaatgagctatgatcgtgccacttcacttcagcctgggtgatggagcaagatcctgtctttttaaaaaaattatctcatagcctagtgactgttctccctcaagccaggctcctggctgccagctagctcctctcccgacctcagtctgcccaggggctctgcctgtgattacagagcgctctgtgtgccttgatcttgctgttgtgtgtccattgcacacacgaggagactgaggcttagggagggtgaggttcatagcttcatgcctaagtttacagtagctaggcaggaaaggcacaaccagccttggacccctcaggtggctggcacgggagggacggacaccagggccccctctgctttccctccccttatcctggccgcaggctcagcccacagagacgggaccctgctgtgctcccaggatagagcttcctgtccaaccccacatcccccacttcctgcaatgcaactttccctccttccttcccctgggcttgggacagggcttaggacacaccaggcaggggctgccatggacaaagcaggaagcagcacattggagttccatagacctgggctctaattccagccctgcccccgctttgctgaatgaccctggacaagttatgtcacctcctggaacctcacctttcccatctaccaaatgggggtgacagcagggtggggggtggagtggtatccactgagtggttgctgagtagggtcagttagatgctctggagaggcaggcccctgctttgggcctggctcctaacaggggctcaaaagtaaaactaggagggatagcccaaggttcagtggctggcagcacccagcacgtgtggcaggggcctggcaagctttgagaaatagcaaatgggggccaggctcagtggctcacgcctgtaatcccaacactttggaaggccaaggcgggtggatcgcttgaagtcaggagtttgagaccagcctggccaacatggtgaaaccccggctctactaaaatgcaaaaattacttgagtgtggtggtgggcacctgtagtcccagctactcgagaggctgaggcaggagaatcacttgaacccaggaggtggaggctgtagtgagccgagatcacaccactgcactccagcctgggcaacatagggagacaccgtctcaaaaacaaacaaaagagcaaatgggagaatgaaaccagtgagtgaatgagtgaatgagtgtaccagtaatggggttgggggggtgcgggtggaggaactgtcctgggcggggccaggtggagcctgattggcggggcagggaggctggtctgccccctgctgctgggcctgaacctcagcgcctccactattgcagcctccctgattccagaaggtaccttggtgagtggggctgctggggccggggtgggtccttgggatttccagagggcaagccacaggaatcctaatccctcctcatctttaagtcagagaaactgaggcccagagttttggactatgatctggtccacgactctgccctgttttctgctgggtcgctgagaagtgggaggctgacaggggtgcttgaggctgaggcaggtttgtgctttgtgggcggcccagcccctactccttttggggttgaaggcaggccccccgggcctcaggaggggtacagataacactctaagtgatgactatgtgctggttgcttgcttagaaaagttctccagagaatggctaggggcgcctccagctttctcattttacagatgaaaaactgagactccatgagggttcttggcttgtctgaaatcacagtgctggattgaaacacaggcttgagggtcccagagcctgaggttggtgctggggcccctccatggctgccgggtttattccgctttgcaagctttctgatttgttacttaaatgtgttaagtaacaaattaactaacacatttgttaacgtgttgacaattattgcctttttttgagatggggcctcactctggtcacccaggctgaagtgcagtggtgcgaccttggctcacggtaacctctgccaacctctgccccccaggctcaagcaatcctcccacctcagcataacttttccagggcatagtgtgtgtgaccctcccccagggggactgactccagggacagtggtgataatgcatgcatgtagttattagcacatgtgacttttcctggcctcaggggcccagggcgcagtgcagtgtgagccaggacagaagtggaaagttcgggacaagagtagaaagtttggggacctggttttgcagctaaggggaggtgggaaggccctccaagccgcagctctctcctggactccagttcctcagcctgaactgcccccgttacagtccatgtctcagtcccctgctgatagggttgccacgcttagatcatcccccaaaacaaataacaacaggctgggcacagtggctcatgcctataatcccagcactttgggaggctgaggtgggaggattgcttgagtccaggagttcaataccagcctgggcatctcaaaaataataatataaaataaatattaaaaatgtataaaaatgggccaggcgcggtggctcatgcctgtaactctagcactttgggaggccaaggtgggcagatcacaaggtcaggagatcaagatgatcctggccaacatggtgaaactccatctctactaaaaatacaaaaattagctgggtgtggtggcgtgtgcctgtaatcccagctacttgggaggctgaggcaggagaatcgcttgaaccggggagtcaaaggttgcagggagccgagattgcgccacagcactccagcctggcgacagagcgagactccgtctcaaaaaaataaaataaacatataaaaatgaaaaaataaggaaataacaacaaaacctccaggacatctagttaagtttgaagtttgaataattttttttttttgagacagagtctcactctgtcacccaggcaggaatacagtggcaccatcttggctcactgcaacctctacctcctgggttcaagcgattcttctacctcagcctcctgagtaactgggactataggggcatgccaccacacccagctagtttttgtattttcagtagagacagaatttcaccatattggtcaggcttgtctcaaactcctgacctcaggtgatccaccctcctcggcctcccaaagtgctaggattacaggcgtgagccaccgtgcccagccttggaataatttttagtataactatgtcccatgcaatatttggaatatacttatactgtaaaaaattgtattcattgtttatctgaaatttgaatttaactgagcaaccctacaggctggctttggaaagggggtgggtatgtgagcagaggcctggccagctgtgtgacttgagccttcttcttcccagctgtgagcctcagtttctccatcagccaccctacagactctaggggcacaagggccagtgaggagagaagagccctgggctcttactgggcacctcacctgtaccactgtggggtgggtggcacaggggacaggtagggctggctgtgccttctgaaaggtgaaggattcaggccccagagagcccttggccttccctatagacagccctggcccaggcctgggggagggagggcccagggcacggggagagggtcttcttttccttcttatttatttgtttatttatttgagacagggtctcactgtgtcatccagagctggagtgcagcggcacagtcatggctcactggaactcaggctcaagcaatcctcccgcctcagccttccaagtaactaggactacaggcatgtgccaccacgcctggtgaatttttttaaattttgttttgtagagataggttctcaccatatgcccaggctggtcttgaactcctggctcaagcactccgcctgcctcatcctcccaaagtgctgggatgacaggtgtgagccactgtgcccaggctttttccttttgccttagaaaagtgtttcatggtcgggcgcgatggctcacgtctgtcatcccagcaatttgggaggccaaggcgggcggattgcttgagcccaggagttcgagaccagcctaggcaacgtggcaaaactgtctctatttttaaaaaaattaaaatataaaaaaggaaataatttcattatgatgccctaaaaggtataaaaaataacttcaggatctgggagaatttgaagtaaaattgaagaatgttttaaatatatatttttataaaaaatacaagaaaaagaaacagaagaaaaaataagataactaaaaaaaaaaaggtaataagtattagttgtctcattttttcttctgtttctttttcttttatttcttatcagccagcttaaaaattaaaaggagtggaaagtttggggccctggttctctacccggttcattttaatttttaaattttaaccttctccctgccccattccccaacctctctttgagttgcttcacccaccgtctctgcaaccctggggctgtctctttccaggtggttgtcagtctatctctccagtgcctctctgtccctgtctggaccctgctggggggccacagagcaggcaaaggcagggctcaactgccaggcctgggcagccttgggggctccctggggccagcttcctgacacttgccctactctgtgctgcccctgcaggtgtctgagatgctgccaccgcagaagaagccctgggagtccatggctaaggggctggtgctgggcgcgctcttcactagtttcctgctgctggtgtactcctatgccgtgcccccgctgcatgccggcctggcctccacgtgagtggcccagctgggcagatgatgggtgtaggtgggggagggatggcaccccacccatggggccctgtaattaccacagggcctgccccagtgatcttgggtagccaccctgcaccctgcaccatgctgtgccagacagaaattccactgatggcagagctcgccgtgtaactctgagccttggtgtgcccatctgtgaagtggggataaaaacagagcctttcggctgggcacggcaactcacgcctgtaatcctagcactttgggaggcccaggcgggcagatcgtctgaggtcaggagttcgagaccagcctggccaacatggtgaaaccccatctctactaaaaatacaaaaattagctgggcatggtggtgtgtgtctgtaatctcagctacttgggagtctgaggcaggagaattgcttgaacccggaaggcagaagttgcagtgagccgagatggcaccactgtactccaggctgggccaacggagtgagactgtctcaaaacaaaaagaaaaacaaacaaacaaaaaaaacccagagccttcctcatagagcatgagaatcaggtaacactgtctgtacagggctcagatttgcatcatctgagatgccaacggggccctggtgggggcgacctgtccatgggcatggattcaaacgggtcggtctgactccaggcccagctcctgagaaccaggctctcccgtttcctcatggggctgctctgcacagacccagcacagcctgtagctggtgcaataccagtggttggtgatattaacttgacacagaggaaactgcaaatttctgcaaaaatggaccatttttgtcctataaaatggcagtttcatgtggcccaagctgatactgattcatggtacatgcaacacagtggccatgaatgagaggagagcctgtgtgcctcatctcacaacaccgtgaatagggcctgttatcgtccccatttcacagaagaggaacatgaggctagagaggccaaaccacccgcgcaggcacccagcctggactcaggccccacaggtctcactatcccagctcctttgccaccactgttcccaataatctccctctctaactaccagagtcctgagggccccagcacccccctgaggctctccccacctctctgtccccagcaggaccccggaggccgcagcgtcctgctctccacctgcactcgagccagaggcagtgatccgggccaacggctcggcgggggagtgccagccgcggcgcaacatcgtgttcttgaagacgcacaagacggccagcagcaccctgctcaacatcctgttccgcttcggccagaagcaccggctcaagttcgccttccctaacggccgcaatgacttcgactacccgaccttcttcgcccgcagcctggtgcaggactatcggcccggggcctgcttcaacatcatctgcaaccacatgcgcttccactacgacgaggtgcgcggcctggtgccgaccaacgccatcttcatcacggtgctccgcgaccccgcccgcttgttcgagtcctccttccactacttcgggccggtggtgcccctcacgtggaagctctcggccggcgacaagctgaccgagttcctgcaagacccggatcgctactacgaccccaacggcttcaatgcccactacctccgaaacctgctcttcttcgacctgggctatgacaacagcctggaccccagcagcccgcaggtgcaggagcacatcctggaggtggagcgtcgcttccacctggtgctccttcaagagtacttcgacgagtcgctggtgctgctgaaggacctgctgtgctgggagctggaggacgtgctctacttcaagctcaacgcccgccgcgactcgcccgtgccgcggctctcgggggagctgtatgggcgcgccaccgcctggaacatgctggactcccacctctaccgccacttcaacgccagcttctggcgcaaggtggaggccttcgggcgggagcgcatggcccgcgaggtggccgccctgcgccatgccaacgagcgcatgcggaccatctgcatcgacgggggccacgccgtggacgccgccgccatccaggacgaggccatgcagccctggcagccgctgggcaccaagtccatcctgggctacaacctcaagaagagcatcgggcagcggcacgcgcagctctgccggcgcatgctcacgcccgagatccagtacctgatggacctcggcgccaacctgtgggtcaccaagctctggaagttcattcgcgatttcctgcggtggtgacgtcccaccgcccagcggcttgcctgcctgctcgctccctgcagaggggctgagcaggacgccgctggtgctggccgcccccagccccctcctggtgccacctcagaccccggggtgagggggggctccctggggggaggcagccagccaagactgggcccatgaacacagagagggcctaaccgagatcagtatttaactaattataccagtttttattaaacccctttccctccccgataaagaatgttctatttctgcctccccttaaaggggagacctcagaagtaaaggaatttgatgttgtgtttttgttaa

SEQ ID NO: 635 - RNA, 6142 nt, synthetic construct
  source 1..6142: mol_type = other RNA
actattgcagcctccctgattccagaaggtaccttggtgagtggggctgctggggccggggtgggtccttgggatttccagagggcaagccacaggaatcctaatccctcctcatctttaagtcagagaaactgaggcccagagttttggactatgatctggtccacgactctgccctgttttctgctgggtcgctgagaagtgggaggctgacaggggtgcttgaggctgaggcaggtttgtgctttgtgggcggcccagcccctactccttttggggttgaaggcaggccccccgggcctcaggaggggtacagataacactctaagtgatgactatgtgctggttgcttgcttagaaaagttctccagagaatggctaggggcgcctccagctttctcattttacagatgaaaaactgagactccatgagggttcttggcttgtctgaaatcacagtgctggattgaaacacaggcttgagggtcccagagcctgaggttggtgctggggcccctccatggctgccgggtttattccgctttgcaagctttctgatttgttacttaaatgtgttaagtaacaaattaactaacacatttgttaacgtgttgacaattattgcctttttttgagatggggcctcactctggtcacccaggctgaagtgcagtggtgcgaccttggctcacggtaacctctgccaacctctgccccccaggctcaagcaatcctcccacctcagcataacttttccagggcatagtgtgtgtgaccctcccccagggggactgactccagggacagtggtgataatgcatgcatgtagttattagcacatgtgacttttcctggcctcaggggcccagggcgcagtgcagtgtgagccaggacagaagtggaaagttcgggacaagagtagaaagtttggggacctggttttgcagctaaggggaggtgggaaggccctccaagccgcagctctctcctggactccagttcctcagcctgaactgcccccgttacagtccatgtctcagtcccctgctgatagggttgccacgcttagatcatcccccaaaacaaataacaacaggctgggcacagtggctcatgcctataatcccagcactttgggaggctgaggtgggaggattgcttgagtccaggagttcaataccagcctgggcatctcaaaaataataatataaaataaatattaaaaatgtataaaaatgggccaggcgcggtggctcatgcctgtaactctagcactttgggaggccaaggtgggcagatcacaaggtcaggagatcaagatgatcctggccaacatggtgaaactccatctctactaaaaatacaaaaattagctgggtgtggtggcgtgtgcctgtaatcccagctacttgggaggctgaggcaggagaatcgcttgaaccggggagtcaaaggttgcagggagccgagattgcgccacagcactccagcctggcgacagagcgagactccgtctcaaaaaaataaaataaacatataaaaatgaaaaaataaggaaataacaacaaaacctccaggacatctagttaagtttgaagtttgaataattttttttttttgagacagagtctcactctgtcacccaggcaggaatacagtggcaccatcttggctcactgcaacctctacctcctgggttcaagcgattcttctacctcagcctcctgagtaactgggactataggggcatgccaccacacccagctagtttttgtattttcagtagagacagaatttcaccatattggtcaggcttgtctcaaactcctgacctcaggtgatccaccctcctcggcctcccaaagtgctaggattacaggcgtgagccaccgtgcccagccttggaataatttttagtataactatgtcccatgcaatatttggaatatacttatactgtaaaaaattgtattcattgtttatctgaaatttgaatttaactgagcaaccctacaggctggctttggaaagggggtgggtatgtgagcagaggcctggccagctgtgtgacttgagccttcttcttcccagctgtgagcctcagtttctccatcagccaccctacagactctaggggcacaagggccagtgaggagagaagagccctgggctcttactgggcacctcacctgtaccactgtggggtgggtggcacaggggacaggtagggctggctgtgccttctgaaaggtgaaggattcaggccccagagagcccttggccttccctatagacagccctggcccaggcctgggggagggagggcccagggcacggggagagggtcttcttttccttcttatttatttgtttatttatttgagacagggtctcactgtgtcatccagagctggagtgcagcggcacagtcatggctcactggaactcaggctcaagcaatcctcccgcctcagccttccaagtaactaggactacaggcatgtgccaccacgcctggtgaatttttttaaattttgttttgtagagataggttctcaccatatgcccaggctggtcttgaactcctggctcaagcactccgcctgcctcatcctcccaaagtgctgggatgacaggtgtgagccactgtgcccaggctttttccttttgccttagaaaagtgtttcatggtcgggcgcgatggctcacgtctgtcatcccagcaatttgggaggccaaggcgggcggattgcttgagcccaggagttcgagaccagcctaggcaacgtggcaaaactgtctctatttttaaaaaaattaaaatataaaaaaggaaataatttcattatgatgccctaaaaggtataaaaaataacttcaggatctgggagaatttgaagtaaaattgaagaatgttttaaatatatatttttataaaaaatacaagaaaaagaaacagaagaaaaaataagataactaaaaaaaaaaaggtaataagtattagttgtctcattttttcttctgtttctttttcttttatttcttatcagccagcttaaaaattaaaaggagtggaaagtttggggccctggttctctacccggttcattttaatttttaaattttaaccttctccctgccccattccccaacctctctttgagttgcttcacccaccgtctctgcaaccctggggctgtctctttccaggtggttgtcagtctatctctccagtgcctctctgtccctgtctggaccctgctggggggccacagagcaggcaaaggcagggctcaactgccaggcctgggcagccttgggggctccctggggccagcttcctgacacttgccctactctgtgctgcccctgcaggtgtctgagatgctgccaccgcagaagaagccctgggagtccatggctaaggggctggtgctgggcgcgctcttcactagtttcctgctgctggtgtactcctatgccgtgcccccgctgcatgccggcctggcctccacgtgagtggcccagctgggcagatgatgggtgtaggtgggggagggatggcaccccacccatggggccctgtaattaccacagggcctgccccagtgatcttgggtagccaccctgcaccctgcaccatgctgtgccagacagaaattccactgatggcagagctcgccgtgtaactctgagccttggtgtgcccatctgtgaagtggggataaaaacagagcctttcggctgggcacggcaactcacgcctgtaatcctagcactttgggaggcccaggcgggcagatcgtctgaggtcaggagttcgagaccagcctggccaacatggtgaaaccccatctctactaaaaatacaaaaattagctgggcatggtggtgtgtgtctgtaatctcagctacttgggagtctgaggcaggagaattgcttgaacccggaaggcagaagttgcagtgagccgagatggcaccactgtactccaggctgggccaacggagtgagactgtctcaaaacaaaaagaaaaacaaacaaacaaaaaaaacccagagccttcctcatagagcatgagaatcaggtaacactgtctgtacagggctcagatttgcatcatctgagatgccaacggggccctggtgggggcgacctgtccatgggcatggattcaaacgggtcggtctgactccaggcccagctcctgagaaccaggctctcccgtttcctcatggggctgctctgcacagacccagcacagcctgtagctggtgcaataccagtggttggtgatattaacttgacacagaggaaactgcaaatttctgcaaaaatggaccatttttgtcctataaaatggcagtttcatgtggcccaagctgatactgattcatggtacatgcaacacagtggccatgaatgagaggagagcctgtgtgcctcatctcacaacaccgtgaatagggcctgttatcgtccccatttcacagaagaggaacatgaggctagagaggccaaaccacccgcgcaggcacccagcctggactcaggccccacaggtctcactatcccagctcctttgccaccactgttcccaataatctccctctctaactaccagagtcctgagggccccagcacccccctgaggctctccccacctctctgtccccagcaggaccccggaggccgcagcgtcctgctctccacctgcactcgagccagaggcagtgatccgggccaacggctcggcgggggagtgccagccgcggcgcaacatcgtgttcttgaagacgcacaagacggccagcagcaccctgctcaacatcctgttccgcttcggccagaagcaccggctcaagttcgccttccctaacggccgcaatgacttcgactacccgaccttcttcgcccgcagcctggtgcaggactatcggcccggggcctgcttcaacatcatctgcaaccacatgcgcttccactacgacgaggtgcgcggcctggtgccgaccaacgccatcttcatcacggtgctccgcgaccccgcccgcttgttcgagtcctccttccactacttcgggccggtggtgcccctcacgtggaagctctcggccggcgacaagctgaccgagttcctgcaagacccggatcgctactacgaccccaacggcttcaatgcccactacctccgaaacctgctcttcttcgacctgggctatgacaacagcctggaccccagcagcccgcaggtgcaggagcacatcctggaggtggagcgtcgcttccacctggtgctccttcaagagtacttcgacgagtcgctggtgctgctgaaggacctgctgtgctgggagctggaggacgtgctctacttcaagctcaacgcccgccgcgactcgcccgtgccgcggctctcgggggagctgtatgggcgcgccaccgcctggaacatgctggactcccacctctaccgccacttcaacgccagcttctggcgcaaggtggaggccttcgggcgggagcgcatggcccgcgaggtggccgccctgcgccatgccaacgagcgcatgcggaccatctgcatcgacgggggccacgccgtggacgccgccgccatccaggacgaggccatgcagccctggcagccgctgggcaccaagtccatcctgggctacaacctcaagaagagcatcgggcagcggcacgcgcagctctgccggcgcatgctcacgcccgagatccagtacctgatggacctcggcgccaacctgtgggtcaccaagctctggaagttcattcgcgatttcctgcggtggtgacgtcccaccgcccagcggcttgcctgcctgctcgctccctgcagaggggctgagcaggacgccgctggtgctggccgcccccagccccctcctggtgccacctcagaccccggggtgagggggggctccctggggggaggcagccagccaagactgggcccatgaacacagagagggcctaaccgagatcagtatttaactaattataccagtttttattaaacccctttccctccccgataaagaatgttctatttctgcctccccttaaaggggagacctcagaagtaaaggaatttgatgttgtgtttttgttaa

SEQ ID NO: 636 - protein, 423 aa, synthetic construct
MLPPQKKPWESMAKGLVLGALFTSFLLLVYSYAVPPLHAGLASTTPEAAASCSPPALEPEAVIRANGSAGECQPRRNIVFLKTHKTASSTLLNILFRFGQKHRLKFAFPNGRNDFDYPTFFARSLVQDYRPGACFNIICNHMRFHYDEVRGLVPTNAIFITVLRDPARLFESSFHYFGPVVPLTWKLSAGDKLTEFLQDPDRYYDPNGFNAHYLRNLLFFDLGYDNSLDPSSPQVQEHILEVERRFHLVLLQEYFDESLVLLKDLLCWELEDVLYFKLNARRDSPVPRLSGELYGRATAWNMLDSHLYRHFNASFWRKVEAFGRERMAREVAALRHANERMRTICIDGGHAVDAAAIQDEAMQPWQPLGTKSILGYNLKKSIGQRHAQLCRRMLTPEIQYLMDLGANLWVTKLWKFIRDFLRW

SEQ ID NO: 637 - DNA, 20 bp, synthetic construct
  source 1..20: mol_type = other DNA
gcatgtggttgcagatgatg

SEQ ID NO: 638 - DNA, 20 bp, synthetic construct
  source 1..20: mol_type = other DNA
tcaagttcgccttccctaac

SEQ ID NO: 639 - DNA, 24 bp, synthetic construct
  source 1..24: mol_type = other DNA
cgcaatgacttcgactacccgacc

SEQ ID NO: 640 - DNA, 22 bp, synthetic construct
  source 1..22: mol_type = other DNA
tatcaggacagagttcacatcc

SEQ ID NO: 641 - DNA, 23 bp, synthetic construct
  source 1..23: mol_type = other DNA
gtttctaaagacatggtcacagc

SEQ ID NO: 642 - DNA, 22 bp, synthetic construct
  source 1..22: mol_type = other DNA
aaaggccagccaccagttcagg

SEQ ID NO: 643 - DNA, 19 bp, synthetic construct
  source 1..19: mol_type = other DNA; note = forward primer
aagacccggatcgctacta

SEQ ID NO: 644 - DNA, 20 bp, synthetic construct
  source 1..20: mol_type = other DNA; note = reverse primer
ggtccaggctgttgtcatag

SEQ ID NO: 645 - DNA, 24 bp, synthetic construct
  source 1..24: mol_type = other DNA; note = probe sequence
aatgcccactacctccgaaacctg

(19) United States
(12) Patent Application Publication
(10) Pub. No.: US 2026/0286364 A1
(43) Pub. Date: Sep. 24, 2026

(54) OLIGONUCLEOTIDES TARGETING GAL3ST1

(71) Applicant: Contera Pharma A/S, Hørsholm (DK)

(72) Inventors: Søren Vestergaard Rasmussen, Hørsholm (DK); Peter Hagedorn, Hørsholm (DK); Marianne Lerbech Jensen, Hørsholm (DK); Tue Fryland, Rønde (DK)

(21) Appl. No.: 19/490,871

(22) PCT Filed: Jun. 4, 2024
(86) PCT No.: PCT/EP2024/065312

§ 371 Date: Dec. 5, 2025

(30) Foreign Application Priority Data
Jun. 5, 2023 (EP) 23177340.9

(51) Int. Cl. C12N 15/113; A61K 31/7115; A61K 31/712; A61K 31/7125

(52) U.S. Cl. CPC C12N 15/1137; A61K 31/7115; A61K 31/712; A61K 31/7125; C12N 2310/11; C12N 2310/315; C12N 2310/321; C12N 2310/3231; C12N 2310/3341; C12N 2310/341; C12N 2310/346; C12N 2310/3525; C12N 2310/3533; C12N 2320/30

(57) ABSTRACT

The present invention relates to antisense oligonucleotides (oligomers) complementary to GAL3ST1 pre-mRNA sequences, which are capable of inhibiting the expression of GAL3ST1. Inhibition of GAL3ST1 expression is expected to be beneficial for the treatment of metachromatic leukodystrophy.

### CROSS-REFERENCE TO RELATED APPLICATIONS

This application is a U.S. National Phase of International PCT Application No. PCT/EP2024/065312 filed on Jun. 4, 2024, which claims priority to European Patent Application No. 23177340.9 filed on Jun. 5, 2023, the contents of each application are incorporated herein by reference in their entireties.

### SEQUENCE LISTING

This application incorporates by reference the material in the ST.26 XML file titled COP16958PC Sequence listing.xml, which was created on Jun. 3, 2024 and is 615,308 bytes.

### FIELD OF THE INVENTION

The present invention relates to antisense oligonucleotides (oligomers) complementary to GAL3ST1 pre-mRNA sequences, which are capable of inhibiting the expression of GAL3ST1. Inhibition of GAL3ST1 expression is expected to be beneficial for the treatment of metachromatic leukodystrophy.

### BACKGROUND

Metachromatic leukodystrophy (MLD) is a rare autosomal recessive lysosomal storage disorder caused by a deficiency in the arylsulfatase A (ARSA) enzyme. Mutations in the ARSA gene result in the accumulation of toxic sulfatides in the nervous system (e.g. oligodendrocytes, microglia, some CNS neurons) leading to progressive demyelination and neurological dysfunction.

Clinical manifestation of MLD can vary depending on the age of onset, which can range from infancy to adulthood. The most common form is the late-infantile form, which usually presents between 6 months and 2 years of age. Children with this form typically have developmental delay, followed by regression of skills, muscle weakness, spasticity, seizures, and vision and hearing loss. As the disease progresses, patients may develop feeding difficulties, respiratory distress, and eventually become bedridden. In the juvenile form, onset occurs between 2 and 16 years of age, with symptoms including gait abnormalities, cognitive decline, and behavioral changes. The adult form of MLD is the least common and usually presents after age 16, with symptoms such as cognitive decline, psychiatric symptoms, and motor dysfunction (Polten et al., 1991, Shaimardanova et al., 2020). In all forms of MLD, the disease is progressive, leading to severe disability and for most patients' death.

The prognosis for MLD is poor, with a life expectancy of 5-10 years for the late-infantile form, 10-20 years for the juvenile form, and variable in the adult form. The severity and progression of the disease can be influenced by the age of onset, genotype, and residual ARSA enzyme activity. However, there is currently no cure for MLD, and treatment is largely supportive. Bone marrow or hematopoietic stem cell transplantation has shown some success in slowing disease progression, particularly in patients with the late-infantile form. However, this treatment is associated with significant risks and is only effective if done early in the disease course (Van Rappards et al., 2014, Boucher et al., 2015).

As stated above, the reduction of functional arylsulfatase A leads to an accumulation of toxic sulfatides in CNS. Taken that sulfatide accumulation in various cells is the root cause of MLD, then a therapeutic concept that reduces the amount of sulfatide would be anticipated to have a positive effect on disease severity and progression. As an alternative to reinstating ARSA enzymatic activity for increased degradation of sulfatide, it should be possible to reduce sulfatide accumulation by reducing the biosynthesis. Sulfatides are synthesized in the Golgi apparatus by galactose-3-O-sulfotransferase-1 (GAL3ST1) transferring sulphate from 3-phosphoadenosine-5-phosphosulfate (PAPS) to galactosylceramide (GalC). In the human genome there are four genes (GAL3ST1, -2, -3, -4) are described to have Galactose-3-O-sulfotransferase activity, however they show different substrate specificity and expression pattern. As for the biosynthesis of sulfatides GAL3ST1 appears to be the sole responsible enzyme as Gal3st1 homozygous knock out mice (GAL3ST1^{-/-}) completely lack sulfatides in brain (Honke et al., 2001). Moreover, it has been shown that overexpression of Gal3st1 in Arsa (−/−) mice leads to increased sulfatide storage in the nervous system and augmentation of the MLD like pathology (Ramakrishnan et al., 2007). Moreover, Eckhardt et al. discloses that ASA-deficient mice have been used for more than a decade as an animal model of MLD. Sulfatide storage pattern, as detected by blue staining, in ASA-deficient mice closely resembles the sulfolipid storage pattern observed in MLD patients. Further, Jones E et al. discloses that cerebrosides in vertebrates may be sulphated by the cerebroside sulfotransferase enzyme (encoded by the GAL3ST1 gene) to make sulfatide. Altogether this indicates that inhibition or reduction of GAL3ST1 expression could be an effective therapeutic concept for treating MLD.

There remains a need for means and methods for the efficient down-regulation of the GAL3ST1 gene.

In the studies underlying the present invention, target sequences within the human GAL3ST1 pre-mRNA and potent antisense oligonucleotides targeting the human GAL3ST1 gene for treatment of MLD.

### BRIEF SUMMARY OF THE PRESENT INVENTION

The present invention provides an antisense oligonucleotide comprising a stretch of at least 10 nucleotides which is at least 90% complementary to a target sequence in a GAL3ST1 (Galactosylceramide sulfotransferase) gene.

In a preferred embodiment, the stretch of at least 10 nucleotides is at least 90% complementary to a target sequence selected from the group of target sequences consisting of SEQ ID NO: 522 to SEQ ID NO: 632. The sequences are shown in Table B1 in the Examples section.

In a further preferred embodiment, the stretch of at least 10 nucleotides is at least 90% complementary to a target sequence selected from the group of target sequences consisting of SEQ ID NO: 522, 523, 525, 532, 537, 540, 546, 547, 550, 551, 562, 563, 565, 569, 570, 571, 572, 573, 594, 598, 600, 616, 617, 618, 623, 624, 627, and 629. The sequences are shown in Table B2 in the Examples section.

In a particularly preferred embodiment, the stretch of at least 10 nucleotides is at least 90% complementary to a target sequence selected from the group of target sequences consisting of SEQ ID NO: 522, 532, 537, 540, 546, 547, 562, 565, 569, 570, 573, 600, 617, 624, 627, 629. The sequences are shown in Table B3 in the Examples section.

In an embodiment, the stretch of at least 10 nucleotides is at least 90% complementary to a target sequence as shown in SEQ ID NO: 522.

In an embodiment, the stretch of at least 10 nucleotides is at least 90% complementary to a target sequence as shown in SEQ ID NO: 527.

In an embodiment, the stretch of at least 10 nucleotides is at least 90% complementary to a target sequence as shown in SEQ ID NO: 540.

In an embodiment, the stretch of at least 10 nucleotides is at least 90% complementary to a target sequence as shown in SEQ ID NO: 546.

In an embodiment, the stretch of at least 10 nucleotides is at least 90% complementary to a target sequence as shown in SEQ ID NO: 547.

In an embodiment, the stretch of at least 10 nucleotides is at least 90% complementary to a target sequence as shown in SEQ ID NO: 562.

In an embodiment, the stretch of at least 10 nucleotides is at least 90% complementary to a target sequence as shown in SEQ ID NO: 565.

In an embodiment, the stretch of at least 10 nucleotides is at least 90% complementary to a target sequence as shown in SEQ ID NO: 569.

In an embodiment, the stretch of at least 10 nucleotides is at least 90% complementary to a target sequence as shown in SEQ ID NO: 570.

In an embodiment, the stretch of at least 10 nucleotides is at least 90% complementary to a target sequence as shown in SEQ ID NO: 600.

In an embodiment, the stretch of at least 10 nucleotides is at least 90% complementary to a target sequence as shown in SEQ ID NO: 617.

In an embodiment, the stretch of at least 10 nucleotides is at least 90% complementary to a target sequence as shown in SEQ ID NO: 624.

In an embodiment, the stretch of at least 10 nucleotides is at least 90% complementary to a target sequence as shown in SEQ ID NO: 627.

In an embodiment, the stretch of at least 10 nucleotides is at least 90% complementary to a target sequence as shown in SEQ ID NO: 629.

In an embodiment, the stretch of at least 10 nucleotides is at least 90% complementary to a target sequence as shown in SEQ ID NO: 627.

Preferably, the antisense oligonucleotide comprises a stretch of at least 12, or at least 14 nucleotides which is at least 90% complementary to a target sequence as set forth herein.

In preferred embodiment, said stretch is 95%, in particular 100% complementary to the target sequence.

In a preferred embodiment, the stretch of at least 10 nucleotides is fully complementary to a target sequence comprised by SEQ ID NO: 522.

In another preferred embodiment, the stretch of at least 10 nucleotides is fully complementary to a target sequence comprised by SEQ ID NO: 624.

In yet another preferred embodiment, the stretch of at least 10 nucleotides is fully complementary to a target sequence comprised by SEQ ID NO: 627.

In yet another preferred embodiment, the stretch of at least 10 nucleotides is fully complementary to a target sequence comprised by SEQ ID NO: 629.

Preferably, the antisense oligonucleotide has a length of 12 to 30 nucleotides, more preferably, a length of 14 to 22 nucleotides, and most preferably a length of 16 to 20 nucleotides.

In some embodiments, the antisense oligonucleotide comprises or consists of a nucleic acid sequence as shown in SEQ ID NO: 1 to SEQ ID NO: 521. In some embodiments, the antisense oligonucleotide consists of a nucleic acid sequence as shown in SEQ ID NO: 1 to SEQ ID NO: 521.

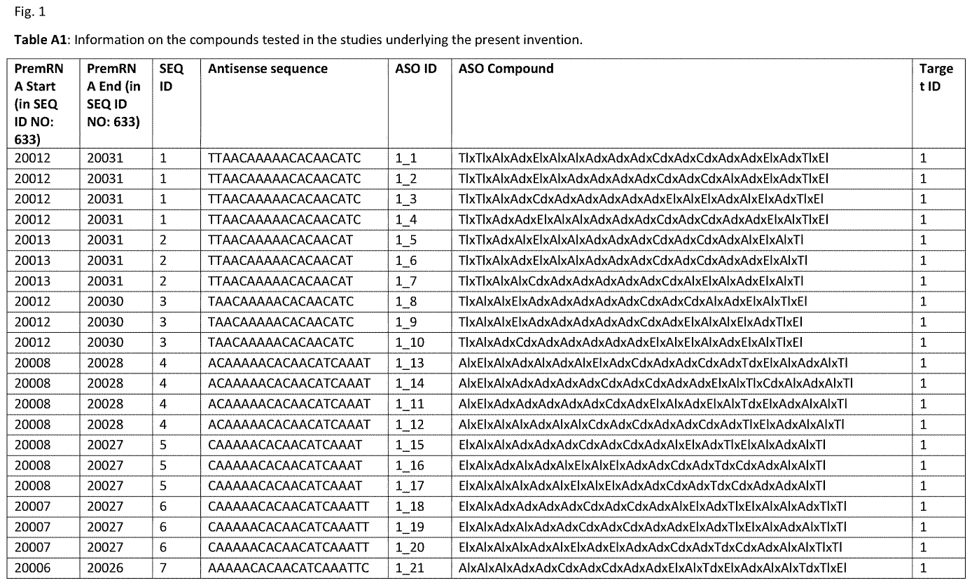

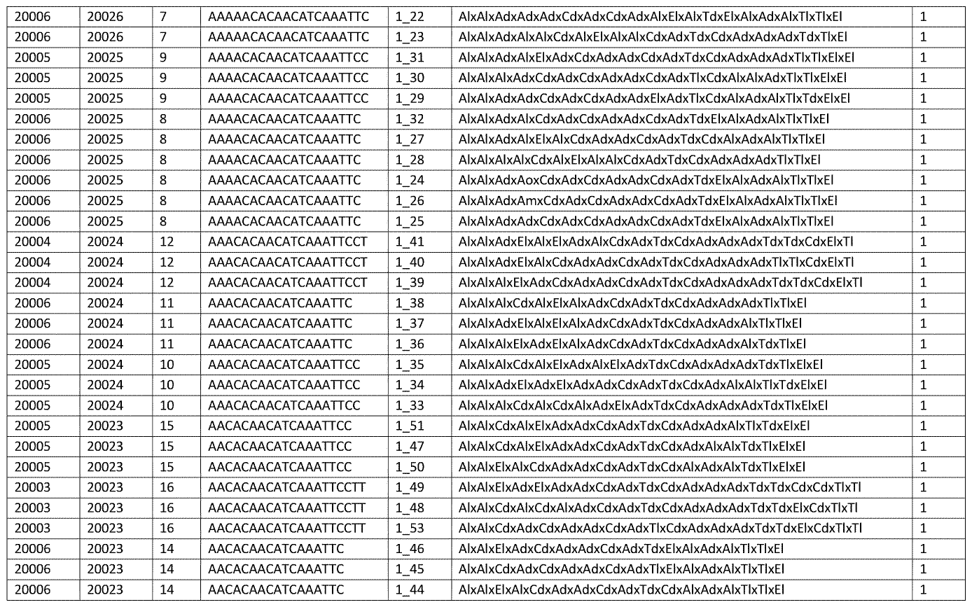

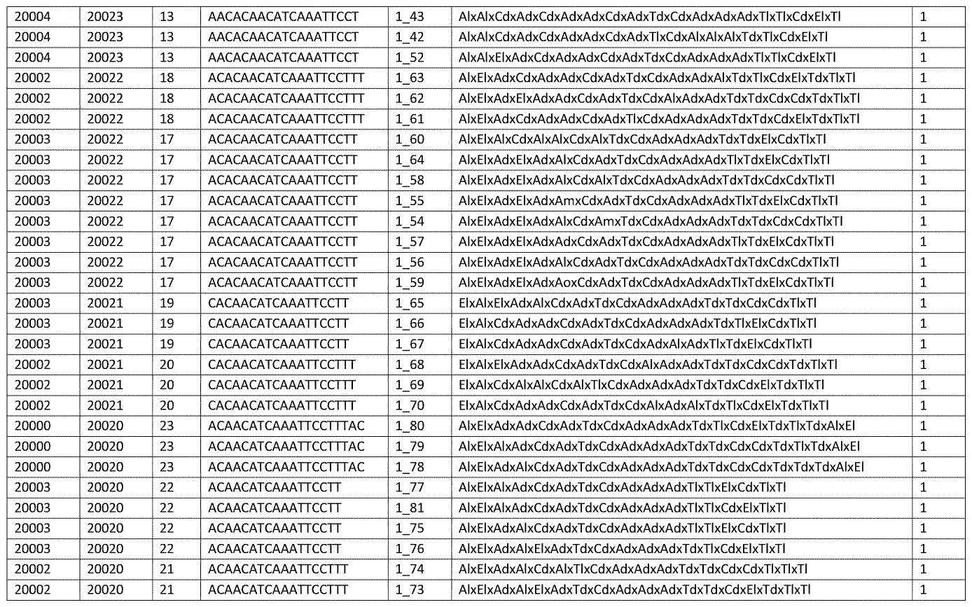

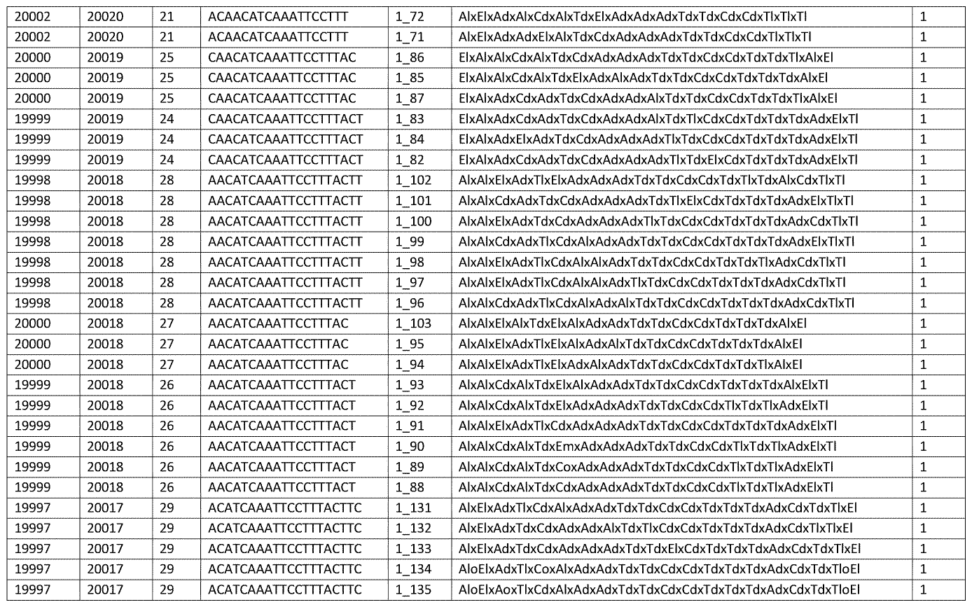

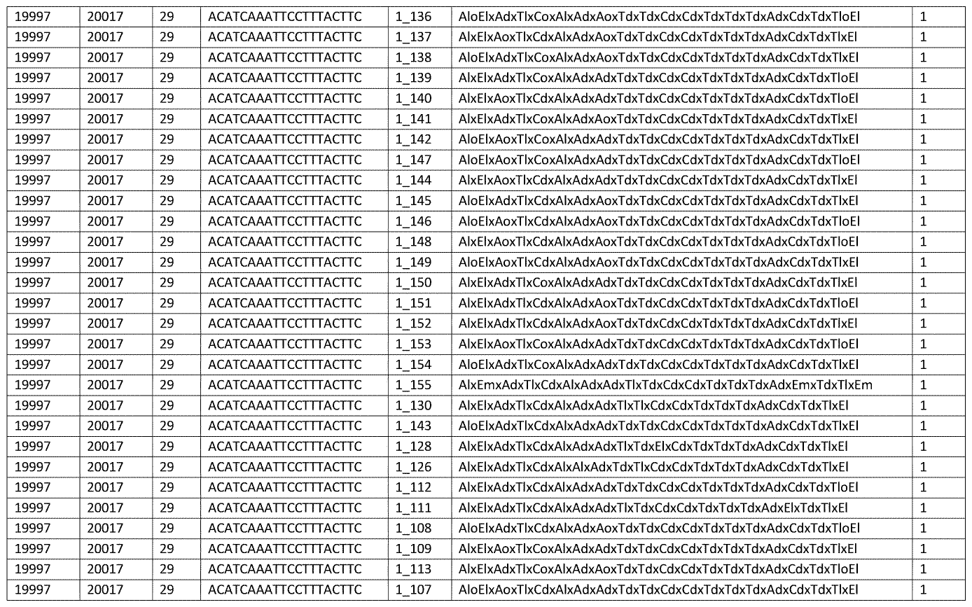

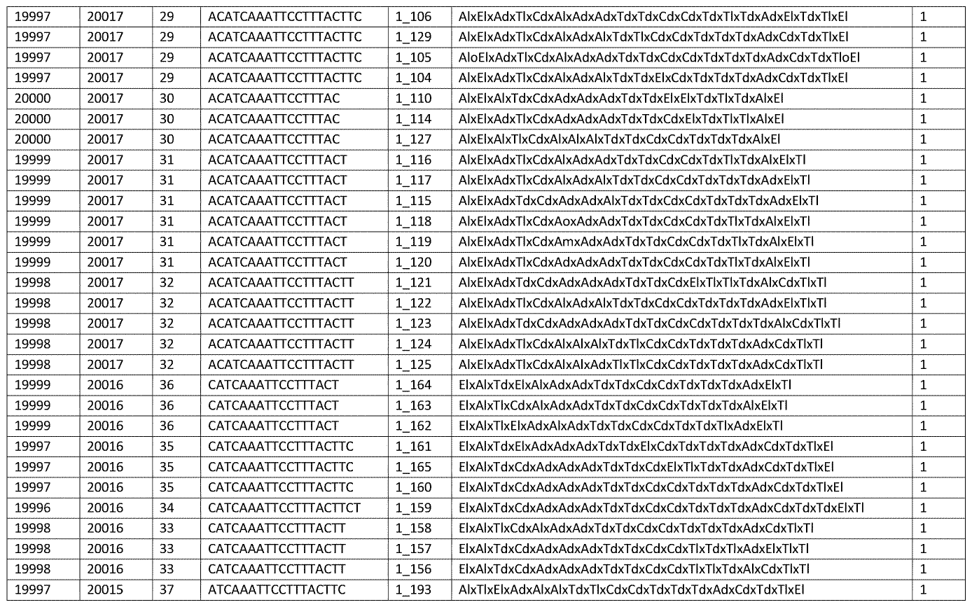

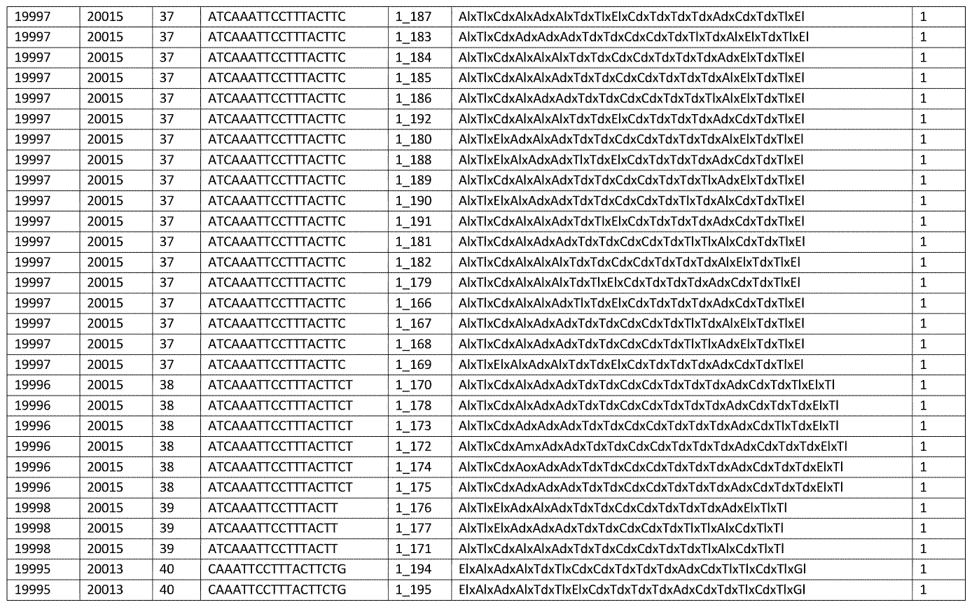

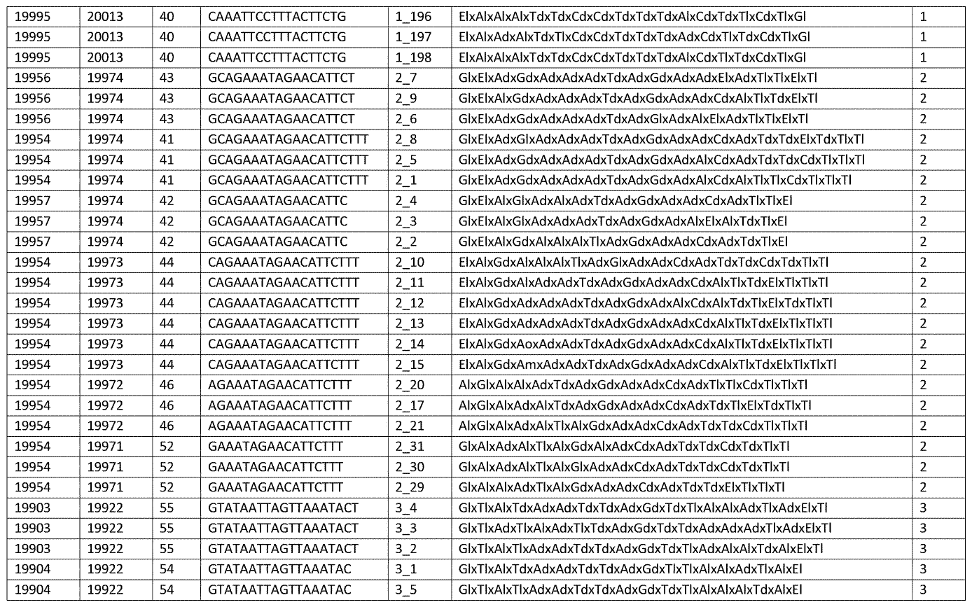

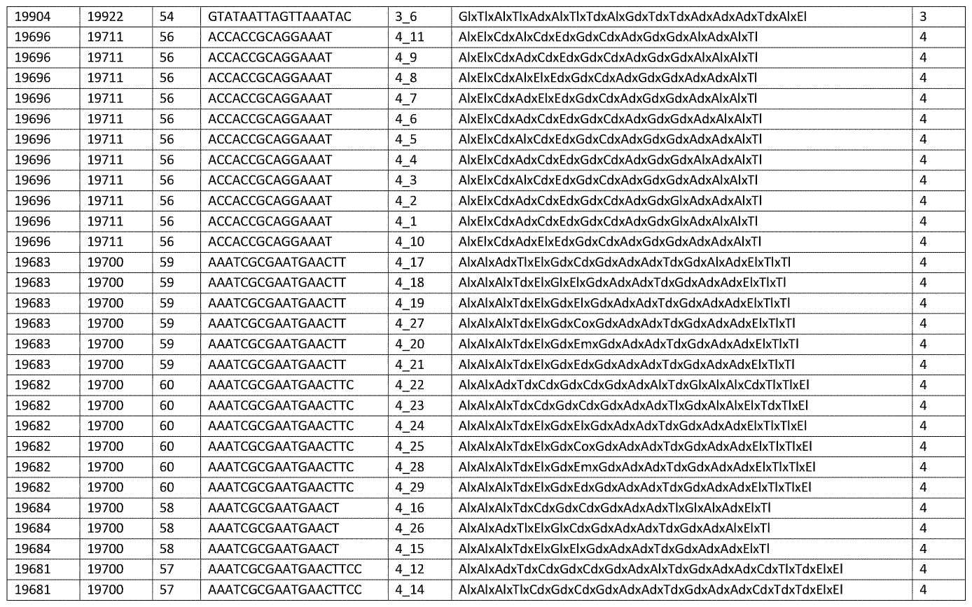

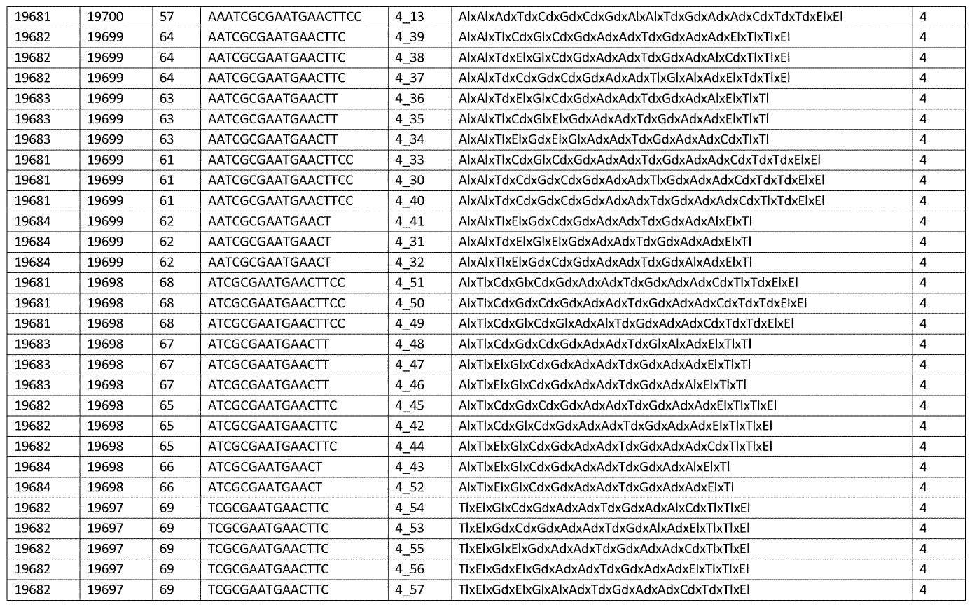

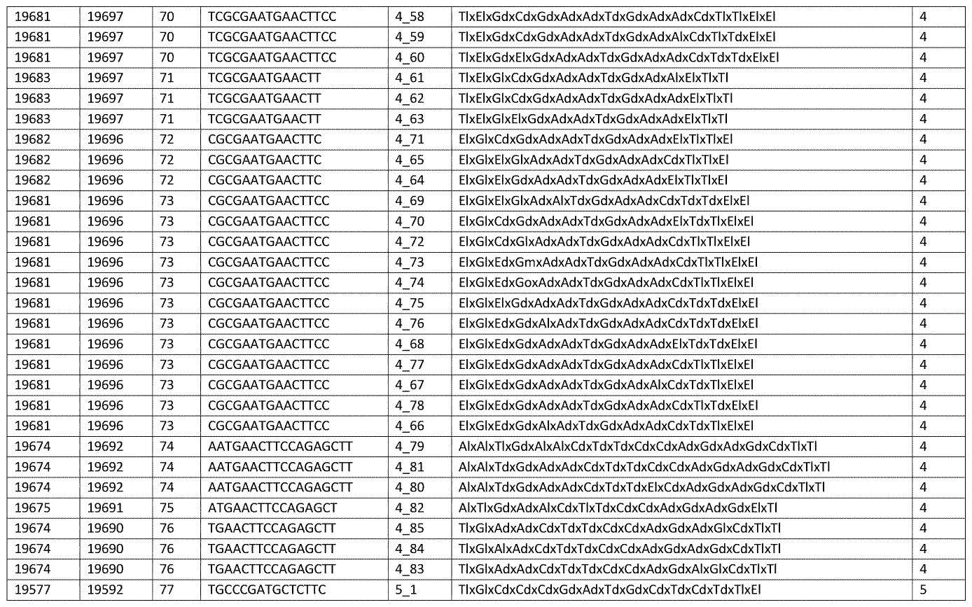

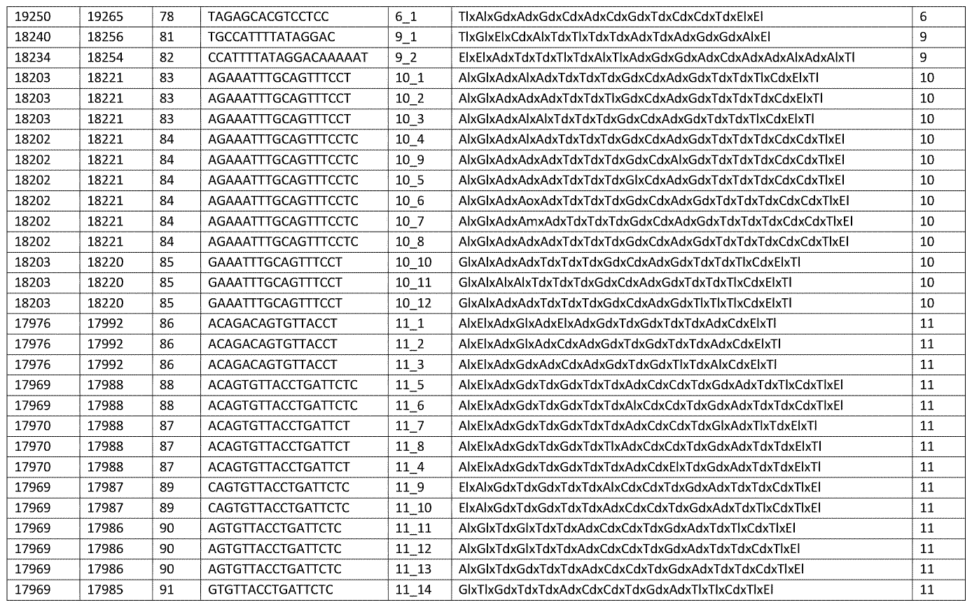

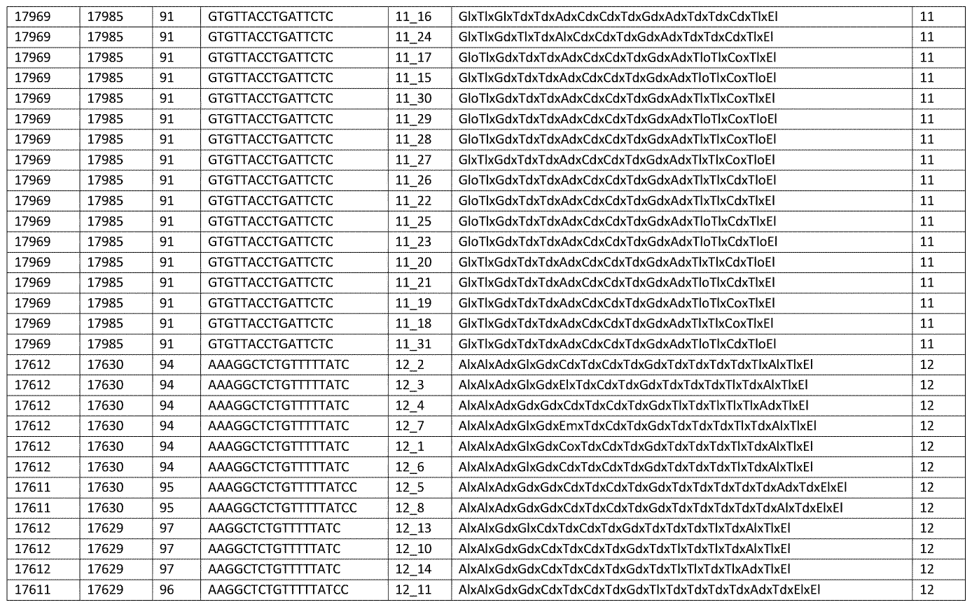

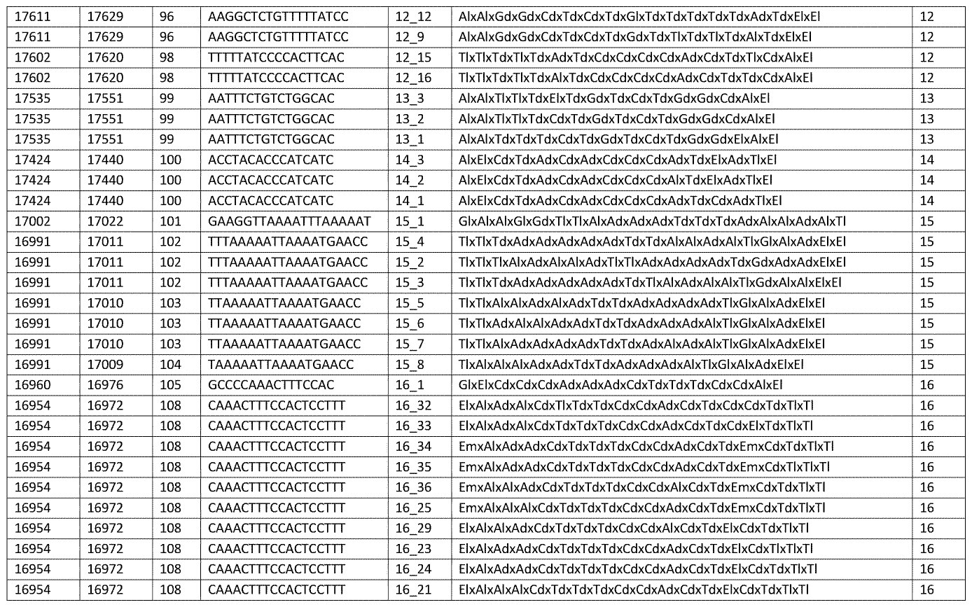

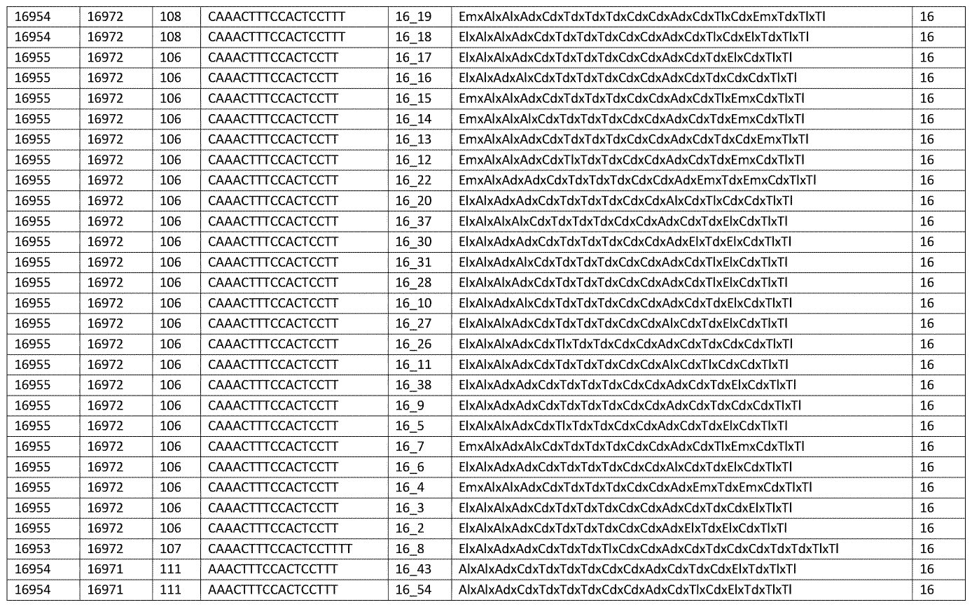

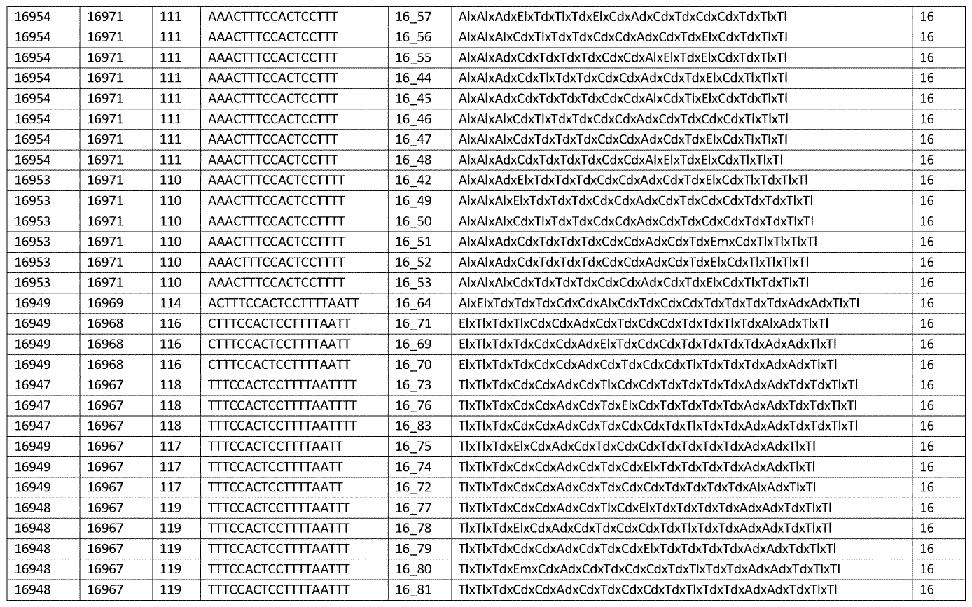

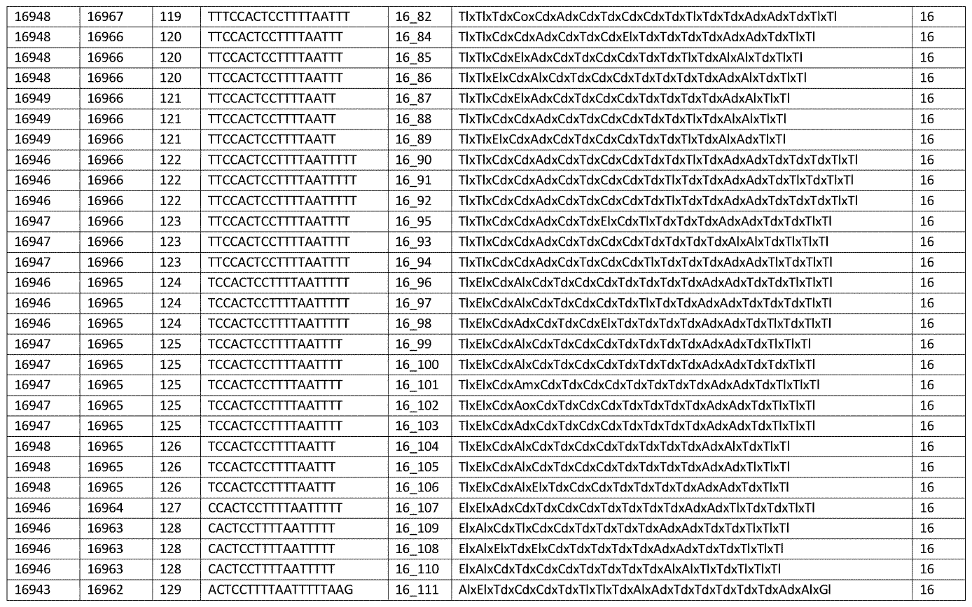

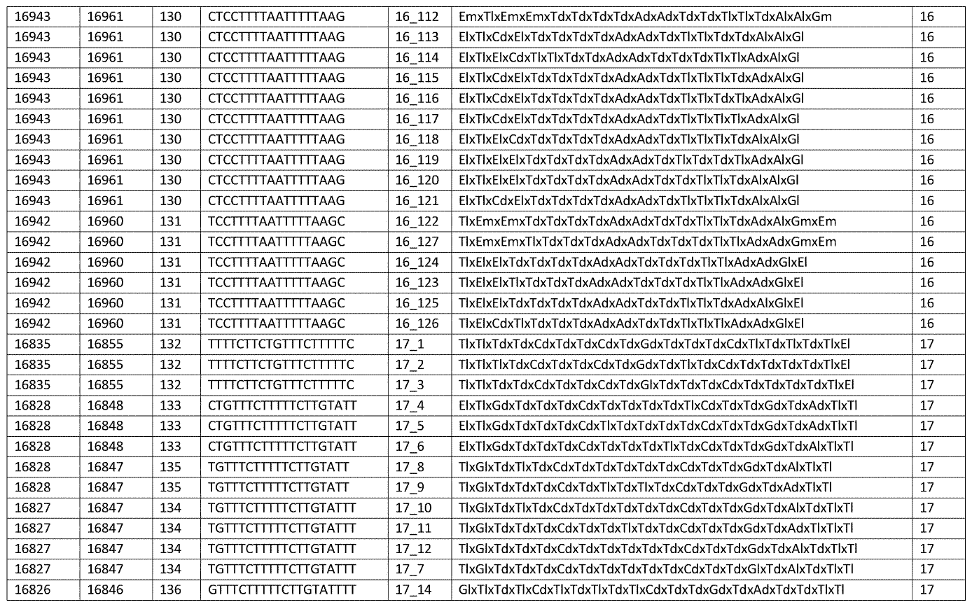

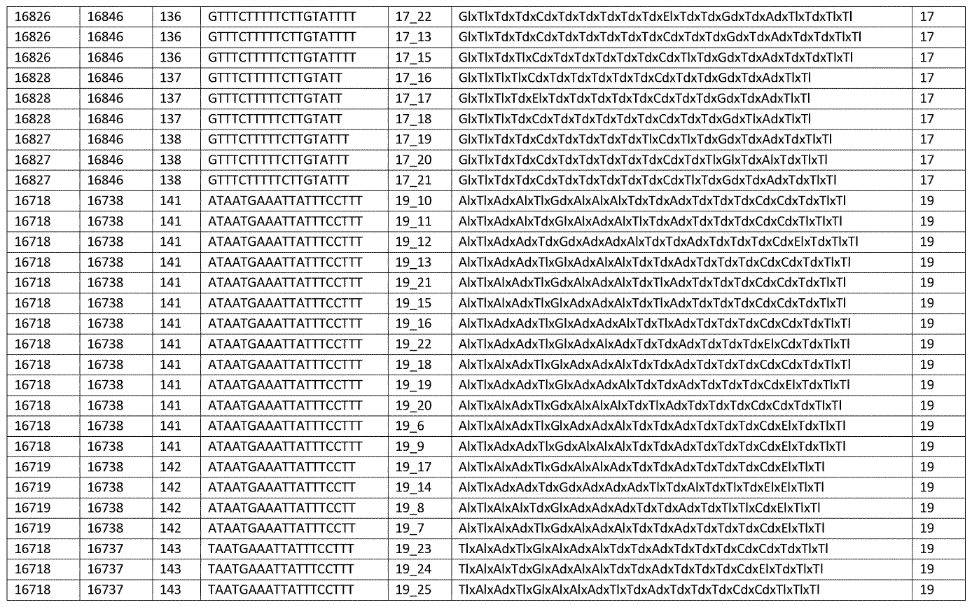

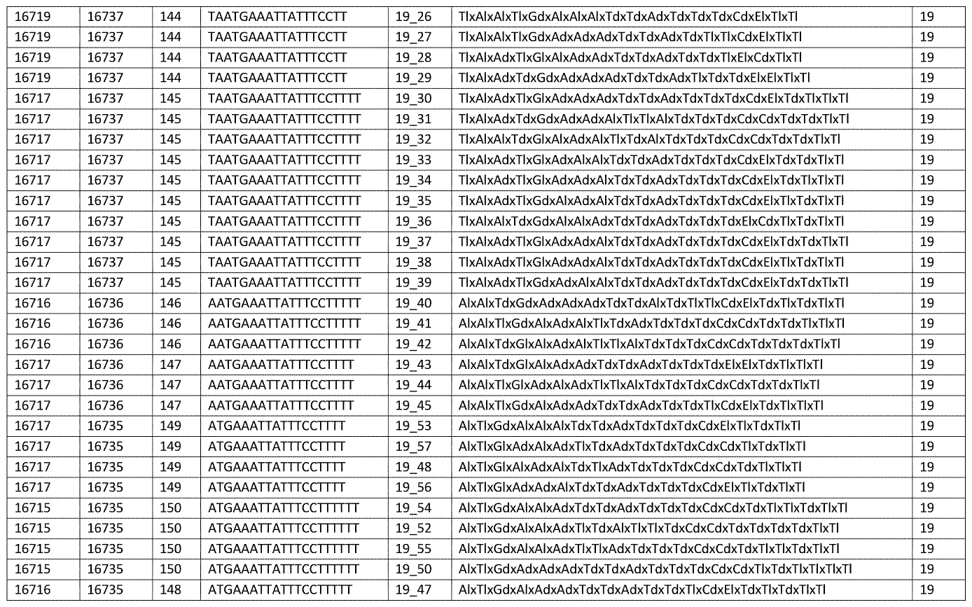

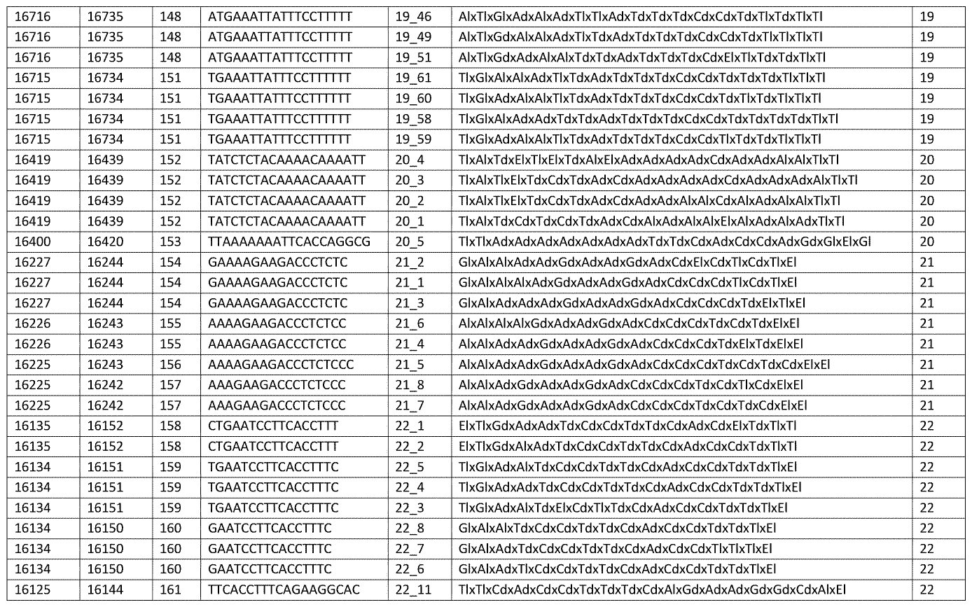

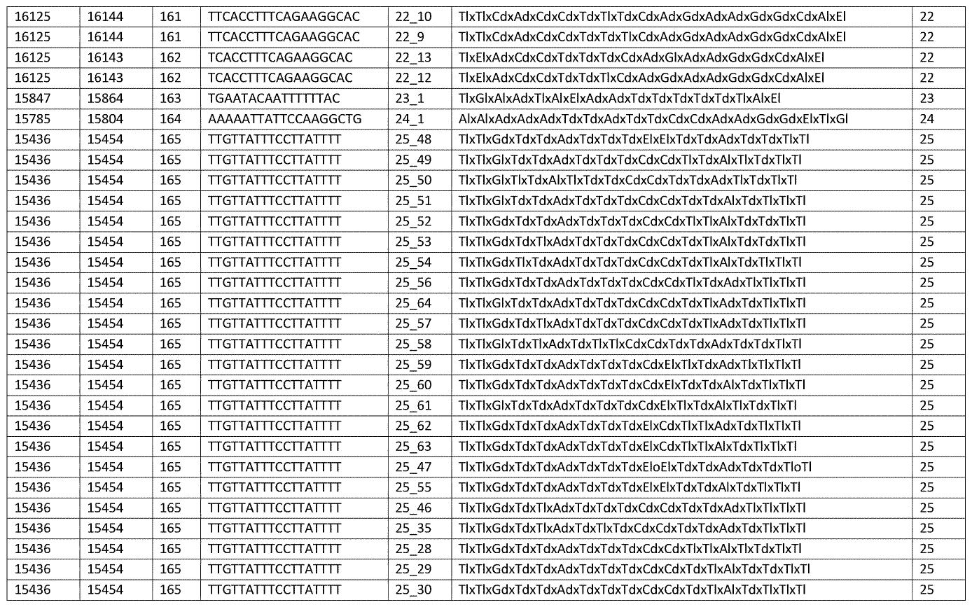

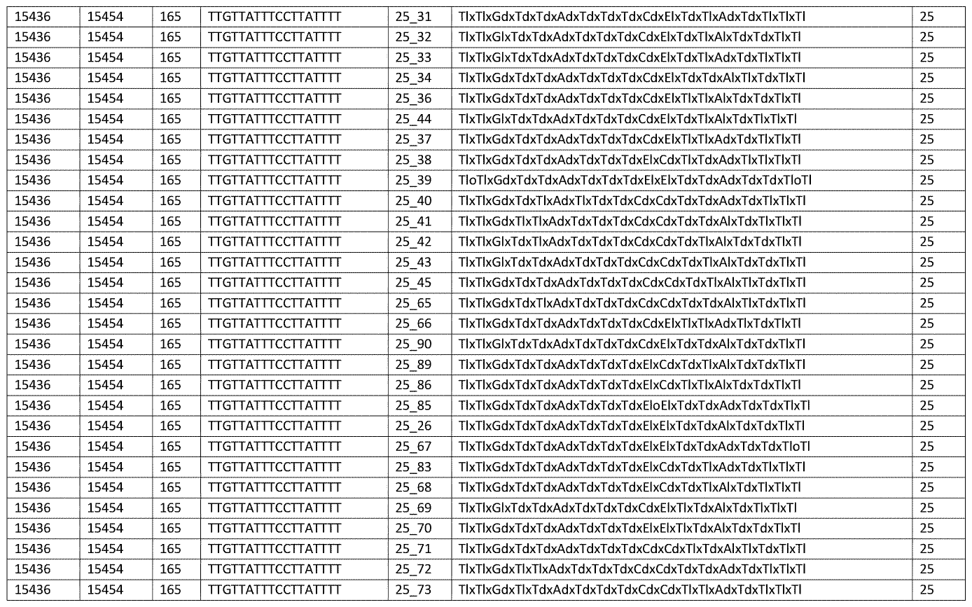

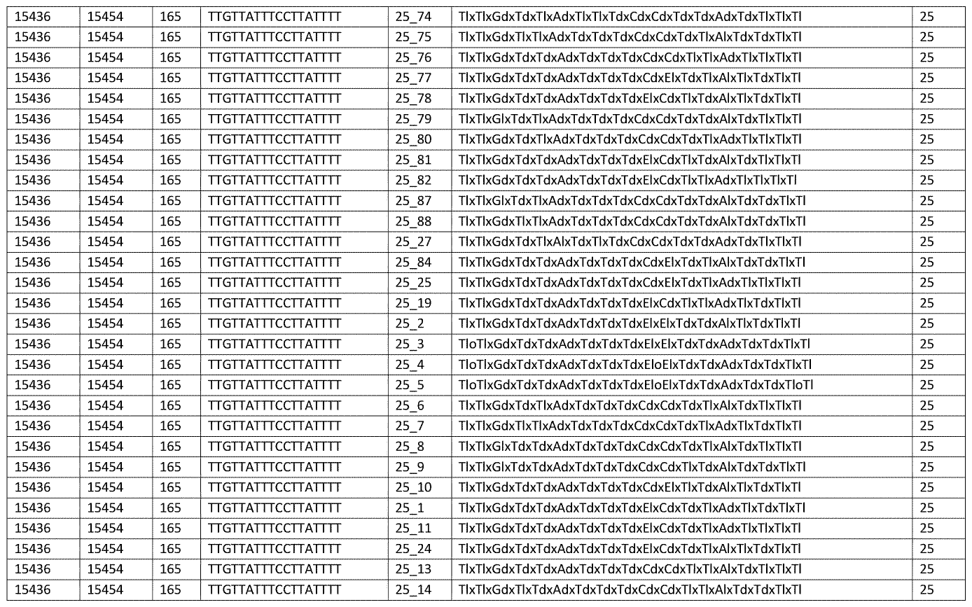

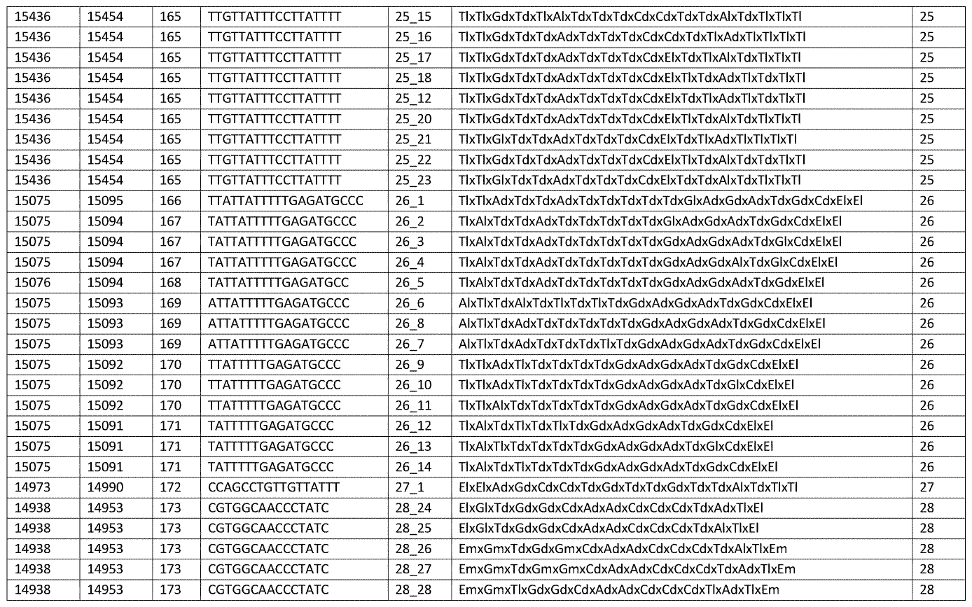

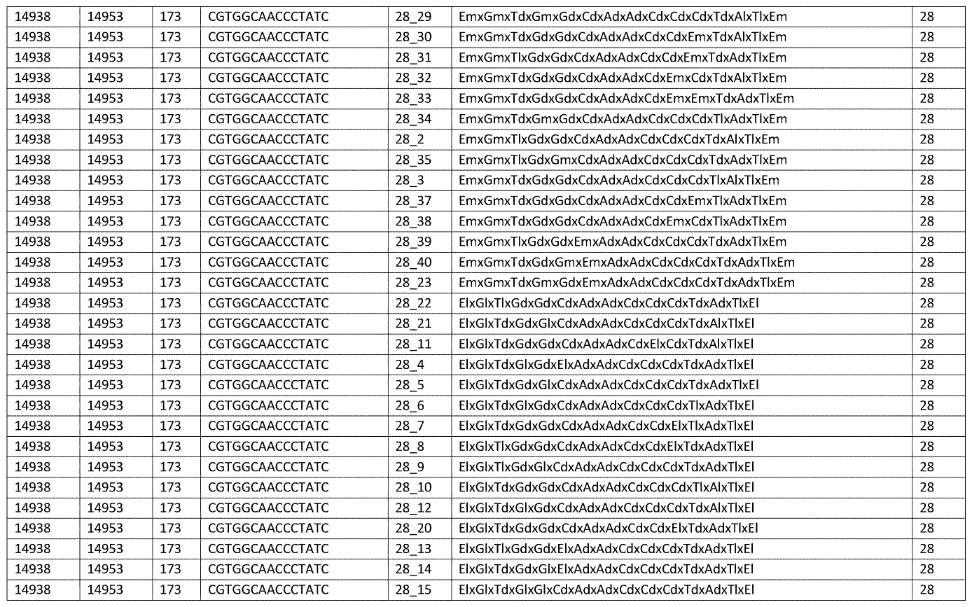

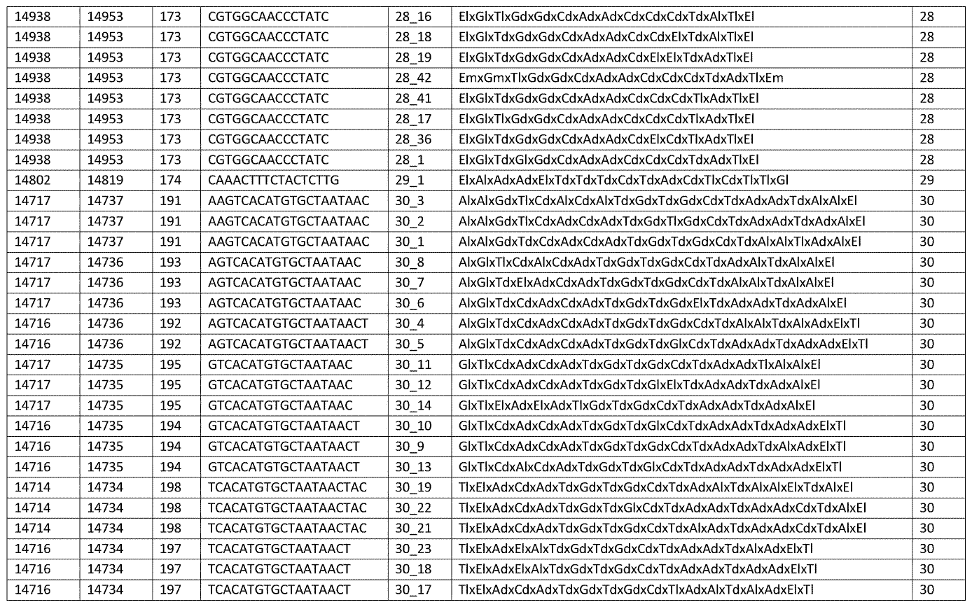

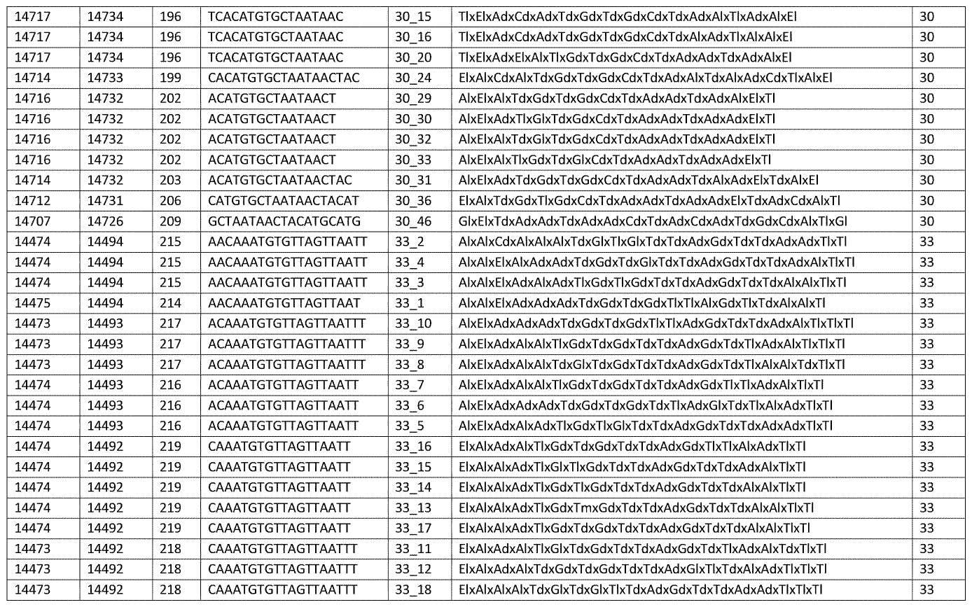

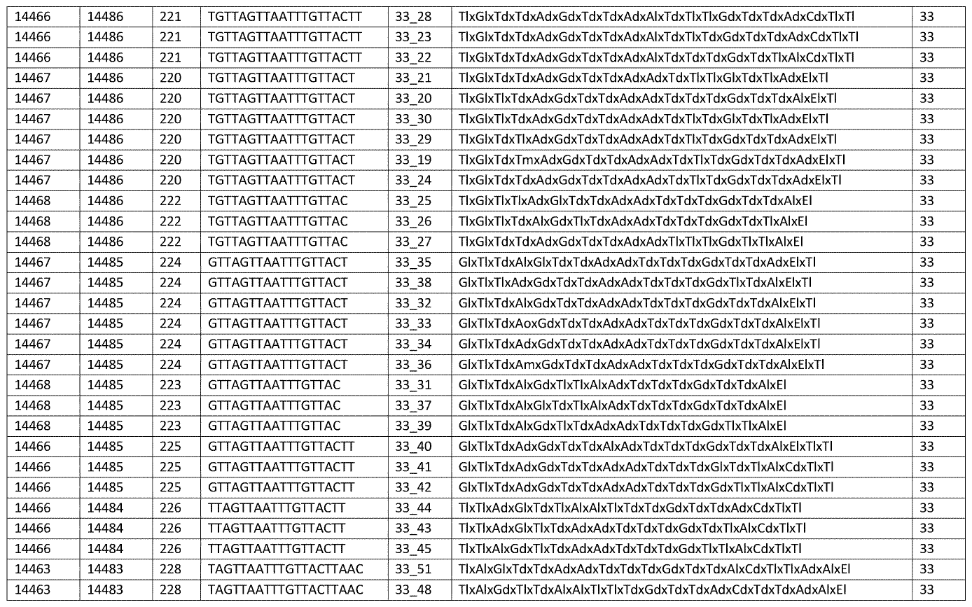

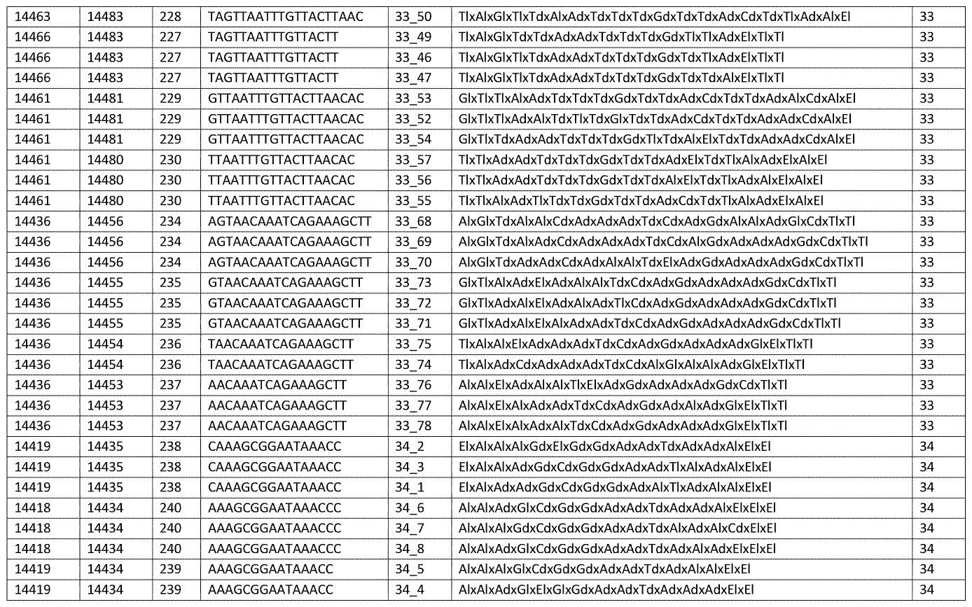

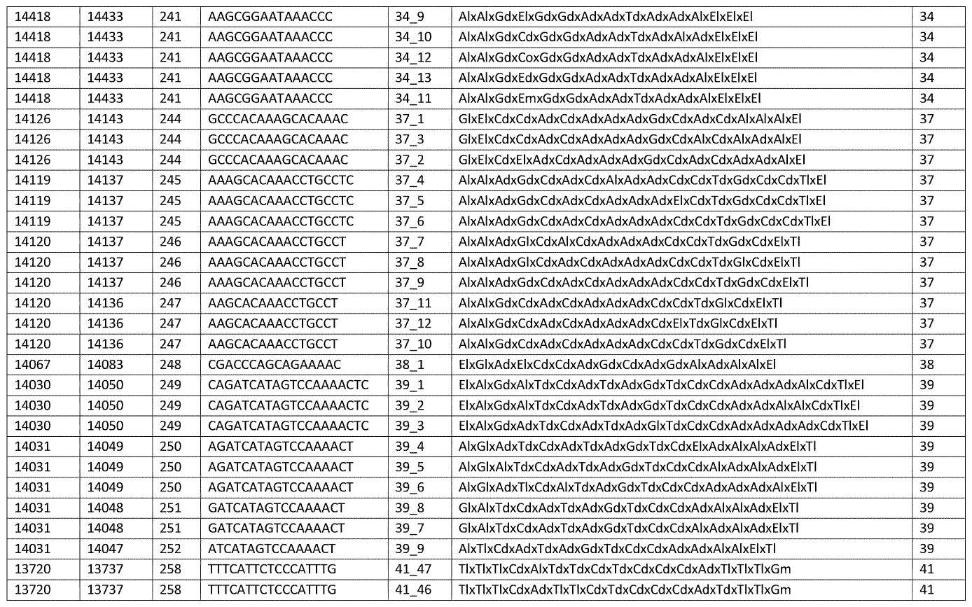

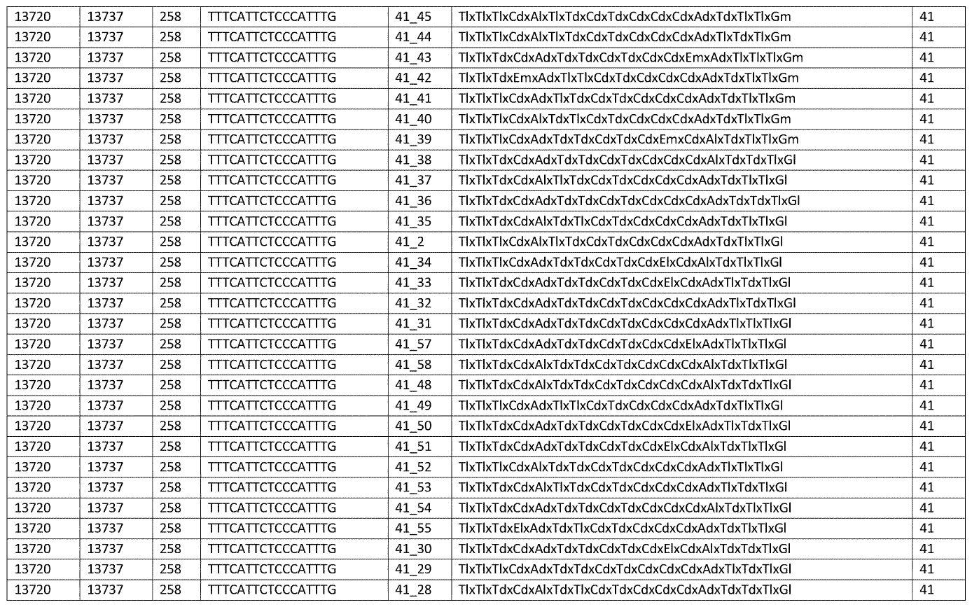

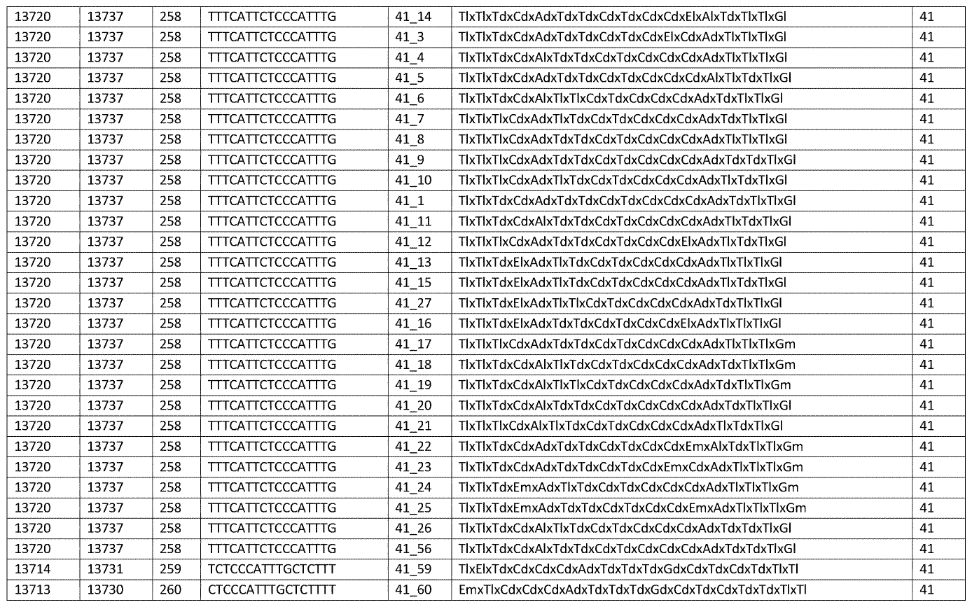

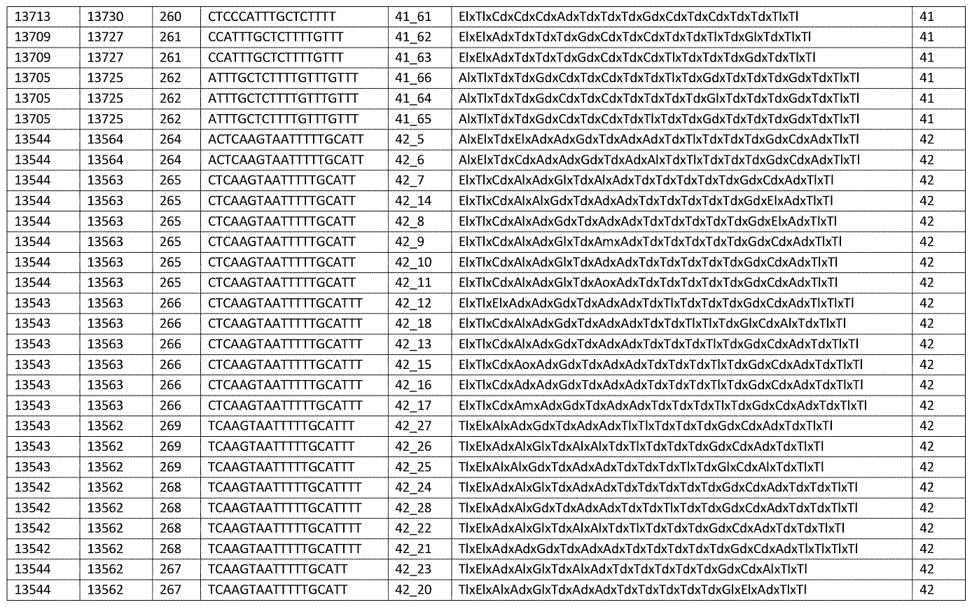

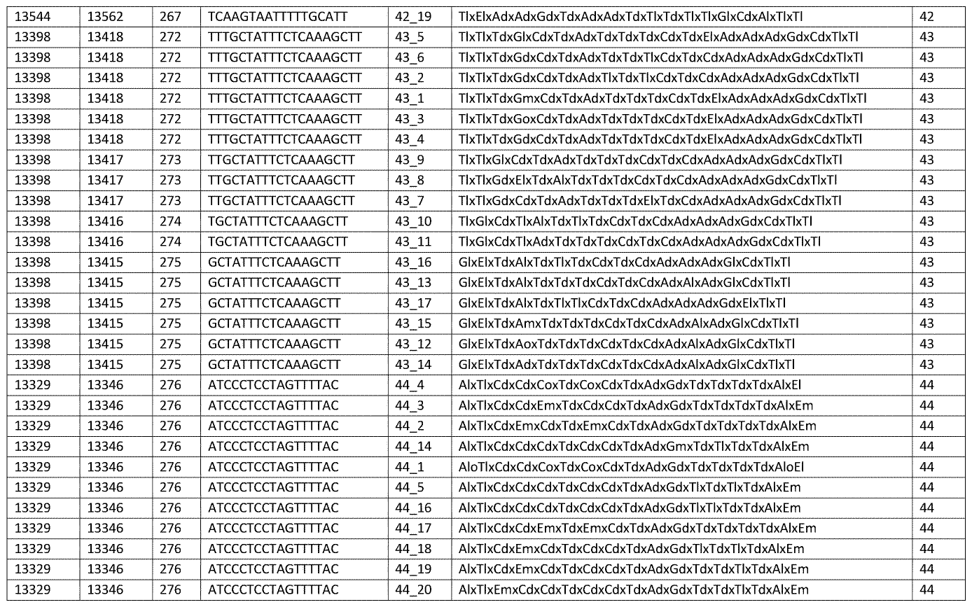

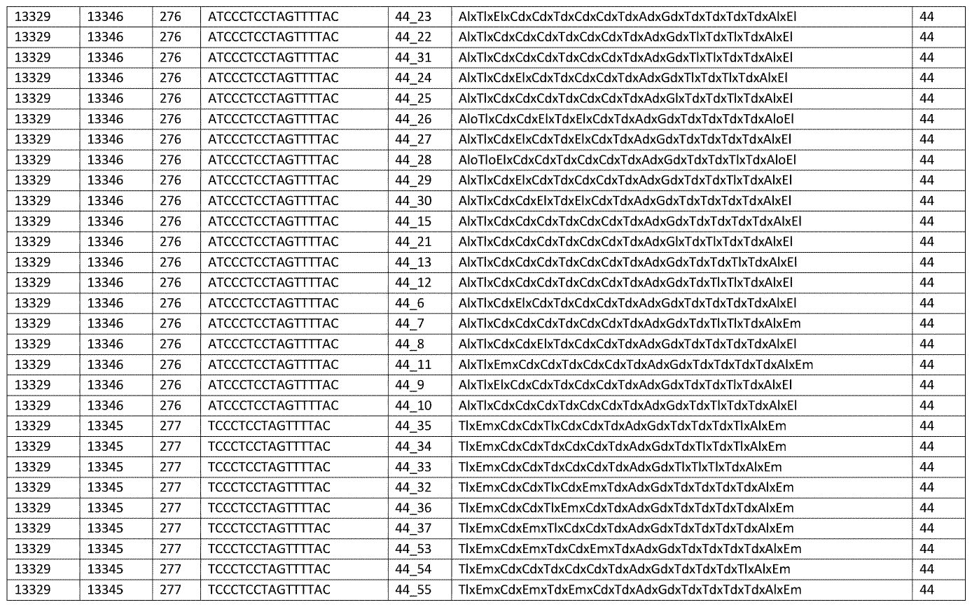

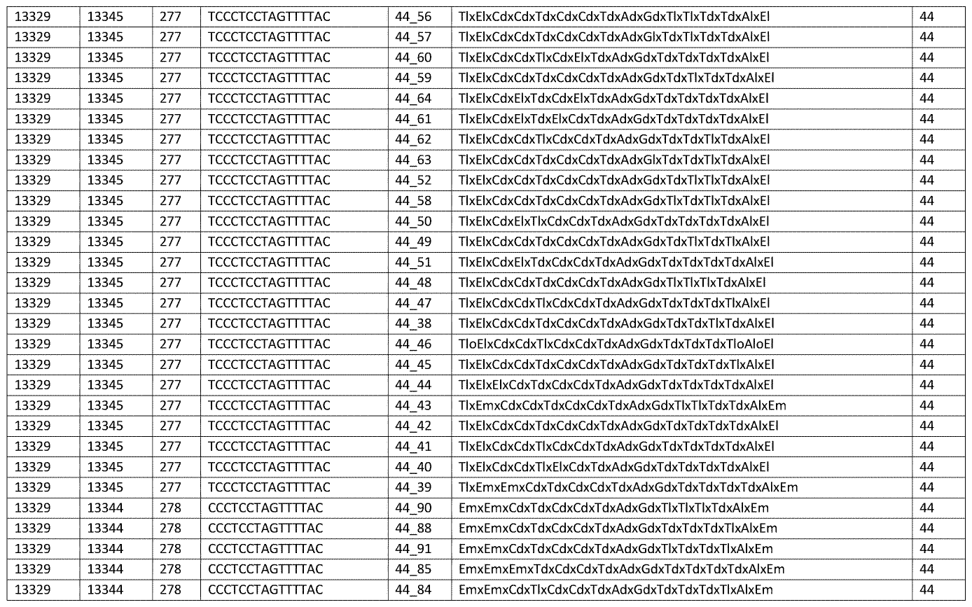

In some embodiments, the antisense oligonucleotide is an antisense compound as shown in Table A1 in FIG. 1, wherein
Adx represents 2′deoxyadenosine-3′-phosphorothioate
Aox represents 2′-O-methyladenosine-3′-phosphorothioate
Amx represents 2′-O-Methoxyethyladenosine-3′-phosphorothioate
Alx represents 2′-O-beta-D-oxy LNA adenosine-3′-phosphorothioate
Cdx represents 2′deoxycytidine-3′-phosphorothioate
Cox represents 2′-O-methylcytidine-3′-phosphorothioate
Edx represents 2′deoxy-5-methylcytidine-3′-phosphorothioate
Emx represents 2′-O-Methoxyethyl-5-methylcytidine-3′-phosphorothioate
Elx represents 2′-O-beta-D-oxy LNA-5-methylcytidine-3′-phosphorothioate
Gdx represents 2′deoxyguanosine-3′-phosphorothioate
Gox represents 2′-O-methylguanosine-3′-phosphorothioate
Gmx represents 2′-O-Methoxyethylguanosine-3′-phosphorothioate
Glx represents 2′-O-beta-D-oxy LNA guanosine-3′-phosphorothioate
Tdx represents 2′deoxythymidine-3′-phosphorothioate
Tmx represents 2′-O-Methoxyethylthymidine-3′-phosphorothioate
Tlx represents 2′-O-beta-D-oxy LNA thymidine-3′-phosphorothioate
Ado represents 2′deoxyadenosine-3′-phosphodiester
Aoo represents 2′-O-methyladenosine-3′-phosphodiester
Amo represents 2′-O-Methoxyethyladenosine-3′-phosphodiester
Alo represents 2′-O-beta-D-oxy LNA adenosine-3′-phosphodiester
Cdo represents 2′deoxycytidine-3′-phosphodiester
Coo represents 2′-O-methylcytidine-3′-phosphodiester
Edo represents 2′deoxy-5-methylcytidine-3′-phosphodiester
Emo represents 2′-O-methoxyethyl-5-methylcytidine-3′-phosphodiester
Elo represents 2′-O-beta-D-oxy LNA-5-methylcytidine-3′-phosphodiester
Gdo represents 2′deoxyguanosine-3′-phosphodiester
Goo represents 2′-O-methylguanosine-3′-phosphodiester
Gmo represents 2′-O-Methoxyethylguanosine-3′-phosphodiester
Glo represents 2′-O-beta-D-oxy LNA guanosine-3′-phosphodiester
Tdo represents 2′deoxythymidine-3′-phosphodiester
Tmo represents 2′-O-Methoxyethylthymidine-3′-phosphodiester
Tlo represents 2′-O-beta-D-oxy LNA thymidine-3′-phosphodiester
Uo represents 2′-O-methyluridine
Ad represents 2′deoxyadenosine
Ao represents 2′-O-methyladenosine
Am represents 2′-O-Methoxyethyladenosine
Al represents 2′-O-beta-D-oxy LNA adenosine
Cd represents 2′deoxycytidine
Co represents 2′-O-methylcytidine
Ed represents 2′deoxy-5-methylcytidine
Em represents 2′-O-Methoxyethyl-5-methylcytidine
El represents 2′-O-beta-D-oxy LNA-5-methylcytidine
Gd represents 2′deoxyguanosine
Go represents 2′-O-methylguanosine
Gm represents 2′-O-Methoxyethylguanosine
Gb represents 2′-O-beta-D-oxy LNA guanosine
Td represents 2′deoxythymidine
Tm represents 2′-O-Methoxyethylthymidine
Tl represents 2′-O-beta-D-oxy LNA thymidine
Uoo represents 2′-O-methyluridine-3′-phosphodiester and
Uox represents 2′-O-methyluridine-3′-phosphorothioate.

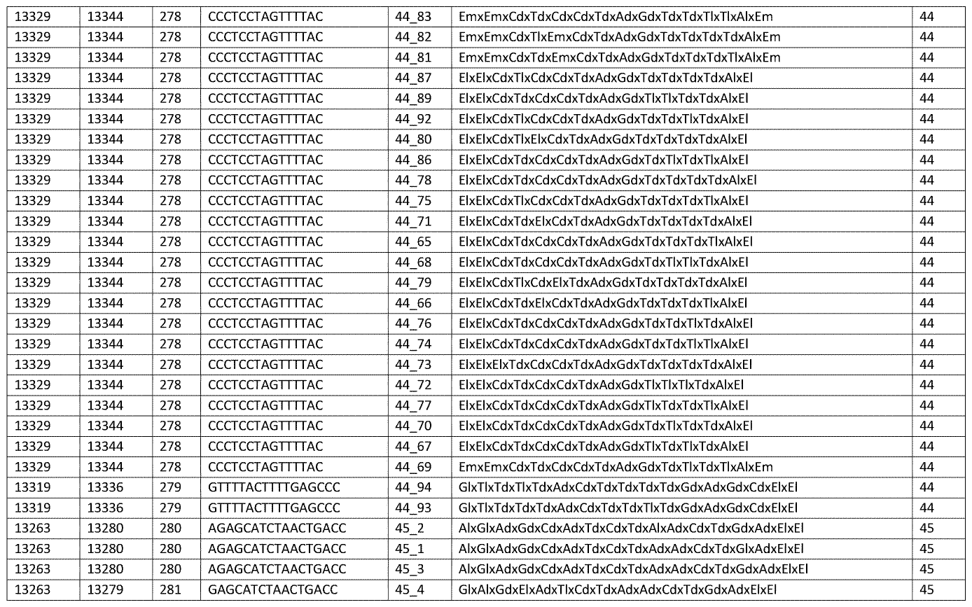

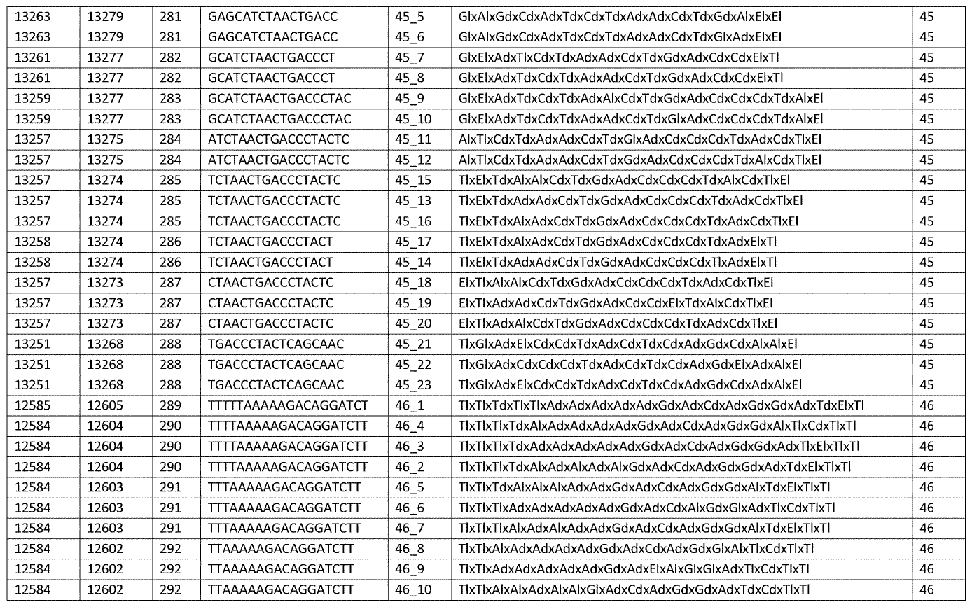

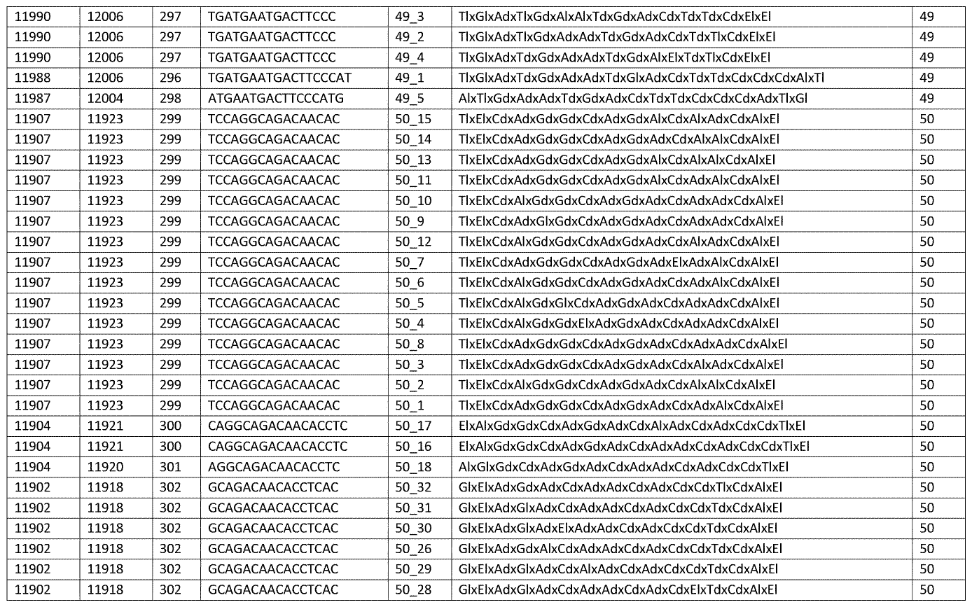

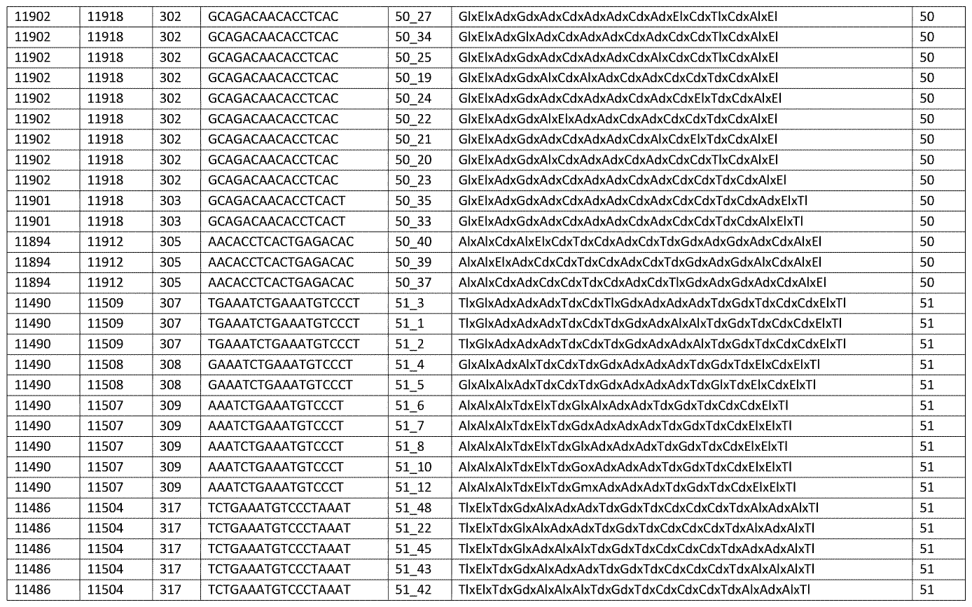

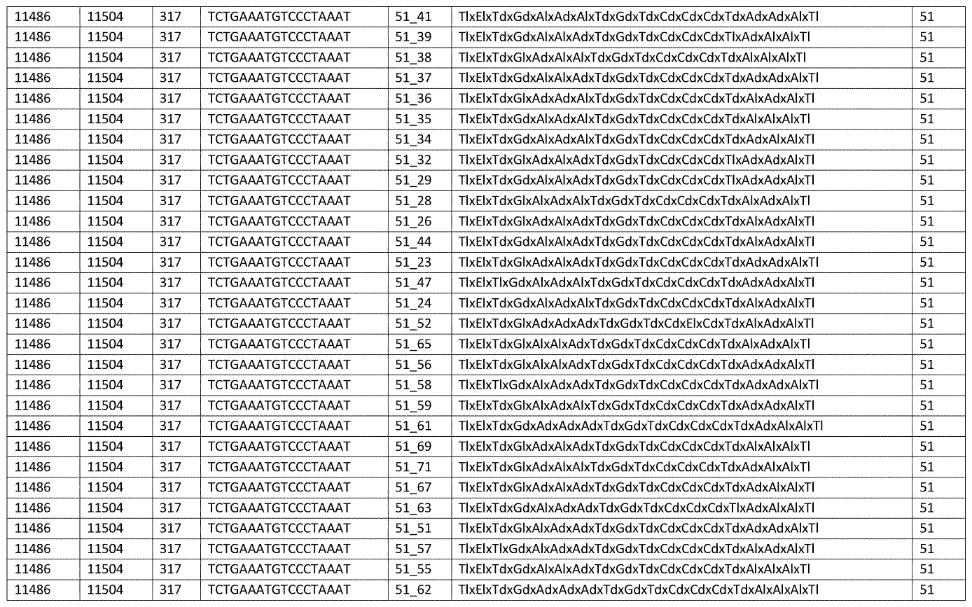

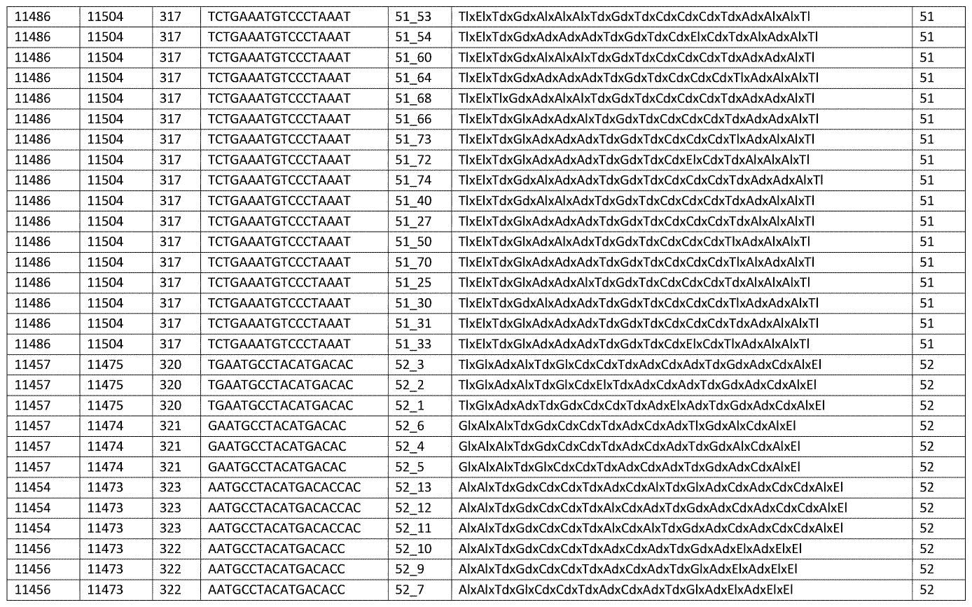

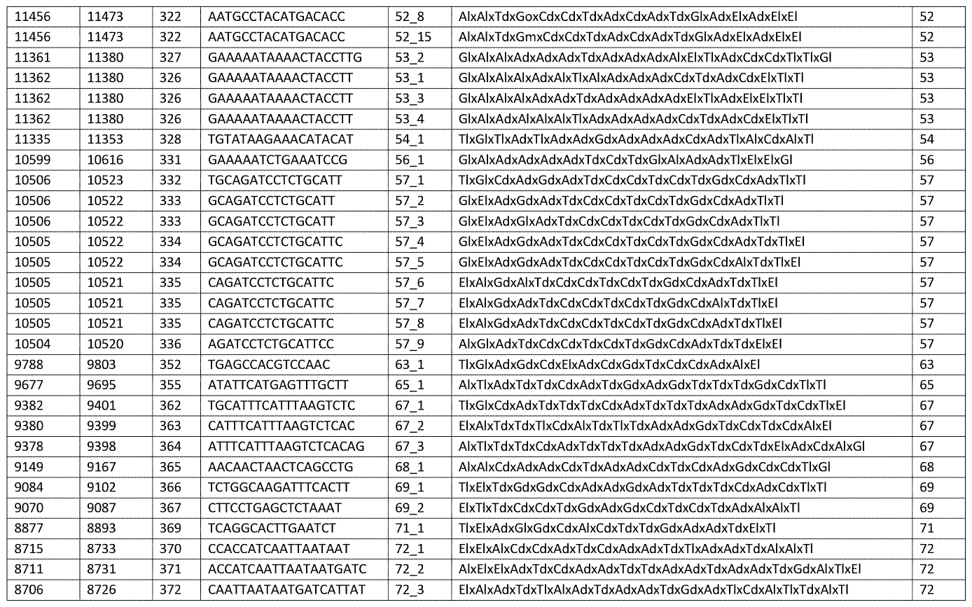

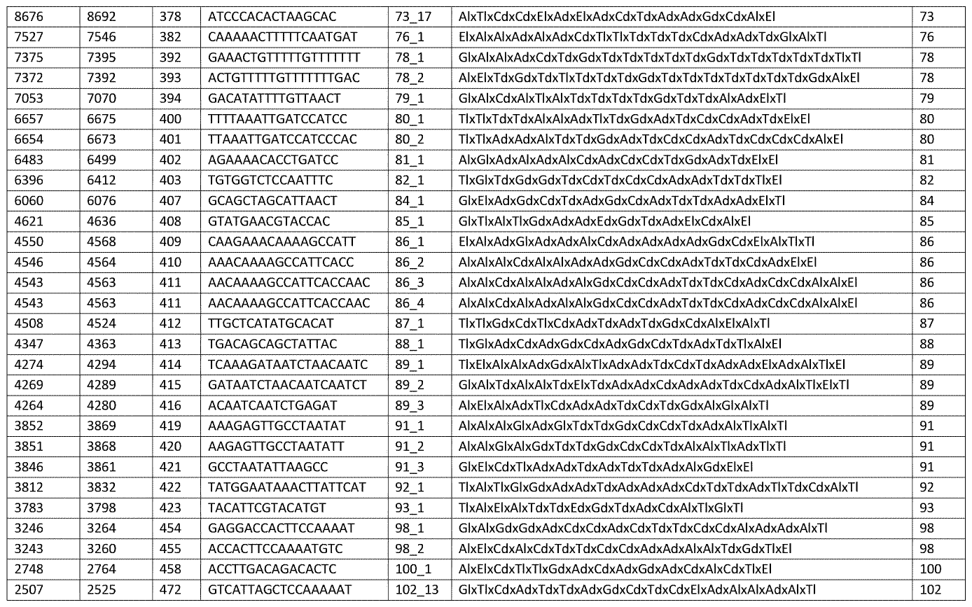

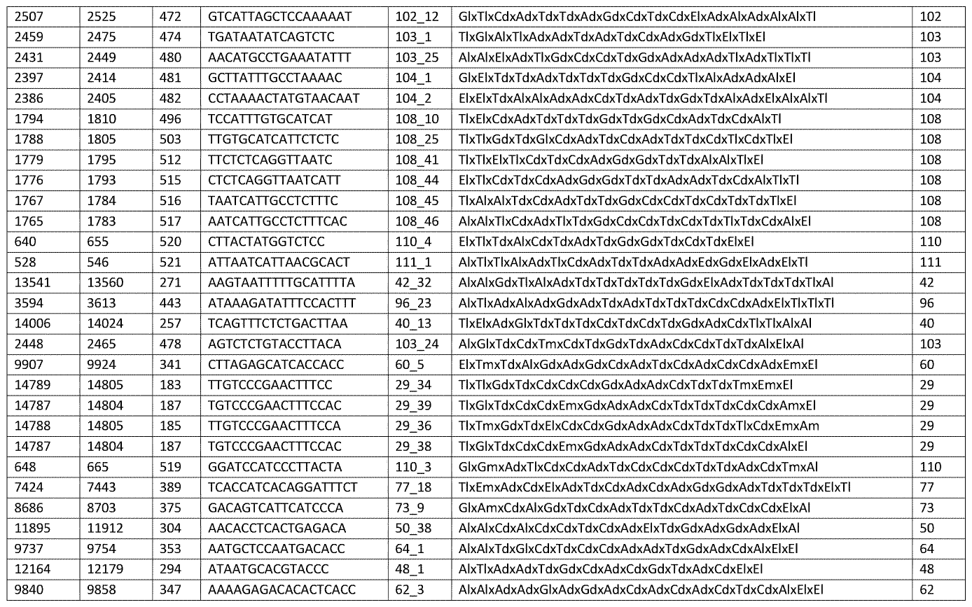

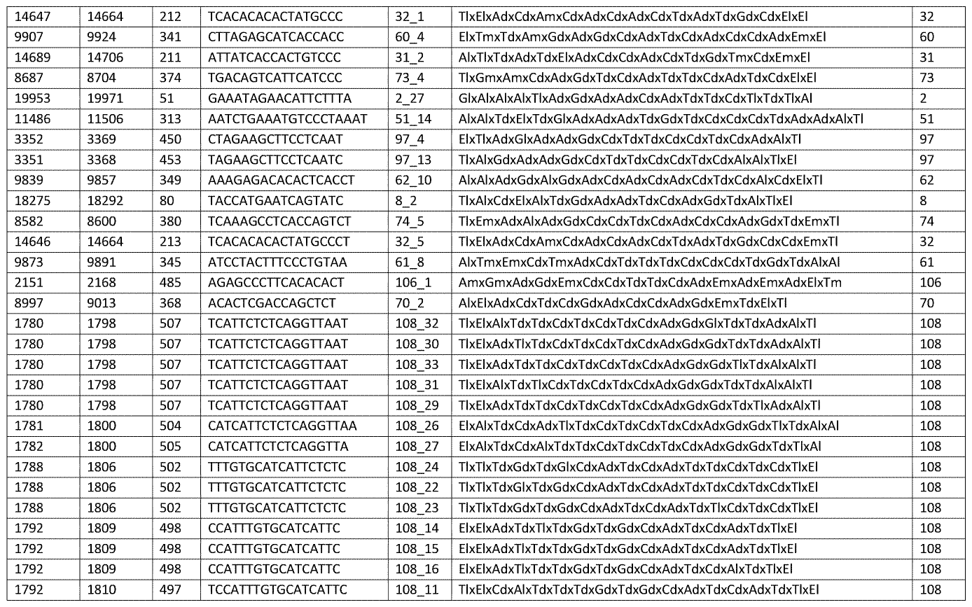

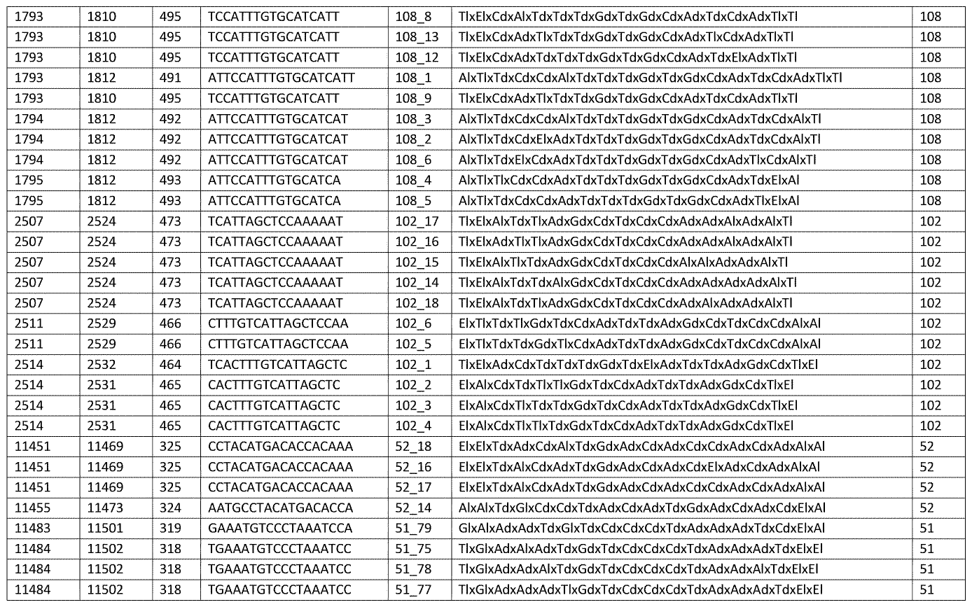

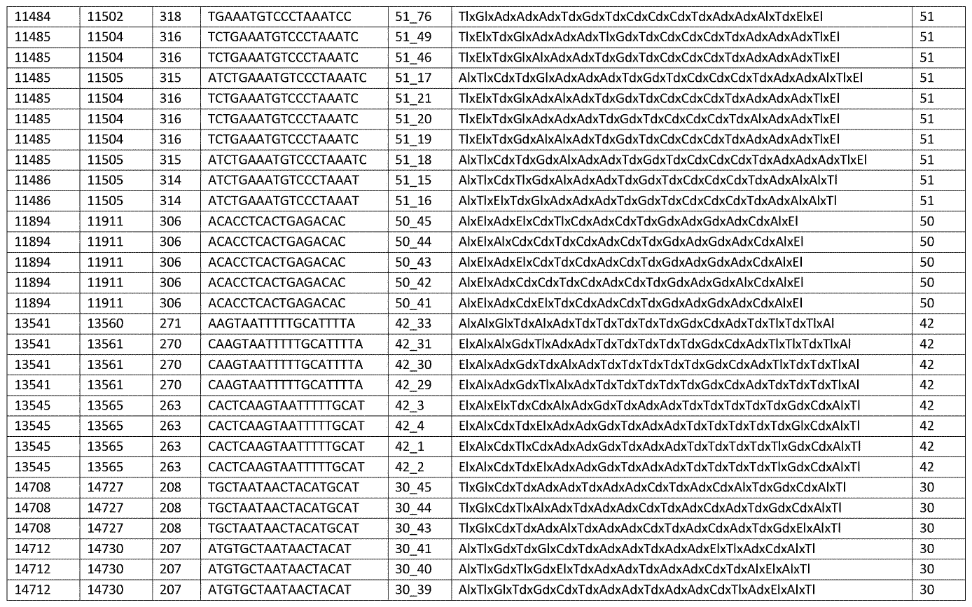

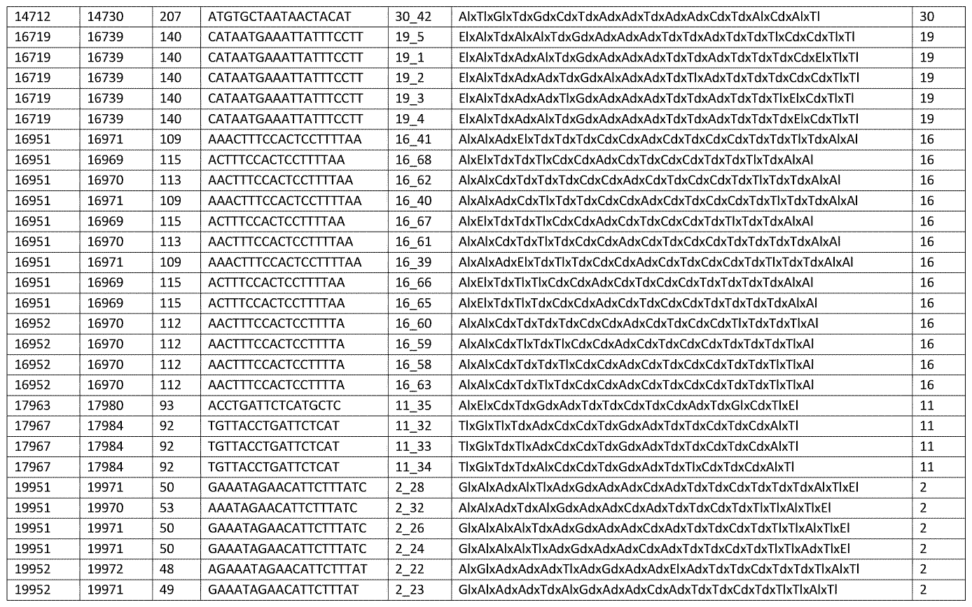

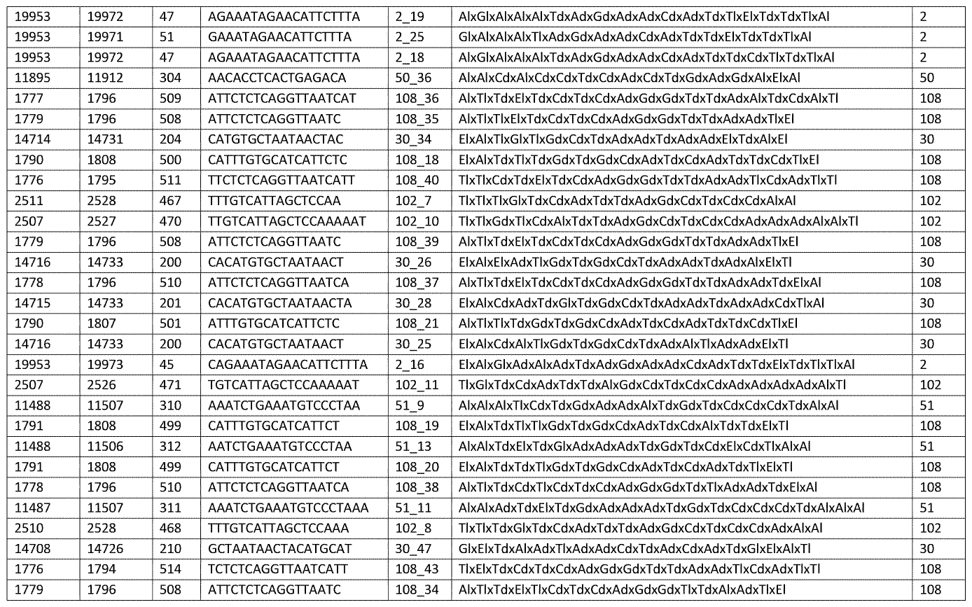

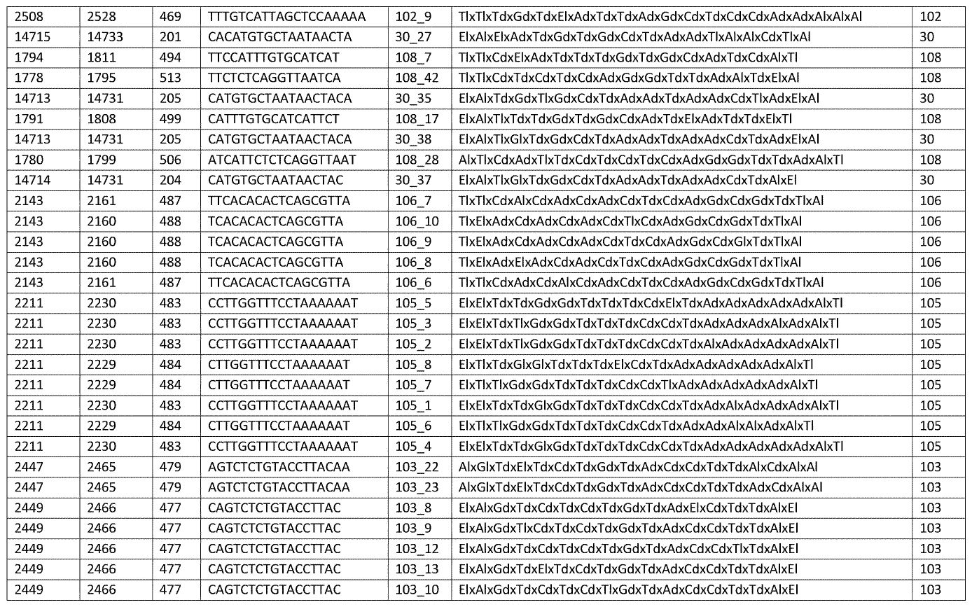

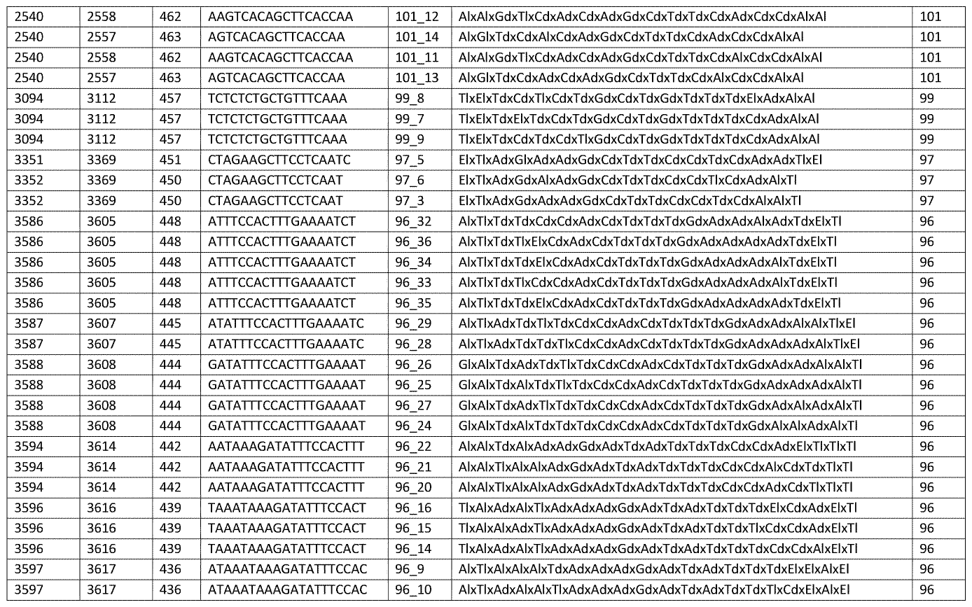

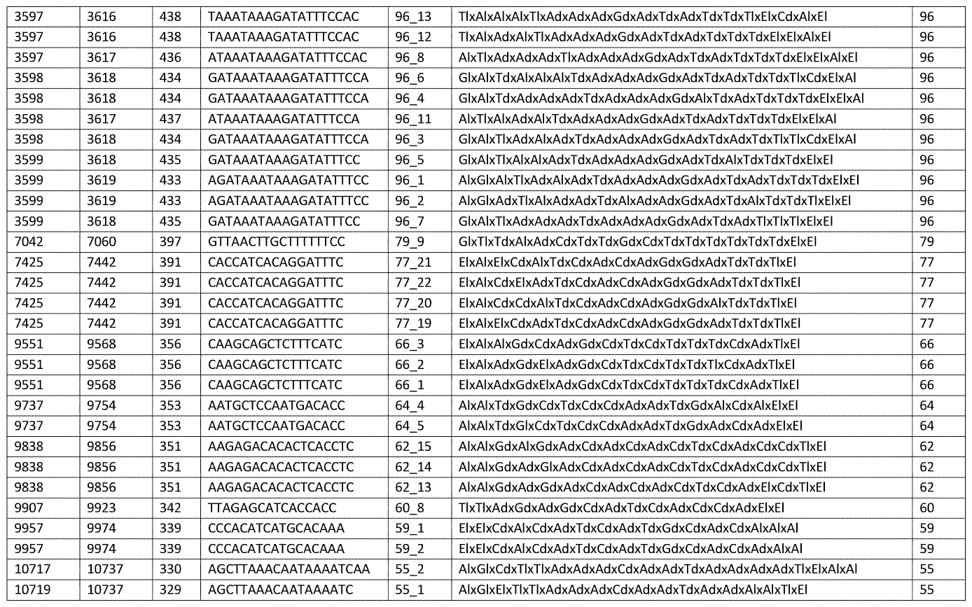

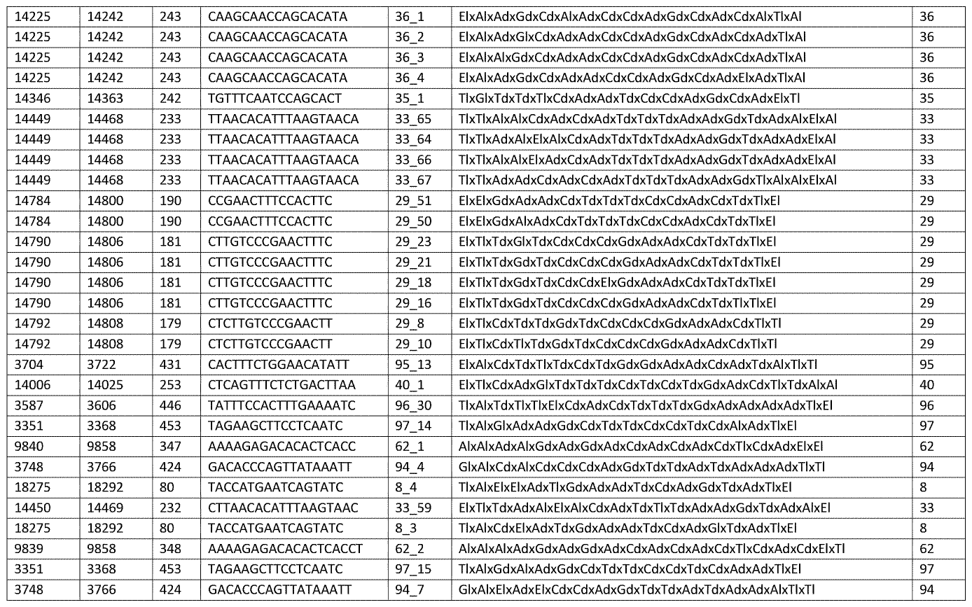

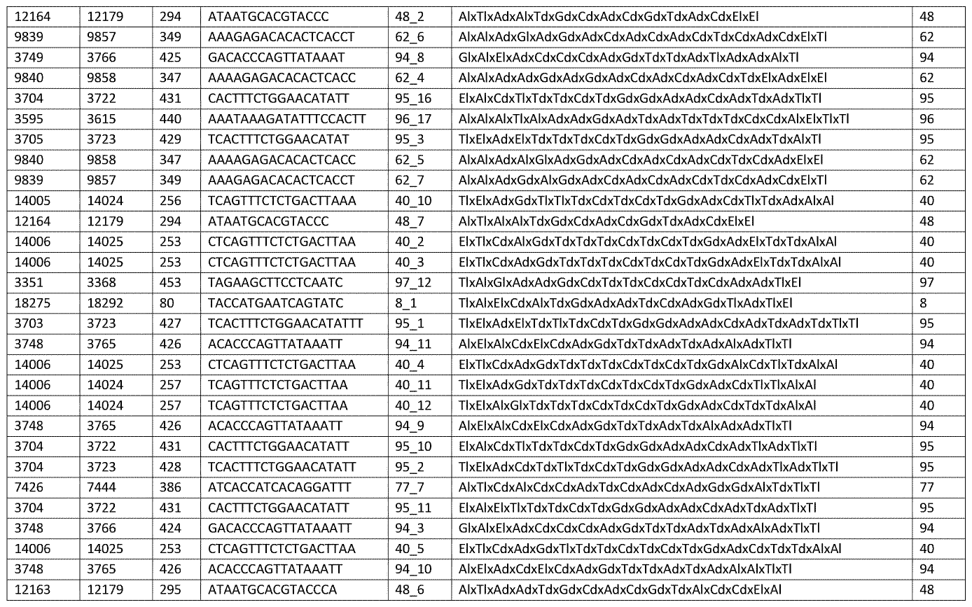

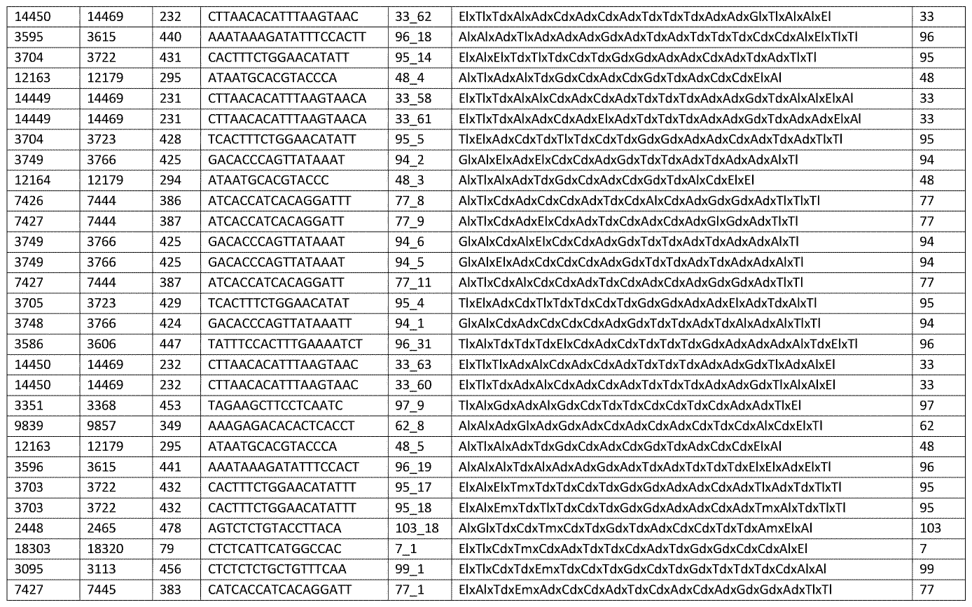

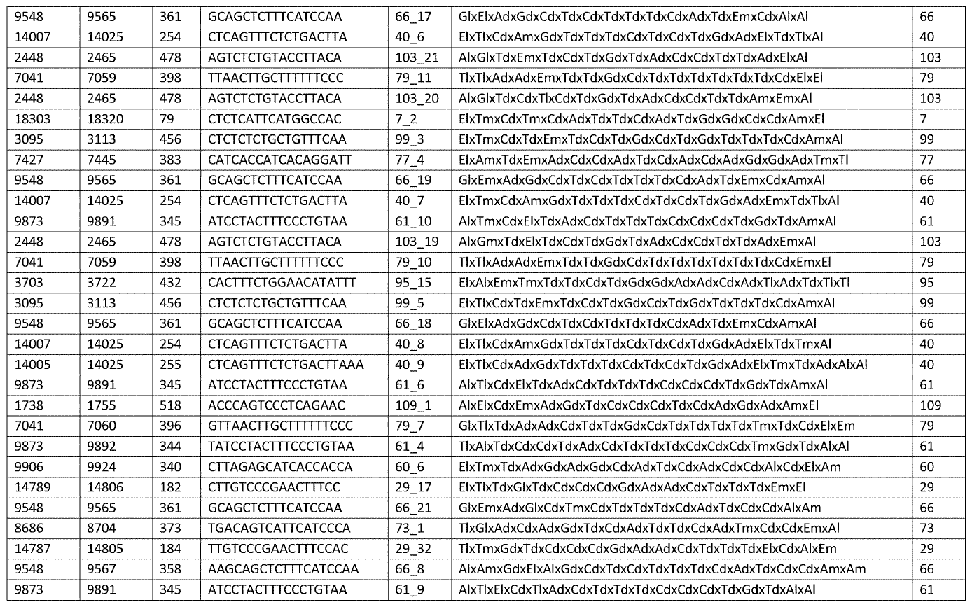

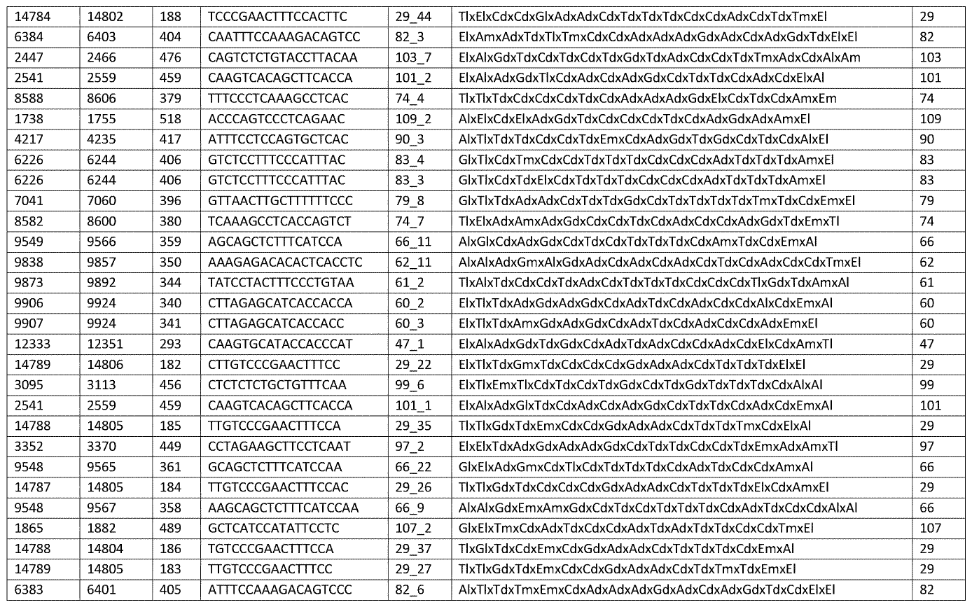

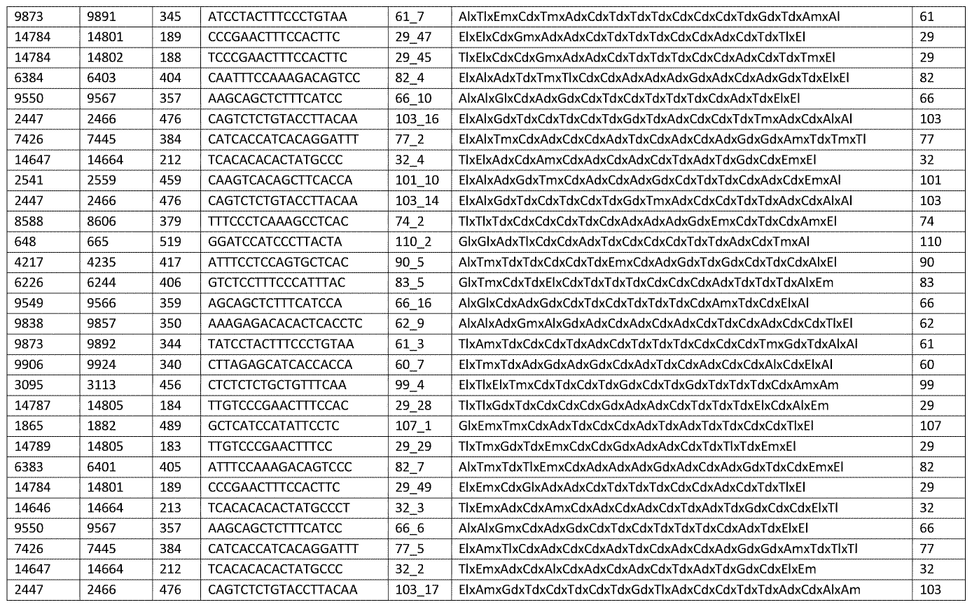

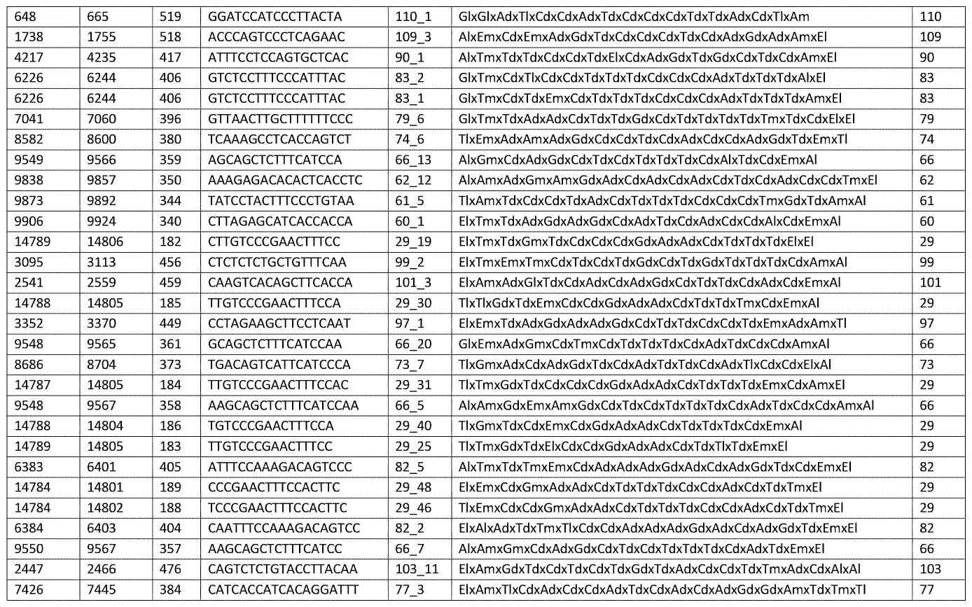

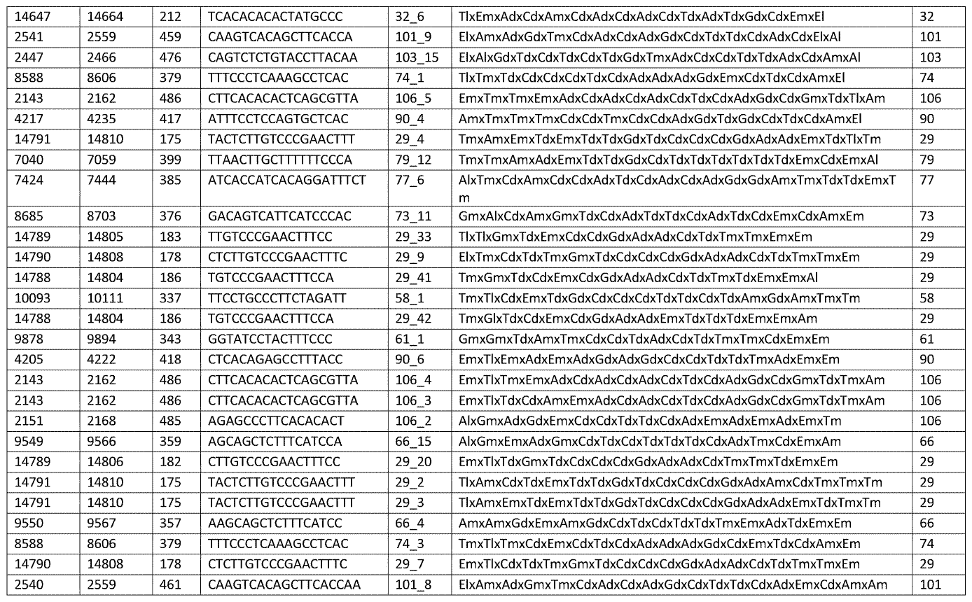

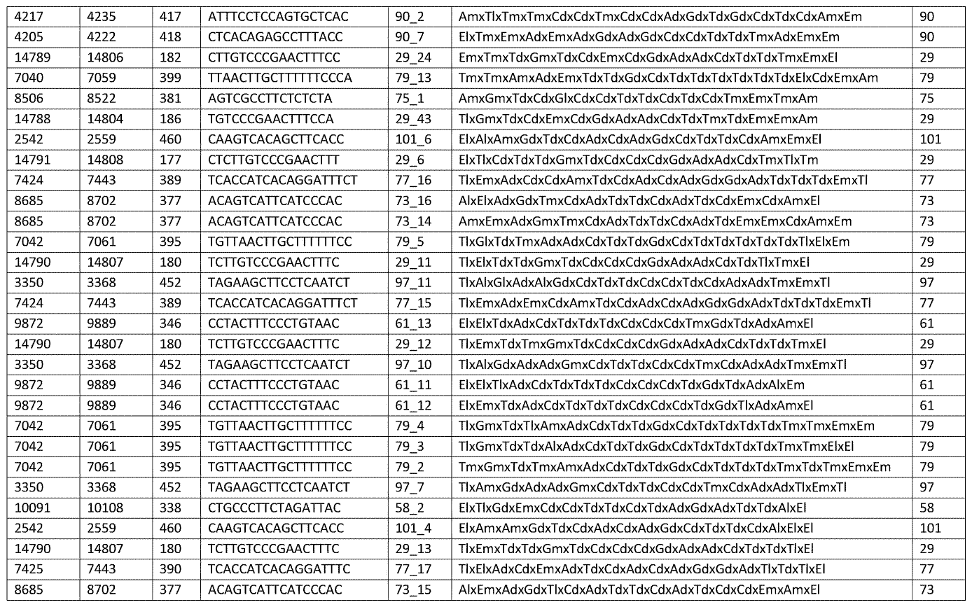

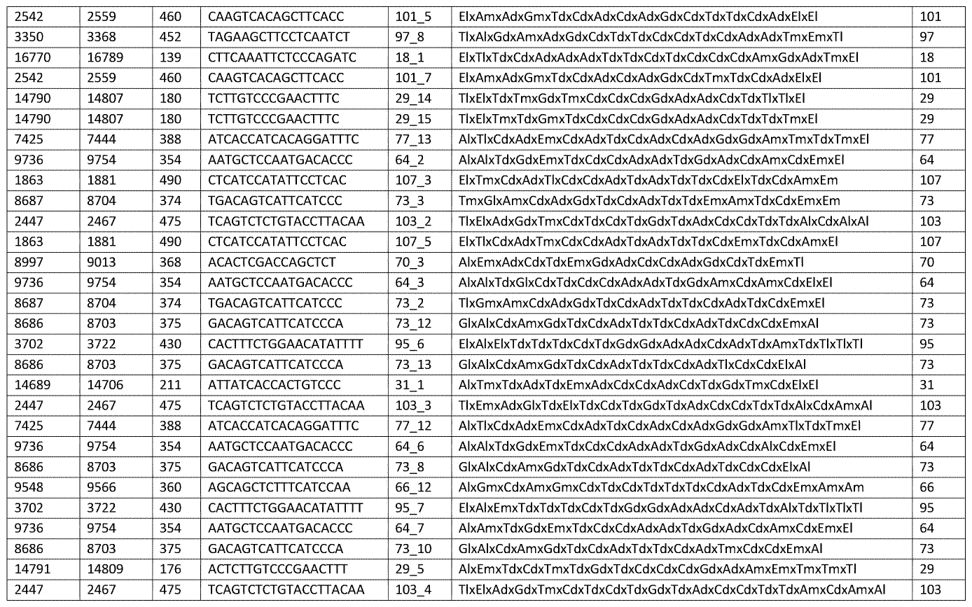

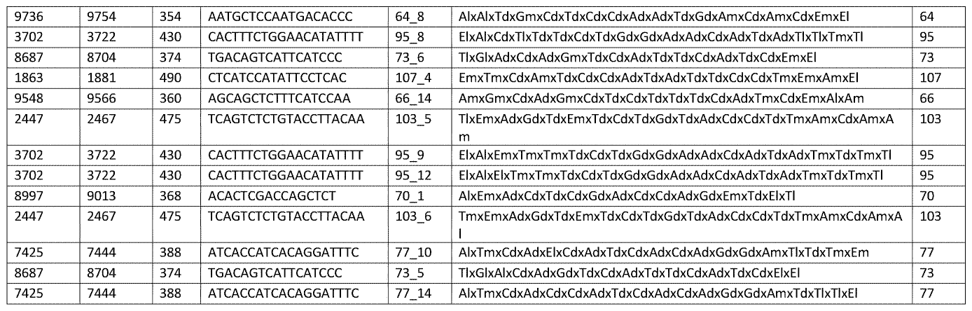

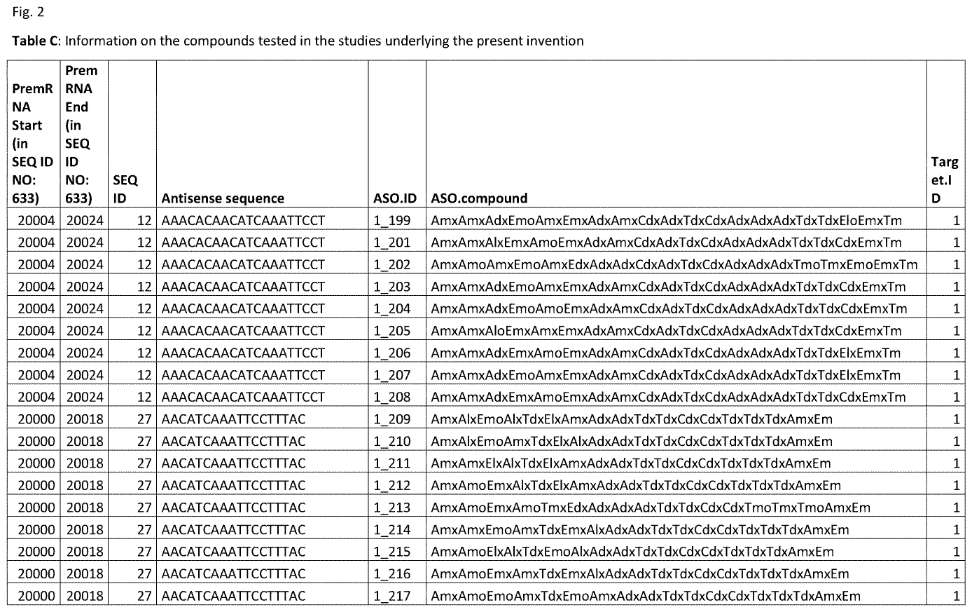

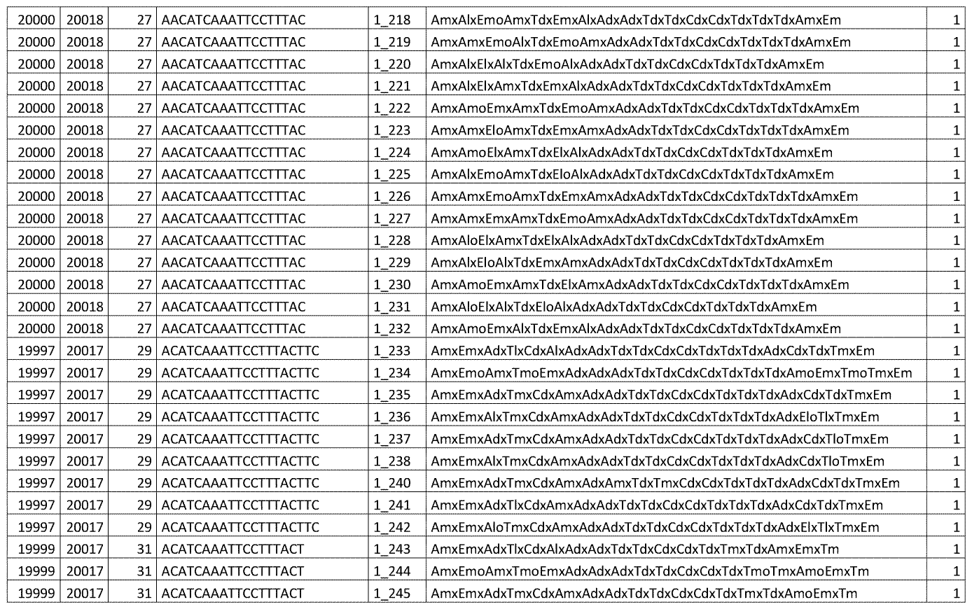

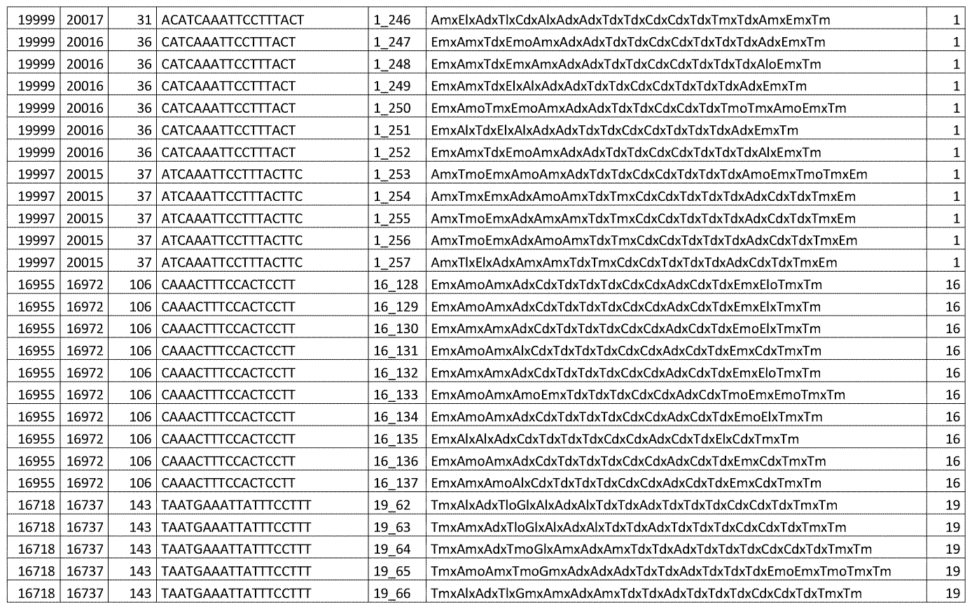

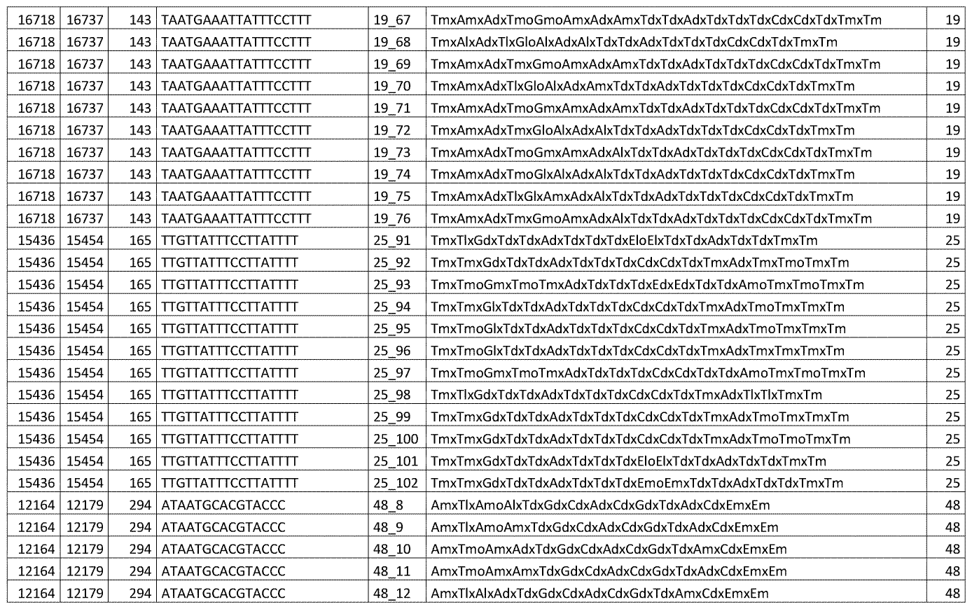

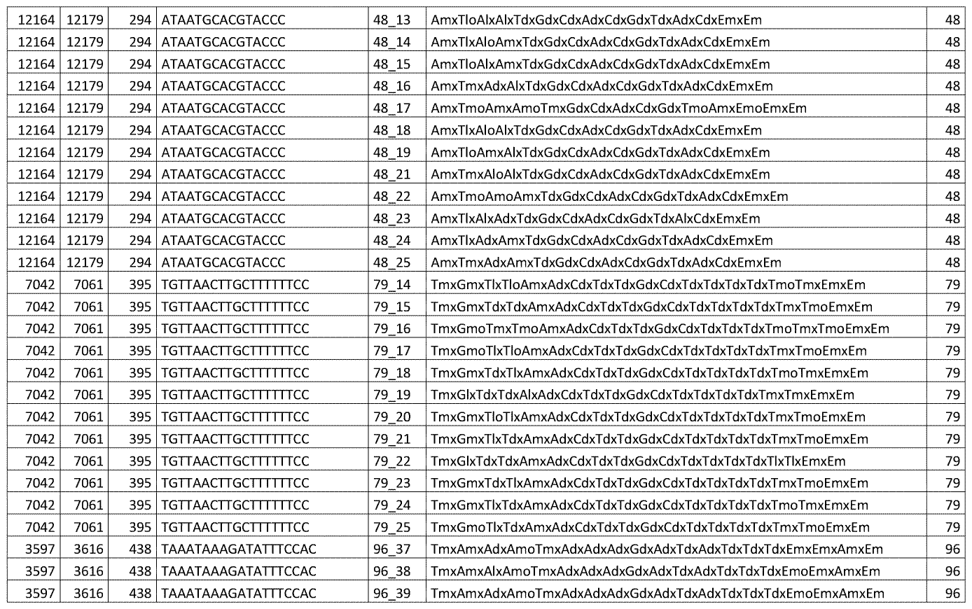

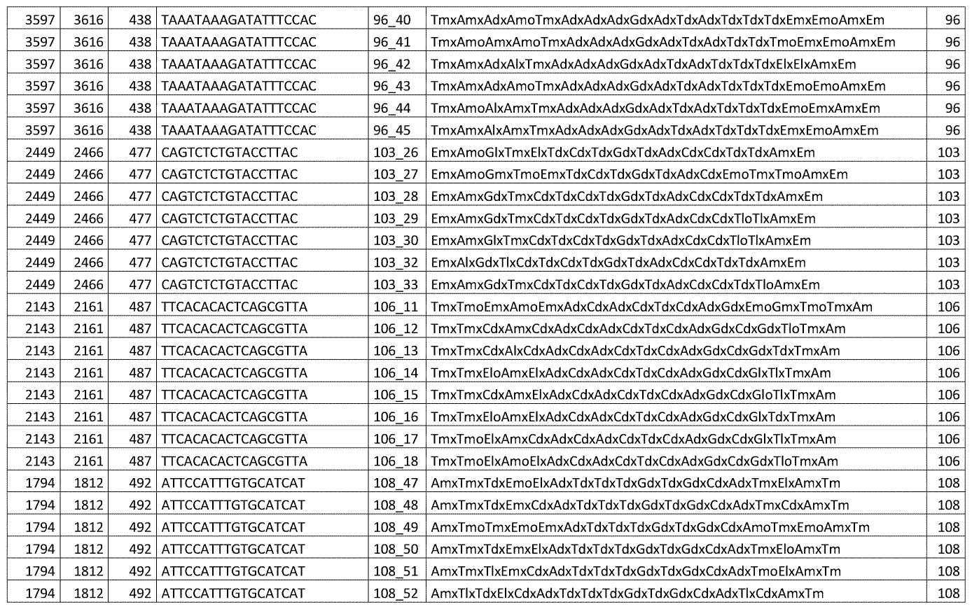

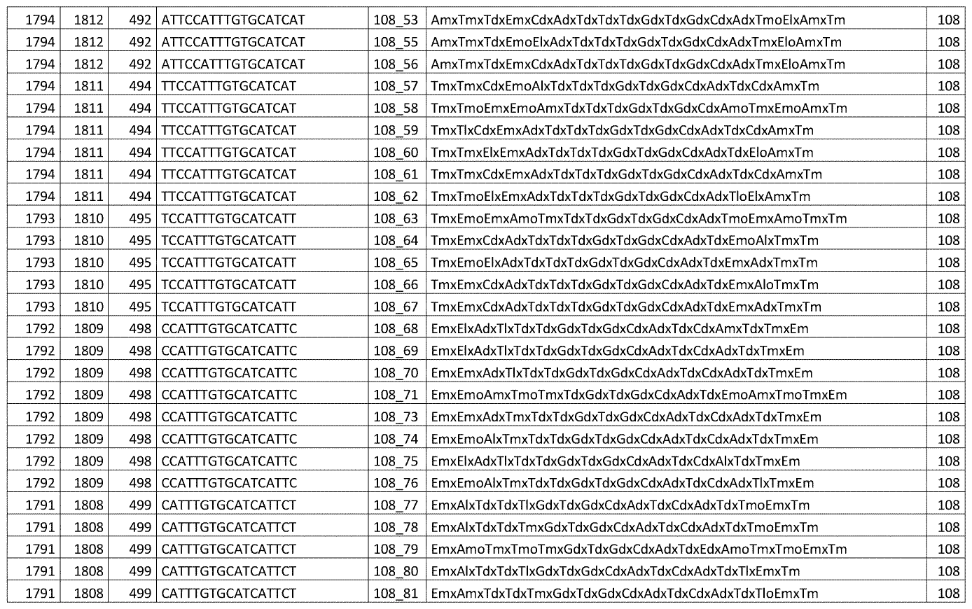

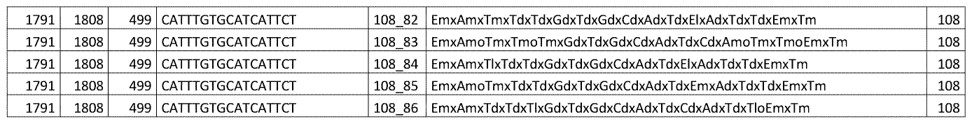

In some embodiments, the antisense oligonucleotide is an antisense compound as shown in Table C in FIG. 2. For the annotation, please see the previous paragraph.

In an embodiment, the antisense oligonucleotide is an antisense oligonucleotide with ASO ID 1_41, 1_103, 1_131, 1_129, 1_116, 1_164, 1_193, 16_17, 19_23, 25_85, 25_16, 108_15, 10816, 108_12, 108_6, 102_4, 108_20, 1087, 108_17, 106_7, 103_10, 96_21, 96_12, 48_2, 48_7, 483, 793, 95_7, and 95_9, as shown in Table A1 in FIG. 1. The ASO compounds with these ASO IDs had a strong effect on the down-regulation of the target gene (see Examples).

In preferred embodiment, the antisense oligonucleotide of the present invention is capable of reducing the amount of GAL3ST1 (Galactosylceramide sulfotransferase) mRNA (typically pre-mRNA) in a host cell expressing said GAL3ST1 mRNA. Typically, the host cell is a mammalian cell, such as a primate cell. In a preferred embodiment, said host cell is human host cell.

Alternatively or additionally, the antisense oligonucleotide of the present invention is capable of reducing the amount of GAL3ST1 the antisense oligonucleotide is capable of reducing galactosylceramide sulfotransferase activity in a host cell, and/or Alternatively or additionally, the antisense oligonucleotide of the present invention is capable of reducing the amount of sulfatide in a host cell.

Preferably, the antisense oligonucleotide is a chemically modified antisense oligonucleotide. A chemically modified antisense oligonucleotide typically comprises modifications of the phosphodiester backbone chemistry, nucleobase modifications and sugar modifications.

In a preferred embodiment, the chemically modified antisense oligonucleotide comprises at least one 2′ modified sugar or bicyclic sugar.

Alternatively or additionally, the chemically modified antisense oligonucleotide contains at least one modified nucleobase. For example, at least one modified nucleobase is 5-methylcytosine.

Alternatively or additionally, the chemically modified antisense oligonucleotide comprises at least one modified nucleoside selected from the group consisting of: 2′-O-Methoxyethyl-RNA, 2′-O-Methyl-RNA, 2′-Fluoro-RNA.

Further, the chemically modified antisense oligonucleotide may comprise at least one modified intemucleoside linkage. In an embodiment, at least five, such as at least 10 intemucleoside linkages are modified intemucleoside linkages. In an embodiment, all intemucleoside linkages are modified internucleoside linkages.

A “modified intemucleoside linkage” as used herein, refers to an intemucleoside linkage other than a phosphodiester linkage. Thus, the antisense oligonucleotide may comprise unmodified intemucleoside linkages (i.e. phosphodiester linkages), modified internucleoside linkages, or a combination thereof.

Preferably, the modified linkage(s) is (are) selected from: a Phosphorothioate intemucleoside linkage, a Phosphorodithioate intemucleoside linkage, a Phophoroamidate intemucleoside linkage, a methyl phosphonate intemucleoside linkage, a phosphotriester internucleoside linkage, a boranophosphate internucleoside linkage and a phosphoryl guanidine internucleoside linkage. Moreover, internucleoside linkage can be stereodefined versions of said linkages

More preferably, the at least one modified linkage is a phosphorothioate linkage. Most preferably, at least 50% of the intemucloside linkages, such as all intemucleoside linkages, are phosphorothioate intemucleoside linkages.

Preferably, the antisense oligonucleotide comprises at least one nucleoside with a modified sugar moiety, typically at least four nucleosides with a modified sugar moiety (herein also referred to as sugar modified nucleosides).

In an embodiment, the antisense oligonucleotide comprises at least one, such as one, two, three, four or more LNA (locked nucleic acid) or MOE (2′-O-Methoxyethyl) nucleosides. For example, the LNA nucleoside is a beta-D-oxy LNA nucleoside.

Moreover, it is envisaged that the antisense oligonucleotide has a gapmer structure, i.e. is a gapmer.

The present invention further relates to a conjugate comprising the antisense oligonucleotide according to the present invention, wherein the said antisense oligonucleotide is covalently attached to a conjugate moiety.

The present invention further relates to pharmaceutical composition comprising an inhibitor of the human GAL3ST1 protein. In particular, the present invention relates to pharmaceutical composition comprising the antisense oligonucleotide according to the present invention or the conjugate according to the present invention. In an embodiment, the composition further comprises diluents and carriers.

The present invention further relates to the inhibitor of human GAL3ST1 or the pharmaceutical composition according to the present invention for use in treating metachromatic leukodystrophy. The inhibitor, in particular, is the antisense oligonucleotide according to the present invention, the conjugate according to the present invention, or the pharmaceutical composition according to the present invention for use in treating metachromatic leukodystrophy.

The present invention further relates to a method for treating metachromatic leukodystrophy, comprising administering to a subject suffering from metachromatic leukodystrophy a pharmaceutically effective amount of inhibitor of human GAL3ST1, in particular the antisense oligonucleotide according to the present invention, the conjugate according to the present invention, or the pharmaceutical composition according to the present invention for use in treating metachromatic leukodystrophy.

In some embodiments, the inhibitor is a siRNA or short-hairpin RNA targeting the GAL3ST1 gene.

In some embodiments, the inhibitor is an antibody, or antigen binding fragment thereof that specifically binds to GAL3ST1.

The present invention further relates to a method for identifying a candidate compound for the treatment of metachromatic leukodystrophy, comprising
a) providing an antisense oligonucleotide according to the present invention,
b) contacting a host cell expressing GAL3ST1 mRNA with said antisense oligonucleotide,
c) determining the amount of GAL3ST1 mRNA in the said host cell, and
d) identifying a candidate compound based on the results of step c).

### DETAILED OVERVIEW ON THE PRESENT INVENTION

Inhibition of GAL3ST1 (Galactosylceramide sulfotransferase) expression is considered as therapeutic concept for treating metachromatic leukodystrophy. In the studies underlying the present invention, target regions within the GAL3ST1 pre-mRNA were identified which—when targeted by antisense oligonucleotides—allow for efficient downregulation of the human GAL3ST1 pre-mRNA (or mRNA) in a host cell expressing said pre-mRNA or mRNA. The sequences of the target regions are shown in Tables B1, B2 and B3. Thus, the invention provides antisense oligonucleotides, which are capable of downregulating GAL3ST1. Preferably, the antisense oligonucleotides (ASOs) comprise a stretch of at least 10 nucleotides which is preferably 90%, more preferably, 95% and most preferably fully complementary (i.e. 100% complementary) to the target region (herein also referred to as target sequence). The antisense oligonucleotides of the present invention are candidates for the treatment of metachromatic leukodystrophy.

Accordingly, the present invention relates to an antisense oligonucleotide comprising a stretch of at least 10 nucleotides which is at least 90% complementary to a target sequence in a GAL3ST1 (Galactosylceramide sulfotransferase) gene.

The term “oligonucleotide” as used herein is well known in the art. As used herein, the term refers to a molecule of at least ten covalently linked nucleotides. Typically, the oligonucleotides as referred to herein are chemically synthesized, for example by solid-phase chemical synthesis. The oligonucleotides as referred to herein shall contain various chemical modifications which typically do not occur in nature. For example, the antisense oligonucleotide may contain at least one 2′ modified sugar. In a preferred embodiment, the antisense oligonucleotides are gapmers. The oligonucleotides of the present invention are antisense oligonucleotides, and in particular single-stranded oligonucleotides. Accordingly, they shall be capable of binding the GAL3ST1 gene, in particular to the GAL3ST1 pre-mRNA, when expressed in a cell, thereby down-regulating the expression of GAL3ST1 gene in the cell. In an embodiment, the cell is a human cell is a cell of the central nervous system (CNS). Typically, the cell is a brain cell.

The GAL3ST1 (Galactosylceramide sulfotransferase-1 or Galactose-3-O-sulfotransferase-1, CST) gene is well known the art. Alternative names are 3′-phosphoadenosine-5′-phosphosulfate:GalCer sulfotransferase gene, 3′-phosphoadenylylsulfate:galactosylceramide 3′-sulfotransferase 1 gene, or cerebroside sulfotransferase or galactose-3-O-sulfotransferase-1 gene. The GAL3ST1 gene is typically the human GAL3ST1 gene. Information on the gene, such as on the nucleic acid sequence, can be found in the known databases, for example, under NCBI Gene ID: 9514).

The human GAL3ST1 gene encodes an enzyme, i.e. protein having galactosylceramide sulfotransferase activity (EC 2.8.2.11). Typically, the enzyme catalyzes the transfer of a sulfate group to position 3 of non-reducing beta-galactosyl residues in glycerolipids and sphingolipids. Typically, the enzyme catalyzes the synthesis of galactosylceramide sulfate (also known as sulfatide). Typically, sulfatides are a major lipid component of the myelin sheath and of monogalactosylalkylacylglycerol sulfate

The catalyzed reaction is as follows:
3′-phosphoadenylyl sulfate+a galactosylceramide=adenosine 3′,5′-bisphosphate+a galactosylceramide sulfate

The protein sequence can be assessed in the Uniprot database under the accession number Q99999 (G3ST1_HUMAN). Typically, the human GAL3ST1 protein has an amino acid sequence as shown in SEQ ID NO: 636.

The GAL3ST1 protein is typically referred to as “Galactosylceramide sulfotransferase”.

Typically, the ASO of the present invention targets the human GAL3ST1 pre-mRNA, i.e. downregulates expression of said pre-mRNA. The sequence of the human GAL3ST1 pre-mRNA can be e.g. assessed in the Ensembl database under accession number in ENST00000406361. Typically, it is encoded by a region on human Chromosome 22: position 30554635-30574665 on the reverse strand (Assembly GRCh38). The sequence of the human pre-mRNA has a sequence as shown in SEQ ID NO: 633. SEQ ID NO: 633 shows the sequence of the sequence of the refseq transcript, NM_001318108 (20.031 bases). In the brain, there are two further pre-mRNAs. The sequences are shown in SEQ ID NO: 634 and 635. SEQ ID NO: 633 includes the pre-mRNA sequence of the other two brain expressed transcripts, i.e. of SEQ ID NO: 634 and 635. Within the cell, the pre-mRNA is further processed, i.e., by splicing, thereby generating a protein coding mRNA (herein also referred to as transcript). In some embodiments, the antisense oligonucleotide of the present invention may also target the processed human GAL3ST1 mRNA (if the target region is located within an exon, either coding or in the 3′- or 5′UTR). SEQ ID NO: 633. 634 and 635 are RNA sequences. In the sequence listing, they are provided as DNA sequences. It is understood by the skilled person that the target RNA sequences have uracil (U) bases instead of thymine bases (T).

As set forth above, the antisense oligonucleotides of the present invention shall be capable of down-regulating, i.e. reducing expression of the GAL3ST1 pre-mRNA in a cell that expresses said pre-mRNA. Preferably, the expression is reduced in a call by antisense oligonucleotides of the present invention by least 30%, at least 40%, at least 50%, at least 60%, at least 70%, at least 80%, or at least 90% as compared to a control cell (i.e. an untreated control cell). How to assess whether the expression is reduced can be assessed by well-known methods, i.e. by measuring the expression level (i.e. the amount of the target mRNA) in ASO treated cells. In an embodiment, the down-regulation of the target gene is assessed as described in the Examples section. As control for down regulation untreated cells can be used. Down-regulating the expression of the GAL3ST1 mRNA, typically, leads down-regulation of the GAL3ST1 protein and thus to reduced levels of sulfatide as compared to a control. Down-regulation of the GAL3ST1 protein can be assessed by e.g. assessing the Galactosylceramide sulfotransferase activity in cells treated with the ASO of the present invention by using well known enzymatic assays or by or quantifying the protein expression, such as by Western Blotting, mass spectrometry or ELISA. Preferably, the Galactosylceramide sulfotransferase activity is reduced in a cell by antisense oligonucleotides of the present invention by at least 30%, at least 40%, at least 50%, at least 60%, at least 70%, at least 80%, or at least 90% as compared to a control cell (i.e. an untreated control cell).

The above assessments can be done in vivo or in vitro. If they are done in vitro, they are typically done in human cells, such as in human cells used in the Examples section. In vivo, it is e.g. envisaged that a down-regulation of the target mRNA or protein, of at least 30%, such as at least 40% is achieved. For example, the down-regulation of the target mRNA or protein may be between 40% to 60%, or between 50% to 60% as compared to a control. As set forth above, target sequences within the GAL3ST1 pre-mRNA were identified which can be efficiently targeted with ASOs. In total, 111 of such target regions/sequences were identified. These regions are shown in the following table. In the table, each identified target region was assigned a so called “Target ID” (Target ID 1 to 111). These IDs are used throughout the application. The terms “target region”, “target sequence” and “target nucleic acid” are used interchangeably herein.

Information on the identified target regions can be found in Table B1 in the Examples section (e.g. the sequence of the target region and the SEQ ID NO). Table B2 lists more preferred target regions. Table B3 lists the most preferred target regions. The target regions in Tables B2 and B3 may be present in the target regions shown in Table B1, but may be shorter. Preferably, the antisense oligonucleotide of the present invention is capable of binding (i.e. hybridizing) to a target region selected from a target region shown in the above table or in Table B1. Thus, the target sequence has a sequence selected from the group of target sequences consisting of SEQ ID NO: 522 to SEQ ID NO: 632.

More preferably, the antisense oligonucleotide is capable of binding (i.e. hybridizing) to a target region selected from a target region shown in Table B2. Accordingly, the target sequence has a sequence selected from the group of target sequences consisting of SEQ ID NO: 522, 523, 525, 532, 537, 540, 546, 547, 550, 551, 562, 563, 565, 569, 570, 571, 572, 573, 594, 598, 600, 616, 617, 618, 623, 624, 627, and 629.

Most preferably, the antisense oligonucleotide is capable of binding (i.e. hybridizing) to a target region selected from a target region shown in Table B3. Accordingly, the target sequence has a sequence selected from the sequences consisting of 522, 532, 537, 540, 546, 547, 562, 565, 569, 570, 573, 600, 617, 624, 627, 629.

Accordingly, the antisense oligonucleotide typically comprises stretch of at least 10 nucleotides which is at least 90% complementary (such as 95% or 100%) to a target sequence selected from the group of target sequences consisting of SEQ ID NO: 522 to SEQ ID NO: 632.

In an embodiment, the target sequence has a sequence as shown in SEQ ID NO: 522.

In an embodiment, the target sequence has a sequence as shown in SEQ ID NO: 532.

In an embodiment, the target sequence has a sequence as shown in SEQ ID NO: 537.

In an embodiment, the target sequence has a sequence as shown in SEQ ID NO: 540.

In an embodiment, the target sequence has a sequence as shown in SEQ ID NO: 546.

In an embodiment, the target sequence has a sequence as shown in SEQ ID NO: 547.

In an embodiment, the target sequence has a sequence as shown in SEQ ID NO: 562.

In an embodiment, the target sequence has a sequence as shown in SEQ ID NO: 565.

In an embodiment, the target sequence has a sequence as shown in SEQ ID NO: 569.

In an embodiment, the target sequence has a sequence as shown in SEQ ID NO: 570.

In an embodiment, the target sequence has a sequence as shown in SEQ ID NO: 573.

In an embodiment, the target sequence has a sequence as shown in SEQ ID NO: 600.

In an embodiment, the target sequence has a sequence as shown in SEQ ID NO: 617.

In an embodiment, the target sequence has a sequence as shown in SEQ ID NO: 624.

In an embodiment, the target sequence has a sequence as shown in SEQ ID NO: 627.

In an embodiment, the target sequence has a sequence as shown in SEQ ID NO: 629.

As set forth above, the antisense oligonucleotides of the present invention are preferably single-stranded antisense oligonucleotides.

In order to bind to a target sequence as referred to herein, the antisense oligonucleotides of the invention shall comprise a “stretch of nucleotides” which is sufficient complementary to a target sequence as referred to herein. In an embodiment, the stretch of nucleotides is at least 90% complementary to a target sequence. In another embodiment, the stretch of nucleotides is at least 95% complementary to a target sequence. In particular preferred embodiment, the stretch of nucleotides is fully complementary (i.e. 100% complementary to the target sequence). The term “complementary” is well known in the art. The percentage of complementary is typically calculated by calculating the proportion of nucleotides (in %) within the stretch of oligonucleotides of the ASO of the present invention which are complementary to the target sequence within the GAL3ST1 gene. A nucleotide present in the ASO of the present invention are considered as complementary if it forms a Watson-Crick base pair with the nucleotide present in the target RNA sequence. Watson Crick base pairs are guanine-cytosine; adenine-uracil, and adenine-thymine, i.e. G-C, A-U or A-T. As will be understood by the skilled person modified nucleotides have also the capacity to form such base pairs. For more information, see e.g. Table A2.

As will be understood by the skilled person, the “stretch of nucleotides” as referred to herein needs to have a certain length in order to allow for the binding of the oligonucleotide of the present invention to the target region. Preferably, the stretch of nucleotides has a length of at least 10 nucleotides, more preferably of at least 12 nucleotides and most preferably of at least 14 nucleotides. Further, the antisense oligonucleotide of the present invention may comprise further nucleotides—i.e. in addition to the stretch of nucleotides as referred to above, such as linker nucleotides. These further nucleotides may be complementary to the target sequence, or not.

In total, the antisense oligonucleotide of the present invention, preferably, has a length of 12 to 30 nucleotides, more preferably, of 14 to 22 nucleotides, and most preferably of 16 to 20 nucleotides. Accordingly, it is envisaged that the antisense oligonucleotide in not longer than 30 nucleotides. In some embodiments, the antisense oligonucleotide in not longer than 22 nucleotides or 20 nucleotides.

In a preferred embodiment, the antisense oligonucleotide comprises a nucleic acid sequence selected from SEQ ID NO: 1 to SEQ ID NO: 521. In a preferred embodiment, the antisense oligonucleotide consists of a nucleic acid sequence selected from SEQ ID NO: 1 to SEQ ID NO: 521.

The sequences and compounds referred to above can be found in the Table A1 in Table A1 in FIG. 1.

Preferably, the antisense oligonucleotide of the present invention is a chemically modified antisense oligonucleotide. Accordingly, it does not occur in nature. As known by the skilled person a wide range of chemical modification can be incorporated into an oligonucleotide, such modification are e.g. reviewed in Crooke et al. which herewith is incorporated by reference in its entirety (Stanley T Crooke, Xue-Hai Liang, Brenda F Baker, Rosanne M Crooke. Review J Biol Chem. 2021 January-June; 296, antisense technology: A review). Preferably, at least one of the nucleotides (herein also referred to a monomer) present in the oligonucleotide comprises a chemical modification. More preferably, at least 30%, such as at least 50% of the nucleotides present in the oligonucleotide comprise a chemical modification. In some embodiments, all of the nucleotides comprise chemical modification. Modifications include modifications of the phosphodiester backbone chemistry (“backbone modifications”), nucleobase modifications and sugar modifications. Preferred chemical modifications are shown in Table A2, see in particular the column “Nucleotide”. The ASOs of the present invention may comprise such nucleotides.

Modifications of the phosphodiester backbone chemistry affect the linkage between the individual monomers of the ASO. Thus, the ASO of the present preferably comprises one or more intemucleoside linkages other than a phosphodiester linkage. More preferably, the antisense oligonucleotide comprises at least five, such as at least ten modified intemucleoside linkages. Most preferably, all internucleoside linkages are modified linkages. However, some linkages may be phosphodiester linkages, such as one, up to two, up to three or up to four, up to six, or up to eight phosphodiester linkages. Preferably, the at least one modified intemucleoside linkage is selected from the group consisting of: at least one Phosphorothioate internucleoside linkage, at least one Phosphorodithioate intemucleoside linkage, at least one Phophoroamidate internucleoside linkage, at least one methyl phosphonate internucleoside linkage, at least one phosphotriester intemucleoside linkage, at least one boranophosphate internucleoside linkage and at least one phosphoryl guanidine internucleoside linkage. Moreover, the at least one modified intemucleoside linkage can be a stereodefined versions of said linkages.

In a preferred embodiment, the oligonucleotide comprises at least one phosphorodithioate intemucleoside linkage.

In another preferred embodiment, the oligonucleotide comprises at least one phosphoryl guanidine internucleoside linkage. Most preferably, all internucleoside linkage are phosphoryl guanidine internucleoside linkages.

In particularly preferred embodiment, the oligonucleotide comprises at least one phosphorothioate internucleoside linkage. Most preferably, at least 40%, such as at least 60% are intemucleoside linkage are phosphorothioate linkages. Most preferably, all intemucleoside linkage are phosphorothioate linkages.

Further, it is envisaged that the backbone may be modified with Morpholino Phosphorodiamidate (PMO) and Peptide Nucleic Acid (PNA).

Modification applied to the sugar group could be acyclic modifications such as UNA (unlocked nucleic acid), FNA (Flexible nucleic acid), (S)- and (R)-GNA (glycol nucleic acid), D- and L-aTNA (threofuranosyl nucleic acids), SNA (Serinol nucleic acids), as described in further details in Bege & Borbas 2021 (Miklós Bege & Anikó Borbás Review Pharmaceuticals (Basel). 2022 Jul. 22; 15(8):909. doi: 10.3390/ph15080909. The Medicinal Chemistry of Artificial Nucleic Acids and Therapeutic).

In a preferred embodiment, the chemically modified antisense oligonucleotide comprises one or more modified nucleosides.

Preferably, the one or more modified nucleosides are sugar modified nucleosides, such as one, two, three, four or more sugar modified nucleosides. Typically, it comprises four sugar modified nucleosides. A sugar modified nucleoside is nucleoside with a modified sugar. In an embodiment, the one or more sugar modified nucleosides are 2′ sugar modified nucleosides, such as 2′O modified sugar nucleosides.

In particular, the 2′O modified sugar is, selected from the group consisting of 2′-O-Me, 2′MOE (2′-O-Methoxyethyl)), 2′-N-propyl, 2′-O-allyl, 2′F RNA, 2′-O-ethylamine.

In an embodiment, the 2′O modified sugar is 2′MOE (2′-O-Methoxyethyl). Thus, the modified nucleosides are 2′MOE nucleosides.

Moreover, the one or more modified nucleotides could be locked nucleic acids such as, beta-D-oxy-LNA, 2′,4′-constrained 2′-O-ethyl (cEt), such as R-cET and S-cEt, Beta-D-amino LNA, Beta-D-thio LNA, alpha-L-oxy LNA, ENA and other modifications as described in Wan & Seth 2016 (W Brad Wan, Punit P Seth, Review J Med Chem. 2016 November 35_10; 59(21):9645-9667. The Medicinal Chemistry of Therapeutic Oligonucleotides). Thus, the oligonucleotide of the invention preferably comprises one more Locked Nucleic Acid Nucleosides (LNA nucleosides) which are well known 2′—modified nucleosides.

Preferably, the one or more modified nucleosides are (S)-6′-methyl-beta-D-oxy-LNA 40 (ScET) LNA nucleosides. More preferably, the one or more modified nucleosides are beta-D-oxy-LNA nucleosides,

Nucleobase modification include, but are not limited to, 5-methyl-cytosine, pseudo-uridine, 5-Methyluridine, 8-Oxoguanine, 2-thio-thymine, Diaminopurine, abasic nucleosides and others as also described in Brad&Seth 2016 and Robert et al., 2020 (Thomas C Roberts, Robert Langer, Matthew J A Wood. Review Nat Rev Drug Discov. 2020 October; 19(10):673-694. Advances in oligonucleotide drug delivery)

Alternatively or additionally, the chemically modified antisense oligonucleotide contains at least one modified nucleobase. For example, the at least one modified nucleobase is 5-methylcytosine. Also, the ASO may comprise at least pseudouridine, or at least one 8-oxoguanine as modified nucleobase.

Alternatively or additionally, the chemically modified antisense oligonucleotide comprises at least one modified nucleoside selected from the group consisting of: 2-O-Methoxyethyl-RNA, 2′-O-Methyl-RNA, 2′-Fluoro-RNA.

In a preferred embodiment, the antisense oligonucleotide of the present invention has a gapmer structure, i.e. is a gapmer. Gapmers are well known in the art. The term refers to (single stranded) DNA antisense oligonucleotide structures with RNA-like segments on both sides (flanking regions). Gapmers bind to the target sequence and down-regulate target gene expression through the induction of RNase H cleavage.

Suitable gapmer designs are well known in the art and are e.g. reviewed in Crooke et al., which herewith is incorporated by reference in its entirety (Stanley T Crooke, Xue-Hai Liang, Brenda F Baker, Rosanne M Crooke. Review J Biol Chem. 2021 Jan-Jun; 296. Antisense technology: A review).

Preferably, the gapmer is a LNA gapmer in which the flanking regions comprise LNA nucleosides, such as D-oxy LNA nucleosides. However, the gapmer may also comprise 2′O-Methoxyethyl (MOE) nucleosides in the flanking regions. Such gapmers are frequently referred to as MOE gapmers.

In a preferred embodiment, the antisense oligonucleotide of the present invention has a gapmer structure and at least one modified internucleoside linkage. In a preferred embodiment, the S oligonucleotide of the present invention has a gapmer structure and at least 10 modified internucleoside linkages. In another preferred embodiment, the antisense oligonucleotide of the present invention has a gapmer structure and, at least 40%, such as at least 60%, in particular all linkages are modified internucleoside linkages. The modified linkages are described herein above. In an embodiment, the modified linkages are 40 phosphorothioate intemucleoside linkages. In another embodiment, the linkages are phosphorodithioates linkages.

In an embodiment, the antisense oligonucleotide of the present invention is a compound selected from the compounds shown in Table A1 in Table A1 in FIG. 1 (see column “Compound”), wherein
Adx represents 2′deoxyadenosine-3′-phosphorothioate
Aox represents 2′-O-methyladenosine-3′-phosphorothioate
Amx represents 2′-O-Methoxyethyladenosine-3′-phosphorothioate
Alx represents 2′-O-beta-D-oxy LNA adenosine-3′-phosphorothioate
Cdx represents 2′deoxycytidine-3′-phosphorothioate
Cox represents 2′-O-methylcytidine-3′-phosphorothioate
Edx represents 2′deoxy-5-methylcytidine-3′-phosphorothioate
Emx represents 2′-O-Methoxyethyl-5-methylcytidine-3′-phosphorothioate
Elx represents 2′-O-beta-D-oxy LNA-5-methylcytidine-3′-phosphorothioate
Gdx represents 2′deoxyguanosine-3′-phosphorothioate
Gox represents 2′-O-methylguanosine-3′-phosphorothioate
Gmx represents 2′-O-Methoxyethylguanosine-3′-phosphorothioate
Glx represents 2′-O-beta-D-oxy LNA guanosine-3′-phosphorothioate
Tdx represents 2′deoxythymidine-3′-phosphorothioate
Tmx represents 2′-O-Methoxyethylthymidine-3′-phosphorothioate
Tlx represents 2′-O-beta-D-oxy LNA thymidine-3′-phosphorothioate
Ado represents 2′deoxyadenosine-3′-phosphodiester
Aoo represents 2′-O-methyladenosine-3′-phosphodiester
Amo represents 2′-O-Methoxyethyladenosine-3′-phosphodiester
Alo represents 2′-O-beta-D-oxy LNA adenosine-3′-phosphodiester
Cdo represents 2′deoxycytidine-3′-phosphodiester
Coo represents 2′-O-methylcytidine-3′-phosphodiester
Edo represents 2′deoxy-5-methylcytidine-3′-phosphodiester
Emo represents 2′-O-Methoxyethyl-5-methylcytidine-3′-phosphodiester
Elo represents 2′-O-beta-D-oxy LNA-5-methylcytidine-3′-phosphodiester
Gdo represents 2′deoxyguanosine-3′-phosphodiester
Goo represents 2′-O-methylguanosine-3′-phosphodiester
Gmo represents 2′-O-Methoxyethylguanosine-3′-phosphodiester
Glo represents 2′-O-beta-D-oxy LNA guanosine-3′-phosphodiester
Tdo represents 2′deoxythymidine-3′-phosphodiester
Tmo represents 2′-O-Methoxyethylthymidine-3′-phosphodiester
Tlo represents 2′-O-beta-D-oxy LNA thymidine-3′-phosphodiester
Uo represents 2′-O-methyluridine
Ad represents 2′deoxyadenosine
Ao represents 2′-O-methyladenosine
Am represents 2′-O-Methoxyethyladenosine
Al represents 2′-O-beta-D-oxy LNA adenosine
Cd represents 2′deoxycytidine
Co represents 2′-O-methylcytidine
Ed represents 2′deoxy-5-methylcytidine
Em represents 2′-O-Methoxyethyl-5-methylcytidine
El represents 2′-O-beta-D-oxy LNA-5-methylcytidine
Gd represents 2′deoxyguanosine
Go represents 2′-O-methylguanosine
Gm represents 2′-O-Methoxyethylguanosine
Gi represents 2′-O-beta-D-oxy LNA guanosine
Td represents 2′deoxythymidine
Tm represents 2′-O-Methoxyethylthymidine
Ti represents 2′-O-beta-D-oxy LNA thymidine
Uoo represents 2′-O-methyluridine-3′-phosphodiester, and
Uox represents 2′-O-methyluridine-3′-phosphorothioate.

In many compounds, all internucleoside linkages present in the compounds shown in Table A1 in FIG. 1 and in Table C in FIG. 2 are phosphorothioate linkages. The presence of an “o” at the third position of the three-letter code (e.g. Aoo) indicates the presence of a phosphodiester internucleoside linkage. The presence of an “x” at the third position of the three-letter code (e.g. Aox) indicates the presence of a Phosphorothioate internucleoside linkage.

In in Table A1 in FIG. 1 and in Table C in FIG. 2, a three-letter code or two-letter code was used in order to describe the modified nucleotides that are present in the oligonucleotide compounds. Additionally, each ofthe compounds in Table A1 in FIG. 1 and in Table C in FIG. 2 contains a two-letter code in the 3′ end, which does not contain the phosphorothioate or phosphodiester group. The base and sugar groups ofthe two-letter code is otherwise identical to the three letter. Table A2 provides atranslation ofthe two or three letter codes to their chemical names.

TABLE A2
Overview on modified nucleotides

| | | | Complementary |
|---|---|---|---|
| | | | nucleotide in |
| | | | the target pre- |
| | | | mRNA |
| | | | (Watson-Crick- |
| Abbreviation | Nucleotide | Sugar | base pairing) |
| Adx | 2′deoxyadenosine-3′-phosphorothioate | DNA | U (,,T” in SEQ |
| | | | ID NO: 633) |
| Aox | 2′-O-methyladenosine-3′-phosphorothioate | 2′Ome | U (,,T” in SEQ |
| | | | ID NO: 633) |
| Amx | 2′-O-Methoxyethyladenosine-3′-phosphorothioate | MOE | U (,,T” in SEQ |
| | | | ID NO: 633) |
| Alx | 2′-O-beta-D-oxy LNA adenosine-3′-phosphorothioate | LNA | U (,,T” in SEQ |
| | | | ID NO: 633) |
| Cdx | 2′deoxycytidine-3′-phosphorothioate | DNA | G |
| Cox | 2′-O-methylcytidine-3′-phosphorothioate | 2′Ome | G |
| Edx | 2′deoxy-5-methylcytidine-3′-phosphorothioate | DNA | G |
| Emx | 2′-O-Methoxyethyl-5-methylcytidine-3′-phosphorothioate | MOE | G |
| Elx | 2′-O-beta-D-oxy LNA-5-methylcytidine-3′-phosphorothioate | LNA | G |
| Gdx | 2′deoxyguanosine-3′-phosphorothioate | DNA | C |
| Gox | 2′-O-methylguanosine-3′-phosphorothioate | 2′Ome | C |
| Gmx | 2′-O-Methoxyethylguanosine-3′-phosphorothioate | MOE | C |
| Glx | 2′-O-beta-D-oxy LNA guanosine-3′-phosphorothioate | LNA | C |
| Tdx | 2′deoxythymidine-3′phosphorothioate | DNA | [symbol] |
| Tmx | 2′-O-Methoxyethylthymidine-3′-phosphorothioate | MOE | A |
| Tlx | 2′-O-beta-D-oxy LNA thymidine-3′-phosphorothioate | LNA | A |
| Ado | 2′deoxyadenosine-3′-phosphorothioate | DNA | U (,,T” in SEQ |
| | | | ID NO: 633) |
| Aoo | 2′-O-methyladenosine-3′-phosphodiester | 2′Ome | U (,,T” in SEQ |
| | | | ID NO: 633) |
| Amo | 2′-O-Methoxyethyladenosine-3′-phosphodiester | MOE | U (,,T” in SEQ |
| | | | ID NO: 633) |
| Alo | 2′-O-beta-D-oxy LNA adenosine-3′-phosphodiester | LNA | U (,,T” in SEQ |
| | | | ID NO: 633) |
| Cdo | 2′deoxycytidine-3′-phosphodiester | DNA | G |
| Coo | 2′-O-methylcytidine-3′-phosphodiester | 2′Ome | G |
| Edo | 2′deoxy-5-methylcytidine-3′-phosphodiester | DNA | G |
| Emo | 2′-O-Methoxyethyl-5-methylcytidine-3′-phosphodiester | MOE | G |
| Elo | 2′-O-beta-D-oxy LNA-5-methylcytidine-3′-phosphodiester | LNA | G |
| Gdo | 2′deoxyguanosine-3′-phosphodiester | DNA | C |
| Goo | 2′-O-methylguanosine-3′-phosphodiester | 2′Ome | C |
| Gmo | 2′-O-Methoxyethylguanosine-3′-phosphodiester | MOE | C |
| Glo | 2′-O-beta-D-oxy LNA guanosine-3′-phosphodiester | LNA | C |
| Tdo | 2′deoxythymidine-3′-phosphodiester | DNA | A |
| Tmo | 2′-O-Methoxyethylthymidine-3′-phosphodiester | MOE | A |
| Tlo | 2′-O-beta-D-oxy LNA thymidine-3′-phosphodiester | LNA | A |
| Uo | 2′-O-methyluridine | 2′Ome | A |
| Ad | 2′deoxyadenosine | DNA | U (,,T” in SEQ |
| | | | ID NO: 633) |
| Ao | 2′-O-methyladenosine | 2′Ome | U (,,T” in SEQ |
| | | | ID NO: 633) |
| Am | 2′-O-Methoxyethyladenosine | MOE | U (,,T” in SEQ |
| | | | ID NO: 633) |
| Al | 2′-O-beta-D-oxy LNA adenosine | LNA | U (,,T” in SEQ |
| | | | ID NO: 6) |
| Cd | 2′deoxycytidine | DNA | G |
| Co | 2′-O-methylcytidine | 2′Ome | G |
| Ed | 2′deoxy-5-methylcytidine | DNA | G |
| Em | 2′-O-Methoxyethyl-5-methylcytidine | MOE | G |
| El | 2′-O-beta-D-oxy LNA-5-methylcytidine | LNA | G |
| Gd | 2′deoxyguanosine | DNA | C |
| Go | 2′-O-methylguanosine | 2′Ome | C |
| Gm | 2′-O-Methoxyethylguanosine | MOE | C |
| Gl | 2′-O-beta-D-oxy LNA guanosine | LNA | C |
| Td | 2′deoxythymidine | DNA | A |
| Tm | 2′-O-Methoxyethylthymidine | MOE | A |
| Tl | 2′-O-beta-D-oxy LNA thymidine | LNA | A |
| Uox | 2′-O-methyluridine-3′-phosphorothioate | 2′Ome | A |
| Uoo | 2′-O-methyluridine-3′-phosphodiester | 2′Ome | A |

Preferably, the compound selected from Table A1 in FIG. 1 is a compound, which resulted in an efficient down-regulation of the target gene in the studies described in the Examples, such as more than 50% or more than 34%.

In an embodiment, the antisense oligonucleotide is an antisense oligonucleotide with ASO ID 1_41, 1_103, 1_131, 1_129, 1_116, 1_164, 1_193, 16_17, 19_23, 25_85, 25_16,108_15, 108_16, 108_12, 108_6, 102_4, 108_20, 108_7, 108_17, 106_7, 103_10, 96_21, 96_12, 482, 48_7, 483, 79_3, 95_7, and 95_9, as shown in Table A1 in FIG. 1.

Preferably, the compound is a compound selected from Table C in FIG. 2. More preferably, the compound is a compound from Table C, which resulted in an efficient down-regulation of the target gene in the studies described in the Examples, such as more than 50% or more than 34%.

Preferably, the antisense oligonucleotide (or composition) of the present invention shall be administered to the CNS, in particular to the brain. Accordingly, the ASO is delivered to CNS through intrathecal injection—as it is e.g. the current state of art for similar ASO e.g. Nusinersen/Spinraza. Thus, the ASO of the present invention or the pharmaceutical composition is, preferably, administered intrathecally. In addition to delivery to the CNS, the ASO can be administered by subcutaneous or intravenous administration either with or with or without a conjugate in order to reach the peripheral nervous system.

The present invention further relates to a conjugate comprising the antisense oligonucleotide of the present invention and a conjugate moiety. Preferably, the conjugate moiety is covalently bound to the antisense oligonucleotide, e.g. via one or more linker nucleotides, such as one, two, three or four linker nucleotides (or more). The linker may be cleaved after administration to the patient.

Preferably, the antisense oligonucleotide of the present invention shall be delivered or administered to the CNS, in particular to the brain. Accordingly, it is envisaged that the conjugate moiety is a moiety that allows the crossing of the conjugate of the blood brain barrier. For example, the moiety can be and antibody or antigen-binding fragment thereof targeting the transferrin receptor.

The antisense oligonucleotides of the present invention can be administered/delivered ‘unassisted’ in saline solution. However, distribution to certain tissues and uptake in cells can be enhanced by conjugates and formulation techniques. Conjugation to ASOs could be, peptides, antibodies and aptamers binding to receptors on target cells or proteins mediating transcytosis e.g. the transferrin receptor. Antisense oligos can also be conjugated to naturally occurring ligands or modifications hereof as exemplified by GalNac conjugation binding with high affinity to asialoglycoprotein receptor 1 (ASGR1, ASPGR) and Alpha-tocopherol conjugation and interaction with transfer protein Alfa-TTP. This could also be small molecules generated through medicinal chemistry with high affinity for known receptors and transporter. Moreover, it could be conjugations to long chained fatty acids that modify the hydrophobicity and protein binding properties of the ASOs, but also could function through their capacity to bind to lipoprotein particles and hence function through the endogenous mechanism for lipid transport and uptake.

In addition to conjugation, tissues delivery and cellular uptake of ASOs of the present invention can be enhanced through formulation with nanocarriers that facilitates crossing of biological barriers such as cellular membranes. Various types of nanocarriers have been described with favorable properties for delivery of nucleic acids e.g. lipid nanoparticles (LNPs) as used for BioNTech mRNA vaccines, LNPs functionalized with peptides, PEGylated lipids, cationic lipids, exomes (lipid bilayers) both artificial and natural exosomes such as milk exosomes and spherical nucleic acids and others as described in further details in Roberts et al., 2020 (Thomas C Roberts, Robert Langer, Matthew J A Wood. Review Nat Rev Drug Discov. 2020 October; 19(10):673-694. Advances in oligonucleotide drug delivery).

The present invention further relates to a pharmaceutical composition comprising the antisense oligonucleotide of the present invention or the conjugate of the present invention.

Typically, a pharmaceutical composition comprises the antisense oligonucleotide or the conjugate of the present invention together with a pharmaceutically acceptable carrier and/or, in particular, a pharmaceutically acceptable excipient. The term “pharmaceutically acceptable”, as used herein, refers to the non-toxicity of a material which, in certain exemplary embodiments, does not interact with the action of the oligonucleotide or the conjugate present in the pharmaceutical composition.

The term “carrier”, as used herein, refers to an organic or inorganic component, of a natural or synthetic nature, in which the active component is combined in order to facilitate, enhance or enable application.

The term “excipient”, as used herein, is intended to include all substances which may be present in a pharmaceutical composition and which are not active ingredients, such as salts, binders (e.g., lactose, dextrose, sucrose, trehalose, sorbitol, mannitol), fillers, lubricants, thickeners, surface active agents, preservatives, emulsifiers or buffer substances.

The form of the pharmaceutical composition, the route of administration, the dosage and the regimen naturally depend upon the condition to be treated, the severity of the illness, the age, weight, and gender of the patient, etc.

In an embodiment, the pharmaceutical composition can be formulated for intrathecal administration. Thus, the antisense oligonucleotide or conjugate of the present invention is preferably administered by intrathecal administration a route of administration for drugs via an injection into the spinal canal. Thereby, it reaches the cerebrospinal fluid and the brain.

The present invention further relates to the antisense oligonucleotide according to the present invention, the conjugate according to the present invention, or the pharmaceutical composition according to the present invention for use in medicine.

Accordingly, the present invention relates to the antisense oligonucleotide according to the present invention, the conjugate according to the present invention, or the pharmaceutical composition according to the present invention for use in treating metachromatic leukodystrophy (MLD).

Further, the present invention relates to the use of the antisense oligonucleotide according to the present invention, the conjugate according to the present invention, or the pharmaceutical composition according to the present invention for the manufacture of a medicament for treating metachromatic leukodystrophy. Metachromatic leukodystrophy is a lysosomal storage disease. MLD is typically listed in the family of leukodystrophies as well as among the sphingolipidoses as it affects the metabolism of sphingolipids. Leukodystrophies affect the growth and/or development of myelin, the fatty covering which acts as an insulator around nerve fibers throughout the central and peripheral nervous systems. MLD involves cerebroside sulfate accumulation. MLD is degenerative disease that is associated with a progressive damage to brain cells. The disease is inherited in an autosomal recessive manner.

Accordingly, the present invention relates to the antisense oligonucleotide according to the present invention, the conjugate according to the present invention, or the pharmaceutical composition according to the present invention for use treating metachromatic leukodystrophy.

Further, the present invention relates to the use of the antisense oligonucleotide according to the present invention, the conjugate according to the present invention, or the pharmaceutical composition according to the present invention for the manufacture of a medicament for treating metachromatic leukodystrophy.

Further, the present invention relates to a method of treating metachromatic leukodystrophy, comprising administering pharmaceutically effective amount of the antisense oligonucleotide according to the present invention, the conjugate according to the present invention, or the pharmaceutical composition according to the present invention to a subject suffering from metachromatic leukodystrophy.

The term “treating” or “treatment”, as used herein, refers to the administration of a compound or composition or a combination of compounds or compositions to a subject in order to ameliorate metachromatic leukodystrophy. Thus, the term encompasses both the amelioration of one or more symptoms of the metachromatic leukodystrophy or prevention of the worsening of one or more symptoms, i.e. prophylaxis. The amelioration of symptoms also includes the reduction of one or more symptoms. Thus, the term, preferably, refers to the reduction of one or more symptoms of the disease. In other words, the treatment is typically a disease modifying treatment that reduces one or more symptoms of metachromatic leukodystrophy. In an embodiment, the development of disease pathology is inhibited. It will therefore be recognized that treatment as referred to herein may, in some embodiments, be prophylactic or disease modifying. It is to be understood that the treatment does not allow a complete cure of metachromatic leukodystrophy.

Metachromatic leukodystrophy is known to decrease the life expectancy. Thus, the term “treatment” also includes increasing the life expectancy of a subject (as compared to an untreated subject).

In accordance with the present invention, the subject to be treated is a subject suffering from metachromatic leukodystrophy. However, the patient might not yet show symptoms of metachromatic leukodystrophy at the time of the treatment. Typically, the subject shows symptoms of metachromatic leukodystrophy. Clinical manifestation of MLD can vary depending on the age of onset, which can range from infancy to adulthood. Symptoms of metachromatic leukodystrophy are well known in the art and include (but are not limited to) one or more of include muscle wasting and weakness, muscle rigidity, developmental delays, progressive loss of vision leading to blindness, convulsions, impaired swallowing, paralysis, and dementia. Preferably, the subject has been diagnosed through genetic testing to suffer from metachromatic leukodystrophy. The most common and severe form of MLD is diagnosed in the infant age. Also typically, the diagnosis involves genetic testing.

There are various forms of MLD: late infantile, juvenile, and adult MLD. The most common form is the late-infantile form, which usually presents between 6 months and 2 years of age. In accordance with the present invention, MLD typically is, thus, late infantile, juvenile, and adult MLD. In particular, late infantile MLD is treated. Thus, the subject may be e.g. between 6 months and 2 years of age.

The terms “subject” and “patient” are used interchangeably herein. The “subject” or “patient” may be a vertebrate. The term includes both humans and other animals, particularly mammals, and other organisms. In some embodiments, the subject is a mammal. In some embodiments, the subject is a primate. Preferably, the subject is a human subject suffering from metachromatic leukodystrophy.

The present invention further relates to a method for identifying a candidate compound for the treatment of metachromatic leukodystrophy, comprising
a) providing an antisense oligonucleotide according to the present invention,
b) contacting a host cell expressing GAL3ST1 mRNA with said antisense oligonucleotide,
c) determining the amount of GAL3ST1 mRNA in the said host cell, and
d) identifying a candidate compound based on the results of step c).

The antisense oligonucleotide to be provided in step a) is preferably the antisense oligonucleotide of the present invention. Accordingly, it shall comprise a stretch of at least nucleotides which is at least 90% complementary to a target sequence in the human GAL3ST1 gene (i.e. mRNA or premRNA). The definitions provided herein above preferably apply Mutatis mutandis. Preferably, the provided antisense oligonucleotide targets a sequence disclosed in Table B1, more preferably, a sequence disclosed in Table B2 and most preferably a sequence disclosed in Table B3.

Preferably, said method is an in vitro method. In step a) of the above method of the present invention an antisense oligonucleotide of the present invention is provided. Preferably, the antisense oligonucleotides are complementary to a target region as set forth herein elsewhere. In step b) the antisense oligonucleotide shall be contacted with a host cell. Said host cell shall express the GAL3ST1 gene. Preferably, an antisense olignonucleotide which leads to a reduction of the amount of GAL3ST1 mRNA (such as GAL3ST1 mRNA) is considered as candidate compound.

Finally, the present invention relates to an inhibitor for GAL3ST1, or the pharmaceutical composition comprising said inhibitor for use in treating metachromatic leukodystrophy.

In an embodiment, the inhibitor is an inhibitory RNA, such as a siRNA or short-hairpin RNA, which is capable of binding to GAL3ST1 mRNA, such as the pre-mRNA and which is capable of downregulating the GAL3ST1 mRNA in a cell.

In another embodiment, the inhibitor is an antisense oligonucleotide, which is capable of binding to GAL3ST1 mRNA, such as the pre-mRNA and which is capable of downregulating the GAL3ST1 mRNA in a cell, such as the antisense oligonucleotide of the present invention.

In another embodiment, the inhibitor is an antibody, or antigen binding fragment thereof which specifically binds to the GAL3ST1 protein, and which is capable of downregulating the GAL3ST1 protein in a cell.

In accordance with the present invention, it is also envisaged to apply a combination therapy of MLD. For example, the inhibitor, such as the antisense oligonucleotide can be used in combination with Libmeldy stem cell therapy. Libmeldy stem cell therapy is an autologous haematopoietic stem cell (HSC) gene therapy product currently used for late infantile or early juvenile forms of the MLD.

The Figures show

FIG. 1: Table A1, Compounds tested in the Examples section (e.g. in Example 1)

FIG. 2: Table C, Compounds tested in the Examples section (e.g. in Example 5)

All patents, patent applications, and publications or public disclosures referred to or cited herein are incorporated by reference in their entirety.

The invention will be further described with reference to the examples described herein; however, it is to be understood that the invention is not limited to such examples.

### EXAMPLES

### Oligonucleotide Synthesis

Oligonucleotide synthesis, a well-known technique in the field, was utilized for the experiments described herein. The oligonucleotides were procured from Biosearch Technologies (Lystrup, Denmark). Following solid support cleavage, the oligonucleotides were subjected to cartridge purification utilizing ammonium acetate. Subsequently, the oligonucleotides were dissolved to a concentration of 750 μM in PBS, and purity was assessed by LC/MS with a minimum threshold of >80% purity.

FIG. 1 showcases the compounds produced by the aforementioned methodology, all of which underwent testing in either A549 cells (Example 1) or CaCo2 cells (Example 2). Subsequently, select ASO compounds that displayed efficient downregulation of GAL3ST1 underwent further assessment, as detailed in Example 3, where EC50 values were determined.

### Example 1: Testing In Vitro Efficacy of Antisense Oligonucleotides Targeting GAL3ST1 In A549 Cells at Single Test Concentration

To assess the efficacy of the designed and synthesized ASOs, a cell-based screening assay was developed in cells showing endogenous expression of GAL3ST1 premRNA. Various cell densities, compound incubation periods, and concentrations were optimized prior to establishing the assay conditions, as delineated below.

The A549 cells were cultured and expanded as per the supplier's instructions (ECACC, acquired from Merck, 86012804-1VL). The cells were grown to 70-80% confluency, trypsinized, and suspended in growth media. Viable cells were counted using trypan blue and a Countess 3 automatic cell counter. The requisite number of cells were diluted in complete growth media, mixed via gentle pipetting, added to reagent reservoirs, and dispensed into 96-well plates using a multichannel pipette in a total volume of 195 μl/well. To mitigate evaporation and potential plate effect, sterile PBS was added to the moats of the 96-well culture plates (Nunc™ Edge™ 96-Well, Nunclon Delta-Treated, Flat-Bottom Microplates). After incubating for 24 hours, GAL3ST1 ASOs (refer to Table 3, columns “ASO ID”; the compounds are listed in FIG. 1) were added directly to the growth media from a 20-fold stock dilution in PBS, resulting in a final ASO concentration of either 10 or 25 μM. Table 1 provides a summary of the crucial parameters pertaining to the cellular work.

TABLE 1
Information on cell line A549

| | | | | Incubation | |
|---|---|---|---|---|---|
| | | | | prior | Incubation |
| Cell | Provi- | Culturing | Seeding density | to ASO | time with |
| Line | der | conditions | (cells/well) | addition | ASO |
| A549 | ATCC | 5% CO₂ | 10,000 cells/well | 24 h | 48 h |
| | | and 95% | in 195 μl of | | |
| | | humidity | media: Ham's F12K | | |
| | | at 37° C. | with 10% FBS and | | |
| | | | 2 mM Glutamine | | |

In addition to the ASOs designed to target GAL3ST1, each plate contained 7 PBS controls (5 μL each), a positive control (N=2 per plate) targeting the ATXN3 gene, and 30 non-targeting gapmer controls randomly distributed across all screening plates to monitor potential false positives.

After 48 hours of incubation, cells were harvested by gently aspirating the media and RNA was extracted using the Macherey-Nagel NucleoSpin 96 RNA Kit, following the manufacturer's instructions, and eluted in 75 μl of water.

For qPCR-based expression analysis, 10 μl of the RNA-containing eluate was transferred to a new plate and diluted 10-fold in RNase-free water then heat chocked by heating to 90° C. for 40 s and immediately placing on ice. The diluted and heat-shocked RNA was used as input template for aPCR, using aScript™ XLT One-Step RT-aPCR ToughMix® (cat #95134-500) from QuantaBio and duplexed PCR reaction with probe based qPCR assays from Integrated DNA technologies (IDT) listed in Table 2.

TABLE 2
qPCR assay used for screening GAL3ST1 targeting ASOs.

| Target | Forward Primer | Reverse Primer | Probe sequence | Probe type |
|---|---|---|---|---|
| GAL3ST1 | 5-′GCA TGT | 5′-TCA AGT | 5′-CGC AAT | FAM with |
| (GOI) | GGT TGC AGA | TCG CCT | GAC TTC GAC | ZEN/IBFQ |
| | TGA TG-3′ | TCC CTA AC- | TAC CCG ACC- | double |
| | (SEQ ID NO: | 3′ (SEQ ID | 3′ (SEQ ID NO: | quencher |
| | 637) | NO: 638) | 639) | |
| ATXN3 | 5′-TAT CAG | 5′-GTT TCT | 5′AAA GGC CAG | hex with |
| (Normalization) | GAC AGA GTT | AAA GAC | CCA CCA GTT | ZEN/IBFQ |
| | CAC ATC C-3′ | ATG GTC | CAG G-3′ (SEQ | double |
| | (SEQ ID NO: | ACA GC-3′ | ID NO: 642) | quencher |
| | 640) | (SEQ ID NO: | | |
| | | 641) | | |

The qPCR reaction was performed in 384 wells using a QuantStudio 7 Flex instrument (Applied Biosystems by Thermo Fisher Scientific). Quantification of GAL3ST1 mRNA was carried out using the ddCT method, with the median value of all the PBS-treated wells within the same plate serving as the untreated control.

The expression level of GAL3ST1 following ASO treatment (Table 3) is presented as a percentage of the PBS-treated wells.

The screening process yielded several ASOs with a high level of knockdown (e.g., more than 70%) targeting various regions of the GAL3ST1 premRNA. These sequences and their target regions are of particular interest for further compound optimization.

TABLE 3A
Expression level of GAL3ST1 in relation to PBS treated control cells (in %) a549 25 μM

| | % PBS | | % PBS | | % PBS | | % PBS | | % PBS | | % PBS | | % PBS |
|---|---|---|---|---|---|---|---|---|---|---|---|---|---|
| | (a549_25 | | (a549_25 | | (a549_25 | | (a549_25 | | (a549_25 | | (a549_25 | | (a549_25 |
| | uM GAL3ST1 norm | | uM GAL3ST1 norm | | uM GAL3ST1 norm | | uM GAL3ST1 norm | | uM GAL3ST1 norm | | uM GAL3ST1 norm | | uM GAL3ST1 norm |
| ASO ID | ATXN3) | ASO ID | ATXN3) | ASO ID | ATXN3) | ASO ID | ATXN3) | ASO ID | ATXN3) | ASO ID | ATXN3) | ASO ID | ATXN3) |
| 1_1 | 55.825648 | 1_164 | 29.771421 | 4_82 | 70.61272 | 16_94 | 106.28957 | 24_1 | 94.278454 | 33_52 | 100.27764 | 45_15 | 61.047326 |
| 1_2 | 73.509367 | 1_163 | 24.333234 | 4_85 | 89.78211 | 16_96 | 65.565101 | 25_48 | 14.12184 | 33_54 | 124.31497 | 45_13 | 92.851685 |
| 1_3 | 72.648252 | 1_162 | 59.460356 | 4_84 | 75.549804 | 16_97 | 73.866903 | 25_49 | 22.656548 | 33_56 | 109.42937 | 45_16 | 89.564567 |
| 1_5 | 46.073209 | 1_161 | 26.152123 | 4_83 | 99.447017 | 16_98 | 69.640574 | 25_50 | 90.524574 | 33_55 | 112.81821 | 45_17 | 121.27261 |
| 1_6 | 58.035196 | 1_165 | 95.06591 | 5_1 | 99.542284 | 16_99 | 72.653791 | 26_1 | 94.849398 | 33_68 | 108.97521 | 45_14 | 128.25366 |
| 1_7 | 89.875513 | 1_160 | 124.83305 | 6_1 | 83.508792 | 16_100 | 60.040171 | 26_2 | 70.076372 | 33_69 | 81.056512 | 45_18 | 92.338231 |
| 1_8 | 55.825648 | 1_159 | 108.52294 | 9_1 | 79.66401 | 16_104 | 70.956168 | 26_3 | 72.24652 | 33_70 | 83.450928 | 45_19 | 113.76052 |
| 1_9 | 62.33006 | 1_158 | 46.025328 | 9_2 | 85.263489 | 16_105 | 91.319825 | 26_4 | 75.68084 | 33_73 | 102.62494 | 45_20 | 62.850669 |
| 1_10 | 92.08257 | 1_157 | 118.93061 | 10_1 | 77.485593 | 16_106 | 100.87019 | 26_5 | 104.39119 | 33_72 | 182.26026 | 45_21 | 89.100159 |
| 1_13 | 64.394081 | 1_156 | 85.737604 | 10_2 | 92.242269 | 16_107 | 79.719248 | 26_6 | 47.565914 | 33_71 | 97.332937 | 45_22 | 103.67011 |
| 1_14 | 62.416527 | 1_193 | 25.82786 | 10_3 | 86.453723 | 16_109 | 53.329289 | 26_8 | 86.296945 | 33_75 | 119.99714 | 45_23 | 114.79024 |
| 1_11 | 98.760086 | 1_187 | 25.578437 | 10_4 | 78.29543 | 16_108 | 82.188019 | 26_7 | 89.44049 | 33_74 | 133.65371 | 46_1 | 86.693756 |
| 1_15 | 59.229236 | 1_183 | 71.103871 | 10_9 | 87.744548 | 16_110 | 124.14275 | 26_9 | 48.330305 | 33_76 | 54.942711 | 46_4 | 90.187538 |
| 1_16 | 95.660816 | 1_170 | 45.913808 | 10_5 | 95.727146 | 16_111 | 83.184197 | 26_10 | 53.812006 | 33_77 | 103.74199 | 46_3 | 114.42708 |
| 1_17 | 96.126093 | 1_178 | 68.586664 | 10_10 | 80.162533 | 17_1 | 131.49428 | 26_11 | 64.886938 | 33_78 | 119.03463 | 46_2 | 131.85936 |
| 1_18 | 51.978972 | 1_173 | 85.027416 | 10_11 | 117.20223 | 17_2 | 123.83176 | 26_12 | 43.137067 | 34_2 | 73.305838 | 46_5 | 121.84103 |
| 1_19 | 58.723099 | 1_176 | 49.930733 | 10_12 | 112.03892 | 17_3 | 136.03709 | 26_13 | 33.774352 | 34_3 | 67.924314 | 46_6 | 119.69653 |
| 1_20 | 77.970484 | 1_177 | 43.077308 | 11_1 | 43.196909 | 17_4 | 125.21274 | 26_14 | 71.946679 | 34_1 | 130.40507 | 46_7 | 117.55871 |
| 1_21 | 59.049633 | 1_171 | 74.690673 | 11_2 | 29.999285 | 17_5 | 110.64974 | 27_1 | 90.496958 | 34_6 | 65.610563 | 46_8 | 123.79903 |
| 1_22 | 72.497342 | 2_7 | 80.944222 | 11_3 | 92.466328 | 17_6 | 146.61168 | 28_24 | 58.100094 | 34_7 | 121.84103 | 46_9 | 135.32059 |
| 1_23 | 53.737459 | 2_9 | 93.303299 | 11_5 | 81.146318 | 17_8 | 115.49436 | 28_25 | 103.26363 | 34_8 | 93.56235 | 46_10 | 168.76316 |
| 1_31 | 58.317468 | 2_6 | 122.94389 | 11_6 | 85.32261 | 17_9 | 127.36775 | 29_1 | 99.274832 | 34_5 | 102.27366 | 49_3 | 35.135468 |
| 1_30 | 64.662447 | 2_8 | 76.950361 | 11_7 | 64.886938 | 17_10 | 109.30667 | 30_3 | 61.344249 | 34_4 | 104.17434 | 49_2 | 29.812722 |
| 1_29 | 73.255044 | 2_5 | 88.576752 | 11_8 | 64.394081 | 17_11 | 115.13938 | 30_2 | 82.245007 | 34_9 | 97.738577 | 49_4 | 78.186964 |
| 1_32 | 56.526528 | 2_1 | 113.91834 | 11_4 | 75.890963 | 17_12 | 129.95391 | 30_1 | 67.642412 | 34_10 | 109.01636 | 49_1 | 33.056365 |
| 1_27 | 96.861819 | 2_4 | 71.054602 | 11_9 | 46.878555 | 17_7 | 128.25366 | 30_8 | 53.826234 | 37_1 | 102.45568 | 49_5 | 112.74004 |
| 1_28 | 91.763988 | 2_3 | 72.12143 | 11_10 | 52.231788 | 17_14 | 110.41988 | 30_7 | 77.916458 | 37_3 | 99.930709 | 50_17 | 89.112795 |
| 1_41 | 39.039325 | 2_2 | 52.668052 | 11_11 | 44.565525 | 17_22 | 126.13774 | 30_6 | 113.8695 | 37_2 | 102.88267 | 50_16 | 82.587766 |
| 1_40 | 56.840849 | 2_10 | 60.123462 | 11_12 | 61.72813 | 17_13 | 132.77652 | 30_4 | 90.250073 | 37_4 | 89.564567 | 50_18 | 79.794149 |
| 1_39 | 87.964908 | 2_11 | 110.64974 | 11_13 | 83.566696 | 17_15 | 115.06906 | 30_5 | 121.87469 | 37_5 | 104.60849 | 50_32 | 81.359425 |
| 1_38 | 60.793964 | 2_12 | 120.08034 | 11_14 | 20.718623 | 17_16 | 118.42716 | 30_11 | 54.22387 | 37_6 | 107.92282 | 50_31 | 49.358874 |
| 1_37 | 83.0114 | 2_20 | 88.63817 | 11_16 | 53.998828 | 17_17 | 101.39595 | 30_12 | 75.943584 | 37_7 | 61.985385 | 50_30 | 66.388258 |
| 1_36 | 85.678195 | 2_17 | 111.83575 | 11_24 | 65.022007 | 17_18 | 114.86984 | 30_14 | 113.60293 | 37_8 | 101.67747 | 50_35 | 84.808561 |
| 1_35 | 69.979293 | 2_21 | 112.58386 | 12_2 | 100.13873 | 17_19 | 124.40117 | 30_10 | 91.891288 | 37_9 | 133.14516 | 50_33 | 81.366109 |
| 1_34 | 62.546454 | 2_31 | 52.231788 | 12_3 | 113.13145 | 17_20 | 114.07637 | 30_9 | 53.77472 | 37_11 | 125.20662 | 50_40 | 81.846918 |
| 1_33 | 90.312651 | 2_30 | 53.886657 | 12_4 | 130.58598 | 17_21 | 108.2975 | 30_13 | 47.237352 | 37_12 | 111.5261 | 50_39 | 102.88267 |
| 1_51 | 57.774305 | 2_29 | 101.25548 | 12_5 | 124.91961 | 19_10 | 68.065706 | 30_19 | 50.662845 | 37_10 | 108.8997 | 50_37 | 121.41949 |
| 1_47 | 74.535519 | 3_4 | 67.22175 | 12_8 | 135.37863 | 19_11 | 95.826721 | 30_22 | 53.551541 | 38_1 | 126.66343 | 51_3 | 89.937831 |
| 1_50 | 83.682624 | 3_3 | 107.92282 | 12_13 | 107.54944 | 19_12 | 78.404045 | 30_21 | 61.557221 | 39_1 | 124.66012 | 51_1 | 95.06591 |
| 1_49 | 53.961412 | 3_2 | 105.7751 | 12_10 | 132.68451 | 19_17 | 56.801463 | 30_23 | 72.54761 | 39_2 | 113.33754 | 51_2 | 100.17343 |
| 1_48 | 60.332196 | 3_1 | 120.41374 | 12_14 | 118.18115 | 19_14 | 86.874219 | 30_18 | 79.629858 | 39_3 | 103.52649 | 51_4 | 76.597813 |
| 1_53 | 47.335682 | 3_5 | 109.73319 | 12_11 | 121.58793 | 19_8 | 112.84804 | 30_17 | 78.105709 | 39_4 | 110.88006 | 51_5 | 91.319825 |
| 1_46 | 83.335321 | 3_6 | 126.75125 | 12_12 | 117.69067 | 19_23 | 68.467912 | 30_15 | 56.624563 | 39_5 | 134.07126 | 51_6 | 39.107031 |
| 1_45 | 100 | 4_17 | 79.44344 | 12_9 | 111.3716 | 19_24 | 81.790206 | 30_16 | 61.942435 | 39_6 | 141.0298 | 51_7 | 60.793964 |
| 1_44 | 80.051481 | 4_18 | 117.77228 | 12_15 | 100.55606 | 19_25 | 129.68396 | 30_24 | 58.357905 | 39_8 | 108.16377 | 51_8 | 70.563792 |
| 1_43 | 53.700224 | 4_19 | 129.05625 | 12_16 | 91.446509 | 19_26 | 65.338263 | 30_29 | 115.53518 | 39_7 | 104.68103 | 51_48 | 82.645032 |
| 1_42 | 86.723802 | 4_22 | 122.43364 | 13_3 | 72.24652 | 19_27 | 98.580991 | 30_30 | 113.99733 | 39_9 | 106.98952 | 52_3 | 25.82786 |
| 1_52 | 68.018543 | 4_23 | 113.16136 | 13_2 | 107.32603 | 19_28 | 125.70134 | 30_32 | 96.794703 | 41_59 | 68.307206 | 52_2 | 55.095256 |
| 1_63 | 71.449707 | 4_24 | 119.41632 | 13_1 | 85.559503 | 19_30 | 78.132788 | 30_33 | 113.8394 | 41_62 | 39.474694 | 52_1 | 68.112902 |
| 1_62 | 67.361679 | 4_16 | 88.2703 | 14_3 | 103.81393 | 19_31 | 117.56836 | 30_31 | 81.228467 | 41_63 | 93.801322 | 52_6 | 33.915108 |
| 1_61 | 66.896378 | 4_26 | 123.54186 | 14_2 | 74.226179 | 19_32 | 134.35034 | 30_36 | 101.98673 | 41_66 | 91.256549 | 52_4 | 40.567686 |
| 1_60 | 39.174859 | 4_15 | 123.37072 | 14_1 | 109.80928 | 19_40 | 110.83253 | 30_46 | 110.19051 | 41_64 | 113.02639 | 52_5 | 52.704571 |
| 1_64 | 67.712773 | 4_12 | 85.049897 | 15_1 | 125.70134 | 19_41 | 93.432735 | 33_2 | 103.67011 | 41_65 | 85.737604 | 52_13 | 63.639747 |
| 1_58 | 95.927552 | 4_14 | 84.089642 | 15_4 | 89.44049 | 19_42 | 115.14884 | 33_4 | 119.25089 | 42_5 | 82.931955 | 52_12 | 76.31296 |
| 1_65 | 80.218117 | 4_13 | 93.692145 | 15_2 | 82.13107 | 19_43 | 96.392981 | 33_3 | 148.04133 | 42_6 | 114.19794 | 52_11 | 45.093769 |
| 1_66 | 54.186298 | 4_39 | 97.738577 | 15_3 | 109.76221 | 19_44 | 133.55013 | 33_1 | 119.25089 | 42_7 | 60.725795 | 52_10 | 39.980859 |
| 1_67 | 85.263489 | 4_38 | 134.00521 | 15_5 | 66.914065 | 19_45 | 128.69892 | 33_10 | 89.595608 | 42_14 | 106.72202 | 52_9 | 54.676773 |
| 1_68 | 57.038186 | 4_37 | 105.84844 | 15_6 | 62.286871 | 19_53 | 53.961412 | 33_9 | 106.8806 | 42_8 | 78.29543 | 52_7 | 78.676245 |
| 1_69 | 59.79099 | 4_36 | 74.710421 | 15_7 | 96.151508 | 19_57 | 55.363229 | 33_8 | 98.62327 | 42_12 | 60.767902 | 53_2 | 91.700404 |
| 1_70 | 76.950361 | 4_35 | 110.26692 | 15_8 | 81.648989 | 19_48 | 59.008717 | 33_7 | 112.19435 | 42_18 | 74.587201 | 53_1 | 72.698626 |
| 1_80 | 48.146403 | 4_34 | 123.28523 | 16_1 | 95.187877 | 19_54 | 81.366109 | 33_6 | 93.75711 | 42_13 | 62.071375 | 53_3 | 111.26675 |
| 1_79 | 69.610719 | 4_33 | 70.956168 | 16_32 | 48.700213 | 19_52 | 105.84844 | 33_5 | 121.92551 | 42_27 | 44.844408 | 53_4 | 75.753339 |
| 1_78 | 95.992926 | 4_30 | 87.175824 | 16_33 | 38.867457 | 19_55 | 119.16826 | 33_16 | 84.873224 | 42_26 | 61.471943 | 54_1 | 111.03388 |
| 1_77 | 57.67428 | 4_40 | 93.044966 | 16_17 | 24.965367 | 19_47 | 60.431866 | 33_15 | 103.16832 | 42_25 | 120.99941 | 56_1 | 122.72094 |
| 1_81 | 44.627349 | 4_41 | 74.865938 | 16_16 | 60.991807 | 19_46 | 86.814023 | 33_14 | 65.792726 | 42_24 | 55.748311 | 57_1 | 91.312726 |
| 1_75 | 75.523629 | 4_31 | 159.1073 | 16_8 | 77.384228 | 19_49 | 68.586664 | 33_11 | 97.063447 | 42_28 | 64.93193 | 57_2 | 97.394518 |
| 1_74 | 65.338263 | 4_32 | 120.1636 | 16_43 | 61.275463 | 19_61 | 66.850025 | 33_12 | 111.99089 | 42_22 | 56.978909 | 57_3 | 105.62856 |
| 1_73 | 41.609937 | 4_51 | 63.966187 | 16_54 | 39.392694 | 19_60 | 76.122045 | 33_18 | 134.6656 | 42_21 | 100 | 57_4 | 98.745957 |
| 1_72 | 66.572581 | 4_50 | 74.380688 | 16_57 | 99.930709 | 19_58 | 112.34999 | 33_28 | 124.57374 | 42_23 | 72.799477 | 57_5 | 96.326189 |
| 1_86 | 55.748311 | 4_49 | 100.06934 | 16_42 | 38.182937 | 20_4 | 123.45626 | 33_23 | 127.98724 | 42_20 | 130.40507 | 57_6 | 88.454044 |
| 1_85 | 64.707283 | 4_48 | 95.975867 | 16_49 | 42.161556 | 20_3 | 117.52763 | 33_22 | 107.84804 | 42_19 | 101.11521 | 57_7 | 122.51853 |
| 1_87 | 102.17201 | 4_47 | 88.791896 | 16_50 | 50.94456 | 20_2 | 367.05595 | 33_21 | 95.462101 | 43_5 | 100.66066 | 57_8 | 105.40914 |
| 1_83 | 74.794288 | 4_46 | 91.446509 | 16_64 | 81.31977 | 20_1 | 131.49428 | 33_20 | 112.34999 | 43_6 | 103.4189 | 57_9 | 100.81622 |
| 1_84 | 70.027816 | 4_45 | 66.204446 | 16_71 | 67.03563 | 20_5 | 109.50525 | 33_30 | 119.74787 | 43_2 | 97.501743 | 63_1 | 103.19559 |
| 1_82 | 72.723821 | 4_42 | 104.10216 | 16_69 | 79.685072 | 21_2 | 73.560337 | 33_29 | 93.952275 | 43_9 | 92.851685 | 65_1 | 95.329855 |
| 1_102 | 49.827013 | 4_44 | 81.394319 | 16_70 | 66.942763 | 21_1 | 94.278454 | 33_25 | 93.457438 | 43_8 | 86.874219 | 67_1 | 77.593085 |
| 1_101 | 68.634222 | 4_43 | 78.730798 | 16_73 | 58.845337 | 21_3 | 100.24289 | 33_26 | 103.52649 | 43_7 | 95.859944 | 67_2 | 79.058912 |
| 1_100 | 63.243977 | 4_52 | 92.723055 | 16_76 | 70.563792 | 21_6 | 97.362244 | 33_27 | 99.47331 | 43_10 | 94.846329 | 67_3 | 89.969001 |
| 1_103 | 39.174859 | 4_54 | 88.515376 | 16_83 | 84.323111 | 21_4 | 101.39595 | 33_35 | 94.605765 | 43_11 | 95.727146 | 68_1 | 140.05563 |
| 1_95 | 74.794288 | 4_53 | 101.74797 | 16_75 | 68.872502 | 21_5 | 118.54717 | 33_38 | 94.409242 | 43_16 | 117.28349 | 69_1 | 75.366745 |
| 1_94 | 70.637193 | 4_55 | 131.4379 | 16_74 | 100.41675 | 21_8 | 109.71968 | 33_32 | 127.80994 | 43_13 | 146.20574 | 69_2 | 167.4813 |
| 1_93 | 67.082111 | 4_58 | 89.13105 | 16_72 | 111.34216 | 21_7 | 152.30887 | 33_37 | 104.78131 | 43_17 | 112.84804 | 71_1 | 68.065706 |
| 1_92 | 70.533542 | 4_59 | 65.610563 | 16_77 | 67.501899 | 22_1 | 127.44019 | 33_39 | 122.51853 | 44_94 | 31.797825 | 72_1 | 80.720107 |
| 1_91 | 79.829839 | 4_60 | 104.63615 | 16_78 | 96.459818 | 22_2 | 111.96129 | 33_40 | 88.025901 | 44_93 | 58.308734 | 72_2 | 128.60975 |
| 1_131 | 23.423036 | 4_61 | 79.60881 | 16_79 | 112.46686 | 22_5 | 95.222961 | 33_41 | 90.877812 | 45_2 | 81.988866 | 72_3 | 103.91338 |
| 1_132 | 46.105156 | 4_62 | 79.66401 | 16_84 | 105.15372 | 22_4 | 110.95695 | 33_42 | 106.14232 | 45_1 | 73.560337 | 73_17 | 108.25997 |
| 1_133 | 97.738167 | 4_63 | 149.48492 | 16_85 | 115.26863 | 22_3 | 159.65968 | 33_44 | 82.017291 | 45_3 | 96.593633 | 76_1 | 124.87632 |
| 1_110 | 45.881994 | 4_71 | 49.03895 | 16_86 | 96.996191 | 22_8 | 116.02063 | 33_43 | 107.47492 | 45_4 | 77.164009 | 78_1 | 91.383145 |
| 1_114 | 107.03851 | 4_65 | 83.798713 | 16_87 | 108.41016 | 22_7 | 122.6359 | 33_45 | 133.82813 | 45_5 | 75.68084 | 78_2 | 80.832087 |
| 1_127 | 66.250351 | 4_64 | 113.11014 | 16_88 | 119.58198 | 22_6 | 155.83292 | 33_51 | 90.626194 | 45_6 | 59.79099 | 79_1 | 107.69864 |
| 1_116 | 36.843957 | 4_69 | 93.724616 | 16_89 | 130.94854 | 22_11 | 117.64022 | 33_48 | 95.859944 | 45_7 | 71.161388 | | |
| 1_117 | 40.556358 | 4_70 | 58.92697 | 16_90 | 91.509917 | 22_10 | 121.36744 | 33_50 | 102.10121 | 45_8 | 79.940583 | | |
| 1_115 | 92.530443 | 4_72 | 103.0254 | 16_91 | 102.31375 | 22_9 | 122.94389 | 33_49 | 115.34856 | 45_9 | 102.81138 | | |
| 1_121 | 63.463544 | 4_79 | 79.333384 | 16_92 | 123.79903 | 22_13 | 102.86939 | 33_46 | 147.83624 | 45_10 | 114.6659 | | |
| 1_122 | 44.627349 | 4_80 | 110.64974 | 16_95 | 86.035263 | 22_12 | 95.992926 | 33_47 | 132.96071 | 45_11 | 114.26483 | | |
| 1_123 | 96.284895 | 4_81 | 95.859944 | 16_93 | 96.326189 | 23_1 | 100.12072 | 33_53 | 127.72138 | 45_12 | 110.89765 | | |

TABLE 3B
Expression level of GAL3ST1 in relation to PBS treated control cells (in %) a549 10 μM

| | % PBS | | % PBS | | % PBS | | % PBS | | % PBS | | % PBS | | % PBS |
|---|---|---|---|---|---|---|---|---|---|---|---|---|---|
| | (a549_10 | | (a549_10 | | (a549_10 | | (a549_10 | | (a549_10 | | (a549_10 | | (a549_10 |
| | uM GAL3ST1 norm | | uM GAL3ST1 norm | | uM GAL3ST1 norm | | uM GAL3ST1 norm | | uM GAL3ST1 norm | | uM GAL3ST1 norm | | uM GAL3ST1 norm |
| ASO ID | ATXN3) | ASO ID | ATXN3) | ASO ID | ATXN3) | ASO ID | ATXN3) | ASO ID | ATXN3) | ASO ID | ATXN3) | ASO ID | ATXN3) |
| 1_1 | 54.638887 | 1_164 | 40.697159 | 4_82 | 103.45476 | 16_94 | 93.887175 | 24_1 | 102.74014 | 33_47 | 137.745 | 45_10 | 109.36054 |
| 1_2 | 61.685358 | 1_163 | 44.782284 | 4_85 | 62.816633 | 16_96 | 60.290391 | 25_48 | 14.063231 | 33_53 | 103.95794 | 45_11 | 80.290817 |
| 1_3 | 81.846918 | 1_162 | 67.595542 | 4_84 | 73.113946 | 16_97 | 60.920513 | 25_49 | 24.350107 | 33_52 | 113.20989 | 45_12 | 101.59851 |
| 1_5 | 56.880261 | 1_161 | 37.761815 | 4_83 | 134.63001 | 16_98 | 74.94998 | 25_50 | 87.636108 | 33_54 | 118.09927 | 45_15 | 95.528294 |
| 1_6 | 73.713461 | 1_165 | 73.969375 | 5_1 | 81.16887 | 16_99 | 61.892412 | 26_1 | 87.102044 | 33_57 | 95.859944 | 45_13 | 99.654026 |
| 1_7 | 79.278414 | 1_160 | 131.49428 | 6_1 | 110.88006 | 16_100 | 72.096444 | 26_2 | 68.491649 | 33_56 | 100 | 45_16 | 124.66012 |
| 1_8 | 71.847009 | 1_159 | 110.19051 | 9_1 | 60.206869 | 16_104 | 78.949389 | 26_3 | 86.693756 | 33_55 | 122.09465 | 45_17 | 110.94232 |
| 1_9 | 81.507233 | 1_158 | 54.722973 | 9_2 | 118.42716 | 16_105 | 91.827616 | 26_4 | 102.17201 | 33_68 | 86.453723 | 45_14 | 105.99528 |
| 1_10 | 88.761134 | 1_157 | 97.727713 | 10_1 | 73.662384 | 16_106 | 97.482727 | 26_5 | 129.32489 | 33_69 | 88.331505 | 45_18 | 93.75711 |
| 1_13 | 48.971015 | 1_156 | 82.989459 | 10_2 | 78.036367 | 16_107 | 104.46358 | 26_6 | 55.825648 | 33_70 | 108.22246 | 45_19 | 100.55606 |
| 1_14 | 73.509367 | 1_193 | 30.971217 | 10_3 | 78.186964 | 16_109 | 66.06692 | 26_8 | 78.487279 | 33_73 | 82.427723 | 45_20 | 133.79276 |
| 1_11 | 94.737007 | 1_187 | 35.30636 | 10_4 | 68.968046 | 16_108 | 91.003882 | 26_7 | 81.676899 | 33_72 | 86.274134 | 45_21 | 70.427878 |
| 1_15 | 59.561574 | 1_183 | 60.625641 | 10_9 | 79.844173 | 16_110 | 115.58867 | 26_9 | 60.165151 | 33_71 | 92.274249 | 45_22 | 93.49752 |
| 1_16 | 80.720107 | 1_170 | 45.659913 | 10_5 | 108.59819 | 16_111 | 50.105862 | 26_10 | 66.434291 | 33_75 | 119.91399 | 45_23 | 97.467963 |
| 1_17 | 87.660572 | 1_178 | 71.350725 | 10_10 | 78.024548 | 17_1 | 116.63494 | 26_11 | 72.497342 | 33_74 | 136.90912 | 46_1 | 109.50525 |
| 1_18 | 71.449707 | 1_173 | 80.106988 | 10_11 | 79.774524 | 17_2 | 118.98784 | 26_12 | 39.229205 | 33_76 | 74.12335 | 46_4 | 72.799477 |
| 1_19 | 74.277646 | 1_176 | 46.393674 | 10_12 | 86.934456 | 17_3 | 128.07599 | 26_13 | 58.27706 | 33_77 | 99.102956 | 46_3 | 117.77894 |
| 1_20 | 92.08257 | 1_177 | 47.863573 | 11_1 | 26.868729 | 17_4 | 98.964414 | 26_14 | 91.003882 | 33_78 | 114.97837 | 46_2 | 121.25128 |
| 1_21 | 73.611343 | 1_171 | 60.920513 | 11_2 | 34.364717 | 17_5 | 107.84804 | 27_1 | 79.706517 | 34_2 | 73.866903 | 46_5 | 119.99714 |
| 1_22 | 76.897042 | 2_7 | 46.683997 | 11_3 | 60.962755 | 17_6 | 129.86386 | 28_24 | 58.636975 | 34_3 | 77.431903 | 46_6 | 132.17417 |
| 1_23 | 77.970484 | 2_9 | 75.6284 | 11_5 | 78.401223 | 17_8 | 98.887909 | 28_25 | 91.383145 | 34_1 | 84.440089 | 46_7 | 132.1892 |
| 1_31 | 55.440032 | 2_6 | 113.99733 | 11_6 | 72.597914 | 17_9 | 107.25166 | 29_1 | 80.676394 | 34_6 | 56.683471 | 46_8 | 117.85394 |
| 1_30 | 71.103871 | 2_8 | 66.526452 | 11_7 | 69.255473 | 17_10 | 103.3434 | 30_1 | 76.365875 | 34_7 | 91.066983 | 46_9 | 118.84373 |
| 1_29 | 79.996013 | 2_5 | 74.432263 | 11_8 | 83.450928 | 17_11 | 105.40243 | 30_2 | 80.888135 | 34_8 | 105.55537 | 46_10 | 121.50368 |
| 1_32 | 67.22175 | 2_1 | 77.808518 | 11_4 | 95.727146 | 17_12 | 111.96129 | 30_1 | 81.28156 | 34_5 | 91.302749 | 49_3 | 29.463485 |
| 1_27 | 72.096444 | 2_4 | 68.872502 | 11_9 | 54.714685 | 17_7 | 118.50928 | 30_8 | 61.989997 | 34_4 | 89.254697 | 49_2 | 31.952484 |
| 1_28 | 100.62578 | 2_3 | 72.458082 | 11_10 | 57.554475 | 17_14 | 105.92183 | 30_7 | 80.888135 | 34_9 | 80.496614 | 49_4 | 74.432263 |
| 1_41 | 69.979293 | 2_2 | 72.849956 | 11_11 | 37.423075 | 17_22 | 106.80654 | 30_6 | 96.634103 | 34_10 | 99.589686 | 49_1 | 73.102872 |
| 1_40 | 79.058912 | 2_10 | 52.704571 | 11_12 | 60.457784 | 17_13 | 113.36694 | 30_4 | 81.620305 | 37_1 | 92.274249 | 49_5 | 93.56235 |
| 1_39 | 123.11444 | 2_11 | 108.59819 | 11_13 | 94.343825 | 17_15 | 245.24411 | 30_5 | 113.04993 | 37_3 | 93.044966 | 50_17 | 89.117797 |
| 1_38 | 57.67428 | 2_12 | 130.04401 | 11_14 | 25.348987 | 17_16 | 88.576752 | 30_11 | 68.444191 | 37_2 | 96.928982 | 50_16 | 90.125046 |
| 1_37 | 80.943016 | 2_20 | 78.621729 | 11_16 | 65.338263 | 17_17 | 93.303299 | 30_12 | 85.263489 | 37_4 | 78.840017 | 50_18 | 107.41472 |
| 1_36 | 100.41675 | 2_17 | 114.11379 | 11_24 | 71.996566 | 17_18 | 108.8997 | 30_14 | 109.65716 | 37_5 | 111.41937 | 50_32 | 52.285919 |
| 1_35 | 64.662447 | 2_21 | 125.09291 | 12_2 | 86.874219 | 17_19 | 64.438732 | 30_10 | 65.838346 | 37_6 | 112.74004 | 50_31 | 53.196087 |
| 1_34 | 72.19646 | 2_31 | 59.584129 | 12_3 | 87.78218 | 17_20 | 101.46625 | 30_9 | 73.102872 | 37_7 | 61.471943 | 50_30 | 74.329149 |
| 1_33 | 88.576752 | 2_30 | 62.633222 | 12_4 | 108.59819 | 17_21 | 114.07637 | 30_13 | 74.846149 | 37_8 | 100.34717 | 50_35 | 75.744511 |
| 1_51 | 57.483448 | 2_29 | 93.49752 | 12_5 | 104.39119 | 19_10 | 68.207392 | 30_19 | 67.548704 | 37_9 | 111.49662 | 50_33 | 69.217969 |
| 1_47 | 65.292989 | 3_4 | 75.785828 | 12_8 | 114.23462 | 19_11 | 93.966506 | 30_22 | 75.001949 | 37_11 | 116.85448 | 50_40 | 72.698626 |
| 1_50 | 71.747477 | 3_3 | 80.496614 | 12_13 | 75.785828 | 19_12 | 118.09927 | 30_21 | 83.047002 | 37_12 | 102.18256 | 50_39 | 73.764573 |
| 1_49 | 53.700224 | 3_2 | 101.11521 | 12_10 | 80.608283 | 19_17 | 44.074008 | 30_23 | 74.638919 | 37_10 | 103.74199 | 50_37 | 107.02887 |
| 1_48 | 58.075437 | 3_1 | 96.928982 | 12_14 | 121.08331 | 19_14 | 82.017291 | 30_18 | 77.81656 | 38_1 | 104.46358 | 51_3 | 77.862469 |
| 1_53 | 80.106988 | 3_5 | 108.3726 | 12_11 | 102.17201 | 19_8 | 108.95712 | 30_17 | 77.874263 | 39_1 | 92.658806 | 51_1 | 84.206295 |
| 1_46 | 67.128625 | 3_6 | 124.66012 | 12_12 | 109.42937 | 19_23 | 33.569397 | 30_15 | 72.558599 | 39_2 | 122.33205 | 51_2 | 117.13876 |
| 1_45 | 86.573657 | 4_17 | 76.471814 | 12_9 | 123.27025 | 19_24 | 76.950361 | 30_16 | 73.611343 | 39_3 | 138.12744 | 51_4 | 84.58543 |
| 1_44 | 112.34999 | 4_18 | 102.88267 | 12_15 | 85.145371 | 19_25 | 94.934212 | 30_20 | 74.277646 | 39_4 | 102.81138 | 51_5 | 98.350207 |
| 1_43 | 80.832087 | 4_19 | 123.71325 | 12_16 | 86.934456 | 19_26 | 68.82478 | 30_24 | 77.217513 | 39_5 | 113.44555 | 51_6 | 45.4773 |
| 1_42 | 96.140653 | 4_22 | 65.929681 | 13_3 | 78.186964 | 19_27 | 84.85529 | 30_29 | 93.309137 | 39_6 | 123.19981 | 51_7 | 54.980807 |
| 1_52 | 103.38307 | 4_23 | 88.774058 | 13_2 | 120.3303 | 19_28 | 89.44049 | 30_30 | 84.38158 | 39_8 | 84.318929 | 51_8 | 70.222244 |
| 1_63 | 57.514595 | 4_24 | 104.10216 | 13_1 | 150.62905 | 19_30 | 87.055056 | 30_32 | 93.432735 | 39_7 | 95.594532 | 51_48 | 82.587766 |
| 1_62 | 71.697762 | 4_16 | 97.670853 | 14_3 | 66.618741 | 19_31 | 98.706604 | 30_33 | 110.19051 | 39_9 | 81.035418 | 52_3 | 40.781875 |
| 1_61 | 78.132788 | 4_26 | 108.14748 | 14_2 | 93.174043 | 19_32 | 120.24692 | 30_31 | 85.136968 | 41_59 | 72.329623 | 52_2 | 58.723099 |
| 1_60 | 51.548416 | 4_15 | 148.14398 | 14_1 | 127.80994 | 19_40 | 78.86539 | 30_36 | 75.134336 | 41_62 | 33.797771 | 52_1 | 79.11373 |
| 1_64 | 81.181253 | 4_12 | 71.222687 | 15_1 | 122.94389 | 19_41 | 79.553648 | 30_46 | 122.26403 | 41_63 | 75.463938 | 52_6 | 32.308821 |
| 1_58 | 93.034767 | 4_14 | 89.688816 | 15_4 | 67.128625 | 19_42 | 124.85196 | 33_2 | 105.55537 | 41_66 | 84.85079 | 52_4 | 60.165255 |
| 1_65 | 67.548704 | 4_13 | 92.787348 | 15_2 | 92.018765 | 19_43 | 60.709744 | 33_4 | 115.02919 | 41_64 | 113.77979 | 52_5 | 54.866597 |
| 1_66 | 72.296615 | 4_39 | 76.471814 | 15_3 | 109.39021 | 19_44 | 125.34746 | 33_3 | 143.29617 | 41_65 | 102.24285 | 52_13 | 59.213581 |
| 1_67 | 98.897092 | 4_38 | 102.38077 | 15_5 | 69.581569 | 19_45 | 133.32987 | 33_1 | 83.508792 | 42_5 | 62.981499 | 52_12 | 73.713461 |
| 1_68 | 56.565723 | 4_37 | 101.04514 | 15_6 | 75.158074 | 19_53 | 59.254639 | 33_10 | 87.370522 | 42_6 | 124.14232 | 52_11 | 79.719248 |
| 1_69 | 70.368419 | 4_36 | 93.559152 | 15_7 | 91.560665 | 19_57 | 73.102872 | 33_9 | 102.38469 | 42_7 | 59.881732 | 52_10 | 29.758368 |
| 1_70 | 76.524838 | 4_35 | 100 | 15_8 | 88.322135 | 19_48 | 76.365875 | 33_8 | 107.77331 | 42_14 | 92.108844 | 52_9 | 64.528125 |
| 1_80 | 56.495917 | 4_34 | 110.11416 | 16_1 | 102.93537 | 19_54 | 82.371577 | 33_7 | 95.528294 | 42_8 | 84.38158 | 52_7 | 83.393104 |
| 1_79 | 78.503569 | 4_33 | 78.949389 | 16_32 | 42.48425 | 19_52 | 97.94203 | 33_6 | 104.46358 | 42_12 | 58.213807 | 53_2 | 111.41937 |
| 1_78 | 99.654026 | 4_30 | 85.797053 | 16_33 | 49.872777 | 19_55 | 103.16832 | 33_5 | 104.97167 | 42_18 | 72.698626 | 53_1 | 80.664176 |
| 1_77 | 54.942711 | 4_40 | 96.66061 | 16_17 | 46.619324 | 19_47 | 54.016574 | 33_16 | 73.46422 | 42_13 | 87.843047 | 53_3 | 91.64049 |
| 1_81 | 61.386784 | 4_41 | 76.409735 | 16_16 | 66.690172 | 19_46 | 69.59232 | 33_15 | 96.392981 | 42_27 | 68.112902 | 53_4 | 91.17818 |
| 1_75 | 77.164009 | 4_31 | 111.03388 | 16_8 | 86.374695 | 19_49 | 76.524838 | 33_14 | 112.97472 | 42_26 | 73.204285 | 54_1 | 81.846918 |
| 1_74 | 56.644194 | 4_32 | 116.63494 | 16_43 | 60.62133 | 19_61 | 54.487606 | 33_11 | 74.535519 | 42_25 | 95.793522 | 56_1 | 97.892613 |
| 1_73 | 60.793964 | 4_51 | 60.305733 | 16_54 | 68.112902 | 19_60 | 70.423941 | 33_12 | 95.356869 | 42_24 | 53.700224 | 57_1 | 82.128355 |
| 1_72 | 65.022007 | 4_50 | 96.459818 | 16_57 | 108.8997 | 19_58 | 77.217513 | 33_18 | 112.33914 | 42_28 | 64.707283 | 57_2 | 102.1032 |
| 1_86 | 56.487361 | 4_49 | 107.17735 | 16_42 | 44.47295 | 20_4 | 111.49662 | 33_28 | 105.92183 | 42_22 | 67.793484 | 57_3 | 114.3931 |
| 1_85 | 62.589823 | 4_48 | 71.553127 | 16_49 | 55.516942 | 20_3 | 113.05306 | 33_23 | 115.98997 | 42_21 | 80.552429 | 57_4 | 78.904554 |
| 1_87 | 109.12639 | 4_47 | 107.41672 | 16_50 | 74.483873 | 20_2 | 125.44022 | 33_22 | 117.20223 | 42_23 | 68.16013 | 57_5 | 79.333384 |
| 1_83 | 42.543186 | 4_46 | 124.22883 | 16_64 | 81.115364 | 20_1 | 127.54444 | 33_21 | 82.87449 | 42_20 | 68.82478 | 57_6 | 84.147948 |
| 1_84 | 72.497342 | 4_45 | 72.648252 | 16_71 | 63.993621 | 20_5 | 97.806347 | 33_20 | 96.192745 | 42_19 | 115.02919 | 57_7 | 93.692145 |
| 1_82 | 94.554532 | 4_42 | 95.06591 | 16_69 | 79.370604 | 21_2 | 74.535519 | 33_30 | 107.32603 | 43_5 | 80.956483 | 57_8 | 114.15547 |
| 1_102 | 54.714685 | 4_44 | 98.486644 | 16_70 | 90.689033 | 21_1 | 110.64974 | 33_29 | 134.63001 | 43_6 | 87.128615 | 57_9 | 78.602888 |
| 1_101 | 83.104586 | 4_43 | 60.457784 | 16_73 | 79.60881 | 21_3 | 114.17276 | 33_25 | 85.508116 | 43_2 | 119.93216 | 63_1 | 80.123795 |
| 1_100 | 87.296759 | 4_52 | 83.798713 | 16_76 | 89.254697 | 21_6 | 87.4379 | 33_26 | 104.46358 | 43_9 | 89.751005 | 65_1 | 104.89893 |
| 1_103 | 33.962157 | 4_54 | 91.891288 | 16_83 | 97.738577 | 21_4 | 97.738577 | 33_27 | 117.39827 | 43_8 | 90.000193 | 67_1 | 80.3294 |
| 1_95 | 45.156326 | 4_53 | 107.77331 | 16_75 | 101.60701 | 21_5 | 96.915377 | 33_35 | 99.447017 | 43_7 | 94.343825 | 67_2 | 64.528125 |
| 1_94 | 78.307289 | 4_55 | 118.52801 | 16_74 | 103.23985 | 21_8 | 90.629873 | 33_38 | 106.95471 | 43_10 | 74.660629 | 67_3 | 86.706888 |
| 1_93 | 47.434216 | 4_58 | 68.53914 | 16_72 | 114.79024 | 21_7 | 135.4725 | 33_32 | 131.768 | 43_11 | 87.296759 | 68_1 | 101.39595 |
| 1_92 | 81.240652 | 4_59 | 76.897042 | 16_77 | 81.394319 | 22_1 | 102.68244 | 33_31 | 86.072425 | 43_16 | 103.52649 | 69_1 | 94.802697 |
| 1_91 | 148.55529 | 4_60 | 119.99615 | 16_78 | 102.52672 | 22_2 | 128.52063 | 33_37 | 97.205051 | 43_13 | 106.43702 | 69_2 | 109.42937 |
| 1_131 | 30.020086 | 4_61 | 65.428904 | 16_79 | 114.25193 | 22_5 | 105.29578 | 33_39 | 99.723125 | 43_17 | 117.52588 | 71_1 | 53.40327 |
| 1_132 | 50.278029 | 4_62 | 77.324634 | 16_84 | 77.712497 | 22_4 | 115.50858 | 33_40 | 83.740649 | 44_94 | 42.809414 | 72_1 | 90.000193 |
| 1_133 | 87.79535 | 4_63 | 125.00623 | 16_85 | 90.437938 | 22_3 | 123.11444 | 33_41 | 90.500646 | 44_93 | 63.96788 | 72_2 | 102.59781 |
| 1_110 | 50.312891 | 4_71 | 59.419155 | 16_86 | 91.573369 | 22_8 | 99.08149 | 33_42 | 129.77388 | 45_2 | 84.394361 | 72_3 | 113.63705 |
| 1_114 | 70.312728 | 4_65 | 81.563749 | 16_87 | 85.453916 | 22_7 | 104.56792 | 33_44 | 88.945899 | 45_1 | 85.263489 | 73_17 | 106.7487 |
| 1_127 | 71.499249 | 4_64 | 100.94947 | 16_88 | 106.28957 | 22_6 | 141.22544 | 33_43 | 102.10121 | 45_3 | 108.14748 | 76_1 | 92.544459 |
| 1_116 | 47.705203 | 4_69 | 67.981708 | 16_89 | 124.22883 | 22_11 | 100.28296 | 33_45 | 136.1235 | 45_4 | 74.226179 | 78_1 | 93.044966 |
| 1_117 | 60.625641 | 4_70 | 72.849956 | 16_90 | 74.898047 | 22_10 | 116.87233 | 33_51 | 73.102872 | 45_5 | 86.333956 | 78_2 | 91.446509 |
| 1_115 | 97.738577 | 4_72 | 84.147948 | 16_91 | 94.671363 | 22_9 | 121.67224 | 33_48 | 75.105996 | 45_6 | 87.843047 | 79_1 | 95.992926 |
| 1_121 | 63.77222 | 4_79 | 66.664934 | 16_92 | 98.009942 | 22_13 | 98.130137 | 33_50 | 120.08034 | 45_7 | 75.389112 | | |
| 1_122 | 65.112209 | 4_81 | 82.87449 | 16_95 | 64.21579 | 22_12 | 111.26501 | 33_49 | 99.447017 | 45_8 | 110.19051 | | |
| 1_123 | 72.412874 | 4_80 | 118.42716 | 16_93 | 86.573657 | 23_1 | 90.968241 | 33_46 | 134.81677 | 45_9 | 78.949389 | | |

### Example 2 Testing In Vitro Efficacy of Antisense Oligonucleotides Targeting GAL3ST1 in CACO2 Cells at Single Test Concentration

Following the screening results from example 1, additional antisense oligonucleotides (ASOs) were synthesized to target the GAL3ST1 gene. These ASOs were designed based on the sequences identified in example 1 as potential high efficacy targets. To optimize the screening process, the new ASOs were tested at a lower concentration of 1 μM compared to the initial screening concentration of 10 μM.

To assess the efficacy of the new ASOs, a cell-based screening assay was developed using Caco2 cells and in SK-N-AS cells. The cells were cultured and expanded according to the supplier's recommendations. When reaching 70-80% confluency the cells are trypsinized and resuspended in growth media. Viable cells are counted using trypan blue and a Vi-CELL automatic cell counter (Beckman Coulter). A predetermined number of cells were diluted in growth media, added to 96-well plates, and treated with the ASOs. To minimize plate effect and evaporation, sterile PBS was added to the moats of the culture plates.

After 24 hours of incubation, the GAL3ST1 ASOs (listed in Table 5 and 6, under the column “ASO ID”) were added directly to the growth media from a 20-fold stock dilution in PBS to achieve a final treatment ASO concentration. The most important parameters for the cellular work are summarized in Table 4.

TABLE 4
Information on Caco2 and SK-N-AS cells

| | | | | Incubation | |
|---|---|---|---|---|---|
| | | | | prior to | Incubation |
| Cell | Provi- | Culturing | Seeding density | ASO | time with |
| Line | der | conditions | (cells/well) | addition | ASO |
| Caco2 | ATCC | 5% CO₂ | 8,000 cells/well | 24 h | 72 h |
| | | and 95% | in 190 μl of | | |
| | | humidity | media: DMEM + | | |
| | | at 37° C. | 10% FBS + | | |
| | | | Sodium | | |
| | | | pyruvate + Non | | |
| | | | essential | | |
| | | | aminoacids. | | |
| SK- | ATCC | 5% CO₂ | 8,000 cells/well | 24 h | 96 h |
| N-AS | | and 95% | in 190 μl of | | |
| | | humidity | media: DMEM + | | |
| | | at 37° C. | 10% FBS + | | |
| | | | Sodium | | |
| | | | pyruvate + Non | | |
| | | | essential | | |
| | | | aminoacids. | | |

Each plate contained antisense oligonucleotides (ASOs) designed to target GALST1, as well as 10 PBS controls (10 μL), 4 GAL3ST1 positive controls, and 2 controls targeting the ATXN3 gene. After 72 hours, the Caco2 cells were harvested by gently aspirating the growth media, and RNA was extracted using the Macherey-Nagel NucleoSpin 96 RNA Kit according to the manufacturer's instructions. The RNA was eluted in 75 μl of water.

Quantitative PCR (qPCR) experiments were performed as described in Example 1. The amount of GAL3ST1 mRNA was calculated using the ddCT method, with the median of all the PBS-treated wells on the same plate serving as the untreated control. The expression level of GAL3ST1 following ASO treatment (listed in Table 6) was expressed as a percentage of the PBS-treated wells. Taken together, the data demonstrate that additional high efficacy compounds can be generated at previously identified target sites (Example 1) and in nearby regions.

TABLE 5
Expression level of GAL3ST1 in relation to PBS treated control cells (in %) Caco2 10 μM

| | % PBS | | % PBS | | % PBS | | % PBS | | % PBS | | % PBS |
|---|---|---|---|---|---|---|---|---|---|---|---|
| | (Caco2 | | (Caco2 | | (Caco2 | | (Caco2 | | (Caco2 | | (Caco2 |
| | GAL3ST1 | | GAL3ST1 | | GAL3ST1 | | GAL3ST1 | | GAL3ST1 | | GAL3ST1 |
| ASO | norm | ASO | norm | ASO | norm | ASO | norm | ASO | norm | ASO | norm |
| ID | ATXN3) | ID | ATXN3) | ID | ATXN3) | ID | ATXN3) | ID | ATXN3) | ID | ATXN3) |
| 1_4 | 53.22 | 4_29 | 91.17 | 19_9 | 61.03 | 28_33 | 109.62 | 42_17 | 94.55 | 50_8 | 95.06 |
| 1_12 | 78.29 | 4_38 | 99.28 | 19_7 | 73.21 | 28_34 | 90.12 | 43_1 | 92.19 | 50_3 | 86.29 |
| 1_24 | 73.71 | 4_48 | 74.26 | 19_23 | 47.12 | 28_2 | 85.64 | 43_3 | 89.79 | 50_2 | 63.83 |
| 1_26 | 76.45 | 4_56 | 82.75 | 19_29 | 54.86 | 28_35 | 88.67 | 43_4 | 81.27 | 50_1 | 96.79 |
| 1_25 | 67.99 | 4_57 | 93.33 | 19_33 | 75.20 | 28_3 | 93.23 | 43_15 | 109.40 | 50_26 | 69.70 |
| 1_41 | 69.24 | 4_64 | 105.33 | 19_34 | 91.15 | 28_37 | 89.84 | 43_12 | 101.29 | 50_29 | 68.61 |
| 1_60 | 51.81 | 4_73 | 86.21 | 19_35 | 82.41 | 28_38 | 88.52 | 43_14 | 102.64 | 50_28 | 54.22 |
| 1_58 | 105.44 | 4_74 | 66.85 | 19_36 | 69.38 | 28_39 | 86.26 | 44_4 | 103.21 | 50_27 | 71.41 |
| 1_55 | 77.38 | 4_75 | 85.61 | 19_37 | 66.84 | 28_40 | 100.58 | 44_3 | 88.04 | 50_34 | 56.77 |
| 1_54 | 79.26 | 4_76 | 68.13 | 19_38 | 72.67 | 28_23 | 92.66 | 44_2 | 91.29 | 50_25 | 60.87 |
| 1_57 | 61.25 | 4_68 | 82.65 | 19_39 | 71.86 | 28_22 | 81.75 | 44_14 | 89.81 | 50_19 | 56.81 |
| 1_56 | 77.98 | 4_77 | 78.91 | 19_56 | 65.10 | 28_21 | 73.68 | 44_1 | 87.39 | 50_24 | 66.69 |
| 1_59 | 66.85 | 4_67 | 78.67 | 19_50 | 74.86 | 28_11 | 82.25 | 44_5 | 87.84 | 50_22 | 66.80 |
| 1_76 | 49.29 | 4_78 | 74.80 | 19_51 | 55.03 | 28_4 | 101.78 | 44_16 | 96.93 | 50_21 | 60.62 |
| 1_71 | 46.36 | 4_66 | 66.89 | 19_59 | 69.61 | 28_5 | 83.16 | 44_17 | 95.73 | 50_20 | 47.23 |
| 1_99 | 56.41 | 10_6 | 109.72 | 25_48 | 27.02 | 28_6 | 85.11 | 44_18 | 94.08 | 50_23 | 69.73 |
| 1_98 | 71.63 | 10_7 | 91.91 | 25_49 | 25.42 | 28_7 | 81.61 | 44_19 | 98.63 | 51_6 | 58.67 |
| 1_97 | 59.90 | 10_8 | 93.01 | 25_51 | 34.27 | 28_8 | 79.79 | 44_20 | 88.38 | 51_10 | 74.73 |
| 1_96 | 47.94 | 11_1 | 76.47 | 25_52 | 37.78 | 28_9 | 92.85 | 44_23 | 80.83 | 51_12 | 93.49 |
| 1_103 | 48.85 | 11_2 | 59.08 | 25_53 | 44.77 | 28_10 | 68.48 | 44_22 | 87.27 | 51_22 | 60.65 |
| 1_90 | 74.06 | 11_5 | 94.52 | 25_54 | 45.32 | 28_12 | 70.98 | 44_31 | 100.31 | 51_45 | 64.44 |
| 1_89 | 78.41 | 11_14 | 62.04 | 25_56 | 27.73 | 28_20 | 76.40 | 44_24 | 91.59 | 51_43 | 84.82 |
| 1_88 | 66.58 | 11_17 | 72.25 | 25_64 | 18.92 | 28_13 | 99.46 | 44_25 | 86.64 | 51_42 | 51.05 |
| 1_133 | 93.45 | 11_15 | 70.96 | 25_57 | 25.46 | 28_14 | 90.14 | 44_26 | 73.04 | 51_41 | 61.28 |
| 1_134 | 76.86 | 11_30 | 63.92 | 25_58 | 62.02 | 28_15 | 79.06 | 44_27 | 72.63 | 51_39 | 61.69 |
| 1_135 | 74.31 | 11_29 | 89.25 | 25_59 | 21.70 | 28_16 | 88.57 | 44_28 | 77.40 | 51_38 | 60.01 |
| 1_136 | 50.62 | 11_28 | 71.30 | 25_60 | 43.72 | 28_18 | 82.45 | 44_29 | 85.70 | 51_37 | 70.22 |
| 1_137 | 57.41 | 11_27 | 63.43 | 25_61 | 27.23 | 28_19 | 94.06 | 44_30 | 87.69 | 51_36 | 64.29 |
| 1_138 | 70.71 | 11_26 | 77.86 | 25_62 | 30.88 | 28_42 | 83.88 | 44_15 | 88.47 | 51_35 | 66.17 |
| 1_139 | 56.05 | 11_22 | 55.36 | 25_63 | 47.61 | 28_41 | 75.88 | 44_21 | 88.82 | 51_34 | 69.62 |
| 1_140 | 60.49 | 11_25 | 50.06 | 25_47 | 20.64 | 28_17 | 71.65 | 44_13 | 108.67 | 51_32 | 73.80 |
| 1_141 | 58.99 | 11_23 | 72.59 | 25_55 | 35.22 | 28_36 | 71.18 | 44_12 | 93.64 | 51_29 | 74.71 |
| 1_142 | 64.92 | 11_20 | 59.32 | 25_46 | 24.65 | 28_1 | 80.49 | 44_6 | 70.21 | 51_28 | 58.95 |
| 1_147 | 64.98 | 11_21 | 76.22 | 25_35 | 32.99 | 30_29 | 110.82 | 44_7 | 98.45 | 51_26 | 58.20 |
| 1_144 | 49.00 | 11_19 | 52.60 | 25_28 | 36.96 | 33_13 | 103.72 | 44_8 | 83.80 | 51_44 | 66.58 |
| 1_145 | 55.09 | 11_18 | 49.65 | 25_29 | 50.80 | 33_17 | 68.20 | 44_11 | 88.04 | 51_23 | 68.62 |
| 1_146 | 91.59 | 11_31 | 61.06 | 25_30 | 37.07 | 33_19 | 99.68 | 44_9 | 73.54 | 51_47 | 73.33 |
| 1_148 | 57.61 | 12_7 | 95.82 | 25_31 | 27.01 | 33_24 | 101.17 | 44_10 | 86.56 | 51_24 | 61.11 |
| 1_149 | 66.51 | 12_1 | 97.61 | 25_32 | 26.08 | 33_33 | 116.53 | 44_35 | 104.28 | 51_52 | 96.65 |
| 1_150 | 41.46 | 12_6 | 92.50 | 25_33 | 25.95 | 33_34 | 98.48 | 44_34 | 85.41 | 51_65 | 49.00 |
| 1_151 | 52.84 | 16_33 | 52.95 | 25_34 | 30.81 | 33_36 | 115.49 | 44_33 | 85.75 | 51_56 | 64.93 |
| 1_152 | 49.71 | 16_34 | 85.49 | 25_36 | 29.12 | 34_5 | 104.44 | 44_32 | 113.73 | 51_58 | 74.10 |
| 1_153 | 42.10 | 16_35 | 68.49 | 25_44 | 38.14 | 34_12 | 84.53 | 44_36 | 95.73 | 51_59 | 48.90 |
| 1_154 | 47.82 | 16_36 | 66.27 | 25_37 | 28.89 | 34_13 | 80.42 | 44_37 | 118.62 | 51_61 | 101.47 |
| 1_155 | 80.40 | 16_25 | 53.12 | 25_38 | 28.79 | 34_11 | 92.13 | 44_53 | 95.66 | 51_69 | 62.22 |
| 1_130 | 45.77 | 16_29 | 75.74 | 25_39 | 43.22 | 37_11 | 88.14 | 44_54 | 91.40 | 51_71 | 55.32 |
| 1_143 | 53.00 | 16_23 | 50.63 | 25_40 | 20.80 | 39_2 | 97.51 | 44_55 | 105.47 | 51_67 | 52.27 |
| 1_128 | 40.70 | 16_24 | 66.43 | 25_41 | 32.29 | 41_47 | 41.67 | 44_56 | 91.14 | 51_63 | 82.44 |
| 1_126 | 37.86 | 16_21 | 37.64 | 25_42 | 37.61 | 41_46 | 63.65 | 44_57 | 83.99 | 51_51 | 70.44 |
| 1_112 | 50.17 | 16_19 | 58.63 | 25_43 | 30.69 | 41_45 | 61.98 | 44_60 | 86.17 | 51_57 | 72.29 |
| 1_111 | 66.91 | 16_18 | 40.00 | 25_45 | 32.63 | 41_44 | 55.40 | 44_59 | 90.62 | 51_55 | 60.02 |
| 1_108 | 65.44 | 16_15 | 54.91 | 25_65 | 25.92 | 41_43 | 93.19 | 44_64 | 86.94 | 51_62 | 86.68 |
| 1_109 | 41.17 | 16_14 | 39.52 | 25_66 | 22.03 | 41_42 | 79.27 | 44_61 | 85.87 | 51_53 | 61.32 |
| 1_113 | 48.45 | 16_13 | 76.38 | 25_90 | 20.84 | 41_41 | 63.01 | 44_62 | 79.43 | 51_54 | 89.62 |
| 1_107 | 55.06 | 16_12 | 55.79 | 25_89 | 31.72 | 41_40 | 69.48 | 44_63 | 93.18 | 51_60 | 51.59 |
| 1_106 | 50.20 | 16_22 | 84.73 | 25_86 | 29.51 | 41_39 | 84.23 | 44_52 | 70.26 | 51_64 | 92.42 |
| 1_129 | 27.34 | 16_20 | 68.21 | 25_85 | 20.93 | 41_38 | 88.90 | 44_58 | 76.28 | 51_68 | 51.08 |
| 1_105 | 54.84 | 16_37 | 23.50 | 25_26 | 17.48 | 41_37 | 86.55 | 44_50 | 86.88 | 51_66 | 60.53 |
| 1_104 | 38.22 | 16_30 | 57.96 | 25_67 | 27.53 | 41_36 | 116.11 | 44_49 | 68.16 | 51_73 | 74.63 |
| 1_114 | 100.99 | 16_31 | 40.24 | 25_83 | 28.44 | 41_35 | 76.29 | 44_51 | 70.36 | 51_72 | 93.79 |
| 1_116 | 45.13 | 16_28 | 41.50 | 25_68 | 29.26 | 41_2 | 58.06 | 44_48 | 78.04 | 51_74 | 86.03 |
| 1_118 | 51.42 | 16_10 | 29.84 | 25_69 | 31.01 | 41_34 | 90.77 | 44_47 | 81.30 | 51_40 | 57.59 |
| 1_119 | 62.14 | 16_27 | 55.63 | 25_70 | 31.37 | 41_33 | 90.80 | 44_38 | 73.94 | 51_27 | 83.38 |
| 1_120 | 54.33 | 16_26 | 57.22 | 25_71 | 32.13 | 41_32 | 83.72 | 44_46 | 102.66 | 51_50 | 55.40 |
| 1_124 | 71.21 | 16_11 | 63.22 | 25_72 | 32.54 | 41_31 | 82.84 | 44_45 | 81.06 | 51_70 | 69.44 |
| 1_125 | 73.55 | 16_38 | 63.48 | 25_73 | 37.93 | 41_57 | 87.73 | 44_44 | 73.70 | 51_25 | 54.72 |
| 1_164 | 49.45 | 16_9 | 76.44 | 25_74 | 38.14 | 41_58 | 81.78 | 44_43 | 84.88 | 51_30 | 79.30 |
| 1_163 | 49.63 | 16_5 | 37.30 | 25_75 | 39.33 | 41_48 | 95.14 | 44_42 | 71.09 | 51_31 | 79.47 |
| 1_161 | 37.75 | 16_7 | 39.49 | 25_76 | 40.54 | 41_49 | 60.91 | 44_41 | 76.89 | 51_33 | 86.14 |
| 1_157 | 110.84 | 16_6 | 62.66 | 25_77 | 41.93 | 41_50 | 81.43 | 44_40 | 79.50 | 52_3 | 49.49 |
| 1_193 | 32.65 | 16_4 | 63.85 | 25_78 | 43.02 | 41_51 | 71.19 | 44_39 | 82.35 | 52_4 | 60.43 |
| 1_187 | 44.60 | 16_3 | 39.58 | 25_79 | 43.65 | 41_52 | 46.22 | 44_90 | 105.98 | 52_10 | 60.17 |
| 1_184 | 57.05 | 16_2 | 43.44 | 25_80 | 44.18 | 41_53 | 68.94 | 44_88 | 102.59 | 52_8 | 54.65 |
| 1_185 | 62.80 | 16_56 | 40.47 | 25_81 | 48.06 | 41_54 | 81.34 | 44_91 | 94.77 | 52_15 | 73.27 |
| 1_186 | 48.96 | 16_55 | 55.49 | 25_82 | 66.14 | 41_55 | 82.64 | 44_85 | 96.56 | 53_3 | 109.45 |
| 1_192 | 32.24 | 16_44 | 48.27 | 25_87 | 27.93 | 41_30 | 83.68 | 44_84 | 101.73 | 80_1 | 63.99 |
| 1_180 | 55.35 | 16_45 | 57.81 | 25_88 | 23.70 | 41_29 | 84.55 | 44_83 | 99.58 | 80_2 | 88.05 |
| 1_188 | 37.21 | 16_46 | 77.71 | 25_27 | 29.27 | 41_28 | 87.40 | 44_82 | 103.23 | 81_1 | 73.31 |
| 1_189 | 67.53 | 16_47 | 71.14 | 25_84 | 35.28 | 41_14 | 87.56 | 44_81 | 96.22 | 82_1 | 59.47 |
| 1_190 | 52.58 | 16_48 | 67.66 | 25_25 | 23.75 | 41_3 | 97.75 | 44_87 | 89.29 | 84_1 | 65.70 |
| 1_191 | 52.78 | 16_42 | 38.15 | 25_19 | 41.41 | 41_4 | 74.70 | 44_89 | 94.93 | 85_1 | 91.88 |
| 1_181 | 57.45 | 16_51 | 65.42 | 25_2 | 38.08 | 41_5 | 97.70 | 44_92 | 81.63 | 86_1 | 88.72 |
| 1_182 | 74.12 | 16_52 | 52.26 | 25_3 | 35.99 | 41_6 | 78.51 | 44_80 | 94.31 | 86_2 | 51.52 |
| 1_179 | 42.62 | 16_53 | 43.97 | 25_4 | 38.61 | 41_7 | 57.33 | 44_86 | 75.87 | 86_3 | 58.25 |
| 1_166 | 40.97 | 16_80 | 90.10 | 25_5 | 36.58 | 41_8 | 44.61 | 44_78 | 75.10 | 86_4 | 52.98 |
| 1_167 | 52.01 | 16_81 | 92.69 | 25_6 | 32.42 | 41_9 | 60.21 | 44_75 | 74.16 | 87_1 | 74.61 |
| 1_168 | 37.07 | 16_82 | 88.43 | 25_7 | 33.27 | 41_10 | 69.74 | 44_71 | 86.82 | 88_1 | 59.94 |
| 1_169 | 28.31 | 16_101 | 69.46 | 25_8 | 23.12 | 41_1 | 88.78 | 44_65 | 57.71 | 89_1 | 57.52 |
| 1_172 | 92.17 | 16_102 | 70.80 | 25_9 | 25.04 | 41_11 | 86.76 | 44_68 | 59.29 | 89_2 | 95.87 |
| 1_174 | 101.92 | 16_103 | 78.87 | 25_10 | 28.70 | 41_12 | 85.37 | 44_79 | 88.11 | 89_3 | 81.97 |
| 1_175 | 81.97 | 16_112 | 96.64 | 25_1 | 25.46 | 41_13 | 62.42 | 44_66 | 87.75 | 91_1 | 61.05 |
| 1_194 | 53.40 | 16_113 | 93.58 | 25_11 | 24.06 | 41_15 | 52.80 | 44_76 | 57.65 | 91_2 | 89.74 |
| 1_195 | 45.29 | 16_114 | 91.08 | 25_24 | 20.92 | 41_27 | 80.67 | 44_74 | 71.93 | 91_3 | 85.07 |
| 1_196 | 67.20 | 16_115 | 93.69 | 25_13 | 29.32 | 41_16 | 93.38 | 44_73 | 77.22 | 92_1 | 103.07 |
| 1_197 | 68.25 | 16_116 | 72.65 | 25_14 | 33.54 | 41_17 | 50.01 | 44_72 | 77.99 | 93_1 | 100.11 |
| 1_198 | 86.15 | 16_117 | 87.81 | 25_15 | 44.29 | 41_18 | 77.99 | 44_77 | 90.48 | 98_1 | 93.96 |
| 2_13 | 94.19 | 16_118 | 94.47 | 25_16 | 18.70 | 41_19 | 74.82 | 44_70 | 88.85 | 98_2 | 85.72 |
| 2_14 | 90.60 | 16_119 | 95.39 | 25_17 | 35.37 | 41_20 | 74.36 | 44_67 | 92.55 | 100_1 | 73.73 |
| 2_15 | 98.22 | 16_120 | 87.27 | 25_18 | 32.65 | 41_21 | 50.59 | 44_69 | 94.92 | 102_13 | 40.73 |
| 4_11 | 97.94 | 16_121 | 101.57 | 25_12 | 24.67 | 41_22 | 91.13 | 44_94 | 45.93 | 102_12 | 42.59 |
| 4_9 | 97.77 | 16_122 | 80.81 | 25_20 | 30.72 | 41_23 | 82.81 | 46_3 | 105.38 | 103_1 | 67.09 |
| 4_8 | 91.59 | 16_127 | 80.60 | 25_21 | 25.82 | 41_24 | 67.42 | 49_3 | 35.70 | 103_25 | 88.76 |
| 4_7 | 109.54 | 16_124 | 58.47 | 25_22 | 29.80 | 41_25 | 83.70 | 49_2 | 31.70 | 104_1 | 65.32 |
| 4_6 | 83.95 | 16_123 | 70.68 | 25_23 | 26.57 | 41_26 | 73.12 | 49_1 | 56.01 | 104_2 | 57.71 |
| 4_5 | 105.08 | 16_125 | 62.73 | 26_1 | 86.67 | 41_56 | 72.00 | 50_15 | 113.26 | 108_10 | 34.29 |
| 4_4 | 98.95 | 16_126 | 59.08 | 26_12 | 63.15 | 41_60 | 87.09 | 50_14 | 80.57 | 108_25 | 49.83 |
| 4_3 | 116.30 | 19_13 | 55.41 | 26_13 | 71.23 | 41_61 | 67.14 | 50_13 | 89.76 | 108_41 | 52.66 |
| 4_2 | 115.86 | 19_21 | 105.63 | 28_25 | 99.78 | 41_62 | 52.32 | 50_11 | 102.96 | 108_44 | 69.40 |
| 4_1 | 109.14 | 19_15 | 119.05 | 28_26 | 97.96 | 41_64 | 108.72 | 50_10 | 81.06 | 108_45 | 77.40 |
| 4_10 | 94.79 | 19_16 | 92.45 | 28_27 | 91.08 | 42_14 | 104.19 | 50_9 | 80.14 | 108_46 | 61.77 |
| 4_27 | 97.25 | 19_22 | 49.24 | 28_28 | 84.47 | 42_9 | 63.42 | 50_12 | 79.22 | 110_4 | 63.73 |
| 4_20 | 91.25 | 19_18 | 76.79 | 28_29 | 81.80 | 42_10 | 72.23 | 50_7 | 84.39 | 111_1 | 89.81 |
| 4_21 | 88.33 | 19_19 | 55.37 | 28_30 | 106.37 | 42_11 | 68.05 | 50_6 | 79.00 | | |
| 4_25 | 90.47 | 19_20 | 94.84 | 28_31 | 87.34 | 42_15 | 98.89 | 50_5 | 86.02 | | |
| 4_28 | 97.71 | 19_6 | 58.45 | 28_32 | 114.03 | 42_16 | 103.63 | 50_4 | 84.56 | | |

TABLE 6
Expression level of GAL3ST1 in relation to PBS treated control cells (in %) Caco2 10 μM (further analysis)

| | % UTC | | % UTC | | % UTC | | % UTC | | % UTC |
|---|---|---|---|---|---|---|---|---|---|
| ASO | (Caco2_10 | ASO | (Caco2_10 | ASO | (Caco2_10 | ASO | (Caco2_10 | ASO | (Caco2_10 |
| ID | uM_2) | ID | uM_2) | ID | uM_2) | ID | uM_2) | ID | uM_2) |
| 1_193 | 30.06 | 19_4 | 93.4 | 96_13 | 49.2 | 95_18 | 106.6 | 66_5 | 90.6 |
| 16_17 | 41.09 | 16_41 | 60.5 | 96_12 | 27.0 | 103_18 | 39.7 | 29_40 | 82.3 |
| 25_85 | 20.66 | 16_68 | 60.4 | 96_8 | 54.2 | 7_1 | 85.4 | 29_25 | 64.2 |
| 42_32 | 85.8 | 16_62 | 96.4 | 96_6 | 41.3 | 99_1 | 67.7 | 82_5 | 92.4 |
| 96_23 | 55.5 | 16_40 | 76.2 | 96_4 | 97.2 | 77_1 | 72.0 | 29_48 | 79.4 |
| 40_13 | 107.5 | 16_67 | 49.8 | 96_11 | 103.0 | 66_17 | 73.2 | 29_46 | 88.7 |
| 103_24 | 51.6 | 16_61 | 55.9 | 96_3 | 67.8 | 40_6 | 100.9 | 82_2 | 69.8 |
| 60_5 | 88.1 | 16_39 | 62.7 | 96_5 | 38.6 | 103_21 | 49.5 | 66_7 | 85.6 |
| 29_34 | 83.6 | 16_66 | 43.3 | 96_1 | 56.6 | 79_11 | 51.2 | 103_11 | 43.6 |
| 29_39 | 124.5 | 16_65 | 50.2 | 96_2 | 73.6 | 103_20 | 40.0 | 77_3 | 76.1 |
| 29_36 | 75.9 | 16_60 | 55.8 | 96_7 | 68.1 | 7_2 | 82.0 | 32_6 | 71.0 |
| 29_38 | 78.8 | 16_59 | 48.9 | 79_9 | 57.0 | 99_3 | 81.8 | 101_9 | 73.9 |
| 110_3 | 55.8 | 16_58 | 66.3 | 77_21 | 42.1 | 77_4 | 63.5 | 103_15 | 75.3 |
| 77_18 | 75.5 | 16_63 | 67.8 | 77_22 | 63.1 | 66_19 | 84.4 | 74_1 | 93.3 |
| 73_9 | 90.5 | 11_35 | 52.7 | 77_20 | 76.9 | 40_7 | 95.7 | 106_5 | 80.5 |
| 50_38 | 133.0 | 11_32 | 91.8 | 77_19 | 76.0 | 61_10 | 80.0 | 90_4 | 83.0 |
| 64_1 | 73.2 | 11_33 | 72.8 | 66_3 | 64.1 | 103_19 | 35.6 | 29_4 | 86.4 |
| 48_1 | 94.7 | 11_34 | 93.9 | 66_2 | 111.3 | 79_10 | 57.9 | 79_12 | 82.7 |
| 62_3 | 70.8 | 2_28 | 67.6 | 66_1 | 90.8 | 95_15 | 74.6 | 77_6 | 78.4 |
| 32_1 | 54.5 | 2_32 | 76.3 | 64_4 | 61.4 | 99_5 | 80.4 | 73_11 | 69.8 |
| 60_4 | 62.7 | 2_26 | 75.3 | 64_5 | 66.8 | 66_18 | 83.8 | 29_33 | 66.9 |
| 31_2 | 60.2 | 2_24 | 70.8 | 62_15 | 69.4 | 40_8 | 108.0 | 29_9 | 98.0 |
| 73_4 | 87.0 | 2_22 | 66.7 | 62_14 | 85.7 | 40_9 | 90.9 | 29_41 | 87.9 |
| 2_27 | 58.6 | 2_23 | 62.7 | 62_13 | 85.9 | 61_6 | 79.6 | 58_1 | 66.5 |
| 51_14 | 73.5 | 2_19 | 82.9 | 60_8 | 93.7 | 109_1 | 76.4 | 29_42 | 67.1 |
| 97_4 | 82.1 | 2_25 | 34.9 | 59_1 | 57.3 | 79_7 | 48.7 | 61_1 | 81.9 |
| 97_13 | 73.4 | 2_18 | 60.6 | 59_2 | 58.9 | 61_4 | 75.8 | 90_6 | 112.7 |
| 62_10 | 78.3 | 50_36 | 73.6 | 55_2 | 105.0 | 60_6 | 70.1 | 106_4 | 91.2 |
| 8_2 | 87.2 | 108_36 | 90.5 | 55_1 | 104.6 | 29_17 | 64.3 | 106_3 | 95.9 |
| 74_5 | 68.1 | 108_35 | 62.3 | 36_1 | 85.4 | 66_21 | 92.5 | 106_2 | 78.8 |
| 32_5 | 51.9 | 30_34 | 80.0 | 36_2 | 72.7 | 73_1 | 73.1 | 66_15 | 80.4 |
| 61_8 | 76.7 | 108_18 | 61.0 | 36_3 | 67.1 | 29_32 | 111.9 | 29_20 | 80.3 |
| 106_1 | 94.9 | 108_40 | 92.2 | 36_4 | 55.5 | 66_8 | 102.6 | 29_2 | 85.3 |
| 70_2 | 74.5 | 102_7 | 73.7 | 35_1 | 50.9 | 61_9 | 70.7 | 29_3 | 88.0 |
| 108_32 | 45.3 | 102_10 | 61.9 | 33_65 | 88.5 | 29_44 | 59.5 | 66_4 | 83.5 |
| 108_30 | 56.1 | 108_39 | 82.4 | 33_64 | 76.4 | 82_3 | 82.8 | 74_3 | 79.4 |
| 108_33 | 53.8 | 30_26 | 80.1 | 33_66 | 104.2 | 103_7 | 74.8 | 29_7 | 82.5 |
| 108_31 | 70.0 | 108_37 | 77.8 | 33_67 | 70.4 | 101_2 | 53.0 | 101_8 | 82.2 |
| 108_29 | 82.7 | 30_28 | 57.8 | 29_51 | 58.1 | 74_4 | 97.7 | 90_2 | 72.8 |
| 108_26 | 87.9 | 108_21 | 65.7 | 29_50 | 42.4 | 109_2 | 84.9 | 90_7 | 124.2 |
| 108_27 | 68.3 | 30_25 | 119.4 | 29_23 | 54.9 | 90_3 | 80.4 | 29_24 | 115.4 |
| 108_24 | 67.1 | 2_16 | 66.4 | 29_21 | 77.5 | 83_4 | 77.6 | 79_13 | 88.2 |
| 108_22 | 45.9 | 102_11 | 86.9 | 29_18 | 76.6 | 83_3 | 53.6 | 75_1 | 57.2 |
| 108_23 | 82.3 | 51_9 | 79.0 | 29_16 | 73.6 | 79_8 | 52.6 | 29_43 | 89.0 |
| 108_14 | 35.2 | 108_19 | 41.9 | 29_8 | 82.4 | 74_7 | 60.3 | 101_6 | 70.3 |
| 108_15 | 23.7 | 51_13 | 97.3 | 29_10 | 67.4 | 66_11 | 104.1 | 29_6 | 63.7 |
| 108_16 | 22.7 | 108_20 | 28.1 | 95_13 | 72.5 | 62_11 | 97.6 | 77_16 | 67.7 |
| 108_11 | 39.6 | 108_38 | 79.4 | 40_1 | 97.9 | 61_2 | 92.7 | 73_16 | 57.7 |
| 108_8 | 32.7 | 51_11 | 89.8 | 96_30 | 40.5 | 60_2 | 77.1 | 73_14 | 81.6 |
| 108_13 | 32.9 | 102_8 | 81.5 | 97_14 | 58.1 | 60_3 | 75.6 | 79_5 | 54.3 |
| 108_12 | 21.6 | 30_47 | 103.9 | 62_1 | 75.2 | 47_1 | 77.5 | 29_11 | 76.1 |
| 108_1 | 86.6 | 108_43 | 98.4 | 94_4 | 97.4 | 29_22 | 66.0 | 97_11 | 76.1 |
| 108_9 | 32.4 | 108_34 | 92.8 | 8_4 | 101.1 | 99_6 | 79.6 | 77_15 | 61.2 |
| 108_3 | 87.2 | 102_9 | 65.5 | 33_59 | 103.1 | 101_1 | 65.8 | 61_13 | 80.2 |
| 108_2 | 57.7 | 30_27 | 54.4 | 8_3 | 81.4 | 29_35 | 51.8 | 29_12 | 105.3 |
| 108_6 | 29.7 | 108_7 | 26.4 | 62_2 | 76.6 | 97_2 | 74.3 | 97_10 | 97.5 |
| 108_4 | 31.3 | 108_42 | 68.7 | 97_15 | 59.0 | 66_22 | 120.4 | 61_11 | 73.9 |
| 108_5 | 50.3 | 30_35 | 89.8 | 94_7 | 113.8 | 29_26 | 98.4 | 61_12 | 99.3 |
| 102_17 | 68.3 | 108_17 | 27.9 | 48_2 | 27.8 | 66_9 | 79.6 | 79_4 | 55.8 |
| 102_16 | 39.3 | 30_38 | 87.6 | 62_6 | 63.5 | 107_2 | 64.8 | 79_3 | 27.6 |
| 102_15 | 51.4 | 108_28 | 102.2 | 94_8 | 92.6 | 29_37 | 83.9 | 79_2 | 86.1 |
| 102_14 | 60.9 | 30_37 | 74.5 | 62_4 | 88.6 | 29_27 | 71.8 | 97_7 | 47.0 |
| 102_18 | 53.8 | 106_7 | 28.3 | 95_16 | 74.6 | 82_6 | 70.4 | 58_2 | 73.8 |
| 102_6 | 63.1 | 106_10 | 85.3 | 96_17 | 46.3 | 61_7 | 73.1 | 101_4 | 72.7 |
| 102_5 | 52.0 | 106_9 | 67.6 | 95_3 | 78.6 | 29_47 | 43.3 | 29_13 | 88.3 |
| 102_1 | 43.0 | 106_8 | 54.6 | 62_5 | 62.1 | 29_45 | 55.7 | 77_17 | 80.0 |
| 102_2 | 49.5 | 106_6 | 73.2 | 62_7 | 58.8 | 82_4 | 72.8 | 73_15 | 67.3 |
| 102_3 | 35.1 | 105_5 | 75.7 | 40_10 | 86.3 | 66_10 | 69.8 | 101_5 | 80.8 |
| 102_4 | 46.5 | 105_3 | 65.9 | 48_7 | 18.5 | 103_16 | 66.6 | 97_8 | 81.6 |
| 52_18 | 65.6 | 105_2 | 106.0 | 40_2 | 109.7 | 77_2 | 45.7 | 18_1 | 84.8 |
| 52_16 | 65.3 | 105_8 | 127.7 | 40_3 | 91.6 | 32_4 | 85.9 | 101_7 | 87.7 |
| 52_17 | 50.2 | 105_7 | 94.7 | 97_12 | 77.7 | 101_10 | 71.2 | 29_14 | 63.9 |
| 52_14 | 41.5 | 105_1 | 105.5 | 8_1 | 79.9 | 103_14 | 61.3 | 29_15 | 80.5 |
| 51_79 | 71.6 | 105_6 | 77.2 | 95_1 | 81.0 | 74_2 | 89.2 | 77_13 | 75.9 |
| 51_75 | 68.4 | 105_4 | 95.3 | 94_11 | 91.6 | 110_2 | 100.0 | 64_2 | 92.3 |
| 51_78 | 65.2 | 103_22 | 34.9 | 40_4 | 83.7 | 90_5 | 80.8 | 107_3 | 113.8 |
| 51_77 | 84.6 | 103_23 | 31.2 | 40_11 | 74.1 | 83_5 | 73.8 | 73_3 | 101.6 |
| 51_76 | 74.8 | 103_8 | 63.9 | 40_12 | 82.2 | 66_16 | 93.2 | 103_2 | 49.4 |
| 51_49 | 83.4 | 103_9 | 27.4 | 94_9 | 83.0 | 62_9 | 117.9 | 107_5 | 75.2 |
| 51_46 | 64.1 | 103_12 | 52.7 | 95_10 | 58.3 | 61_3 | 85.9 | 70_3 | 108.2 |
| 51_17 | 75.1 | 103_13 | 31.9 | 95_2 | 67.9 | 60_7 | 72.2 | 64_3 | 70.4 |
| 51_21 | 97.6 | 103_10 | 59.9 | 77_7 | 57.5 | 99_4 | 78.0 | 73_2 | 75.7 |
| 51_20 | 126.5 | 101_12 | 69.6 | 95_11 | 47.3 | 29_28 | 132.7 | 73_12 | 50.8 |
| 51_19 | 79.0 | 101_14 | 78.9 | 94_3 | 94.5 | 107_1 | 93.4 | 95_6 | 76.8 |
| 51_18 | 113.3 | 101_11 | 70.6 | 40_5 | 87.4 | 29_29 | 77.4 | 73_13 | 53.0 |
| 51_15 | 57.4 | 101_13 | 83.4 | 94_10 | 85.2 | 82_7 | 88.1 | 31_1 | 77.4 |
| 51_16 | 56.0 | 99_8 | 74.0 | 48_6 | 77.4 | 29_49 | 73.2 | 103_3 | 36.2 |
| 50_45 | 95.0 | 99_7 | 64.1 | 33_62 | 76.1 | 32_3 | 138.9 | 77_12 | 70.2 |
| 50_44 | 99.2 | 99_9 | 80.8 | 96_18 | 59.2 | 66_6 | 94.4 | 64_6 | 92.0 |
| 50_43 | 74.4 | 97_5 | 87.0 | 95_14 | 62.2 | 77_5 | 68.4 | 73_8 | 44.6 |
| 50_42 | 101.3 | 97_6 | 78.2 | 48_4 | 50.8 | 32_2 | 77.3 | 66_12 | 71.7 |
| 50_41 | 82.9 | 97_3 | 78.1 | 33_58 | 96.8 | 103_17 | 85.4 | 95_7 | 76.8 |
| 42_33 | 74.2 | 96_32 | 90.9 | 33_61 | 98.7 | 110_1 | 96.8 | 64_7 | 83.6 |
| 42_31 | 83.3 | 96_36 | 45.2 | 95_5 | 56.4 | 109_3 | 67.5 | 73_10 | 44.7 |
| 42_30 | 96.6 | 96_34 | 62.9 | 94_2 | 88.1 | 90_1 | 74.5 | 29_5 | 91.1 |
| 42_29 | 60.1 | 96_33 | 67.5 | 48_3 | 28.2 | 83_2 | 96.3 | 103_4 | 45.9 |
| 42_3 | 114.5 | 96_35 | 60.9 | 77_8 | 61.5 | 83_1 | 88.8 | 64_8 | 75.1 |
| 42_4 | 128.3 | 96_29 | 68.5 | 77_9 | 70.3 | 79_6 | 60.7 | 95_8 | 72.8 |
| 42_1 | 67 | 96_28 | 77.8 | 94_6 | 75.6 | 74_6 | 104 | 73_6 | 71.1 |
| 42_2 | 82.6 | 96_26 | 75 | 94_5 | 73.6 | 66_13 | 80.5 | 107_4 | 85.3 |
| 30_45 | 97.1 | 96_25 | 71.4 | 77_11 | 74.7 | 62_12 | 84.1 | 66_14 | 78.7 |
| 30_44 | 68.1 | 96_27 | 68 | 95_4 | 53.2 | 61_5 | 86.4 | 103_5 | 83.8 |
| 30_43 | 71.7 | 96_24 | 67.3 | 94_1 | 104.1 | 60_1 | 97.5 | 95_9 | 89.4 |
| 30_41 | 78.9 | 96_22 | 92.2 | 96_31 | 69.5 | 29_19 | 91.6 | 95_12 | 69.8 |
| 30_40 | 84.8 | 96_21 | 74 | 33_63 | 90.9 | 99_2 | 67.8 | 70_1 | 80.4 |
| 30_39 | 108.4 | 96_20 | 88.7 | 33_60 | 152.5 | 101_3 | 82.1 | 103_6 | 98 |
| 30_42 | 112.6 | 96_16 | 74.8 | 97_9 | 88.1 | 29_30 | 72.9 | 77_10 | 71.5 |
| 19_5 | 44 | 96_15 | 78.5 | 62_8 | 57.3 | 97_1 | 75.1 | 73_5 | 45.7 |
| 19_1 | 76.7 | 96_14 | 70.9 | 48_5 | 53.3 | 66_20 | 85.5 | 77_14 | 74.5 |
| 19_2 | 66.9 | 96_9 | 35.6 | 96_19 | 41.6 | 73_7 | 69.9 | | |
| 19_3 | 57.6 | 96_10 | 43.4 | 95_17 | 75.2 | 29_31 | 94.6 | | |

### Example 3: Determination of EC50 Values of GAL3ST1 Targeting ASOs in Caco2 and SK-N-AS Cells

To validate the hits identified in single concentration screens (Example 1 and 2) and to rank compounds on their potency a concentration response experiments were subsequently carried out for compounds showing high level of knock down.

Using the same method as describe in example 2 but incubating the cells with different concentrations of ASO (0.01; 0.0316; 0.1; 0.316; 1.0; 3.16; 10; 31.6 μM) allowed the generation of concentration response curves and EC50 values.

Concentration response curves were generated using the GraphPad prism software version9 using the “log(inhibitor) vs. response—Variable slope (four parameters)” fit with bottom constrained to >0 and top=100. EC50 values are shown in Table 7.

To confirm the hits identified in single concentration screens (Example 1 and 2) and to determine the potency of the compounds, concentration response experiments were performed for compounds showing high levels of knockdown. The cells were incubated with varying concentrations of ASOs (0.01, 0.0316, 0.1, 0.316, 1.0, 3.16, 10, and 31.6 μM), and concentration response curves and EC₅₀ values were generated using the same method as described in Example 2.

The concentration response curves were generated using GraphPad Prism software version 9 with the “log(inhibitor) vs. response—Variable slope (four parameters)” fit, with the bottom constrained to >0 and top set to 100. The EC₅₀ values are presented in Table 7.

TABLE 7
EC50 level of selected compounds

| | | Caco2 cells | Caco2 cells |
|---|---|---|---|
| | SK-N-AS | EC50 [M] | EC50 [M] |
| ASO ID | EC50 [M] | Experiment1 | Experiment2 |
| 1_41 | 4.34E−07 | | |
| 1_60 | 4.05E−07 | | |
| 1_103 | 7.12E−07 | | |
| 1_131 | 4.70E−07 | | |
| 1_129 | | 1.11E−06 | |
| 1_116 | 6.07E−07 | | |
| 1_164 | 8.40E−07 | | |
| 1_163 | 3.82E−07 | | |
| 1_161 | 3.71E−07 | | |
| 1_193 | 1.72E−07 | | |
| 1_187 | 9.99E−07 | | |
| 1_169 | | 1.39E−06 | |
| 4_48 | 4.39E−06 | | |
| 11_1 | 5.69E−07 | | |
| 11_2 | 4.40E−07 | | |
| 11_5 | 6.10E−07 | | |
| 11_14 | 5.53E−07 | | |
| 16_33 | 3.53E−07 | | |
| 16_17 | 3.51E−07 | | |
| 16_37 | | 8.76E−07 | |
| 16_10 | | 7.03E−06 | |
| 16_42 | 3.77E−07 | | |
| 19_23 | 3.72E−07 | | |
| 25_48 | 2.25E−07 | 1.48E−06 | |
| 25_49 | 3.55E−07 | 1.26E−06 | |
| 25_64 | | 1.50E−06 | |
| 25_57 | | 1.36E−06 | |
| 25_59 | | 9.70E−07 | |
| 25_61 | | 7.08E−07 | |
| 25_47 | | 1.01E−06 | |
| 25_46 | | 1.23E−06 | |
| 25_31 | | 1.30E−06 | |
| 25_32 | | 1.07E−06 | |
| 25_40 | | 1.23E−06 | |
| 25_65 | | 2.14E−06 | |
| 25_66 | | 1.23E−06 | |
| 25_90 | | 1.25E−06 | |
| 25_85 | | 7.75E−07 | 1.37E−06 |
| 25_26 | | 7.15E−07 | |
| 25_87 | | 1.40E−06 | |
| 25_88 | | 1.50E−06 | |
| 25_9 | | 1.36E−06 | |
| 25_11 | | 8.72E−07 | |
| 25_24 | | 7.43E−07 | |
| 25_13 | | 1.56E−06 | |
| 25_16 | | 1.31E−06 | |
| 25_12 | | 1.84E−06 | |
| 25_22 | | 9.26E−07 | |
| 26_12 | 1.41E−06 | | |
| 26_13 | 1.81E−06 | | |
| 41_62 | 5.02E−07 | | |
| 44_94 | 2.76E−07 | | |
| 49_3 | 4.39E−07 | | |
| 49_2 | 3.38E−07 | | |
| 49_1 | 5.07E−07 | | |
| 51_6 | 2.34E−07 | | |
| 52_3 | 8.69E−07 | | |
| 52_4 | 5.77E−07 | | |
| 52_10 | 1.00E−06 | | |
| 108_15 | | | 8.83E−07 |
| 108_16 | | | 9.91E−07 |
| 108_12 | | | 2.30E−06 |
| 108_6 | | | 1.31E−06 |
| 102_4 | | | 2.56E−06 |
| 108_20 | | | 1.67E−06 |
| 108_7 | | | 1.02E−06 |
| 108_17 | | | 8.34E−07 |
| 96_21 | | | 2.95E−05 |
| 96_12 | | | 2.04E−06 |
| 48_2 | | | 2.22E−06 |
| 48_7 | | | 2.57E−06 |
| 48_3 | | | 1.19E−06 |
| 79_3 | | | 1.54E−06 |

In conclusion, the concentration response experiments confirmed the hits identified in the single concentration screens of examples 1 and 2, and demonstrated that highly potent compounds were identified.

### Example 4: Overview on Identified Target Regions within the GA3ST1 Pre-mRNA Sequence (SEQ ID NO: 1) which Allow for Efficiently Downregulating GAL3ST1

Target sequences that were found in the studies underlying the present invention are shown in Table.

TABLE B1
Target sequences

| | Prem | Prem | | | |
|---|---|---|---|---|---|
| | RNA | RNA | | | |
| | Start | End | | | |
| | (in | (in | | | |
| | SEQ | SEQ | | | |
| | ID | ID | | | SEQ |
| Chr | NO: | NO: | | Target | ID |
| End | 633) | 633) | Pre-mRNA Sequence (5′ to 3′) | ID | NO |
| 30554670 | 19995 | 20031 | CAGAAGTAAAGGAATTTGATGTTGTGTTTT | 1 | 522 |
| | | | TGTTAA | | |
| 30554714 | 19951 | 19974 | GATAAAGAATGTTCTATTTCTGC | 2 | 523 |
| 30554762 | 19903 | 19922 | AGTATTTAACTAATTATAC | 3 | 524 |
| 30554991 | 19674 | 19711 | AAGCTCTGGAAGTTCATTCGCGATTTCCTG | 4 | 525 |
| | | | CGGTGGT | | |
| 30555088 | 19577 | 19592 | GAAGAGCATCGGGCA | 5 | 526 |
| 30555415 | 19250 | 19265 | GGAGGACGTGCTCTA | 6 | 527 |
| 30556362 | 18303 | 18320 | GTGGCCATGAATGAGAG | 7 | 528 |
| 30556390 | 18275 | 18292 | GATACTGATTCATGGTA | 8 | 529 |
| 30556431 | 18234 | 18256 | ATTTTTGTCCTATAAAATGGCA | 9 | 530 |
| 30556463 | 18202 | 18221 | GAGGAAACTGCAAATTTCT | 10 | 531 |
| 30556702 | 17963 | 17992 | GAGCATGAGAATCAGGTAACACTGTCTGT | 11 | 532 |
| 30557063 | 17602 | 17630 | GTGAAGTGGGGATAAAAACAGAGCCTTT | 12 | 533 |
| 30557130 | 17535 | 17551 | GTGCCAGACAGAAATT | 13 | 534 |
| 30557241 | 17424 | 17440 | GATGATGGGTGTAGGT | 14 | 535 |
| 30557674 | 16991 | 17022 | GGTTCATTTTAATTTTTAAATTTTAACCTT | 15 | 536 |
| | | | C | | |
| 30557723 | 16942 | 16976 | GCTTAAAAATTAAAAGGAGTGGAAAGTTTG | 16 | 537 |
| | | | GGGC | | |
| 30557839 | 16826 | 16855 | AAAATACAAGAAAAAGAAACAGAAGAAAA | 17 | 538 |
| 30557895 | 16770 | 16789 | GATCTGGGAGAATTTGAAG | 18 | 539 |
| 30557950 | 16715 | 16739 | AAAAAAGGAAATAATTTCATTATG | 19 | 540 |
| 30558265 | 16400 | 16439 | CGCCTGGTGAATTTTTTTAAATTTTGTTTT | 20 | 541 |
| | | | GTAGAGATA | | |
| 30558440 | 16225 | 16244 | GGGAGAGGGTCTTCTTTTC | 21 | 542 |
| 30558540 | 16125 | 16152 | GTGCCTTCTGAAAGGTGAAGGATTCAG | 22 | 543 |
| 30558818 | 15847 | 15864 | GTAAAAAATTGTATTCA | 23 | 544 |
| 30558880 | 15785 | 15804 | CAGCCTTGGAATAATTTTT | 24 | 545 |
| 30559229 | 15436 | 15454 | AAAATAAGGAAATAACAA | 25 | 546 |
| 30559590 | 15075 | 15095 | GGGCATCTCAAAAATAATAA | 26 | 547 |
| 30559692 | 14973 | 14990 | AAATAACAACAGGCTGG | 27 | 548 |
| 30559727 | 14938 | 14953 | GATAGGGTTGCCACG | 28 | 549 |
| 30559881 | 14784 | 14819 | GAAGTGGAAAGTTCGGGACAAGAGTAGAAA | 29 | 550 |
| | | | GTTTG | | |
| 30559958 | 14707 | 14737 | CATGCATGTAGTTATTAGCACATGTGACTT | 30 | 551 |
| 30559976 | 14689 | 14706 | GGGACAGTGGTGATAAT | 31 | 552 |
| 30560019 | 14646 | 14664 | AGGGCATAGTGTGTGTGA | 32 | 553 |
| 30560229 | 14436 | 14494 | AAGCTTTCTGATTTGTTACTTAAATGTGTT | 33 | 554 |
| | | | AAGTAACAAATTAACTAACACATTTGTT | | |
| 30560247 | 14418 | 14435 | GGGTTTATTCCGCTTTG | 34 | 555 |
| 30560319 | 14346 | 14363 | AGTGCTGGATTGAAACA | 35 | 556 |
| 30560440 | 14225 | 14242 | TATGTGCTGGTTGCTTG | 36 | 557 |
| 30560546 | 14119 | 14143 | GAGGCAGGTTTGTGCTTTGTGGGC | 37 | 558 |
| 30560598 | 14067 | 14083 | GTTTTCTGCTGGGTCG | 38 | 559 |
| 30560635 | 14030 | 14050 | GAGTTTTGGACTATGATCTG | 39 | 560 |
| 30560660 | 14005 | 14025 | TTTAAGTCAGAGAAACTGAG | 40 | 561 |
| 30560960 | 13705 | 13737 | AAACAAACAAAAGAGCAAATGGGAGAATGA | 41 | 562 |
| | | | AA | | |
| 30561124 | 13541 | 13565 | TAAAATGCAAAAATTACTTGAGTG | 42 | 563 |
| 30561267 | 13398 | 13418 | AAGCTTTGAGAAATAGCAAA | 43 | 564 |
| 30561346 | 13319 | 13346 | GGGCTCAAAAGTAAAACTAGGAGGGAT | 44 | 565 |
| 30561414 | 13251 | 13280 | GTTGCTGAGTAGGGTCAGTTAGATGCTCT | 45 | 566 |
| 30562081 | 12584 | 12605 | AAGATCCTGTCTTTTTAAAAA | 46 | 567 |
| 30562332 | 12333 | 12351 | ATGGGTGGTATGCACTTG | 47 | 568 |
| 30562502 | 12163 | 12179 | TGGGTACGTGCATTAT | 48 | 569 |
| 30562678 | 11987 | 12006 | CATGGGAAGTCATTCATCA | 49 | 570 |
| 30562771 | 11894 | 11923 | GTGTCTCAGTGAGGTGTTGTCTGCCTGGA | 50 | 571 |
| 30563182 | 11483 | 11509 | TGGATTTAGGGACATTTCAGATTTCA | 51 | 572 |
| 30563214 | 11451 | 11475 | TTTGTGGTGTCATGTAGGCATTCA | 52 | 573 |
| 30563304 | 11361 | 11380 | CAAGGTAGTTTTATTTTTC | 53 | 574 |
| 30563330 | 11335 | 11353 | ATGTATGTTTCTTATACA | 54 | 575 |
| 30563948 | 10717 | 10737 | TTGATTTTATTGTTTAAGCT | 55 | 576 |
| 30564066 | 10599 | 10616 | CGGATTTCAGATTTTTC | 56 | 577 |
| 30564161 | 10504 | 10523 | GGAATGCAGAGGATCTGCA | 57 | 578 |
| 30564574 | 10091 | 10111 | GTAATCTAGAAGGGCAGGAA | 58 | 579 |
| 30564708 | 9957 | 9974 | TTTGTGCATGATGTGGG | 59 | 580 |
| 30564759 | 9906 | 9924 | TGGTGGTGATGCTCTAAG | 60 | 581 |
| 30564793 | 9872 | 9894 | GTTACAGGGAAAGTAGGATACC | 61 | 582 |
| 30564827 | 9838 | 9858 | GAGGTGAGTGTGTCTCTTTT | 62 | 583 |
| 30564877 | 9788 | 9803 | GTTGGACGTGGCTCA | 63 | 584 |
| 30564929 | 9736 | 9754 | GGGTGTCATTGGAGCATT | 64 | 585 |
| 30564988 | 9677 | 9695 | AAGCAAACTCATGAATAT | 65 | 586 |
| 30565117 | 9548 | 9568 | TTGGATGAAAGAGCTGCTTG | 66 | 587 |
| 30565287 | 9378 | 9401 | CTGTGAGACTTAAATGAAATGCA | 67 | 588 |
| 30565516 | 9149 | 9167 | CAGGCTGAGTTAGTTGTT | 68 | 589 |
| 30565595 | 9070 | 9102 | ATTTAGAGCTCAGGAAGTGAAATCTTGCCA | 69 | 590 |
| | | | GA | | |
| 30565668 | 8997 | 9013 | AGAGCTGGTCGAGTGT | 70 | 591 |
| 30565788 | 8877 | 8893 | AGATTCAAGTGCCTGA | 71 | 592 |
| 30565959 | 8706 | 8733 | ATAATGATCATTATTAATTGATGGTGG | 72 | 593 |
| 30565989 | 8676 | 8704 | GTGCTTAGTGTGGGATGAATGACTGTCA | 73 | 594 |
| 30566083 | 8582 | 8606 | AGACTGGTGAGGCTTTGAGGGAAA | 74 | 595 |
| 30566159 | 8506 | 8522 | TAGAGAGAAGGCGACT | 75 | 596 |
| 30567138 | 7527 | 7546 | ATCATTGAAAAAGTTTTTG | 76 | 597 |
| 30567241 | 7424 | 7445 | AGAAATCCTGTGATGGTGATG | 77 | 598 |
| 30567293 | 7372 | 7395 | GTCAAAAAAACAAAAACAGTTTC | 78 | 599 |
| 30567625 | 7040 | 7070 | TGGGAAAAAAGCAAGTTAACAAAATATGTC | 79 | 600 |
| 30568011 | 6654 | 6675 | GTGGGATGGATCAATTTAAAA | 80 | 601 |
| 30568182 | 6483 | 6499 | GGATCAGGTGTTTTCT | 81 | 602 |
| 30568282 | 6383 | 6412 | GGGACTGTCTTTGGAAATTGGAGACCACA | 82 | 603 |
| 30568439 | 6226 | 6244 | GTAAATGGGAAAGGAGAC | 83 | 604 |
| 30568605 | 6060 | 6076 | AGTTAATGCTAGCTGC | 84 | 605 |
| 30570044 | 4621 | 4636 | GTGGTACGTTCATAC | 85 | 606 |
| 30570122 | 4543 | 4568 | GTTGGTGAATGGCTTTTGTTTCTTG | 86 | 607 |
| 30570157 | 4508 | 4524 | ATGTGCATATGAGCAA | 87 | 608 |
| 30570318 | 4347 | 4363 | GTAATAGCTGCTGTCA | 88 | 609 |
| 30570401 | 4264 | 4294 | ATCTCAGATTGATTGTTAGATTATCTTTGA | 89 | 610 |
| 30570460 | 4205 | 4235 | GGTAAAGGCTCTGTGAGCACTGGAGGAAAT | 90 | 611 |
| 30570819 | 3846 | 3869 | GGCTTAATATTAGGCAACTCTTT | 91 | 612 |
| 30570853 | 3812 | 3832 | ATGAATAAGTTTATTCCATA | 92 | 613 |
| 30570882 | 3783 | 3798 | ACATGTACGAATGTA | 93 | 614 |
| 30570917 | 3748 | 3766 | AATTTATAACTGGGTGTC | 94 | 615 |
| 30570963 | 3702 | 3723 | AAAATATGTTCCAGAAAGTGA | 95 | 616 |
| 30571079 | 3586 | 3619 | AGATTTTCAAAGTGGAAATATCTTTATTTA | 96 | 617 |
| | | | TCT | | |
| 30571315 | 3350 | 3370 | AGATTGAGGAAGCTTCTAGG | 97 | 618 |
| 30571422 | 3243 | 3264 | GACATTTTGGAAGTGGTCCTC | 98 | 619 |
| 30571571 | 3094 | 3113 | TTTGAAACAGCAGAGAGAG | 99 | 620 |
| 30571917 | 2748 | 2764 | GAGTGTCTGTCAAGGT | 100 | 621 |
| 30572125 | 2540 | 2559 | TTGGTGAAGCTGTGACTTG | 101 | 622 |
| 30572158 | 2507 | 2532 | ATTTTTGGAGCTAATGACAAAGTGA | 102 | 623 |
| 30572234 | 2431 | 2475 | AAATATTTCAGGCATGTTGTAAGGTACAGA | 103 | 624 |
| | | | GACTGATATTATCA | | |
| 30572279 | 2386 | 2414 | ATTGTTACATAGTTTTAGGCAAATAAGC | 104 | 625 |
| 30572454 | 2211 | 2230 | ATTTTTTAGGAAACCAAGG | 105 | 626 |
| 30572522 | 2143 | 2168 | TAACGCTGAGTGTGTGAAGGGCTCT | 106 | 627 |
| 30572802 | 1863 | 1882 | GTGAGGAATATGGATGAGC | 107 | 628 |
| 30572900 | 1765 | 1812 | GTGAAAGAGGCAATGATTAACCTGAGAGAA | 108 | 629 |
| | | | TGATGCACAAATGGAAT | | |
| 30572927 | 1738 | 1755 | GTTCTGAGGGACTGGGT | 109 | 630 |
| 30574025 | 640 | 665 | GGAGACCATAGTAAGGGATGGATCC | 110 | 631 |
| 30574137 | 528 | 546 | AGTGCGTTAATGATTAAT | 111 | 632 |

TABLE B2
Targeting sequences as shown in Table B2 allowed
for a very efficient down-regulation of the
target gene.

| | Geno | Geno | Prem | Prem | | | SEQ |
|---|---|---|---|---|---|---|---|
| | mic | mic | RNA | RNA | | Target | ID |
| Chr | Start | End | Start | End | Pre-mRNA Sequence (5′ to 3′) | ID | NO |
| 22 | 30554635 | 30554670 | 19995 | 20031 | CAGAAGTAAAGGAATTTGATGTTGTGTTT | 1 | 522 |
| | | | | | TTGTTAA | | |
| 22 | 30554692 | 30554714 | 19951 | 19974 | GATAAAGAATGTTCTATTTCTGC | 2 | 523 |
| 22 | 30554955 | 30554991 | 19674 | 19711 | AAGCTCTGGAAGTTCATTCGCGATTTCCTG | 4 | 525 |
| | | | | | CGGTGGT | | |
| 22 | 30556674 | 30556702 | 17963 | 17992 | GAGCATGAGAATCAGGTAACACTGTCTGT | 11 | 532 |
| 22 | 30557690 | 30557723 | 16942 | 16976 | GCTTAAAAATTAAAAGGAGTGGAAAGTTT | 16 | 537 |
| | | | | | GGGGC | | |
| 22 | 30557927 | 30557950 | 16715 | 16739 | AAAAAAGGAAATAATTTCATTATG | 19 | 540 |
| 22 | 30559212 | 30559229 | 15436 | 15454 | AAAATAAGGAAATAACAA | 25 | 546 |
| 22 | 30559571 | 30559590 | 15075 | 15095 | GGGCATCTCAAAAATAATAA | 26 | 547 |
| 22 | 30559847 | 30559881 | 14784 | 14819 | GAAGTGGAAAGTTCGGGACAAGAGTAGA | 29 | 550 |
| | | | | | AAGTTTG | | |
| 22 | 30559929 | 30559958 | 14707 | 14737 | CATGCATGTAGTTATTAGCACATGTGACTT | 30 | 551 |
| 22 | 30560929 | 30560960 | 13705 | 13737 | AAACAAACAAAAGAGCAAATGGGAGAAT | 41 | 562 |
| | | | | | GAAA | | |
| 22 | 30561101 | 30561124 | 13541 | 13565 | TAAAATGCAAAAATTACTTGAGTG | 42 | 563 |
| 22 | 30561320 | 30561346 | 13319 | 13346 | GGGCTCAAAAGTAAAACTAGGAGGGAT | 44 | 565 |
| 22 | 30562487 | 30562502 | 12163 | 12179 | TGGGTACGTGCATTAT | 48 | 569 |
| 22 | 30562660 | 30562678 | 11987 | 12006 | CATGGGAAGTCATTCATCA | 49 | 570 |
| 22 | 30562743 | 30562771 | 11894 | 11923 | GTGTCTCAGTGAGGTGTTGTCTGCCTGGA | 50 | 571 |
| 22 | 30563157 | 30563182 | 11483 | 11509 | TGGATTTAGGGACATTTCAGATTTCA | 51 | 572 |
| 22 | 30563191 | 30563214 | 11451 | 11475 | TTTGTGGTGTCATGTAGGCATTCA | 52 | 573 |
| 22 | 30565962 | 30565989 | 8676 | 8704 | GTGCTTAGTGTGGGATGAATGACTGTCA | 73 | 594 |
| 22 | 30567221 | 30567241 | 7424 | 7445 | AGAAATCCTGTGATGGTGATG | 77 | 598 |
| 22 | 30567596 | 30567625 | 7040 | 7070 | TGGGAAAAAAGCAAGTTAACAAAATATGT | 79 | 600 |
| | | | | | C | | |
| 22 | 30570943 | 30570963 | 3702 | 3723 | AAAATATGTTCCAGAAAGTGA | 95 | 616 |
| 22 | 30571047 | 30571079 | 3586 | 3619 | AGATTTTCAAAGTGGAAATATCTTTATTTA | 96 | 617 |
| | | | | | TCT | | |
| 22 | 30571296 | 30571315 | 3350 | 3370 | AGATTGAGGAAGCTTCTAGG | 97 | 618 |
| 22 | 30572134 | 30572158 | 2507 | 2532 | ATTTTTGGAGCTAATGACAAAGTGA | 102 | 623 |
| 22 | 30572191 | 30572234 | 2431 | 2475 | AAATATTTCAGGCATGTTGTAAGGTACAG | 103 | 624 |
| | | | | | AGACTGATATTATCA | | |
| 22 | 30572498 | 30572522 | 2143 | 2168 | TAACGCTGAGTGTGTGAAGGGCTCT | 106 | 627 |
| 22 | 30572854 | 30572900 | 1765 | 1812 | GTGAAAGAGGCAATGATTAACCTGAGAG | 108 | 629 |
| | | | | | AATGATGCACAAATGGAAT | | |

TABLE B3
Targeting sequences as shown in Table B3 allowed for the most
efficient down-regulation of the target gene.

| | | | Prem | Prem | | | SEQ |
|---|---|---|---|---|---|---|---|
| | Genomic | Genomic | RNA | RNA | | Target | ID |
| Chr | Start | End | Start | End | Pre-mRNA Sequence (5′ to 3′) | ID | NO |
| 22 | 30554635 | 30554670 | 19995 | 20031 | CAGAAGTAAAGGAATTTGATGTTGTGTTT | 1 | 522 |
| | | | | | TTGTTAA | | |
| 22 | 30556674 | 30556702 | 17963 | 17992 | GAGCATGAGAATCAGGTAACACTGTCTGT | 11 | 532 |
| 22 | 30557690 | 30557723 | 16942 | 16976 | GCTTAAAAATTAAAAGGAGTGGAAAGTTT | 16 | 537 |
| | | | | | GGGGC | | |
| 22 | 30557927 | 30557950 | 16715 | 16739 | AAAAAAGGAAATAATTTCATTATG | 19 | 540 |
| 22 | 30559212 | 30559229 | 15436 | 15454 | AAAATAAGGAAATAACAA | 25 | 546 |
| 22 | 30559571 | 30559590 | 15075 | 15095 | GGGCATCTCAAAAATAATAA | 26 | 547 |
| 22 | 30560929 | 30560960 | 13705 | 13737 | AAACAAACAAAAGAGCAAATGGGAGAAT | 41 | 562 |
| | | | | | GAAA | | |
| 22 | 30561320 | 30561346 | 13319 | 13346 | GGGCTCAAAAGTAAAACTAGGAGGGAT | 44 | 565 |
| 22 | 30562487 | 30562502 | 12163 | 12179 | TGGGTACGTGCATTAT | 48 | 569 |
| 22 | 30562660 | 30562678 | 11987 | 12006 | CATGGGAAGTCATTCATCA | 49 | 570 |
| 22 | 30563191 | 30563214 | 11451 | 11475 | TTTGTGGTGTCATGTAGGCATTCA | 52 | 573 |
| 22 | 30567596 | 30567625 | 7040 | 7070 | TGGGAAAAAAGCAAGTTAACAAAATATGT | 79 | 600 |
| | | | | | C | | |
| 22 | 30571047 | 30571079 | 3586 | 3619 | AGATTTTCAAAGTGGAAATATCTTTATTTA | 96 | 617 |
| | | | | | TCT | | |
| 22 | 30572191 | 30572234 | 2431 | 2475 | AAATATTTCAGGCATGTTGTAAGGTACAG | 103 | 624 |
| | | | | | AGACTGATATTATCA | | |
| 22 | 30572498 | 30572522 | 2143 | 2168 | TAACGCTGAGTGTGTGAAGGGCTCT | 106 | 627 |
| 22 | 30572854 | 30572900 | 1765 | 1812 | GTGAAAGAGGCAATGATTAACCTGAGAG | 108 | 629 |
| | | | | | AATGATGCACAAATGGAAT | | |

### Example 5. Testing In Vitro Efficacy of Additional Antisense Oligonucleotides Targeting GAL3ST1 in CACO2 Cells at Single Test Concentration

For the GAL3ST1 target regions identified in Example 1 to 4 above, further ASOs were designed with different sugar and back-bone modifications. An overview on the compounds is provided in Table C in FIG. 2. As for Table A1, the annotation for the individual nucleotides is provided in Table A2. The new ASOs were tested for activity in Caco2 cells at a single concentration of 10 μM. In addition, some compounds that were tested in Examples 1 to 4 were included in the tests. The protocol described in Example 2 was used. The results are shown in Table 8. The results for compounds that have been already tested in Examples 1 to 4 (such as the compound with ASO ID 1_41) are given in bold.

TABLE 8
Expression level of GAL3ST1 in relation to PBS
treated control cells (% UTC) Caco2 10 μM

| ASO ID | Caco2, 10 μM % UTC |
|---|---|
| 1_41 | 59.2 |
| 1_103 | 51.9 |
| 1_116 | 41.4 |
| 1_129 | 39.5 |
| 1_131 | 30.9 |
| 1_164 | 40.2 |
| 1_193 | 33.5 |
| 1_199 | 77.5 |
| 1_201 | 87.3 |
| 1_202 | 88.4 |
| 1_203 | 92.8 |
| 1_204 | 90.5 |
| 1_205 | 86.7 |
| 1_206 | 82.7 |
| 1_207 | 81.8 |
| 1_208 | 85.9 |
| 1_209 | 57.8 |
| 1_210 | 56.0 |
| 1_211 | 58.4 |
| 1_212 | 75.3 |
| 1_213 | 84.4 |
| 1_214 | 80.9 |
| 1_215 | 47.8 |
| 1_216 | 86.6 |
| 1_217 | 86.1 |
| 1_218 | 74.3 |
| 1_219 | 96.0 |
| 1_220 | 56.4 |
| 1_221 | 52.8 |
| 1_222 | 90.5 |
| 1_223 | 81.3 |
| 1_224 | 93.3 |
| 1_225 | 57.9 |
| 1_226 | 106.1 |
| 1_227 | 92.8 |
| 1_228 | 39.1 |
| 1_229 | 64.1 |
| 1_230 | 80.1 |
| 1_231 | 59.5 |
| 1_232 | 56.4 |
| 1_233 | 84.1 |
| 1_234 | 72.8 |
| 1_235 | 88.4 |
| 1_236 | 69.4 |
| 1_237 | 94.3 |
| 1_238 | 66.7 |
| 1_240 | 86.7 |
| 1_241 | 95.8 |
| 1_242 | 68.7 |
| 1_243 | 78.6 |
| 1_244 | 81.1 |
| 1_245 | 97.7 |
| 1_246 | 61.6 |
| 1_247 | 93.6 |
| 1_248 | 84.0 |
| 1_249 | 65.6 |
| 1_250 | 99.0 |
| 1_251 | 66.5 |
| 1_252 | 97.6 |
| 1_253 | 87.2 |
| 1_254 | 87.3 |
| 1_255 | 92.5 |
| 1_256 | 99.0 |
| 1_257 | 55.2 |
| 16_17 | 46.2 |
| 16_128 | 54.6 |
| 16_129 | 56.1 |
| 16_130 | 69.0 |
| 16_131 | 60.4 |
| 16_132 | 60.3 |
| 16_133 | 55.6 |
| 16_134 | 57.7 |
| 16_135 | 56.2 |
| 16_136 | 76.3 |
| 16_137 | 58.1 |
| 19_23 | 54.3 |
| 19_62 | 62.1 |
| 19_63 | 79.5 |
| 19_64 | 93.2 |
| 19_65 | 106.9 |
| 19_66 | 106.0 |
| 19_67 | 94.5 |
| 19_68 | 45.4 |
| 19_69 | 108.2 |
| 19_70 | 88.5 |
| 19_71 | 103.1 |
| 19_72 | 81.9 |
| 19_73 | 98.8 |
| 19_74 | 80.7 |
| 19_75 | 88.4 |
| 19_76 | 92.8 |
| 25_16 | 23.9 |
| 25_85 | 25.3 |
| 25_91 | 48.2 |
| 25_92 | 105.1 |
| 25_93 | 87.7 |
| 25_94 | 97.6 |
| 25_95 | 90.2 |
| 25_96 | 92.4 |
| 25_97 | 91.1 |
| 25_98 | 58.9 |
| 25_99 | 80.4 |
| 25_100 | 79.6 |
| 25_101 | 86.8 |
| 25_102 | 92.5 |
| 48_2 | 38.3 |
| 48_3 | 39.3 |
| 48_7 | 34.3 |
| 48_8 | 66.7 |
| 48_9 | 70.1 |
| 48_10 | 88.4 |
| 48_11 | 96.4 |
| 48_12 | 61.7 |
| 48_13 | 61.3 |
| 48_14 | 67.8 |
| 48_15 | 75.2 |
| 48_16 | 70.5 |
| 48_17 | 101.7 |
| 48_18 | 72.8 |
| 48_19 | 71.7 |
| 48_21 | 62.7 |
| 48_22 | 83.1 |
| 48_23 | 63.6 |
| 48_24 | 76.3 |
| 48_25 | 94.4 |
| 79_3 | 35.0 |
| 79_14 | 74.5 |
| 79_15 | 105.5 |
| 79_16 | 70.4 |
| 79_17 | 59.0 |
| 79_18 | 51.5 |
| 79_19 | 59.8 |
| 79_20 | 73.6 |
| 79_21 | 77.9 |
| 79_22 | 45.9 |
| 79_23 | 67.4 |
| 79_24 | 72.3 |
| 79_25 | 71.6 |
| 96_12 | 40.1 |
| 96_37 | 72.6 |
| 96_38 | 91.4 |
| 96_39 | 81.1 |
| 96_40 | 95.5 |
| 96_41 | 83.8 |
| 96_42 | 77.9 |
| 96_43 | 89.1 |
| 96_44 | 88.8 |
| 96_45 | 103.5 |
| 103_9 | 23.7 |
| 103_26 | 30.7 |
| 103_27 | 62.8 |
| 103_28 | 49.7 |
| 103_29 | 53.6 |
| 103_30 | 39.5 |
| 103_32 | 38.0 |
| 103_33 | 60.8 |
| 106_7 | 54.8 |
| 106_11 | 71.3 |
| 106_12 | 84.0 |
| 106_14 | 50.5 |
| 106_16 | 59.3 |
| 106_17 | 69.7 |
| 106_18 | 65.3 |
| 108_6 | 44.4 |
| 108_7 | 43.0 |
| 108_12 | 33.6 |
| 108_15 | 33.4 |
| 108_16 | 41.7 |
| 108_17 | 43.0 |
| 108_20 | 32.2 |
| 108_47 | 37.9 |
| 108_48 | 82.0 |
| 108_49 | 65.1 |
| 108_50 | 35.0 |
| 108_51 | 42.6 |
| 108_52 | 53.9 |
| 108_53 | 48.9 |
| 108_55 | 36.6 |
| 108_56 | 54.1 |
| 108_57 | 42.6 |
| 108_58 | 84.5 |
| 108_59 | 57.2 |
| 108_60 | 34.2 |
| 108_61 | 203.9 |
| 108_62 | 40.7 |
| 108_63 | 64.8 |
| 108_64 | 72.3 |
| 108_65 | 35.8 |
| 108_66 | 62.5 |
| 108_67 | 72.1 |
| 108_68 | 51.0 |
| 108_69 | 41.3 |
| 108_70 | 60.9 |
| 108_71 | 58.0 |
| 108_73 | 76.9 |
| 108_74 | 61.2 |
| 108_75 | 49.6 |
| 108_76 | 57.1 |
| 108_77 | 62.1 |
| 108_78 | 55.5 |
| 108_79 | 77.6 |
| 108_80 | 53.5 |
| 108_81 | 62.1 |
| 108_82 | 66.7 |
| 108_83 | 70.2 |
| 108_84 | 59.1 |
| 108_85 | 86.2 |

### Example 6: Determination of EC50 Values of GAL3ST1 Targeting ASOs in Caco2 Cells

To validate the hits identified in single concentration screens (Example 5) and to rank compounds on their potency a concentration response experiments were subsequently carried out for compounds showing high level of knock down.

Using the same method as describe in example 2 but incubating the cells with different concentrations of ASO (0.01; 0.0316; 0.1; 0.316; 1.0; 3.16; 10; 31.6 μM) allowed the generation of concentration response curves and EC50 values.

Concentration response curves were generated using the GraphPad prism software version9 using the “log(inhibitor) vs. response—Variable slope (four parameters)” fit with bottom constrained to >0 and top=100. EC50 values are shown in Table 9. The results for compounds that have been already tested in Examples 1 to 4 (such as the compound with ASO ID 1_41) are given in bold.

TABLE 9
EC50 values of selected compounds

| | Caco2 | Caco2 | Caco2 | Caco2 | Caco2 | Caco2 | Caco2 | Caco2 | |
|---|---|---|---|---|---|---|---|---|---|
| ASO | 0.01 μM | 0.032 μM | 0.1 μM | 0.316 μM | 1 μM | 3.16 μM | 10 μM | 31.6 μM | EC50 |
| ID | % UTC | % UTC | % UTC | % UTC | % UTC | % UTC | % UTC | % UTC | [M] |
| 1_41 | 102.6 | 93.7 | 93.4 | 83.4 | 85.3 | 77.3 | 61.4 | 56.1 | 1.8E−06 |
| 1_103 | 90.8 | 98.4 | 83.6 | 79.9 | 70.5 | 49.5 | 43.2 | 32.4 | 9.2E−07 |
| 1_116 | 111.5 | 89.0 | 79.7 | 81.6 | 61.9 | 54.5 | 43.0 | 44.3 | 4.6E−07 |
| 1_131 | 112.2 | 85.2 | 83.5 | 73.2 | 63.2 | 40.9 | 35.9 | 28.1 | 6.1E−07 |
| 1_164 | 102.0 | 92.6 | 87.4 | 82.4 | 66.1 | 45.4 | 24.0 | 18.1 | 1.5E−06 |
| 1_193 | 82.2 | 87.7 | 87.6 | 78.0 | 61.6 | 45.0 | 33.7 | 23.2 | 8.5E−07 |
| 1_210 | 99.9 | 100.5 | 115.3 | 89.4 | 83.7 | 74.7 | 55.8 | 41.7 | 5.0E−06 |
| 1_215 | 104.6 | 92.9 | 90.5 | 96.3 | 79.2 | 64.7 | 56.0 | 32.5 | 3.6E−06 |
| 1_220 | 109.9 | 86.9 | 73.7 | 87.6 | 86.6 | 60.4 | 52.7 | 39.6 | 1.9E−06 |
| 1_221 | 91.2 | 90.2 | 105.9 | 85.0 | 69.5 | 60.0 | 55.2 | 41.1 | 1.2E−06 |
| 1_228 | 89.2 | 88.7 | 91.5 | 68.8 | 73.4 | 54.3 | 43.5 | 34.6 | 6.7E−07 |
| 1_232 | 93.7 | 90.2 | 94.1 | 92.1 | 91.1 | 79.6 | 64.7 | 52.3 | 5.4E−06 |
| 1_238 | 84.2 | 108.1 | 95.7 | 92.0 | 75.0 | 84.0 | 61.4 | 49.9 | 3.3E−06 |
| 1_246 | 101.4 | 96.5 | 91.2 | 93.5 | 87.8 | 59.2 | 62.4 | 48.2 | 2.1E−06 |
| 1_249 | 92.2 | 100.1 | 99.2 | 88.7 | 90.1 | 78.7 | 59.1 | 58.6 | 3.2E−06 |
| 1_251 | 87.5 | 98.5 | 92.1 | 87.6 | 72.7 | 60.2 | 42.8 | 33.6 | 1.8E−06 |
| 1_257 | 94.9 | 109.2 | 104.3 | 95.3 | 77.4 | 63.5 | 45.0 | 38.9 | 2.8E−06 |
| 16_17 | 111.1 | 96.3 | 83.7 | 91.6 | 71.2 | 63.2 | 51.5 | 27.1 | 2.4E−06 |
| 16_128 | 102.7 | 102.1 | 107.1 | 102.8 | 93.7 | 77.7 | 55.1 | 40.6 | 9.5E−06 |
| 16_129 | 104.5 | 93.8 | 109.2 | 100.0 | 85.0 | 67.5 | 50.1 | 37.5 | 4.8E−06 |
| 16_133 | 106.3 | 95.1 | 97.4 | 104.2 | 87.9 | 99.4 | 88.4 | 87.2 | 3.8E−06 |
| 19_23 | 98.6 | 92.1 | 89.1 | 84.6 | 64.1 | 50.5 | 36.9 | 27.2 | 1.2E−06 |
| 19_62 | 95.0 | 90.5 | 93.9 | 91.2 | 71.5 | 56.5 | 40.3 | 24.9 | 2.3E−06 |
| 19_68 | 104.6 | 97.3 | 91.8 | 92.7 | 76.2 | 61.5 | 36.3 | 32.3 | 2.6E−06 |
| 25_16 | 102.3 | 85.7 | 93.1 | 83.6 | 58.7 | 32.0 | 17.1 | 18.6 | 1.1E−06 |
| 25_85 | 105.9 | 112.4 | 94.0 | 78.1 | 59.5 | 27.7 | 21.7 | 13.7 | 1.2E−06 |
| 25_91 | 110.4 | 104.7 | 95.9 | 96.5 | 58.9 | 44.2 | 34.7 | 23.9 | 1.5E−06 |
| 25_98 | 93.1 | 93.9 | 105.5 | 97.5 | 94.3 | 68.9 | 46.8 | 38.0 | 5.8E−06 |
| 48_2 | 100.5 | 104.2 | 99.9 | 77.1 | 59.7 | 45.8 | 28.8 | 20.6 | 1.2E−06 |
| 48_3 | 101.7 | 92.2 | 91.3 | 83.8 | 58.5 | 42.2 | 29.0 | 19.1 | 1.2E−06 |
| 48_7 | 104.2 | 100.2 | 95.5 | 79.4 | 67.8 | 48.2 | 22.5 | 20.8 | 1.7E−06 |
| 48_12 | 99.0 | 104.6 | 99.0 | 97.5 | 82.1 | 68.7 | 52.0 | 34.0 | 4.9E−06 |
| 48_13 | 102.0 | 90.2 | 101.8 | 89.9 | 89.0 | 65.5 | 47.0 | 30.3 | 5.1E−06 |
| 48_16 | 92.5 | 100.4 | 96.1 | 96.5 | 91.8 | 85.9 | 70.1 | 55.1 | 1.0E−05 |
| 48_21 | 98.5 | 92.1 | 94.9 | 91.4 | 73.4 | 60.5 | 52.6 | 31.2 | 2.2E−06 |
| 48_23 | 98.2 | 97.7 | 101.0 | 96.6 | 81.1 | 73.5 | 59.0 | 40.1 | 4.9E−06 |
| 79_3 | 79.7 | 92.1 | 80.8 | 80.9 | 56.5 | 44.6 | 30.6 | 21.2 | 7.8E−07 |
| 79_18 | 95.7 | 103.7 | 96.1 | 98.6 | 88.7 | 65.3 | 57.0 | 43.0 | 3.9E−06 |
| 79_22 | 105.9 | 105.2 | 82.1 | 80.9 | 64.3 | 51.8 | 34.8 | 24.7 | 1.2E−06 |
| 96_37 | 97.9 | 102.4 | 102.6 | 102.6 | 89.8 | 91.0 | 102.4 | 89.7 | 7.2E−07 |
| 103_9 | 100.2 | 97.3 | 87.5 | 79.5 | 67.7 | 43.2 | 20.3 | 15.3 | 1.6E−06 |
| 103_26 | 87.0 | 96.7 | 89.1 | 74.6 | 55.1 | 34.0 | 25.1 | 17.7 | 8.1E−07 |
| 103_30 | 91.4 | 95.4 | 97.4 | 89.6 | 68.7 | 37.9 | 27.0 | 18.8 | 1.8E−06 |
| 103_32 | 86.0 | 92.0 | 86.8 | 81.7 | 55.4 | 40.1 | 26.0 | 15.8 | 1.0E−06 |
| 106_7 | 93.5 | 98.6 | 101.8 | 96.3 | 90.2 | 69.8 | 61.3 | 51.6 | 3.6E−06 |
| 106_14 | 100.5 | 87.4 | 101.5 | 86.2 | 83.8 | 67.7 | 53.1 | 37.1 | 3.5E−06 |
| 106_16 | 79.2 | 102.5 | 98.4 | 102.9 | 81.3 | 79.4 | 53.6 | 35.8 | 8.1E−06 |
| 108_6 | 93.2 | 113.9 | 95.8 | 96.1 | 66.5 | 49.3 | 39.8 | 32.6 | 1.6E−06 |
| 108_7 | 101.9 | 103.9 | 95.1 | 90.9 | 56.9 | 33.9 | 30.6 | 24.2 | 1.2E−06 |
| 108_12 | 98.5 | 91.1 | 96.8 | 70.6 | 58.1 | 30.5 | 26.5 | 23.7 | 7.4E−07 |
| 108_15 | 104.8 | 118.4 | 112.5 | 84.3 | 56.0 | 40.3 | 32.2 | 26.3 | 1.3E−06 |
| 108_16 | 93.7 | 104.4 | 88.1 | 77.0 | 56.7 | 32.9 | 24.9 | 24.1 | 8.2E−07 |
| 108_17 | 107.2 | 103.6 | 93.7 | 75.3 | 61.4 | 41.6 | 31.8 | 29.1 | 8.8E−07 |
| 108_20 | 93.3 | 104.3 | 93.5 | 79.9 | 59.7 | 37.0 | 29.3 | 24.8 | 9.9E−07 |
| 108_47 | 98.1 | 103.5 | 87.2 | 87.1 | 59.4 | 42.6 | 30.4 | 25.9 | 1.1E−06 |
| 108_50 | 96.0 | 96.8 | 96.6 | 67.3 | 47.7 | 35.6 | 28.8 | 25.4 | 5.5E−07 |
| 108_51 | 96.0 | 91.3 | 95.6 | 90.9 | 63.1 | 44.7 | 31.2 | 27.7 | 1.4E−06 |
| 108_52 | 96.3 | 104.6 | 97.1 | 95.4 | 78.3 | 57.3 | 40.2 | 33.9 | 2.6E−06 |
| 108_53 | 97.6 | 92.9 | 93.5 | 92.9 | 81.1 | 53.9 | 39.1 | 26.8 | 2.9E−06 |
| 108_55 | 100.0 | 101.5 | 90.9 | 77.4 | 54.1 | 43.6 | 29.4 | 24.0 | 8.3E−07 |
| 108_56 | 100.5 | 104.1 | 102.1 | 89.6 | 74.9 | 50.8 | 39.3 | 29.8 | 2.1E−06 |
| 108_57 | 95.7 | 93.8 | 94.7 | 98.0 | 70.8 | 54.9 | 42.0 | 23.5 | 2.6E−06 |
| 108_59 | 89.5 | 95.1 | 89.2 | 86.2 | 79.6 | 52.4 | 42.1 | 30.4 | 2.0E−06 |
| 108_60 | 96.5 | 99.3 | 82.4 | 83.5 | 74.6 | 48.0 | 30.5 | 20.3 | 1.9E−06 |
| 108_62 | 106.6 | 95.9 | 97.4 | 88.6 | 70.6 | 52.6 | 33.4 | 32.0 | 1.7E−06 |
| 108_63 | 109.0 | 106.4 | 102.7 | 100.1 | 90.4 | 72.5 | 61.8 | 46.4 | 5.9E−06 |
| 108_65 | 98.7 | 108.2 | 91.5 | 85.6 | 71.2 | 53.5 | 39.5 | 26.2 | 1.9E−06 |
| 108_66 | 94.1 | 101.7 | 109.3 | 98.3 | 87.9 | 60.5 | 49.2 | 40.4 | 3.7E−06 |
| 108_68 | 98.8 | 104.4 | 107.3 | 88.1 | 64.9 | 64.0 | 39.1 | 31.8 | 2.0E−06 |
| 108_69 | 116.5 | 101.9 | 99.7 | 92.1 | 74.6 | 65.9 | 44.1 | 32.1 | 3.1E−06 |
| 108_70 | 99.6 | 103.9 | 101.3 | 95.5 | 87.4 | 73.6 | 59.1 | 44.5 | 5.2E−06 |
| 108_71 | 99.2 | 92.4 | 100.5 | 95.6 | 83.5 | 67.8 | 56.0 | 35.8 | 4.6E−06 |
| 108_74 | 111.0 | 87.9 | 91.6 | 101.9 | 85.9 | 69.9 | 59.8 | 50.2 | 3.2E−06 |
| 108_75 | 94.3 | 93.6 | 84.9 | 94.1 | 63.1 | 53.8 | 39.7 | 33.2 | 1.2E−06 |
| 108_76 | 91.6 | 93.1 | 104.6 | 100.6 | 80.9 | 70.1 | 54.3 | 43.9 | 3.7E−06 |
| 108_78 | 95.3 | 88.7 | 90.2 | 95.2 | 90.0 | 77.7 | 58.0 | 45.7 | 5.8E−06 |
| 108_80 | 100.8 | 104.1 | 97.1 | 84.6 | 75.1 | 56.1 | 35.6 | 32.7 | 2.0E−06 |
| 108_82 | 104.9 | 96.1 | 103.8 | 82.6 | 89.6 | 67.6 | 48.3 | 48.2 | 2.7E−06 |
| 108_84 | 96.1 | 86.1 | 97.6 | 97.1 | 90.0 | 59.1 | 45.4 | 39.1 | 3.4E−06 |
| 108_86 | 100.8 | 100.9 | 90.5 | 86.0 | 79.2 | 61.4 | 52.6 | 38.9 | 1.8E−06 |

In conclusion, the concentration response experiments confirmed the hits identified in the single concentration screens in the above example. In addition, the experiments demonstrated that highly potent compounds were identified.

### Example 7. Testing In Vitro Efficacy and Potency of Antisense Oligonucleotides Targeting GAL3ST1 in iPSC Derived Human Neurons at Multiple Test Concentrations

The activity of preferred ASOs were tested in iPSC derived human neurons to confirm activity and potency in a relevant cellular model.

The iPSC derived neurons were maintained as recommended by the supplier (Fuji film, 01279) and RNA were purified and analyzed by microarray as described in example 8.

TABLE 10
Effect on GAL3ST1 as evaluated by microarray
ASO knockdown expressed as % PBS.

ASO Concentration

| ASO ID | 0.2 μM | 1 μM | 5 μM | 25 μM |
|---|---|---|---|---|
| 19_23 | 98 | 58 | 57 | 41 |
| 1_129 | 69 | 64 | 55 | 68 |
| 25_16 | 78 | 52 | 36 | 34 |
| 1_41 | 72 | 58 | 46 | 47 |
| 1_103 | 97 | 70 | 49 | 48 |
| 1_193 | 73 | 73 | 52 | 45 |
| 1_116 | 82 | 60 | 60 | 45 |
| 16_17 | 73 | 48 | 45 | 39 |

Evaluation of ASO in vitro activity in Caco2 cells and iPSC derived neurons shows good activity for all ASOs leading to significant reduction of GAL3ST1 mRNA.

### Example 8: Off-Target Effects of Selected Gapmers In Vitro Evaluated by Microarrays

Modified oligonucleotide gapmers complementary to human GAL3ST1 pre-mRNA were designed and tested for their transcriptome-wide off-target effects in human neurons by unassisted uptake. The gapmers were tested at four different concentrations (0.2, 1, 5, and 25 μM) and compared to PBS-treated controls in a series of experiments under similar conditions. For each concentration typically three replicates were done.

Human glutamatergic-enriched cortical neurons derived from induced pluripotent stem cells (iCell GlutaNeurons; FUJIFILM Cellular Dynamics, Inc.) were plated at a density of 80000 cells per well. Media and supplements were according to manufacturer's specifications and half media change was performed on day 1 and 4 after plating the cells. Gapmers were added 4 days after plating and incubated with the cells for 4 days. After 4 days of incubation, total RNA was isolated from the cells and DNase-treated using RNeasy kit (QIAGEN) according to the manufacturer's instructions.

For microarray analysis, first strand cDNA was synthesized from total RNA with a combination of a Poly-dT and random primers containing a 5′-adaptor sequence. Next, a 3′-adaptor was added to the single stranded cDNA followed by low-cycle PCR amplification. The amplified cDNA was used as template for in vitro transcription to produce amplified amounts of complementary mRNA (cRNA). The cRNA was then used as input for a second round of cDNA synthesis, producing double stranded cDNA. After fragmentation, denaturation and end-labeling, the cDNA was hybridized to Human Clariom GO Screen 384-array plates, stained, and imaged on a GeneTitan Multi-Channel Instrument.

Probe intensities from the imager were summarized and corrected for technical variation between arrays by Robust Multichip Average (RMA) normalization. In total 19654 probe sets corresponding to unique genes were judged to be expressed in the neurons and analyzed further.

For selected genes, concentration-response curves (CRCs) of RNA levels (as percent of PBS) after treatment with gapmer at four different concentrations were analyzed by nonlinear least-squares fitting of a two-parameter logistic function (fitting parameters were slope and half-maximal effective concentration, EC50).

Genes were selected for CRC analysis if they (1) showed significant and at least 25% reduction in RNA levels between PBS-treated samples and samples treated with 25 μM gapmer (by empirical Bayes-moderated t-statistics with a false discovery rate, FDR, below 5%), (2) showed general concentration-dependent reductions by gapmer treatment (by one-sided multiple regression analysis, treating the concentrations of gapmer at 0 (PBS), 0.2, 1, 5 or 25 μM as the ordinal independent variable, with p-value <0.005 from the resulting t-statistic for the linear trend), and (3) were not identified in 1 and 2 across multiple different gapmers with different nucleobase sequence.

Those genes identified in 1 and 2 as significantly affected across gapmers with different nucleobase sequences were judged to be involved in a general transcriptional program responding to gapmer-treatment in human neurons. Only exception being GAL3ST1, which all gapmers target.

Consequently, genes for which CRCs were fitted and EC50s estimated were exclusively seen for individual gapmers or for groups of gapmers with identical or near-identical nucleobase sequence. Such genes could therefore be hybridization-based off-targets or genes affected downstream of perturbing such off-targets.

For each gapmer, EC50 ratios between potential off-targets and the on-target (GAL3ST1) were calculated, grouped, and counted as shown in the table below. The weaker the effect on the off-target compared to the on-target, the larger the EC50 ratio.

The results are shown in Table 11. As seen in the table, some gapmers have strong (EC50 ratio <10) effects on only a few identified off-targets, whereas others have strong effects on hundreds of off-targets.

TABLE 11
Number of potential off-targets stratified by their EC50 relative
to GAL3ST1 as evaluated by microarray. Potential off-targets with
EC50 values no more than 10-fold weaker than the GAL3ST1 EC50 are
shown in the ‘<10’ column, those with EC50 values between
10- and 20-fold weaker in the ‘10 to 20’ column, and so forth.

| | EC50 ratio range | | | | | | |
|---|---|---|---|---|---|---|---|
| ASO | | 10 | 20 | 30 | 40 | 50 | |
| ID | <10 | to 20 | to 30 | to 40 | to 50 | to 100 | >100 |
| 19_23 | 3 | 1 | 0 | 0 | 0 | 0 | 0 |
| 1_129 | 7 | 3349 | 8 | 7 | 3 | 13 | 21 |
| 25_16 | 8 | 11 | 13 | 3 | 3 | 4 | 4 |
| 1_41 | 11 | 4 | 0 | 0 | 0 | 0 | 1 |
| 1_103 | 24 | 1 | 0 | 0 | 0 | 0 | 1 |
| 1_193 | 32 | 1 | 0 | 0 | 0 | 0 | 0 |
| 1_116 | 37 | 1 | 0 | 0 | 0 | 0 | 0 |
| 16_17 | 197 | 4 | 0 | 2 | 1 | 1 | 1 |

### Example 9—Single Dose In Vivo Efficacy Test in Heterozygous GAL3ST1 Humanized Mice of ASOs

Selected ASOs with good activity in vitro were evaluated in a humanized mice model for in vivo activity by intracerebroventricular dosing.

Experimental mice: In vivo acute tolerability of the antisense oligonucleotides were tested in mice, with 5-6 mice per ASO group. Mice at 8-10 weeks of age were housed in European IVC cages type IIL with TAPVEI aspen bedding (Tapvei Eatonis Oü, Estonia). The cages were enriched with nesting material, wooden sticks and hiding material. The light cycle was 12-hour dark and 12-hour light. Diet was pelleted complete diet (Altromin 1324, Brogaarden), and municipal drinking water. Diet and water were administered ad libitum.

All animals were inspected on daily basis for their general health condition. Any clinical signs or behavioral abnormalities was recorded. Humane endpoints and premature termination: Any animal showing clinical signs of moderate pain or moderate distress, or any degree of suffering was handled as appropriate, as discontinuation of the administration of test articles or euthanizing of the animal. Animals exhibiting clinical signs were humanely euthanized if they exceeded the limits of the study specific humane endpoints according to the European and Danish legislation on animals in experimentation.

Administration by ICV injection: Pre-dosing analgesia was given at least 30 min before dosing was initiated; all animals received preventive pain treatment with Meloxicam (2 mg/kg SC). The mice were anesthetized with isoflurane before dosing.

For injection, the G23 needle was mounted on a stand so it precisely penetrated 3.9 mm through the mouse's skull. The dose volume of 5 μL was injected over 30 seconds and the animal was then placed back in its cage.

Following dosing, the animals were observed closely. The assessment was performed according to a scoring scheme where a score of 0 to 4 was given for the following five parameters: activity level (increased or decreased activity, respectively), motor function, posture/presentation, and muscle tremors/cramps as outlined in table 12. Scoring was performed before dose, 30 min and 60 min after dosing.

TABLE 12
Scoring scheme for vivo acute tolerability

| Category | Score 0 | Score 1 | Score 2 | Score 3 | Score 4 |
|---|---|---|---|---|---|
| Hyperactivity, | Normal | Very slightly | Increase home | Moderately | Marked |
| stereotypies | | increased home | cage exploration | increased home | hyperactivity. |
| and home cage | | cage exploration | (incl. e.g. digging, | cage activity. | Marked |
| behavior | | or rearing | burying) | Detectable | stereotypies. |
| | | compared to | Increased | stereotypies (eg. | |
| | | controls | grooming | circling, repetitive | |
| | | | | behaviours) | |
| Decreased | Normal | Some | Drowsiness | Stupor (reduced | Coma (does |
| vigilance, | | reduction in | Slightly reduced | responsiveness, | not respond to |
| exploration, | | exploratory | response to touch | decreased corneal | stimulation e.g. |
| and | | activity | or handling. | reflex) | pinch), no |
| responsiveness | | Responds | | | corneal reflex |
| | | normally to | | | |
| | | stimulation. | | | |
| Motor | Normal | Mild changes | Reduced grip | Highly reduced | Severe ataxia |
| coordination | | to gait or grip | strength (falls in | grip strength (falls | (e.g. crawling; |
| and strength | | strength (falls | less than 5 sec) | in less than 2 sec) | fails to grip bar) |
| | | between 5-10 | Mild ataxia (e.g. | Ataxia (e.g. | No ability to |
| | | sec) | slow righting | staggering, falling; | right |
| | | No falls, | response; | impaired walking) | |
| | | normal righting | swaying) | | |
| | | response | | | |
| Posture, | Normal | Very slightly | Slightly | Moderately | Markedly |
| appearance | | abnormal | abnormal posture | abnormal posture | abnormal |
| and breathing | | posture (subtle). | (e.g. hunched, | (e.g. ventral | posture (e.g. |
| | | | extended, low | recumbency) | lateral |
| | | | posture, tail | Shallow breathing | recumbency) |
| | | | position eg. | | Facial |
| | | | Straub tail) | | paralysis (e.g. |
| | | | Piloerection or | | drooling, |
| | | | unkept coat | | protruding |
| | | | Ptosis | | tongue) |
| | | | | | Laboured |
| | | | | | breathing |
| Tremor, | Normal | Detectable | Hyper- | Few or partial | Repeated or |
| hyper- | | tremor | responsive to | seizures, rearing & | continuous |
| reactivity, | | | stimuli (e.g. | falling as a part of | seizure |
| convulsion | | | noise) | convulsing | (running, |
| | | | Marked tremor | | bouncing, tonic |
| | | | | | and/or clonic) |

Average neurobehavioral scoring at 1 hour post dose for 100 g dose is summarized in table 14. If the mice had to be euthanized before 1 hour post dosing, they got a score of 20.

To evaluate the efficacy of a single dose, the most preferred ASOs were dosed ICV to a mouse model containing the human version of GAL3ST1, a humanized mice model. In vivo efficacy in the CNS was evaluated 4 weeks after dosing by necropsy and harvest of brain tissues.

Mice were sacrificed by cervical dislocation and brain tissue was sampled from striatum, hippocampus, cortex right, cortex left and cerebellum and dissected tissue was snap frozen in Precellys tube (CK14, 2 mL, Cat.no. P000912-LYSK0-A) by submerging in liquid nitrogen. The tissue was stored at −80° C. until RNA purification. At the day of RNA extraction, the tissue sample (30-80 mg) was homogenized in 280 μL RLT buffer (Qiagen No.: 79216) using a Precellys 24 tissue homogenizer. The RNA was then extracted using TRIzol (ThermoFisher, Cat.no. 15596023) in combination with a Qiacube (Qiagen) for automated RNA extraction. After extraction RNA concentrations were normalized to 2 ng/pl.

The diluted RNA was used as input template for the qPCR, using TaqPath™ 1-Step RT-qPCR Master Mix, CG (ThermoFisher cat. No. A15299) and qPCR assays from Integrated DNA technologies (IDT) custom made GAL3ST1 assay as seen in Table 13 for human GAL3ST1 and Ppia-Mm.PT.39a.2gs as endogenous normalizer. The qPCR reaction was run in singleplex on a Quantstudio 6 qPCR machine (ThermoFisher).

TABLE 13
GAL3ST1 qPCR assay used for RNA
quantification of the GAL3ST1
humanized mice.

| | Forward | Reverse | Probe |
|---|---|---|---|
| Target | Primer | Primer | sequence |
| GAL3S | 5′-AAGACCC | 5′-GGTCCAG | 5′-FAM- |
| T1 | GGATCGCT | GCTGTTGT | AATGCCCAC |
| | ACTA-3′ | CATAG-3′ | TACCTCCGA |
| | (SEQ ID | (SEQ ID | AACCTG- |
| | NO: 643) | NO: 644) | IBFQ-3′ |
| | | | (SEQ ID |
| | | | NO: 645) |

The expression level of human GAL3ST1 was calculated using the ddCt approach and normalized to the average level of GAL3ST1 expression in samples from saline treated mice. A single 100 μg dose of ASO administered intracerebroventricular show significant reduction of human GAL3ST1 relative to mouse ppia as endogenous control in the brain regions striatum, hippocampus, cortex right, cortex left and cerebellum of the humanized mice as seen in Table 14, as well as having an acute tox score below 4.

TABLE 14
Levels of human GAL3ST1 mRNA (normalized to
mouse ppia) after ASO treatment (4-weeks) expressed
as % of saline treated mice (% UTC).

| | | Hippo- | Cortex | Cortex | Cere- | Acute tox |
|---|---|---|---|---|---|---|
| ASO | Striatum | campus | Right | Left | bellum | score |
| ID | % UTC | % UTC | % UTC | % UTC | % UTC | 1 hour |
| 1_41 | 79.9 | 48.6 | 48.2 | 58.2 | 36.5 | 1.8 |
| 1_103 | 49.7 | 31.6 | 37.2 | 42.3 | 23.8 | 1.5 |
| 1_116 | 64.7 | 43.2 | 47.7 | 60.5 | 24.1 | 1.8 |
| 1_129 | 48.7 | 34.7 | 36.5 | 40.6 | 31.2 | 1.0 |
| 1_193 | 38.1 | 26.0 | 24.9 | 32.1 | 22.5 | 1.2 |
| 16_17 | 38.8 | 41.6 | 37.6 | 39.9 | 15.7 | 1.8 |
| 19_23 | 60.4 | 45.5 | 49.9 | 55.4 | 23.5 | 3.5 |
| 25_16 | 74.9 | 44.9 | 51.0 | 56.5 | 34.8 | 8.0 |
| 48_7 | 35.8 | 89.7 | 32.9 | 79.5 | 71.3 | 9.0 |
| 96_12 | 69.7 | 69.0 | 54.3 | 63.0 | 45.9 | 1.0 |
| 103_9 | 8.2 | 59.5 | 16.6 | 26.9 | 10.1 | 2.0 |
| 108_6 | 42.4 | 61.8 | 47.0 | 65.8 | 48.1 | 6.3 |
| 108_12 | 52.4 | 43.6 | 42.7 | 52.6 | 19.7 | 4.3 |
| 108_17 | 44.3 | 33.9 | 48.1 | 52.6 | 20.1 | 4.8 |

### REFERENCES

Boucher A A, Miller W, Shanley R, Ziegler R, Lund T, Raymond G, Orchard PJ. Orphanet J Rare Dis. 2015 Aug. 7; 10:94. doi: 10.1186/s13023-015-0313-y. Long-term outcomes after allogeneic hematopoietic stem cell transplantation for metachromatic leukodystrophy: the largest single-institution cohort report.
Eckhardt M. The role and metabolism of sulfatide in the nervous system. Mol Neurobiol. 2008 April-June; 37(2-3):93-103. doi: 10.1007/s12035-008-8022-3. Epub 2008 May 9. PMID: 18465098.
Honke K, Hirahara Y, Dupree J, Suzuki K, Popko B, Fukushima K, Fukushima J, Nagasawa T, Yoshida N, Wada Y, Taniguchi N. Proc Natl Acad Sci USA. 2002 Apr. 2; 99(7):4227-32. doi: 10.1073/pnas.032068299. Epub 2002 Mar. 26. Paranodal junction formation and spermatogenesis require sulfoglycolipids.
Jones E, Mead S. Genetic risk factors for Creutzfeldt-Jakob disease. Neurobiol Dis. 2020 August; 142:104973. doi: 10.1016/j.nbd.2020.104973. Epub 2020 Jun. 18. PMID: 32565065.
Polten A, Fluharty A L, Fluharty C B, Kappler J, von Figura K, Gieselmann V. N Engl J Med. 1991 Jan. 3; 324(1):18-22. doi: 10.1056/NEJM199101033240104. Molecular basis of different forms of metachromatic leukodystrophy.
Ramakrishnan H, Hedayati K K, Lullmann-Rauch R, Wessig C, Fewou C N, Maier H, Goebel H, Gieselmann V, Eckhardt M. J Neurosci. 2007 Aug. 29; 27(35):9482-90. Increasing sulfatide synthesis in myelin-forming cells of arylsulfatase A-deficient mice causes demyelination and neurological symptoms reminiscent of human metachromatic leukodystrophy
Shaimardanova A, Chulpanova S V, Solovyeva V V, Mullagulova A I, Kitaeva K V, Allegrucci C, Rizvanov A A. Review Front Med (Lausanne). 2020 Oct. 20; 7:576221. doi: 10.3389/fmed.2020.576221. eCollection 2020.Metachromatic Leukodystrophy: Diagnosis, Modeling, and Treatment Approaches.
Van Rappard D F, Boelens J J, Wolf N I. Best Pract Res Clin Endocrinol Metab. 2015 March; 29(2):261-73. doi: 10.1016/j.beem.2014.10.001. Epub 2014 Oct 16. Metachromatic leukodystrophy: Disease spectrum and approaches for treatment.

1. An antisense oligonucleotide comprising a stretch of at least 10 nucleotides which is at least 90% complementary to a target sequence in the human GAL3ST1 gene (Galactosylceramide sulfotransferase), such as a target sequence selected from the target sequences shown in Table B1, B2 or B3.

2. The antisense oligonucleotide of claim 1, wherein the stretch of at least 10 nucleotides is at least 90% complementary to a target sequence selected from the group of target sequences consisting of SEQ ID NO: 522 to SEQ ID NO: 632.

3. (canceled)

4. (canceled)

5. The antisense oligonucleotide of claim 1, comprising a stretch of at least 12, or at least 14 nucleotides which is at least 90% complementary to said target sequence.

6. (canceled)

7. The antisense oligonucleotide of claim 1, wherein the antisense oligonucleotide has a length of 12 to 30 nucleotides, preferably, wherein the antisense oligonucleotide has a length of 14 to 22 nucleotides, such as a length of 16 to 20 nucleotides.

8. (canceled)

9. The antisense oligonucleotide of claim 1, wherein the antisense oligonucleotide comprises or consists of a nucleic acid sequence as shown in SEQ ID NO: 1 to SEQ ID NO: 521.

10. The antisense oligonucleotide of claim 1, wherein
the antisense oligonucleotide is capable of reducing the amount of GAL3ST1 mRNA in a host cell expressing said GAL3ST1 mRNA,
the antisense oligonucleotide is capable of reducing galactosylceramide sulfotransferase activity in a host cell, and/or
the antisense oligonucleotide is capable of reducing the amount of sulfatide in a host cell.

11. The antisense oligonucleotide claim 10, wherein the target cell is a human cell, such as a cell of the CNS.

12. The antisense oligonucleotide of claim 1, wherein the human GAL3ST1 gene comprise a sequence selected from SEQ ID NO: 633, 634 or 635, in particular wherein the wherein the human GAL3ST1 gene comprise a sequence as shown in SEQ ID NO: 633.

13. The antisense oligonucleotide of claim 1, wherein the antisense oligonucleotide is a chemically modified antisense oligonucleotide, for example wherein the chemically modified antisense oligonucleotide contains one or more modified nucleosides.

14. The antisense oligonucleotide of claim 13, wherein the one or more modified nucleosides, is a sugar modified nucleoside, such as a 2′ sugar modified nucleoside.

15. The antisense oligonucleotide of claim 11, wherein the chemically modified antisense oligonucleotide contains at least one modified nucleobase.

16. The antisense oligonucleotide of claim 13, wherein the at least one modified nucleobase is 5-methylcytosine.

17. The antisense oligonucleotide of claim 1, wherein the chemically modified antisense oligonucleotide comprises at least one modified nucleoside selected from the group consisting of: 2′-O-Methoxyethyl-RNA, 2′-O-Methyl-RNA, 2′-Fluoro-RNA.

18. The antisense oligonucleotide of claim 1, wherein the antisense oligonucleotide comprises at least one modified internucleoside linkage, such as at least one Phosphorothioate internucleoside linkage, at least one Phosphorodithioate internucleoside linkage, at least one Phophoroamidate internucleoside linkage, at least one methyl phosphonate internucleoside linkage, at least one phosphotriester internucleoside linkage, at least one boranophosphate internucleoside linkage or at least one phosphoryl guanidine internucleoside linkage.

19. The antisense oligonucleotide of claim 18, wherein at least 40%, such as at least 60%, in particular all internucleoside linkages are modified internucleoside linkages, such as Phosphorothioate internucleoside linkages.

20. The antisense oligonucleotide of claim 1, wherein the antisense oligonucleotide comprises one or more modified nucleosides being LNA (locked nucleic acid) nucleosides.

21. The antisense oligonucleotide of claim 20, wherein the LNA nucleoside(s) is (are) a beta-D-oxy LNA nucleosides.

22. The antisense oligonucleotide of claim 1, wherein the antisense oligonucleotide has a gapmer structure.

23. (canceled)

24. (canceled)

25. (canceled)

26. A method for treating Metachromatic leukodystrophy (MILD) comprising administering an antisense oligonucleotide of claim 1.

27. A method for identifying a candidate compound for the treatment of Metachromatic leukodystrophy (MLD), comprising
a) providing an antisense oligonucleotide as defined in claim 1,
b) contacting a host cell expressing GAL3ST1 mRNA with said antisense oligonucleotide,
c) determining the amount of GAL3ST1 mRNA in the said host cell, and
d) identifying a candidate compound based on the results of step c).

28. (canceled)

29. (canceled)
